(19) United States
(12) Patent Application Publication
(10) Pub. No.: US 2026/0217721 A1
(43) Pub. Date: Jul. 30, 2026

(54) COVALENT HETEROCYCLES AND USES THEREOF

(71) Applicant: Kumquat Biosciences Inc., San Diego, CA (US)

(72) Inventors: Pingda Ren, San Diego, CA (US); Xiaohui He, San Diego, CA (US); Rasmus Hansen, San Diego, CA (US)

(21) Appl. No.: 19/306,039

(22) Filed: Aug. 21, 2025

Related U.S. Application Data
(63) Continuation of application No. 19/009,057, filed on Jan. 3, 2025, now abandoned.
Application No. 19/009,057 is a continuation of application No. 18/659,528, filed on May 9, 2024, now abandoned.
Application No. 18/659,528 is a continuation of application No. 18/394,183, filed on Dec. 22, 2023, now abandoned.
(60) Provisional application No. 63/435,220, filed on Dec. 23, 2022.
(60) Provisional application No. 63/582,309, filed on Sep. 13, 2023.

(51) Int. Cl. C07D 487/04; A61K 31/4196; A61K 31/438; A61K 31/454; A61K 31/519; C07D 401/14; C07D 413/12; C07D 471/10; C07K 14/00

(52) U.S. Cl. CPC C07D 487/04; A61K 31/4196; A61K 31/438; A61K 31/454; A61K 31/519; C07D 401/14; C07D 413/12; C07D 471/10; C07K 14/001

(57) ABSTRACT

The present disclosure provides compounds and pharmaceutically acceptable salts thereof, and methods of using the same. The compounds and methods have a range of utilities as therapeutics, diagnostics, and research tools. In particular, the subject compositions and methods are useful for reducing signaling output of oncogenic proteins.

### CROSS-REFERENCE

This application is a continuation of U.S. application Ser. No. 19/009,057, filed Jan. 3, 2025, which is a continuation of U.S. application Ser. No. 18/659,528, filed May 9, 2024, which is a continuation of U.S. application Ser. No. 18/394,183, filed Dec. 22, 2023, which claims the benefit of U.S. Provisional Application No. 63/435,220, filed Dec. 23, 2022, and U.S. Provisional Application No. 63/582,309, filed Sep. 13, 2023, each incorporated herein by reference in its entirety.

### BACKGROUND

Covalent modification of amino acid residues has emerged as a significant advancement in both medicinal chemistry and chemical biology applications. Of particular interests are functional groups, so called warheads that form covalent bonds with the targeted amino acid, typically the side chains of the amino acids in a protein of interest. Warheads capable of labeling cysteine have been developed, including acrylamides, chlorofluoracetamides, epoxides, and nitriles. β-lactones represent another group of warheads that have been reported to label noncatalytic serines in a protein. However, these conventional warheads suffer from one or more drawbacks. Amongst them are inability to effectively label more than one type of amino acid residue, as well as the undesired propensity to develop drug resistance that limit the therapeutic duration and/or window. In particular, acrylamides are shown to be relatively ineffective in labeling serine residues in a protein. In addition, some of these warheads are overly reactive, causing high background and off-target labeling.

### SUMMARY

In view of the foregoing, there remains a considerable need for a new design of functional groups such as warheads to covalently modify a protein of interest. Of particular interests are warheads capable of effectively labeling an amino acid including a serine residue in a Target Protein. Such compositions and methods are particularly useful for modifying the activity and/or expression of a biological target. Such compositions and methods may also be applied for treating a variety of diseases including, but not limited to, viral infections, cardiovascular, metabolic, inflammatory, autoimmune, and neoplasia conditions that are mediated by a Target Protein or a pathogen. The present disclosure addresses these needs, and provides additional advantages applicable for diagnosis, prognosis, and/or treatment for a wide diversity of diseases.

In certain aspects, the present disclosure provides a compound of Formula (I) comprising a Target Binding Moiety (TBM), a linker (L), and a Serine-Targeting Warhead (STW),

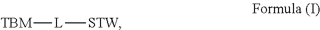

characterized in that:
(i) the compound is capable of forming a covalent bond with a serine residue of a Target Protein, wherein the Target Protein is not a Ras protein and the compound substantially lacks the ability to covalently bind with Ras;
(ii) TBM-L-hydrogen reversibly binds to the Target Protein with a K₁ of less than 3 μM when assessed by a biochemical assay;
(iii) PG-L-STW forms a covalent bond with suitably-protected Cys-OMe at a rate characterized by an intrinsic rate constant (K) that is less than that of PG-L-STW′ when tested at the same conditions and a pH between 7 to 8, wherein STW′ is unsubstituted acrylyl, and wherein if PG is bound to a nitrogen atom of L, then PG is Boc, else PG is hydrogen; and (iv) the compound modulates expression and/or activity of the Target Protein upon binding to the Target Protein.

In certain aspects, the present disclosure provides a compound of Formula (I) comprising a Target Binding Moiety (TBM), a linker (L), and a Serine-Targeting Warhead (STW),

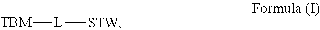

characterized in that:
(i) the compound is capable of forming a covalent bond with a serine residue of a Target Protein, wherein the Target Protein is not a Ras protein and the compound substantially lacks the ability to covalently bind with Ras, and wherein the serine residue resides in an enzymatic active site of the Target Protein and is utilized in an enzymatic reaction performed by the Target Protein;
(ii) TBM-L-hydrogen reversibly binds to the Target Protein with a K₁ of less than 500 μM when assessed by a biochemical assay;
(iii) PG-L-STW forms a covalent bond with suitably-protected Cys-OMe at a rate characterized by an intrinsic rate constant (K) that is less than that of PG-L-STW′ when tested at the same conditions and a pH between 7 to 8, wherein STW′ is unsubstituted acrylyl, and wherein if PG is bound to a nitrogen atom of L, then PG is Boc, else PG is hydrogen; and
(iv) the compound modulates expression and/or activity of the Target Protein upon binding to the Target Protein.

In some embodiments, for a compound of Formula (I), TBM-L-hydrogen reversibly binds to the Target Protein with a K₁ of less than 400 μM, such as less than 250 μM or less than 100 μM. In some embodiments, TBM-L-hydrogen reversibly binds to the Target Protein with a K₁ of less than 2 μM, such as less than 1 μM, when assessed by a biochemical assay. In some embodiments, TBM-L-hydrogen reversibly binds to the Target Protein with a K₁ of less than 500 nM when assessed by a biochemical assay. In some embodiments, the compound exhibits less than 1% covalent modification of a Ras protein after 24 hours of incubation with the Ras protein when assessed by a mass spectrometry assay. In some embodiments, the biochemical assay is an HTRF displacement assay. In some embodiments, the biochemical assay is a mass spectrometry assay. In some embodiments, the Target Protein, or a fragment thereof, is present at a concentration of about 1 μM in the biochemical assay. In some embodiments, K is less than 0.02 min⁻¹. In some embodiments, K is less than 0.005 min⁻¹. In some embodiments, K is less than 0.0005 min⁻¹, such as less than 0.0001 mint-. In some embodiments, L-STW comprises a ureylene functional group. In some embodiments, the ureylene functional group comprises a 5- to 12-membered heteroaryl group. In some embodiments, the ureylene functional group comprises a 5- or 6-membered heteroaryl group comprising one, two, or three ring nitrogen atoms.

In some embodiments, for a compound of Formula (I), STW is a compound of Formula (II):

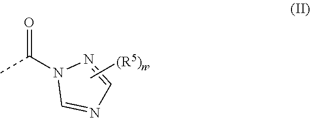

or a pharmaceutically acceptable salt thereof, wherein R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and n is 0, 1, or 2.

In some embodiments, for a compound of Formula (I), STW is a compound selected from

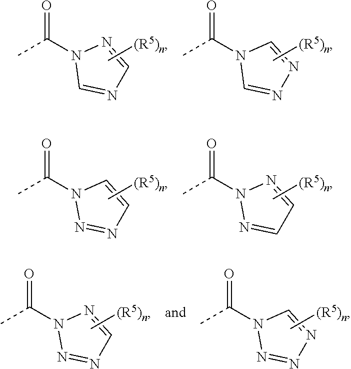

or a pharmaceutically acceptable salt thereof, wherein R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and n is 0, 1, or 2.

In some embodiments, for a compound of Formula (I), L is a compound of Formula (III):

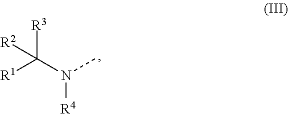

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R³ is selected from hydrogen and R⁶; and wherein:
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, for a compound of Formula (I), L-STW is a compound of Formula (IV):

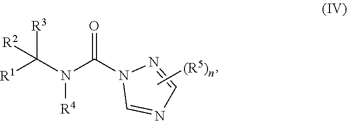

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R³ is selected from hydrogen and R⁶; and wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
n is 0, 1, or 2;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, the compound of Formula (IV) is a compound of Formula (IV-A):

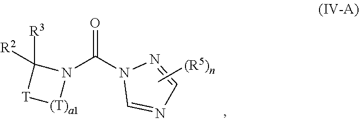

wherein a1 is 1, 2, 3, 4, or 5; T is independently selected at each occurrence from N(R⁹), C(R⁸)₂, C(O), O, S(O), and S(O)₂; R⁸ is independently selected at each occurrence from hydrogen and R⁶; and R⁹ is independently selected at each occurrence from hydrogen and R⁷.

In some embodiments, the compound of Formula (IV) or (IV-A) is a compound of Formula (IV-A1):

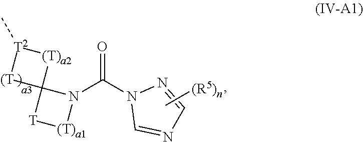

wherein a2 is 0, 1, 2, 3, 4, or 5; a3 is 0, 1, 2, 3, 4, or 5; wherein the sum of a2 and a3 is 2, 3, 4, 5, or 6; and T² is selected from N and C(R⁸).

In some embodiments, the compound of Formula (IV) is a compound of Formula (IV-B):

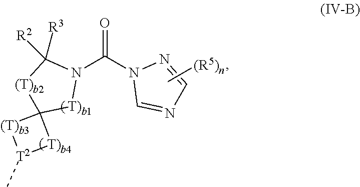

wherein b1 is 1, 2, 3, 4, or 5; b2 is 0, 1, 2, 3, 4, or 5; b3 is 1, 2, 3, 4, or 5; b4 is 1, 2, 3, 4, or 5; wherein the sum of b1, b2, b3, and b4 is less than 9; T is independently selected at each occurrence from N(R⁹), C(R⁸)₂, C(O), O, S(O), and S(O)₂; T² is selected from N and C(R⁸); R⁸ is independently selected at each occurrence from hydrogen and R⁶; and R⁹ is independently selected at each occurrence from hydrogen and R⁷.

In some embodiments, the compound of Formula (IV) is a compound of Formula (IV-C):

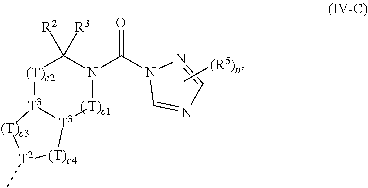

wherein c1 is 0, 1, 2, 3, or 4; c2 is 0, 1, 2, 3, or 4; c3 is 0, 1, 2, 3, or 4; c4 is 0, 1, 2, 3, or 4; wherein the sum of c3 and c4 is at least 1; and the sum of ci, c2, c3, and c4 is less than 8; T is independently selected at each occurrence from N(R⁹), C(R⁸)₂, C(O), O, S(O), and S(O)₂; T² is selected from N and C(R⁸); T³ is independently selected at each occurrence from N and C(R⁸); R⁸ is independently selected at each occurrence from hydrogen and R⁶; and R⁹ is independently selected at each occurrence from hydrogen and R⁷.

In some embodiments, the compound of Formula (IV) is a compound of Formula (IV-D):

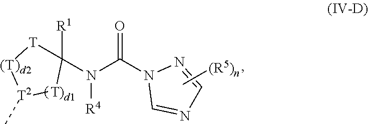

wherein d1 is 0, 1, 2, 3, or 4; d2 is 0, 1, 2, 3, or 4; wherein the sum of d1 and d2 is 1, 2, 3, 4, or 5; T is independently selected at each occurrence from N(R⁹), C(R⁸)₂, C(O), O, S(O), and S(O)₂; T² is selected from N and C(R⁸); R⁸ is independently selected at each occurrence from hydrogen and R⁶; and R⁹ is independently selected at each occurrence from hydrogen and R⁷.

In some embodiments, the compound of Formula (IV) is a compound of Formula (IV-E1) or (IV-E2):

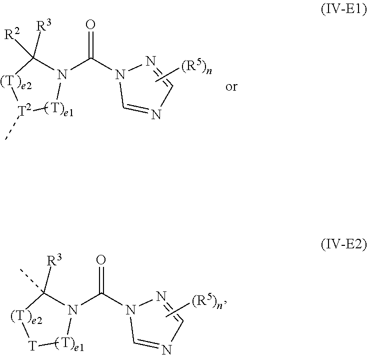

wherein e1 is 0, 1, 2, 3, or 4; e2 is 0, 1, 2, 3, or 4; wherein the sum of e1 and e2 is between 1 and 5; T is independently selected at each occurrence from N(R⁹), C(R⁸)₂, C(O), O, S(O), and S(O)₂; T² is selected from N and C(R⁸); R⁸ is independently selected at each occurrence from hydrogen and R⁶; and R⁹ is independently selected at each occurrence from hydrogen and R⁷.

In some embodiments, for a compound described herein, the TBM is selected from C₆₋₄₀organyl and C₆₋₄₀organoheteryl. In some embodiments, the TBM comprises 1 to 15 nitrogen atoms. In some embodiments, the TBM comprises 1 to 10 oxygen atoms. In some embodiments, the TBM comprises 1 to 10 halogen atoms independently selected from fluorine and chlorine.

In some embodiments, for a compound described herein, the TBM is selected from C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), —S(═O)(═NR²²)N(R²²)(R²³), and —OCH₂C(O)OR²²; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), —S(═O)(═NR²²)N(R²²)(R²³), and R³⁰;
R²¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more R³⁰; or two R²¹ are taken together with the carbon atom to which they are attached to form C₃₋₁₂ carbocycle or 3- to 12-membered heterocycle, each of which is optionally substituted with one or more R³⁰
R²² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more R³⁰
R²³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more R³⁰; or R²² and R²³ attached to the same nitrogen atom form 3- to 10 membered heterocycle optionally substituted with one or more R³⁰
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In some embodiments, for a compound described herein, the TBM is not:

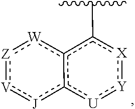

wherein:
W is N, C(R¹⁷), N(R^{17b}), C(R¹⁷)₂, C(O), S(O), or S(O)₂;
Z is N, C(R¹⁷), N(R^{17b}), C(R¹⁷)₂, C(O), S(O), or S(O)₂; wherein W and Z are not both selected from C(O), S(O), and S(O)₂;
V and J are each independently selected from N, C(R¹⁹), C(R¹⁷), N(R¹⁹), N(R^{17b}), C(R¹⁹)(R¹⁷), and C(R¹⁷)₂; wherein exactly one of V and J is C(R¹⁹), N(R¹⁹), or C(R¹⁹)(R¹⁷)
U is N, C(R¹⁷), N(R^{17b}), C(R¹⁷)₂, S(O), S(O)₂, or C(O);
Y is N, C(R¹⁸), N(R^{17b}), C(R¹⁸)(R¹⁷), S(O), S(O)₂, or C(O);
X is N, C(R¹⁷), N(R^{17b}), or C(R¹⁷)₂;
R¹⁷ is independently selected at each occurrence from hydrogen, halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R⁴⁰;
R^{17b} is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OR¹², —SR¹², —C(O)OR¹², —OC(O)N(R¹²)(R¹³), —C(O)R¹², —S(O)R¹², —OC(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R⁴⁰;
R¹⁸ is selected from halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R⁴⁰;
R¹⁹ is selected from C₆₋₁₀ aryl and 5- to 10-membered heteroaryl, each of which is optionally substituted with one, two, three, four, or five R⁴⁰;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R⁴⁰;
R¹³ is independently selected at each occurrence from hydrogen, C₁₋₆ alkyl, and C₁₋₆ haloalkyl; or R¹² and R¹³ attached to the same nitrogen atom form 3- to 10-membered heterocycle optionally substituted with one, two, or three R⁴⁰;
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R⁴⁰;
R⁴⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR⁴², —SR⁴², —N(R⁴²)(R⁴³), ═NR⁴², ═C(R⁴¹)₂, —C(O)OR⁴², —OC(O)N(R⁴²)(R⁴³), —N(R⁴²)C(O)N(R⁴²)(R⁴³), —N(R⁴²)C(O)OR⁴², —N(R⁴²)S(O)₂R⁴², —C(O)R⁴², —S(O)R⁴², —OC(O)R⁴², —C(O)N(R⁴²)(R⁴³), —C(O)C(O)N(R⁴²)(R⁴³), —N(R⁴²)C(O)R⁴², —S(O)₂R⁴², —S(O)(NR⁴²)R⁴², —S(O)₂N(R⁴²)(R⁴³), —S(═O)(═NR⁴²)N(R⁴²)(R⁴³), and —OCH₂C(O)OR⁴²; wherein two R⁴⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR⁴², —SR⁴², —N(R⁴²)(R⁴³), ═NR⁴², ═C(R⁴¹)₂, —C(O)OR⁴², —OC(O)N(R⁴²)(R⁴³), —N(R⁴²)C(O)N(R⁴²)(R⁴³), —N(R⁴²)C(O)OR⁴², —N(R⁴²)S(O)₂R⁴², —C(O)R⁴², —S(O)R⁴², —OC(O)R⁴², —C(O)N(R⁴²)(R⁴³), —C(O)C(O)N(R⁴²)(R⁴³), —N(R⁴²)C(O)R⁴², —S(O)₂R⁴², —S(O)(NR⁴²)R⁴², —S(O)₂N(R⁴²)(R⁴³), and —S(═O)(═NR⁴²)N(R⁴²)(R⁴³);
R⁴¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R⁴¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R⁴² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle);
R⁴³ is independently selected at each occurrence from hydrogen and C₁₋₆ alkyl; or R⁴² and R⁴³ attached to the same nitrogen atom form 3- to 10 membered heterocycle; and
[symbol] indicates a single or double bond such that all valences are satisfied.

In certain aspects, the present disclosure provides a covalently modified serine residue in a non-Ras polypeptide, wherein the modified serine residue is a compound of Formula (V):

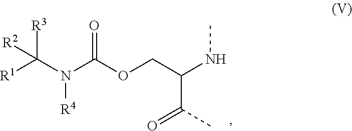

wherein:
the dashed bonds represent peptide bonds between the serine residue and adjacent amino acid residues of the polypeptide, respectively, or, if the serine residue is located at the N-terminus or C-terminus of the polypeptide, the dashed bond to the nitrogen atom represents a bond to a hydrogen atom or the dashed bond to the carbon atom represents a bond to a hydroxy group, respectively;
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶;
(b) R¹ and R⁴, together with the atoms to which they are attached, form a 7- to 12-membered spirocyclic heterocycloalkyl ring which is substituted with TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with TBM and optionally further substituted with one, two, or three R⁶; R² is selected from R⁶ and a bond to TBM; and R³ is selected from hydrogen and R⁶; and wherein:
TBM is selected from C₆₋₄organyl and C₆₋₄organoheteryl;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, for a modified serine residue of Formula (V), the polypeptide exhibits reduced signaling output when relative to the signaling output of the polypeptide prior to modification of the serine residue. In some embodiments, the reduced signaling output is evidenced by a reduction of cell growth or division of a tumor cell expressing the polypeptide. In some embodiments, the modified serine residue is formed by contacting an unmodified serine residue in the polypeptide with a precursor compound, wherein the precursor compound comprises a staying group and a leaving group, and wherein said contacting results in release of the leaving group and formation of said modified serine residue. In some embodiments, the precursor compound is a compound described herein, such as a compound of Formula (I), (II), (III), or (IV). In some embodiments, the leaving group is selected from

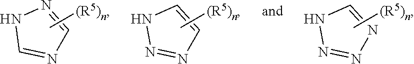

or a salt or tautomer thereof, wherein R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and n is 0, 1, or 2.

In certain aspects, the present disclosure provides a method of modifying a non-Ras Target Protein, comprising contacting the Target Protein with an effective amount of a compound described herein, such as a compound of Formula (I), (II), (III), or (IV), or a pharmaceutically acceptable salt or solvate thereof. In certain aspects, the present disclosure provides a pharmaceutical composition comprising a compound described herein, such as a compound of Formula (I), (II), (III), or (IV), or a pharmaceutically acceptable salt or solvate thereof, and a pharmaceutically acceptable excipient.

A Target Protein described herein may be selected from an oxidoreductase, a transferase, a hydrolase, a lyase, an isomerase, a ligase, and a translocase. In some embodiments, a serine residue targeted by a subject compound or a modified serine residue of Formula (V) is an inactive serine. In some embodiments, a Target Protein of the subject disclosure is a serine hydrolase.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Unless defined otherwise, all technical and scientific terms used herein have the same meaning as is commonly understood by one of skill in the art to which this disclosure belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Chemical structures are named herein according to IUPAC conventions as implemented in ChemDraw® software (Perkin Elmer, Inc., Cambridge, MA). The section headings used herein are for organizational purposes only and are not to be construed as limiting the subject matter described. As used in the specification and claims, the singular forms “a”, “an” and “the” include plural references unless the context clearly dictates otherwise. Furthermore, use of the term “including” as well as other forms, such as “include”, “includes”, and “included”, is not limiting. The section headings used herein are for organizational purposes only and are not to be construed as limiting the subject matter described.

The term “C_{x-y}” or “Cₓ-C_{y}” when used in conjunction with a chemical moiety, such as alkyl, alkenyl, or alkynyl, is meant to include groups that contain from x to y carbons in the chain. For example, the term “C_{x-y} alkyl” refers to substituted or unsubstituted saturated hydrocarbon groups, including straight-chain alkyl and branched-chain alkyl groups, that contain from x to y carbons in the chain.

“Alkyl” refers to substituted or unsubstituted saturated hydrocarbon groups, including linear and branched alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including linear and branched alkenyl groups, containing at least one double bond. An alkenyl group may contain from two to twelve carbon atoms (e.g., C2-12 alkenyl), such as two to eight carbon atoms (C₂₋₈ alkenyl) or two to six carbon atoms (C₂₋₆ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including linear and branched alkynyl groups, containing at least one triple bond. An alkynyl group may contain from two to twelve carbon atoms (e.g., C₂₋₁₂ alkynyl), such as two to eight carbon atoms (C₂₋₈ alkynyl) or two to six carbon atoms (C₂₋₆ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including linear alkylene and branched alkylene groups, that contain from one to twelve carbon atoms (e.g., C₂₋₁₂ alkylene), such as one to eight carbon atoms (C₁₋₈ alkylene) or one to six carbon atoms (C₁₋₆ alkylene). Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons of the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkyl group has a chain length of 3 to 8 atoms. Connection to the rest of the molecule may be through either a heteroatom or a carbon in the heteroalkyl, heteroalkenyl, or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkylene group has a chain length of 3 to 8 atoms. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkylene, heteroalkenylene or heteroalkynylene chain. Unless stated otherwise specifically in the specification, a heteroalkylene, heteroalkenylene, or heteroalkynylene group is optionally substituted by one or more substituents such as those substituents described herein.

“Carbocycle” refers to a saturated, unsaturated or aromatic ring in which each atom of the ring is a carbon atom. Carbocycle may include C₃₋₁₀ monocyclic rings, C₆₋₁₂ bicyclic rings, C₇₋₁₈ polycyclic rings, C₅₋₁₂ spirocyclic rings, and C₆₋₁₂ bridged rings. Each ring of a bicyclic or polycyclic carbocycle may be selected from saturated, unsaturated, and aromatic rings. In some embodiments, the carbocycle is a C₆₋₁₂ aryl group, such as C₆₋₁₀ aryl. In some embodiments, the carbocycle is a C₃₋₁₂ cycloalkyl group. In some embodiments, the carbocycle is a C₅₋₁₂ cycloalkenyl group. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocycle. A carbocycle may comprise a fused ring, a bridged ring, a spirocyclic ring, a saturated ring, an unsaturated ring, an aromatic ring, or any combination thereof. Exemplary carbocycles include cyclopentyl, cyclohexyl, cyclohexenyl, adamantly, phenyl, indanyl, and naphthyl. Unless state otherwise specifically in the specification, a carbocycle is optionally substituted by one or more substituents such as those substituents described herein.

“Heterocycle” refers to a saturated, unsaturated or aromatic ring comprising one or more heteroatoms, for example 1, 2 or 3 heteroatoms selected from O, S and N. Heterocycle may include 3- to 10-membered monocyclic rings, 6- to 12-membered bicyclic rings, 7- to 18-membered polycyclic rings, 5- to 12-membered spirocyclic rings, and 6- to 12-membered bridged rings. Each ring of a bicyclic or polycyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a 5- to 10-membered heteroaryl group, such as 5- or 6-membered heteroaryl. In some embodiments, the heterocycle is a 3- to 12-membered heterocycloalkyl group. A heterocycle may comprise a fused ring, a bridged ring, a spirocyclic ring, a saturated ring, an unsaturated ring, an aromatic ring, or any combination thereof. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Unless stated otherwise specifically in the specification, a heterocycle is optionally substituted by one or more substituents such as those substituents described herein.

“Heteroaryl” refers to an aromatic ring that comprises at least one heteroatom, for example 1, 2 or 3 heteroatoms selected from O, S and N. Heteroaryl may include 5- to 10-membered monocyclic rings, 6- to 12-membered bicyclic rings, 7- to 18-membered polycyclic rings, 5- to 12-membered spirocyclic rings, and 6- to 12-membered bridged rings. As used herein, the heteroaryl ring may be selected from monocyclic or bicyclic-including fused, spirocyclic and bridged ring systems—wherein at least one of the rings in the ring system is aromatic. The heteroatom(s) in the heteroaryl may optionally be oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryl groups include, but are not limited to, azepinyl, benzimidazolyl, benzisothiazolyl, benzisoxazolyl, benzofuranyl, benzothiazolyl, benzothiophenyl, benzoxazolyl, furanyl, imidazolyl, indazolyl, indolyl, isoquinolinyl, isothiazolyl, isoxazolyl, oxadiazolyl, oxazolyl, purinyl, pyrazinyl, pyrazolidinyl, pyrazolyl, pyridazinyl, pyridazolyl, pyridyl, pyrimidinyl, pyrrolyl, quinazolinyl, quinolinyl, quinoxalinyl, tetrahydroquinolinyl, thiadiazolyl, thiazolyl, and thienyl groups. Unless stated otherwise specifically in the specification, a heteroaryl is optionally substituted by one or more substituents such as those substituents described herein.

Unless stated otherwise, hydrogen atoms are implied in structures depicted herein as necessary to satisfy the valence requirement.

A waved line “ [symbol] ” drawn across a bond or a dashed bond “ [symbol] ” are used interchangeably herein to denote where a bond disconnection or attachment occurs. For example, in the structure

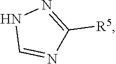

if R⁵ is isopropyl as in

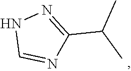

then R⁵ may be depicted as

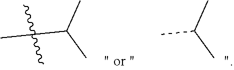

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, heteroatoms such as nitrogen may have any permissible substituents of organic compounds described herein which satisfy the valences of the heteroatoms.

A compound disclosed herein, such as a compound of Formula (I′) or (I), is optionally substituted by one or more, such as 1, 2 or 3 substituents selected from:
halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two substituents attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R³¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R³² is independently selected at each occurrence from hydrogen, C₁₋₆ alkyl, C₁₋₆ haloalkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein —C₀₋₆ alkyl-(C₃₋₁₂ carbocycle) and —C₀₋₆ alkyl-(3- to 12-membered heterocycle) are optionally substituted with one, two, or three groups independently selected from halogen and C₁₋₆ alkyl; and
R³³ is independently selected at each occurrence from hydrogen and C₁₋₆ alkyl; or R³² and R³³ attached to the same nitrogen atom form 3- to 10 membered heterocycle.

In some embodiments, a compound disclosed herein, such as a compound of Formula (I′) or (I), is optionally substituted by one or more, such as 1, 2 or 3 substituents selected from:
halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —OC(O)R³², —C(O)N(R³²)(R³³), —C(O)C(O)N(R³²)(R³³), —N(R³²)C(O)R³², —S(O)₂R³², —S(O)(NR³²)R³², and —S(O)₂N(R³²)(R³³), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², and ═C(R³¹)₂;
R³¹ is independently selected at each occurrence from hydrogen, halogen, C₁₋₆ alkyl, and C₁₋₆ haloalkyl;
R³² is independently selected at each occurrence from hydrogen, C₁₋₆ alkyl, C₁₋₆ haloalkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein —C₀₋₆ alkyl-(C₃₋₁₂ carbocycle) and —C₀₋₆ alkyl-(3- to 12-membered heterocycle) are optionally substituted with one, two, or three groups independently selected from halogen and C₁₋₆ alkyl;
R³³ is independently selected at each occurrence from hydrogen and C₁₋₆ alkyl; or R³² and R³³ attached to the same nitrogen atom form 3- to 10 membered heterocycle.

In some embodiments, a compound disclosed herein, such as a compound of Formula (I′) or (I), is optionally substituted by one or more, such as 1, 2 or 3 substituents selected from halogen, oxo, ═NH, —CN, —NO₂, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OH, —OCH₃, —OCH₂CH₃, —NH₂, —NHCH₃, and —NHCH₂CH₃, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, —NO₂, —CH₃, —CH₂CH₃, —CH(CH₃)₂, —C(CH₃)₃, —OH, —OCH₃, —OCH₂CH₃, —NH₂, —NHCH₃, and —NHCH₂CH₃.

It will be understood by those skilled in the art that substituents can themselves be substituted, if appropriate. Unless specifically stated as “unsubstituted”, references to chemical moieties herein are understood to include substituted variants. For example, reference to a “heteroaryl” group or moiety implicitly includes both substituted and unsubstituted variants.

Where bivalent substituent groups are specified herein by their conventional chemical formulae, written from left to right, they are intended to encompass the isomer that would result from writing the structure from right to left, e.g., —CH₂O— is also intended to encompass —OCH₂—.

“Optional” or “optionally” means that the subsequently described event or circumstances may or may not occur, and that the description includes instances where the event or circumstance occurs and instances in which it does not. For example, an “optionally substituted” group may be either unsubstituted or substituted.

Compounds of the present disclosure also include crystalline and amorphous forms of those compounds, pharmaceutically acceptable salts, and active metabolites having the same type of activity, including, for example, polymorphs, pseudopolymorphs, solvates, hydrates, unsolvated polymorphs (including anhydrates), conformational polymorphs, amorphous forms of the compounds, and mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Examples of isotopes that may be incorporated into compounds of the present disclosure include, but are not limited to, ²H, ³H, ¹³C, ¹⁴C, ¹⁵N, ¹⁸O, ¹⁷O, ³⁵S, ³⁶C1, and ¹⁸F. Of particular interest are compounds of Formula (II) enriched in tritium or carbon-14, which can be used, for example, in tissue distribution studies; compounds of the disclosure enriched in deuterium-especially at a site of metabolism-resulting, for example, in compounds having greater metabolic stability; and compounds of Formula (II) enriched in a positron emitting isotope, such as ¹¹C, ¹⁸F, ¹⁵O and ¹³N, which can be used, for example, in Positron Emission Topography (PET) studies. Isotopically-enriched compounds may be prepared by conventional techniques well known to those skilled in the art.

As used herein, the phrase “of the formula”, “having the formula” or “having the structure” is not intended to be limiting and is used in the same way that the term “comprising” is commonly used. For example, if one structure is depicted, it is understood that all stereoisomer and tautomer forms are encompassed, unless stated otherwise.

Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. In some embodiments, in order to optimize the therapeutic activity of the compounds of the disclosure, e.g., to treat cancer, it may be desirable that the carbon atoms have a particular configuration (e.g., (R,R), (S,S), (S,R), or (R,S)) or are enriched in a stereoisomeric form having such configuration. The compounds of the disclosure may be provided as racemic mixtures. Accordingly, the disclosure relates to racemic mixtures, pure stereoisomers (e.g., enantiomers and diastereomers), stereoisomer-enriched mixtures, and the like, unless otherwise indicated. When a chemical structure is depicted herein without any stereochemistry, it is understood that all possible stereoisomers are encompassed by such structure. Similarly, when a particular stereoisomer is shown or named herein, it will be understood by those skilled in the art that minor amounts of other stereoisomers may be present in the compositions of the disclosure unless otherwise indicated, provided that the utility of the composition as a whole is not eliminated by the presence of such other isomers. Individual stereoisomers may be obtained by numerous methods that are known in the art, including preparation using chiral synthons or chiral reagents, resolution using chiral chromatography using a suitable chiral stationary phase or support, or by chemically converting them into diastereomers, separating the diastereoisomers by conventional means such as chromatography or recrystallization, then regenerating the original stereoisomer.

Additionally, where applicable, all cis-trans or E/Z isomers (geometric isomers), tautomeric forms and topoisomeric forms of the compounds described herein are included with the scope of the disclosure unless otherwise specified.

The term “pharmaceutically acceptable” refers to a material that is not biologically or otherwise unacceptable when used in the subject compositions and methods. For example, the term “pharmaceutically acceptable carrier” refers to a material-such as an adjuvant, excipient, glidant, sweetening agent, diluent, preservative, dye, colorant, flavor enhancer, surfactant, wetting agent, dispersing agent, suspending agent, stabilizer, isotonic agent, solvent or emulsifier—that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The terms “salt” and “pharmaceutically acceptable salt” refer to a salt prepared from a base or an acid. Pharmaceutically acceptable salts are suitable for administration to a patient, such as a mammal (for example, salts having acceptable mammalian safety for a given dosage regime). Salts can be formed from inorganic bases, organic bases, inorganic acids and organic acids. In addition, when a compound contains both a basic moiety, such as an amine, pyridine or imidazole, and an acidic moiety, such as a carboxylic acid or tetrazole, zwitterions may be formed and are included within the term “salt” as used herein. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc., and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfltes, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, “treating” or “treatment” refers to an approach for obtaining beneficial or desired results with respect to a disease, disorder, or medical condition (such as cancer) in a subject, including but not limited to the following: (a) preventing the disease or medical condition from occurring, e.g., preventing the reoccurrence of the disease or medical condition or prophylactic treatment of a subject that is pre-disposed to the disease or medical condition; (b) ameliorating the disease or medical condition, e.g., eliminating or causing regression of the disease or medical condition in a subject; (c) suppressing the disease or medical condition, e.g., slowing or arresting the development of the disease or medical condition in a subject; or (d) alleviating symptoms of the disease or medical condition in a subject. For example, “treating cardiovascular disease” would include preventing cardiovascular disease from occurring, ameliorating cardiovascular disease, suppressing cardiovascular disease, and alleviating the symptoms of cardiovascular disease. Also, a therapeutic benefit is achieved with the eradication or amelioration of one or more of the physiological symptoms associated with the underlying disorder such that an improvement is observed in the subject, notwithstanding that the subject may still be afflicted with the underlying disorder.

A “therapeutic effect”, as that term is used herein, encompasses a therapeutic benefit and/or prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “antagonist” and “inhibitor” are used interchangeably, and they refer to a compound having the ability to inhibit a biological function (e.g., activity, expression, binding, protein-protein interaction) of a target protein (e.g., BTK or MGLL). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition.

The term “selective inhibition” or “selectively inhibit” refers to the ability of a biologically active agent to preferentially reduce the target signaling activity as compared to off-target signaling activity, via direct or indirect interaction with the target.

The terms “subject” and “patient” refer to an animal, such as a mammal, for example a human. The methods described herein can be useful in both human therapeutics and veterinary applications. In some embodiments, the subject is a mammal, such as a human. “Mammal” includes humans and both domestic animals such as laboratory animals and household pets (e.g., cats, dogs, swine, cattle, sheep, goats, horses, rabbits), and non-domestic animals such as wildlife and the like.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

“Prodrug” is meant to indicate a compound that may be converted under physiological conditions or by solvolysis to a biologically active compound described herein (e.g., a compound of Formula (II)). Thus, the term “prodrug” refers to a precursor of a biologically active compound that is pharmaceutically acceptable. In some aspects, a prodrug is inactive when administered to a subject but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press each of which is incorporated in full by reference herein). The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound, and the like.

The term “in vivo” refers to an event that takes place in a subject's body. The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body. The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound disclosed herein to a mammal for a period of time sufficient to yield a metabolic product thereof. Such products are typically identified by administering a radiolabeled compound of the disclosure in a detectable dose to an animal, such as rat, mouse, guinea pig, monkey, or to a human, allowing sufficient time for metabolism to occur, and isolating its conversion products from the urine, blood or other biological samples.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (also used interchangeably herein as K-Ras, K-ras, or Kras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof.

The term “leaving group” is used herein in accordance with its well understood meaning in Chemistry and refers to an atom or group of atoms which breaks away from the rest of the molecule, taking with it the electron pair which used to be the bond between the leaving group and the rest of the molecule.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In embodiments, the degradation enhancer is capable of binding to an E3 ubiquitin ligase protein or a protein complex comprising an E3 ubiquitin ligase protein. In embodiments, the degradation enhancer is capable of binding to an E2 ubiquitin-conjugating enzyme. In embodiments, the degradation enhancer is capable of binding to a protein complex comprising an E2 ubiquitin-conjugating enzyme and an E3 ubiquitin ligase protein.

The term “Target Protein” refers to a protein, polypeptide, or peptide that is not Ras.

In certain aspects, the present disclosure provides a compound of Formula (I′) comprising a Target Binding Moiety (TBM), a linker (L), and an Amino Acid-Targeting Warhead (STW),

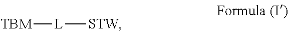

characterized in that:
(i) the compound is capable of forming a covalent bond with an amino acid residue of a Target Protein, wherein the Target Protein is not a Ras protein and the compound substantially lacks the ability to covalently bind with Ras;
(ii) TBM-L-hydrogen reversibly binds to the Target Protein with a K₁ of less than 3 μM when assessed by a biochemical assay;
(iii) PG-L-STW forms a covalent bond with suitably-protected Cys-OMe at a rate characterized by an intrinsic rate constant (K) that is less than that of PG-L-STW′ when tested at the same conditions and a pH between 7 to 8, wherein STW′ is unsubstituted acrylyl, and wherein if PG is bound to a nitrogen atom of L, then PG is Boc, else PG is hydrogen; and
(iv) the compound modulates expression and/or activity of the Target Protein upon binding to the Target Protein.

In certain aspects, the present disclosure provides a compound of Formula (I′) comprising a Target Binding linker (L), and an Amino Acid-Targeting Warhead (STW),

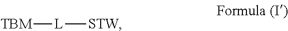

characterized in that:
(i) the compound is capable of forming a covalent bond with an amino acid residue of a Target Protein, wherein the Target Protein is not a Ras protein and the compound substantially lacks the ability to covalently bind with Ras, and wherein the amino acid residue resides in an enzymatic active site of the Target Protein and is utilized in an enzymatic reaction performed by the Target Protein;
(ii) TBM-L-hydrogen reversibly binds to the Target Protein with a K₁ of less than 500 μM when assessed by a biochemical assay;
(iii) PG-L-STW forms a covalent bond with suitably-protected Cys-OMe at a rate characterized by an intrinsic rate constant (K) that is less than that of PG-L-STW′ when tested at the same conditions and a pH between 7 to 8, wherein STW′ is unsubstituted acrylyl, and wherein if PG is bound to a nitrogen atom of L, then PG is Boc, else PG is hydrogen; and (iv) the compound modulates expression and/or activity of the Target Protein upon binding to the Target Protein.

In some embodiments, for a compound of Formula (I), the amino acid is selected from serine, tyrosine, cysteine, lysine, and histidine. In some embodiments, the amino acid is serine. In some embodiments, the amino acid is tyrosine. In some embodiments, the amino acid is cysteine. In some embodiments, the amino acid is lysine. In some embodiments, the amino acid is histidine.

In certain aspects, the present disclosure provides a compound of Formula (I) comprising a Target Binding Moiety (TBM), a linker (L), and a Serine-Targeting Warhead (STW),

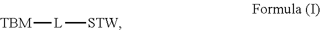

characterized in that:
(i) the compound is capable of forming a covalent bond with a serine residue of a Target Protein, wherein the Target Protein is not a Ras protein and the compound substantially lacks the ability to covalently bind with Ras;
(ii) TBM-L-hydrogen reversibly binds to the Target Protein with a K₁ of less than 3 μM when assessed by a biochemical assay;
(iii) PG-L-STW forms a covalent bond with suitably-protected Cys-OMe at a rate characterized by an intrinsic rate constant (K) that is less than that of PG-L-STW′ when tested at the same conditions and a pH between 7 to 8, wherein STW′ is unsubstituted acrylyl, and wherein if PG is bound to a nitrogen atom of L, then PG is Boc, else PG is hydrogen; and
(iv) the compound modulates expression and/or activity of the Target Protein upon binding to the Target Protein.

In certain aspects, the present disclosure provides a compound of Formula (I) comprising a Target Binding Moiety (TBM), a linker (L), and a Serine-Targeting Warhead (STW),

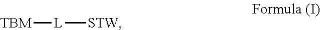

characterized in that:
(i) the compound is capable of forming a covalent bond with a serine residue of a Target Protein, wherein the Target Protein is not a Ras protein and the compound substantially lacks the ability to covalently bind with Ras, and wherein the serine residue resides in an enzymatic active site of the Target Protein and is utilized in an enzymatic reaction performed by the Target Protein;
(ii) TBM-L-hydrogen reversibly binds to the Target Protein with a K₁ of less than 500 μM when assessed by a biochemical assay;
(iii) PG-L-STW forms a covalent bond with suitably-protected Cys-OMe at a rate characterized by an intrinsic rate constant (K) that is less than that of PG-L-STW′ when tested at the same conditions and a pH between 7 to 8, wherein STW′ is unsubstituted acrylyl, and wherein if PG is bound to a nitrogen atom of L, then PG is Boc, else PG is hydrogen; and
(iv) the compound modulates expression and/or activity of the Target Protein upon binding to the Target Protein.

In some embodiments, for a compound of Formula (I′) or (I), TBM-L-hydrogen reversibly binds to the Target Protein with a K₁ of less than 500 μM, such as less than 450 μM, less than 400 μM, less than 350 μM, less than 300 μM, less than 250 μM, less than 200 μM, less than 150 μM, less than 100 μM, less than 90 μM, less than 80 μM, less than 70 μM, less than 60 μM, less than 50 μM, less than 40 μM, less than 30 μM, less than 20 μM, less than 10 μM, less than 5 μM, less than 4 μM, less than 3 μM, less than 2 μM, or less than 1 μM when assessed by a biochemical assay. In some embodiments, TBM-L-hydrogen reversibly binds to the Target Protein with a K₁ of less than 25 μM, such as less than 10 μM. In some embodiments, the amino acid residue (e.g., serine) resides in an enzymatic active site of the Target Protein and is utilized in an enzymatic reaction performed by the Target Protein. In some embodiments, the Target Protein is a serine hydrolase.

In some embodiments, for a compound of Formula (I′) or (I), TBM-L-hydrogen reversibly binds to the Target Protein with a K₁ of less than 3 μM, such as less than 2.5 μM, less than 2 μM, less than 1.5 μM, less than 1 μM, less than 900 nM, less than 800 nM, less than 700 nM, less than 600 nM, less than 500 nM, less than 400 nM, less than 300 nM, less than 200 nM, less than 150 nM, or less than 100 nM when assessed by a biochemical assay. In some embodiments, TBM-L-hydrogen reversibly binds to the Target Protein with a K₁ of less than 500 nM when assessed by a biochemical assay. In some embodiments, the amino acid residue (e.g., serine) is an inactive amino acid (e.g., an inactive serine). In some embodiments, the amino acid residue (e.g., serine) is not utilized in an enzymatic reaction performed by the Target Protein. In some embodiments, the Target Protein is not a serine hydrolase.

In some embodiments, for a compound of Formula (I′) or (I), TBM-L-hydrogen exhibits biochemical activity against the Target Protein as evidenced by an IC₅₀ of less than 2 μM, such as less than 1.5 μM, less than 1 μM, less than 900 nM, less than 800 nM, less than 700 nM, less than 600 nM, less than 500 nM, less than 400 nM, less than 300 nM, less than 200 nM, less than 150 nM, or less than 100 nM when assessed by a biochemical assay, such as a biochemical assay described herein.

In some embodiments, for a compound of Formula (I′) or (I), the biochemical assay is an HTRF displacement assay. In some embodiments, the biochemical assay is a mass spectrometry assay. In some embodiments, the biochemical assay is an assay described in an Example of the present disclosure. In some embodiments, the Target Protein, or a fragment thereof, is present at a concentration of about 1 μM in the biochemical assay.

In some embodiments, for a compound of Formula (I′) or (I), the compound exhibits less than 1% covalent modification of a Ras protein after 24 hours of incubation with the Ras protein when assessed by a mass spectrometry assay. In some embodiments, the compound exhibits less than 1% covalent modification, such as less than 0.5%, less than 0.4%, less than 0.3%, less than 0.2%, less than 0.1%, less than 0.05%, or less than 0.01% of a Ras protein after 24 hours of incubation with the Ras protein when assessed by a mass spectrometry assay. In some embodiments, the compound exhibits no detectable covalent modification of a Ras protein after 24 hours of incubation with the Ras protein when assessed by a mass spectrometry assay. In some embodiments, the mass spectrometry assay is an assay described in an Example of the present disclosure.

In some embodiments, for a compound of Formula (I′) or (I), PG-L-STW forms a covalent bond with suitably-protected Cys-OMe at a rate characterized by an intrinsic rate constant (K) that is less than that of PG-L-STW′, wherein STW′ is unsubstituted acrylyl. Typically, when comparing intrinsic rate constants of PG-L-STW and PG-L-STW′, the assay conditions, PG, and L are kept constant, STW′ is unsubstituted acrylyl, and STW is a substituent described herein. In some embodiments, STW′ is optionally substituted acrylyl. In some embodiments, STW′ is substituted acrylyl. In some embodiments, suitably-protected Cys-OMe is selected from Cys-OMe, Boc-Cys-OMe, Cbz-Cys-OMe, and Fmoc-Cys-OMe. In some embodiments, suitably-protected Cys-OMe is Cys-OMe (i.e., wherein the amine group is unprotected). In some embodiments, a suitably-protected Cys-OMe is selected such that (i) the thiol group is unprotected, (ii) the suitably-protected Cys-OMe does not decompose under the assay conditions, and (iii) the suitably-protected Cys-OMe is soluble under the assay conditions.

In some embodiments, for a compound of Formula (I′) or (I), PG-L-STW forms a covalent bond with suitably-protected Cys-OMe at a rate characterized by an intrinsic rate constant (K) that is less than 0.02 min⁻¹, such as less than 0.01 min⁻¹, less than 0.005 min⁻¹, less than 0.004 min⁻¹, less than 0.003 min⁻¹, less than 0.002 min⁻¹, less than 0.001 min⁻¹, less than 0.0005 min⁻¹, less than 0.0004 min⁻¹, less than 0.0003 min⁻¹, less than 0.0002 min⁻¹, less than 0.0001 min⁻¹, less than 0.00005 min⁻¹, less than 0.00004 min⁻¹, less than 0.00003 min⁻¹, less than 0.00002 min⁻¹, or less than 0.00001 min⁻¹. In some embodiments, PG-L-STW does not detectably form a covalent bond with suitably-protected Cys-OMe. In some embodiments, the intrinsic rate constant of the covalent bond formation is not measurable, or is not measurable over a reasonable time period, such as within 4 hours, 8 hours, 12 hours, 16 hours, 20 hours, or within 24 hours.

In some embodiments, for a compound of Formula (I′) or (I), L-STW comprises a ureylene functional group. In some embodiments, L-STW comprises a urea. Ureylene and urea are both used herein to refer to a functional group comprising N—C(═O)—N. In some embodiments, the ureylene functional group comprises a 5- to 12-membered heteroaryl group, such as a 5- or 6-membered heteroaryl group comprising one, two, or three ring nitrogen atoms. In some embodiments, the ureylene functional group spans L and STW. For example, the STW may comprise a terminal N—C(═O)— group, wherein the bond to L is to a N atom, thus forming N—C(═O)—N.

In some embodiments, for a compound of Formula (I′) or (I), the STW is a compound of Formula (II):

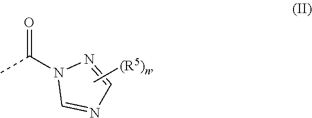

or a pharmaceutically acceptable salt thereof, wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and
n is 0, 1, or 2.

In some embodiments, for a compound of Formula (I′) or (I), the STW is a compound selected from

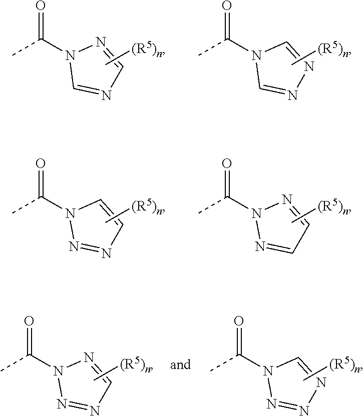

or a pharmaceutically acceptable salt thereof, wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and
n is 0, 1, or 2.

In some embodiments, for a compound of Formula (I), (I), or (II), L is a compound of Formula (III):

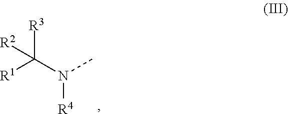

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R³⁰;
(b) R¹ and R⁴, together with the atoms to which they are attached, form a 7- to 12-membered spirocyclic heterocycloalkyl ring which is substituted with the TBM and optionally further substituted with one, two, or three R³⁰; R² is selected from R³⁰; and R³ is selected from hydrogen and R³⁰;
(c) R¹ and R⁴, together with the atoms to which they are attached, form a 7- to 12-membered fused bicyclic heterocycloalkyl ring which is substituted with the TBM and optionally further substituted with one, two, or three R³⁰; and R² and R³ are each independently selected from hydrogen and R³⁰;
(d) R¹ is selected from hydrogen and R³⁰; R² and R³, together with the carbon atom to which they are attached, form a 4- to 8-membered monocyclic heterocycloalkyl ring which is substituted with the TBM and optionally further substituted with one, two, or three R³⁰; and R⁴ is selected from R³⁰; or
(e) R¹ and R⁴, together with the atoms to which they are attached, form a 4- to 8-membered monocyclic heterocycloalkyl ring which is substituted with the TBM and optionally further substituted with one, two, or three R³⁰; R² is selected from R³⁰ and a bond to the TBM; and R³ is selected from hydrogen and R³⁰; and wherein:
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In some embodiments, for a compound of Formula (I′), (I), or (II), L is a compound of Formula (III):

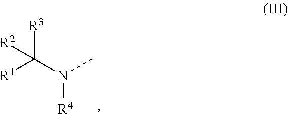

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R³⁰;
(b) R¹ and R⁴, together with the atoms to which they are attached, form a 7- to 12-membered spirocyclic heterocycloalkyl ring which is substituted with the TBM and optionally further substituted with one, two, or three R³⁰; R² is selected from R³⁰; and R³ is selected from hydrogen and R³⁰;
(c) R¹ and R⁴, together with the atoms to which they are attached, form a 7- to 12-membered fused bicyclic heterocycloalkyl ring which is substituted with the TBM and optionally further substituted with one, two, or three R³⁰; and R² and R³ are each independently selected from hydrogen and R³⁰;
(d) R¹ is selected from hydrogen and R³⁰; R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R³⁰; and R⁴ is selected from R³⁰; or
(e) R¹ and R⁴, together with the atoms to which they are attached, form a 4- to 8-membered monocyclic heterocycloalkyl ring which is substituted with the TBM and optionally further substituted with one, two, or three R³⁰; R² is selected from R³⁰ and a bond to the TBM; and R³ is selected from hydrogen and R³⁰; and wherein:
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In some embodiments, for a compound of Formula (I), (I), or (II), L is a compound of Formula (III):

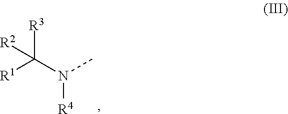

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a 4- to 8-membered monocyclic heterocycloalkyl ring which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R³ is selected from hydrogen and R⁶; and wherein:
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, for a compound of Formula (I), (I), or (II), L is a compound of Formula (III):

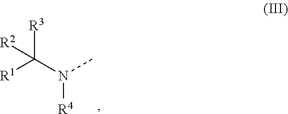

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R³ is selected from hydrogen and R⁶; and wherein:
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, for a compound of Formula (I′), (I), (II), or (III), L-STW is a compound of Formula (IV):

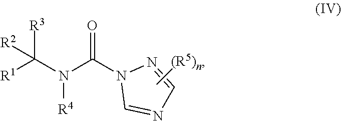

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R³⁰;
(b) R¹ and R⁴, together with the atoms to which they are attached, form a 7- to 12-membered spirocyclic heterocycloalkyl ring which is substituted with the TBM and optionally further substituted with one, two, or three R³⁰; R² is selected from R³⁰; and R³ is selected from hydrogen and R³⁰;
(c) R¹ and R⁴, together with the atoms to which they are attached, form a 7- to 12-membered fused bicyclic heterocycloalkyl ring which is substituted with the TBM and optionally further substituted with one, two, or three R³⁰; and R² and R³ are each independently selected from hydrogen and R³⁰;
(d) R¹ is selected from hydrogen and R³⁰; R² and R³, together with the carbon atom to which they are attached, form a 4- to 8-membered monocyclic heterocycloalkyl ring which is substituted with the TBM and optionally further substituted with one, two, or three R³⁰; and R⁴ is selected from R³⁰; or
(e) R¹ and R⁴, together with the atoms to which they are attached, form a 4- to 8-membered monocyclic heterocycloalkyl ring which is substituted with the TBM and optionally further substituted with one, two, or three R³⁰; R² is selected from R³⁰ and a bond to the TBM; and R³ is selected from hydrogen and R³⁰; and wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
n is 0, 1, or 2;
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In some embodiments, for a compound of Formula (I′), (I), (II), or (III), L-STW is a compound of Formula (IV):

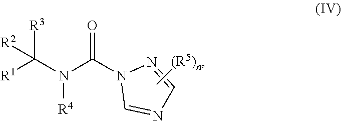

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R³⁰;
(b) R¹ and R⁴, together with the atoms to which they are attached, form a 7- to 12-membered spirocyclic heterocycloalkyl ring which is substituted with the TBM and optionally further substituted with one, two, or three R³⁰; R² is selected from R³⁰; and R³ is selected from hydrogen and R³⁰;
(c) R¹ and R⁴, together with the atoms to which they are attached, form a 7- to 12-membered fused bicyclic heterocycloalkyl ring which is substituted with the TBM and optionally further substituted with one, two, or three R³⁰; and R² and R³ are each independently selected from hydrogen and R³⁰;
(d) R¹ is selected from hydrogen and R³⁰; R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R³⁰; and R⁴ is selected from R³⁰; or
(e) R¹ and R⁴, together with the atoms to which they are attached, form a 4- to 8-membered monocyclic heterocycloalkyl ring which is substituted with the TBM and optionally further substituted with one, two, or three R³⁰; R² is selected from R³⁰ and a bond to the TBM; and R³ is selected from hydrogen and R³⁰; and wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
n is 0, 1, or 2;
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In some embodiments, for a compound of Formula (I′), (I), (II), or (III), L-STW is a compound of Formula (IV):

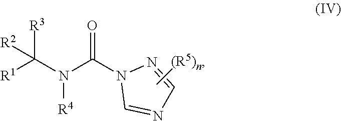

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a 4- to 8-membered monocyclic heterocycloalkyl ring which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R³ is selected from hydrogen and R⁶; and wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
n is 0, 1, or 2;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, for a compound of Formula (I′), (I), (II), or (III), L-STW is a compound of Formula (IV):

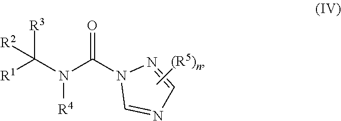

or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R³ is selected from hydrogen and R⁶; and wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
n is 0, 1, or 2;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, for a compound of Formula (I′), (I), (II), (III), or (IV), L-STW is a compound of Formula (IV-A):

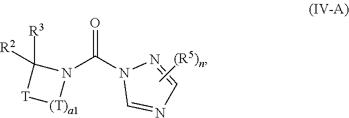

wherein:
a1 is 1,2,3,4, or 5;
T is independently selected at each occurrence from N(R⁹), C(R¹)₂, C(O), O, S(O), and S(O)₂;
R¹ is independently selected at each occurrence from hydrogen and R³⁰; and
R⁹ is independently selected at each occurrence from hydrogen and R³⁰

In some embodiments, for a compound of Formula (I′), (I), (II), (III), or (IV), L-STW is a compound of Formula (IV-A):

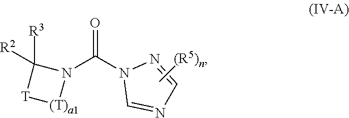

wherein:
a1 is 1,2,3,4, or 5;
T is independently selected at each occurrence from N(R⁹), C(R⁸)₂, C(O), O, S(O), and S(O)₂;
R⁸ is independently selected at each occurrence from hydrogen and R⁶; and
R⁹ is independently selected at each occurrence from hydrogen and R⁷.

In some embodiments, the compound of Formula (IV-A) is a compound of Formula (IV-A1):

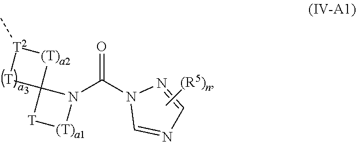

wherein:
a2 is 0, 1, 2, 3, 4, or 5;
a3 is 0, 1, 2, 3, 4, or 5;
wherein the sum of a2 and a3 is 2, 3, 4, 5, or 6; and
T² is selected from N and C(R⁸).

In some embodiments, for a compound of Formula (I′), (I), (II), (III), or (IV), L-STW is a compound of Formula (IV-B):

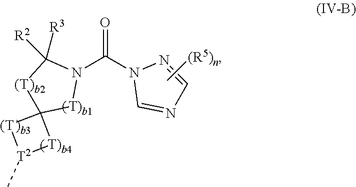

wherein:
b1 is 1, 2, 3, 4, or 5;
b2 is 0, 1,2,3,4, or 5;
b3 is 1, 2, 3, 4, or 5;
b4 is 1, 2, 3, 4, or 5;
wherein the sum of b1, b2, b3, and b4 is less than 9;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R³⁰; and
R⁹ is independently selected at each occurrence from hydrogen and R³⁰

In some embodiments, for a compound of Formula (I′), (I), (II), (III), or (IV), L-STW is a compound of Formula (IV-B):

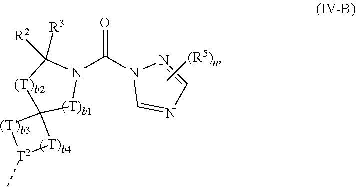

wherein:
b1 is 1, 2, 3, 4, or 5;
b2 is 0, 1,2,3,4, or 5;
b3 is 1, 2, 3, 4, or 5;
b4 is 1, 2, 3, 4, or 5;
wherein the sum of b1, b2, b3, and b4 is less than 9;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R⁶; and
R⁹ is independently selected at each occurrence from hydrogen and R⁷.

In some embodiments, for a compound of Formula (I′), (I), (II), (III), or (IV), L-STW is a compound of Formula (IV-C):

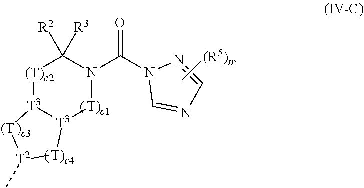

wherein:
c1 is 0, 1, 2, 3, or 4;
c2 is 0, 1, 2, 3, or 4;
c3 is 0, 1, 2, 3, or 4;
c4 is 0, 1, 2, 3, or 4;
wherein the sum of c3 and c4 is at least 1; and the sum of ci, c2, c3, and c4 is less than 8;
T is independently selected at each occurrence from N(R⁹), C(R⁸)₂, C(O), O, S(O), and S(O)₂;
T² is selected from N and C(R⁸);
T³ is independently selected at each occurrence from N and C(R⁸);
R⁸ is independently selected at each occurrence from hydrogen and R³⁰; and
R⁹ is independently selected at each occurrence from hydrogen and R³⁰.

In some embodiments, for a compound of Formula (I′), (I), (II), (III), or (IV), L-STW is a compound of Formula (IV-C):

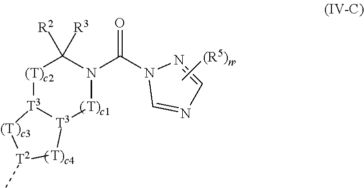

wherein:
c1 is 0, 1, 2, 3, or 4;
c2 is 0, 1, 2, 3, or 4;
c3 is 0, 1, 2, 3, or 4;
c4 is 0, 1, 2, 3, or 4;
wherein the sum of c3 and c4 is at least 1; and the sum of c1, c2, c3, and c4 is less than 8;
T is independently selected at each occurrence from N(R⁹), C(R⁸)₂, C(O), O, S(O), and S(O)₂;
T² is selected from N and C(R⁸);
T³ is independently selected at each occurrence from N and C(R⁸);
R⁸ is independently selected at each occurrence from hydrogen and R⁶; and
R⁹ is independently selected at each occurrence from hydrogen and R⁷.

In some embodiments, for a compound of Formula (I′), (I), (II), (III), or (IV), L-STW is a compound of Formula (IV-D):

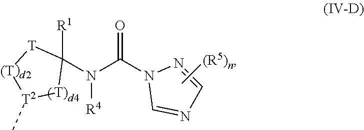

wherein:
d1 is 0, 1, 2, 3, or 4;
d2 is 0, 1, 2, 3, or 4;
wherein the sum of d1 and d2 is 1, 2, 3, 4, or 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R³⁰; and
R⁹ is independently selected at each occurrence from hydrogen and R³⁰.

In some embodiments, for a compound of Formula (I′), (I), (II), (III), or (IV), L-STW is a compound of Formula (IV-D):

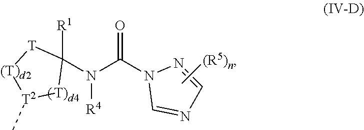

wherein:
d1 is 0, 1, 2, 3, or 4;
d2 is 0, 1, 2, 3, or 4;
wherein the sum of d1 and d2 is 1, 2, 3, 4, or 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R⁶; and
R⁹ is independently selected at each occurrence from hydrogen and R⁷.

In some embodiments, for a compound of Formula (I′), (I), (II), (III), or (IV), L-STW is a compound of Formula (IV-E1) or (IV-E2):

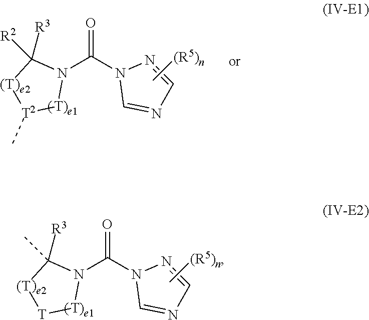

wherein:
e1 is 0, 1, 2, 3, or 4;
e2 is 0, 1, 2, 3, or 4;
wherein the sum of e1 and e2 is between 1 and 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R³⁰; and
R⁹ is independently selected at each occurrence from hydrogen and R³⁰

In some embodiments, for a compound of Formula (I′), (I), (II), (III), or (IV), L-STW is a compound of Formula (IV-E1) or (IV-E2):

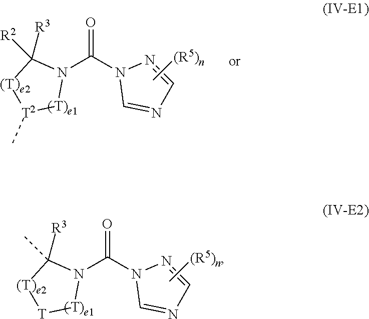

wherein:
e1 is 0, 1, 2, 3, or 4;
e2 is 0, 1, 2, 3, or 4;
wherein the sum of e1 and e2 is between 1 and 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R⁶; and
R⁹ is independently selected at each occurrence from hydrogen and R⁷.

In certain aspects, the present disclosure provides a covalently modified amino acid residue in a non-Ras polypeptide, wherein the modified amino acid residue is a compound of Formula (V′):

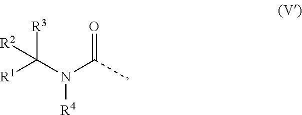

wherein:
the dashed bond represents a bond to the amino acid residue;
(a) R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R³⁰;
(b) R¹ and R⁴, together with the atoms to which they are attached, form a 7- to 12-membered spirocyclic heterocycloalkyl ring which is substituted with TBM and optionally further substituted with one, two, or three R³⁰; R² is selected from R³⁰; and R³ is selected from hydrogen and R³⁰;
(c) R¹ and R⁴, together with the atoms to which they are attached, form a 7- to 12-membered fused bicyclic heterocycloalkyl ring which is substituted with TBM and optionally further substituted with one, two, or three R³⁰; and R² and R³ are each independently selected from hydrogen and R³⁰;
(d) R¹ is selected from hydrogen and R³⁰; R² and R³, together with the carbon atom to which they are attached, form a 4- to 8-membered monocyclic heterocycloalkyl ring which is substituted with TBM and optionally further substituted with one, two, or three R³⁰; and R⁴ is selected from R³⁰; or
(e) R¹ and R⁴, together with the atoms to which they are attached, form a 4- to 8-membered monocyclic heterocycloalkyl ring which is substituted with TBM and optionally further substituted with one, two, or three R³⁰; R² is selected from R³⁰ and a bond to TBM; and R³ is selected from hydrogen and R³⁰; and wherein:
TBM is selected from C₆₋₄₀organyl and C₆₋₄organoheteryl;
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In certain aspects, the present disclosure provides a covalently modified amino acid residue in a non-Ras polypeptide, wherein the modified amino acid residue is a compound of Formula (V′):

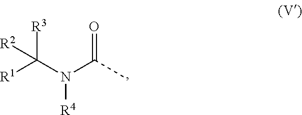

wherein: the dashed bond represents a bond to the amino acid residue;
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶;
(b) R¹ and R⁴, together with the atoms to which they are attached, form a 7- to 12-membered spirocyclic heterocycloalkyl ring which is substituted with TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a 4- to 8-membered monocyclic heterocycloalkyl ring which is substituted with TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with TBM and optionally further substituted with one, two, or three R⁶; R² is selected from R⁶ and a bond to TBM; and R³ is selected from hydrogen and R⁶; and wherein:
TBM is selected from C₆₋₄₀organyl and C₆₋₄organoheteryl;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, for a modified amino acid residue of Formula (V) or (V), the amino acid is selected from serine, tyrosine, cysteine, lysine, and histidine. In some embodiments, the amino acid is serine. In some embodiments, the amino acid is tyrosine. In some embodiments, the amino acid is cysteine. In some embodiments, the amino acid is lysine. In some embodiments, the amino acid is histidine.

In certain aspects, the present disclosure provides a covalently modified serine residue in a non-Ras polypeptide, wherein the modified serine residue is a compound of Formula (V):

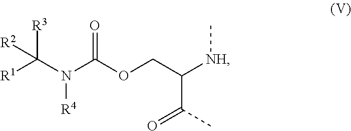

wherein:
the dashed bonds represent peptide bonds between the serine residue and adjacent amino acid residues of the polypeptide, respectively, or, if the serine residue is located at the N-terminus or C-terminus of the polypeptide, the dashed bond to the nitrogen atom represents a bond to a hydrogen atom or the dashed bond to the carbon atom represents a bond to a hydroxy group, respectively;
(a) R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R³⁰;
(b) R¹ and R⁴, together with the atoms to which they are attached, form a 7- to 12-membered spirocyclic heterocycloalkyl ring which is substituted with TBM and optionally further substituted with one, two, or three R³⁰; R² is selected from R³⁰; and R³ is selected from hydrogen and R³⁰;
(c) R¹ and R⁴, together with the atoms to which they are attached, form a 7- to 12-membered fused bicyclic heterocycloalkyl ring which is substituted with TBM and optionally further substituted with one, two, or three R³⁰; and R² and R³ are each independently selected from hydrogen and R³⁰;
(d) R¹ is selected from hydrogen and R³⁰; R² and R³, together with the carbon atom to which they are attached, form a 4- to 8-membered monocyclic heterocycloalkyl ring which is substituted with TBM and optionally further substituted with one, two, or three R³⁰; and R⁴ is selected from R³⁰; or
(e) R¹ and R⁴, together with the atoms to which they are attached, form a 4- to 8-membered monocyclic heterocycloalkyl ring which is substituted with TBM and optionally further substituted with one, two, or three R³⁰; R² is selected from R³⁰ and a bond to TBM; and R³ is selected from hydrogen and R³⁰; and wherein:
TBM is selected from C₆₋₄₀organyl and C₆₋₄organoheteryl;
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In certain aspects, the present disclosure provides a covalently modified serine residue in a non-Ras polypeptide, wherein the modified serine residue is a compound of Formula (V):

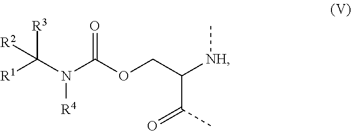

wherein:
the dashed bonds represent peptide bonds between the serine residue and adjacent amino acid residues of the polypeptide, respectively, or, if the serine residue is located at the N-terminus or C-terminus of the polypeptide, the dashed bond to the nitrogen atom represents a bond to a hydrogen atom or the dashed bond to the carbon atom represents a bond to a hydroxy group, respectively;
(a) R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R³⁰;
(b) R¹ and R⁴, together with the atoms to which they are attached, form a 7- to 12-membered spirocyclic heterocycloalkyl ring which is substituted with TBM and optionally further substituted with one, two, or three R³⁰; R² is selected from R³⁰; and R³ is selected from hydrogen and R³⁰;
(c) R¹ and R⁴, together with the atoms to which they are attached, form a 7- to 12-membered fused bicyclic heterocycloalkyl ring which is substituted with TBM and optionally further substituted with one, two, or three R³⁰; and R² and R³ are each independently selected from hydrogen and R³⁰;
(d) R¹ is selected from hydrogen and R³⁰; R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R³⁰; and R⁴ is selected from R³⁰; or
(e) R¹ and R⁴, together with the atoms to which they are attached, form a 4- to 8-membered monocyclic heterocycloalkyl ring which is substituted with TBM and optionally further substituted with one, two, or three R³⁰; R² is selected from R³⁰ and a bond to TBM; and R³ is selected from hydrogen and R³⁰; and wherein:
TBM is selected from C₆₋₄₀organyl and C₆₋₄organoheteryl;
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂—6 alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In certain aspects, the present disclosure provides a covalently modified serine residue in a non-Ras polypeptide, wherein the modified serine residue is a compound of Formula (V):

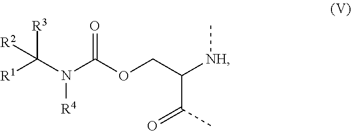

wherein:
the dashed bonds represent peptide bonds between the serine residue and adjacent amino acid residues of the polypeptide, respectively, or, if the serine residue is located at the N-terminus or C-terminus of the polypeptide, the dashed bond to the nitrogen atom represents a bond to a hydrogen atom or the dashed bond to the carbon atom represents a bond to a hydroxy group, respectively;
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶;
(b) R¹ and R⁴, together with the atoms to which they are attached, form a 7- to 12-membered spirocyclic heterocycloalkyl ring which is substituted with TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a 4- to 8-membered monocyclic heterocycloalkyl ring which is substituted with TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with TBM and optionally further substituted with one, two, or three R⁶; R² is selected from R⁶ and a bond to TBM; and R³ is selected from hydrogen and R⁶; and wherein:
TBM is selected from C₆₋₄₀organyl and C₆₋₄organoheteryl;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In certain aspects, the present disclosure provides a covalently modified serine residue in a non-Ras polypeptide, wherein the modified serine residue is a compound of Formula (V):

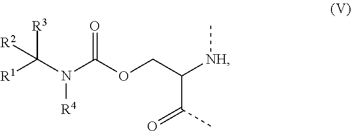

wherein:
the dashed bonds represent peptide bonds between the serine residue and adjacent amino acid residues of the polypeptide, respectively, or, if the serine residue is located at the N-terminus or C-terminus of the polypeptide, the dashed bond to the nitrogen atom represents a bond to a hydrogen atom or the dashed bond to the carbon atom represents a bond to a hydroxy group, respectively;
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶;
(b) R¹ and R⁴, together with the atoms to which they are attached, form a 7- to 12-membered spirocyclic heterocycloalkyl ring which is substituted with TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with TBM and optionally further substituted with one, two, or three R⁶; R² is selected from R⁶ and a bond to TBM; and R³ is selected from hydrogen and R⁶; and wherein:
TBM is selected from C₆₋₄₀organyl and C₆₋₄organoheteryl;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

In some embodiments, the modified serine residue of Formula (V) is modified serine residue of Formula (V-A):

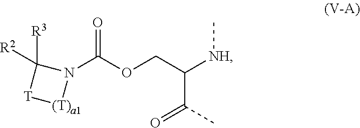

wherein:
a1 is 1,2,3,4, or 5;
T is independently selected at each occurrence from N(R⁹), C(R⁸)₂, C(O), O, S(O), and S(O)₂;
R⁸ is independently selected at each occurrence from hydrogen and R³⁰; and
R⁹ is independently selected at each occurrence from hydrogen and R³⁰.

In some embodiments, the modified serine residue of Formula (V) is modified serine residue of Formula (V-A):

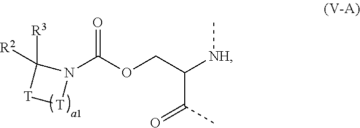

wherein:
a1 is 1, 2, 3, 4, or 5;
T is independently selected at each occurrence from N(R⁹), C(R⁸)₂, C(O), O, S(O), and S(O)₂;
R⁸ is independently selected at each occurrence from hydrogen and R⁶; and
R⁹ is independently selected at each occurrence from hydrogen and R⁷.

In some embodiments, the modified serine residue of Formula (V-A) is modified serine residue of Formula (V-A1):

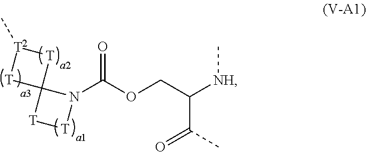

wherein:
a2 is 0, 1, 2, 3, 4, or 5;
a3 is 0, 1, 2, 3, 4, or 5;
wherein the sum of a2 and a3 is 2, 3, 4, 5, or 6; and
T² is selected from N and C(R¹).

In some embodiments, the modified serine residue of Formula (V) is modified serine residue of Formula (V-B):

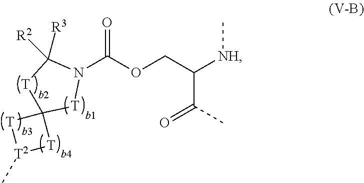

wherein:
b1 is 1, 2, 3, 4, or 5;
b2 is 0, 1,2,3,4, or 5;
b3 is 1, 2, 3, 4, or 5;
b4 is 1, 2, 3, 4, or 5;
wherein the sum of b1, b2, b3, and b4 is less than 9;
T is independently selected at each occurrence from N(R⁹), C(R¹)₂, C(O), O, S(O), and S(O)₂;
T² is selected from N and C(R¹);
R¹ is independently selected at each occurrence from hydrogen and R³⁰; and
R⁹ is independently selected at each occurrence from hydrogen and R³⁰.

In some embodiments, the modified serine residue of Formula (V) is modified serine residue of Formula (V-B):

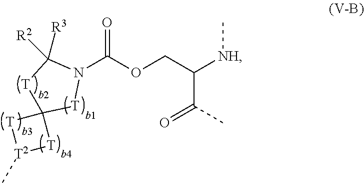

wherein:
b1 is 1, 2, 3, 4, or 5;
b2 is 0, 1,2,3,4, or 5;
b3 is 1, 2, 3, 4, or 5;
b4 is 1, 2, 3, 4, or 5;
wherein the sum of b1, b2, b3, and b4 is less than 9;
T is independently selected at each occurrence from N(R⁹), C(R¹)₂, C(O), O, S(O), and S(O)₂;
T² is selected from N and C(R⁸);
R⁸ is independently selected at each occurrence from hydrogen and R⁶; and
R⁹ is independently selected at each occurrence from hydrogen and R⁷.

In some embodiments, the modified serine residue of Formula (V) is modified serine residue of Formula (V-C):

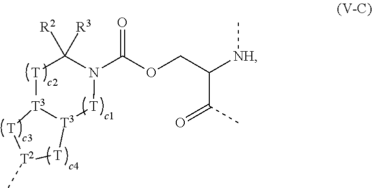

wherein:
c1 is 0, 1,2, 3, or 4;
c2 is 0, 1, 2, 3, or 4;
c3 is 0, 1, 2, 3, or 4;
c4 is 0, 1, 2, 3, or 4;
wherein the sum of c3 and c4 is at least 1; and the sum of ci, c2, c3, and c4 is less than 8;
T is independently selected at each occurrence from N(R⁹), C(R⁸)₂, C(O), O, S(O), and S(O)₂;
T² is selected from N and C(R⁸);
T³ is independently selected at each occurrence from N and C(R⁸);
R⁸ is independently selected at each occurrence from hydrogen and R³⁰; and
R⁹ is independently selected at each occurrence from hydrogen and R³⁰.

In some embodiments, the modified serine residue of Formula (V) is modified serine residue of Formula (V-C):

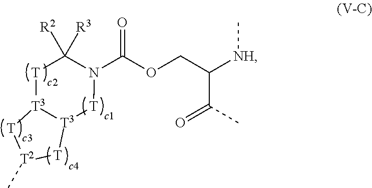

wherein:
c1 is 0, 1,2, 3, or 4;
c2 is 0, 1, 2, 3, or 4;
c3 is 0, 1, 2, 3, or 4;
c4 is 0, 1, 2, 3, or 4;
wherein the sum of c3 and c4 is at least 1; and the sum of c1, c2, c3, and c4 is less than 8;
T is independently selected at each occurrence from N(R⁹), C(R⁸)₂, C(O), O, S(O), and S(O)₂;
T² is selected from N and C(R⁸);
T³ is independently selected at each occurrence from N and C(R⁸);
R⁸ is independently selected at each occurrence from hydrogen and R⁶; and
R⁹ is independently selected at each occurrence from hydrogen and R⁷.

In some embodiments, the modified serine residue of Formula (V) is modified serine residue of Formula (V-D):

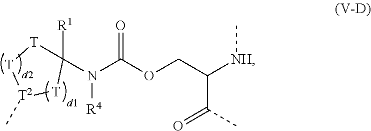

wherein:
d1 is 0, 1, 2, 3, or 4;
d2 is 0, 1, 2, 3, or 4;
wherein the sum of d1 and d2 is 1, 2, 3, 4, or 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R³⁰; and
R⁹ is independently selected at each occurrence from hydrogen and R³⁰

In some embodiments, the modified serine residue of Formula (V) is modified serine residue of Formula (V-D):

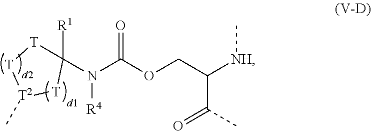

wherein:
d1 is 0, 1, 2, 3, or 4;
d2 is 0, 1, 2, 3, or 4;
wherein the sum of d1 and d2 is 1, 2, 3, 4, or 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R⁶; and
R⁹ is independently selected at each occurrence from hydrogen and R⁷.

In some embodiments, the modified serine residue of Formula (V) is modified serine residue of Formula (V-E1) or (V-E2):

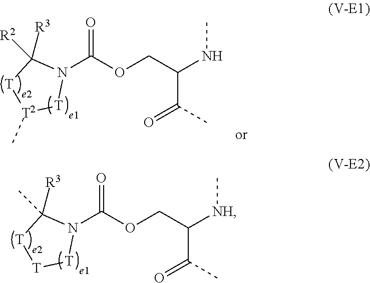

wherein:
e1 is 0, 1, 2, 3, or 4;
e2 is 0, 1, 2, 3, or 4;
wherein the sum of e1 and e2 is between 1 and 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R³⁰; and
R⁹ is independently selected at each occurrence from hydrogen and R³⁰.

In some embodiments, the modified serine residue of Formula (V) is modified serine residue of Formula (V-E1) or (V-E2):

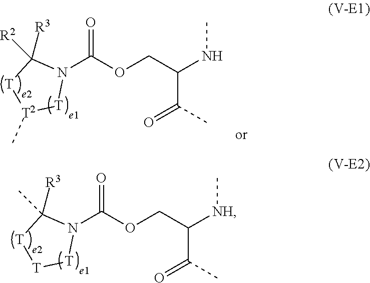

wherein:
e1 is 0, 1, 2, 3, or 4;
e2 is 0, 1,2, 3, or 4;
wherein the sum of e1 and e2 is between 1 and 5;
T is independently selected at each occurrence from N(R⁹), C(R⁸)₂, C(O), O, S(O), and S(O)₂;
T² is selected from N and C(R⁸);
R⁸ is independently selected at each occurrence from hydrogen and R⁶; and
R⁹ is independently selected at each occurrence from hydrogen and R⁷.

In some embodiments, for a modified amino acid (e.g., serine) residue described herein, such as a modified amino acid residue of Formula (V′) or (V), the polypeptide exhibits reduced signaling output relative to the signaling output of the polypeptide prior to modification of the amino acid residue (e.g., seine). In some embodiments, the reduced signaling output is evidenced by a reduction of cell growth or division of tumor cell expressing the polypeptide. In some embodiments, the modified amino acid residue (e.g., serine) is formed by contacting an unmodified residue in the polypeptide with a precursor compound, wherein the precursor compound comprises a staying group and a leaving group, and wherein said contacting results in release of the leaving group and formation of said modified amino acid residue (e.g., serine). In some embodiments, the precursor compound is a compound described herein, such as a compound of Formula (I′) or (I). In some embodiments, the leaving group is selected from

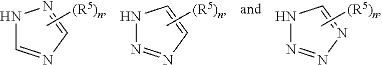

or a salt or tautomer thereof, wherein R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and n is 0, 1, or 2. In some embodiments, R⁵ is independently selected at each occurrence from R³⁰

In some embodiments, the amino acid residue is an active amino acid, such as an active serine. As used herein, the term active amino acid refers to an amino acid residue that resides in an enzymatic active site of a protein and is utilized in an enzymatic reaction performed by the protein. For example, the term active serine refers to a serine residue that resides in an enzymatic active site of the a protein and is utilized in an enzymatic reaction performed by the protein. An active serine may be found in a serine hydrolase or a serine protease. In some embodiments, a Target Protein of the present disclosure is a serine hydrolase. In some embodiments, a Target Protein of the present disclosure is a serine protease. In some embodiments, the amino acid residue is an inactive amino acid, such as an inactive serine. As used herein, the term inactive amino acid refers to an amino acid residue that is not utilized in an enzymatic reaction. In some embodiments, a Target Protein of the present disclosure is not a serine hydrolase. In some embodiments, a Target Protein of the present disclosure is not a serine protease.

In some embodiments, for a compound or modified polypeptide described herein, such as a compound of Formula (I), (I), (II), (III), or (IV) or a modified polypeptide of Formula (V′) or (V), R¹ and R⁴, together with the atoms to which they are attached, form a 4- to 8-membered monocyclic heterocycloalkyl ring which is optionally substituted with one, two, or three R³⁰; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R³⁰. In some embodiments, R¹ and R⁴, together with the atoms to which they are attached, form a 7- to 12-membered spirocyclic heterocycloalkyl ring which is substituted with TBM and optionally further substituted with one, two, or three R³⁰; R² is selected from R³⁰; and R³ is selected from hydrogen and R³⁰. In some embodiments, R¹ and R⁴, together with the atoms to which they are attached, form a 7- to 12-membered fused bicyclic heterocycloalkyl ring which is substituted with TBM and optionally further substituted with one, two, or three R³⁰; and R² and R³ are each independently selected from hydrogen and R³⁰. In some embodiments, R¹ is selected from hydrogen and R³⁰; R² and R³, together with the carbon atom to which they are attached, form a 4- to 8-membered monocyclic heterocycloalkyl ring which is substituted with TBM and optionally further substituted with one, two, or three R³⁰; and R⁴ is selected from R³⁰. In some embodiments, R¹ is selected from hydrogen and R³⁰; R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R³⁰; and R⁴ is selected from R³⁰. In some embodiments, R¹ and R⁴, together with the atoms to which they are attached, form a 4- to 8-membered monocyclic heterocycloalkyl ring which is substituted with TBM and optionally further substituted with one, two, or three R³⁰; R² is selected from R³⁰ and a bond to TBM; and R³ is selected from hydrogen and R³⁰. In some embodiments, R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶. In some embodiments, R¹ and R⁴, together with the atoms to which they are attached, form a 7- to 12-membered spirocyclic heterocycloalkyl ring which is substituted with TBM and optionally further substituted with one, two, or three R⁶; R² is selected from R⁶; and R³ is selected from hydrogen and R⁶. In some embodiments, R¹ and R⁴, together with the atoms to which they are attached, form a 7- to 12-membered fused bicyclic heterocycloalkyl ring which is substituted with TBM and optionally further substituted with one, two, or three R⁶; and R² and R³ are each independently selected from hydrogen and R⁶. In some embodiments, R¹ is selected from hydrogen and R⁶; R² and R³, together with the carbon atom to which they are attached, form a 4- to 8-membered monocyclic heterocycloalkyl ring which is substituted with TBM and optionally further substituted with one, two, or three R⁶; and R⁴ is selected from R⁷. In some embodiments, R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶; and R⁴ is selected from R⁷. In some embodiments, R¹ and R⁴, together with the atoms to which they are attached, form a 4- to 8-membered monocyclic heterocycloalkyl ring which is substituted with TBM and optionally further substituted with one, two, or three R⁶; R² is selected from R⁶ and a bond to TBM; and R³ is selected from hydrogen and R⁶.

In some embodiments, for a compound of Formula (IV-A) or a modified polypeptide of Formula (V-A), each T is independently C(R⁸)₂. In some embodiments, each T is CH₂. In some embodiments, R² and R³, together with the carbon atom to which they are attached, form 4- to 8-membered monocyclic heterocycloalkyl ring substituted with TBM and optionally further substituted with one, two, or three R⁶. In some embodiments, a1 is 1 or 2. In some embodiments, a1 is 1.

In some embodiments, for a compound of Formula (IV-A1) or a modified polypeptide of Formula (V-A1), each T is independently C(R⁸)₂. In some embodiments, each T is CH₂. In some embodiments, one T is O and each remaining T is C(R⁸)₂. In some embodiments, T² is N. In some embodiments, a1 is 1 or 2, such as a1 is 1. In some embodiments, the sum of a2 and a3 is 2, 3, or 4. In some embodiments, a2 is 1 or 2. In some embodiments, a3 is 1 or 2.

In some embodiments, for a compound of Formula (IV-B) or a modified polypeptide of Formula (V-B), each T is independently C(R⁸)₂. In some embodiments, each T is CH₂. In some embodiments, one T is O and each remaining T is C(R⁸)₂. In some embodiments, T² is N. In some embodiments, the sum of b1, b2, b3, and b4 is less than 7, such as the sum of b1, b2, b3, and b4 is 3, 4, 5, or 6. In some embodiments, the sum of b1 and b2 is 1, 2, or 3. In some embodiments, bi is 1 or 2. In some embodiments, b2 is 0, 1, or 2. In some embodiments, b3 is 1, 2, or 3. In some embodiments, b4 is 1, 2, or 3.

In some embodiments, for a compound of Formula (IV-C) or a modified polypeptide of Formula (V-C), each T is independently C(R¹)₂. In some embodiments, each T is CH₂. In some embodiments, T² is N. In some embodiments, each T³ is independently C(R¹). In some embodiments, each T³ is CH. In some embodiments, the sum of c3 and c4 is 1, 2, or 3. In some embodiments, the sum of ci, c2, c3, and c4 is 3, 4, 5 or 6. In some embodiments, c1 is 0, 1, or 2. In some embodiments, c1 is 0. In some embodiments, c2 is 0, 1, 2 or 3. In some embodiments, c3 is 0, 1, or 2. In some embodiments, c4 is 0, 1, or 2.

In some embodiments, for a compound of Formula (IV-D) or a modified polypeptide of Formula (V-D), each T is independently C(R⁸)₂. In some embodiments, each T is CH₂. In some embodiments, T² is N. In some embodiments, T² is C(R⁸), such as CH. In some embodiments, the sum of d1 and d2 is 1, 2, 3, or 4. In some embodiments, d1 is 0, 1, or 2 and d2 is 1, 2, or 3.

In some embodiments, for a compound of Formula (IV-E1) or a modified polypeptide of Formula (V-E1), each T is independently C(R⁸)₂. In some embodiments, each T is CH₂. In some embodiments, T² is N. In some embodiments, T² is C(R⁸), such as CH. In some embodiments, the sum of e1 and e2 is 1, 2, or 3. In some embodiments, the sum of e1 and e2 is 1. In some embodiments, e1 is 0, 1, or 2. In some embodiments, e1 is 0. In some embodiments, e1 is 1. In some embodiments, e1 is 2. In some embodiments, e2 is 0, 2, or 2. In some embodiments, e2 is 0. In some embodiments, e2 is 1.

In some embodiments, for a compound of Formula (IV-E2) or a modified polypeptide of Formula (V-E2), each T is independently C(R⁸)₂. In some embodiments, each T is CH₂. In some embodiments, the sum of e1 and e2 is 1, 2, or 3. In some embodiments, the sum of e1 and e2 is 1. In some embodiments, e1 is 0, 1, or 2. In some embodiments, e1 is 0. In some embodiments, e1 is 1. In some embodiments, e1 is 2. In some embodiments, e2 is 0, 2, or 2. In some embodiments, e2 is 0. In some embodiments, e2 is 1. In some embodiments, e1 is 1 and e2 is 0.

In some embodiments, for a compound of Formula (IV-A), (IV-A1), (IV-B), (IV-C), (IV-D), (IV-E1), or (IV-E2) or a modified polypeptide of Formula (V-A), (V-A1), (V-B), (V-C), (V-D), (V-E1), or (V-E2), R⁸ is independently selected at each occurrence from hydrogen, halogen, —CN, C₁₋₆ alkyl, and C₃₋₆ cycloalkyl. In some embodiments, two R⁸ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R⁸ is independently selected at each occurrence from hydrogen and C₁₋₆ alkyl. In some embodiments, R⁸ is independently selected at each occurrence from hydrogen and C₁₋₃ alkyl. In some embodiments, one or two R⁸ are independently selected from halogen, —CN, C₁₋₆ alkyl, and C₃₋₆ cycloalkyl; and the remaining R⁸ are hydrogen. In some embodiments, one or two R⁸ are independently selected from C₁₋₃ alkyl; and the remaining R⁸ are hydrogen. In some embodiments, one R⁵ is C₁₋₃ alkyl and any remaining R⁸ are hydrogen. In some embodiments, each R⁵ is hydrogen.

In some embodiments, for a compound of Formula (IV-A), (IV-A1), (IV-B), (IV-C), (IV-D), (IV-E1), or (IV-E2) or a modified polypeptide of Formula (V-A), (V-A1), (V-B), (V-C), (V-D), (V-E1), or (V-E2), each R⁹ is independently selected from hydrogen and C₁₋₆ alkyl optionally substituted with one, two, or three substituents selected from halogen, —CN, C₁₋₆ alkyl, C₁₋₆ haloalkyl, —O(C₁₋₆ alkyl), and —O(C₁₋₆ haloalkyl). In some embodiments, each R⁹ is independently selected from hydrogen and C₁₋₃ alkyl. In some embodiments, each R⁹ is hydrogen.

In some embodiments, for a compound or modified polypeptide described herein, such as a compound of Formula (III), or (IV) or a modified polypeptide of Formula (V) or (V), R⁶ is independently selected at each occurrence from halogen, —CN, C₁₋₆ alkyl, and C₃₋₆ cycloalkyl. In some embodiments, two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R⁶ is independently selected at each occurrence from C₁₋₆ alkyl. In some embodiments, R⁶ is independently selected at each occurrence from hydrogen and C1-3 alkyl. In some embodiments, R⁷ is selected from C₁₋₆ alkyl optionally substituted with one, two, or three substituents selected from halogen, —CN, C₁₋₆ alkyl, C₁₋₆ haloalkyl, —O(C₁₋₆ alkyl), and —O(C₁₋₆ haloalkyl). In some embodiments, R⁷ is C₁₋₃ alkyl.

In some embodiments, for a compound or modified polypeptide described herein, such as a compound of Formula (III), or (IV) or a modified polypeptide of Formula (V′) or (V), R¹ is selected from hydrogen, halogen, —CN, C₁₋₆ alkyl, and C₃₋₆ cycloalkyl. In some embodiments, R¹ is selected from hydrogen and C₁₋₆ alkyl. In some embodiments, R¹ is selected from hydrogen and C₁₋₃ alkyl. In some embodiments, R¹ is hydrogen. In some embodiments, R² is selected from halogen, —CN, C₁₋₆ alkyl, and C₃₋₆ cycloalkyl. In some embodiments, R² is selected from C₁₋₆ alkyl, such as C₁₋₃ alkyl. In some embodiments, R² is selected from optionally substituted C₁₋₆ alkyl. In some embodiments, R² is selected from C₁₋₆ alkyl substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl). In some embodiments, R³ is selected from hydrogen, halogen, —CN, C₁₋₆ alkyl, and C₃₋₆ cycloalkyl. In some embodiments, R³ is selected from hydrogen and C₁₋₆ alkyl. In some embodiments, R³ is selected from hydrogen and C₁₋₃ alkyl. In some embodiments, R³ is hydrogen. In some embodiments, R⁴ is selected from C₁₋₆ alkyl optionally substituted with one, two, or three substituents selected from halogen, —CN, C₁₋₆ alkyl, C₁₋₆ haloalkyl, —O(C₁₋₆ alkyl), and —O(C₁₋₆ haloalkyl). In some embodiments, R⁴ is C₃₋₆ cycloalkyl optionally substituted with one, two, or three substituents selected from halogen, —CN, C₁₋₆ alkyl, C₁₋₆ haloalkyl, —O(C₁₋₆ alkyl), and —O(C₁₋₆ haloalkyl). In some embodiments, R⁴ is C₁₋₆ alkyl, such as C₁₋₃ alkyl.

In some embodiments, for a compound described herein, such as a compound of Formula (II), (III), or (IV), R⁵ is selected from hydrogen, halogen, —CH₃, —CH₂F, —CHF₂, and —CF₃. In some embodiments, R⁵ is hydrogen. In some embodiments, R⁵ is halogen. In some embodiments, R⁵ is —CH₃. In some embodiments, R⁵ is —CH₂F. In some embodiments, R⁵ is —CHF₂. In some embodiments, R⁵ is CF₃. In some embodiments, R⁵ is C1. In some embodiments, R⁵ is F.

In some embodiments, for a compound or modified polypeptide described herein, such as a compound of Formula (I′), (I), (II), (III), or (IV) or a modified polypeptide of Formula (V′) or (V), the TBM is selected from C₆₋₁₀₀ organyl and C₆₋₁₀₀ organoheteryl, such as C₆₋₈₀ organyl, C₆₋₈₀ organoheteryl, C₆₋₆₀ organyl, C₆₋₆₀ organoheteryl, C₆₋₅₀ organyl, C₆₋₅₀ organoheteryl, C₆₋₄₀ organyl, C₆₋₄₀ organoheteryl, C₆₋₃₀ organyl, C₆₋₃₀ organoheteryl, C₆₋₂₀ organyl, C₆₋₂₀ organoheteryl, C₁₀₋₁₀₀ organyl, C₁₀₋₁₀₀ organoheteryl, C₁₀₋₈₀ organyl, C₁₀₋₈₀ organoheteryl, C₁₀₋₆₀ organyl, C₁₀₋₆₀ organoheteryl, C₁₀₋₅₀ organyl, C₁₀₋₅₀ organoheteryl, C₁₀₋₄₀ organyl, C₁₀₋₄₀ organoheteryl, C₁₀₋₃₀₀ organyl, C₁₀₋₃₀₀ organoheteryl, C₁₀₋₂₀ organyl, C₁₀₋₂₀ organoheteryl, C₁₅₋₁₀₀ organyl, C₁₅₋₁₀₀ organoheteryl, C₁₅₋₈₀ organyl, C₁₁₋₈₀ organoheteryl, C₁₅₋₆₀ organyl, C₁₅₋₆₀ organoheteryl, C₁₅₋₅₀ organyl, C₁₅₋₅₀ organoheteryl, C₁₅₋₄₀ organyl, C₁₅₋₄₀ organoheteryl, C₁₅₋₃₀ organyl, C₁₅₋₃₀ organoheteryl, C₁₅₋₂₀ organyl, C₁₅₋₅₀ organoheteryl, C₂₀₋₁₀₀ organyl, C₂₀₋₁₀₀ organoheteryl, C₂₀₋₈₀ organyl, C₂₀₋₈₀ organoheteryl, C₂₀₋₆₀ organyl, C₂₀₋₆₀ organoheteryl, C₂₀₋₅₀ organyl, C₂₀₋₅₀ organoheteryl, C₂₀₋₄₀ organyl, C₂₀₋₄₀ organoheteryl, C₂₀₋₃₀ organyl, C₂₀₋₃₀ organoheteryl, C₂₀₋₂₅ organyl, C₂₀₋₂₅ organoheteryl, C₂₅₋₁₀₀ organyl, C₂₅₋₁₀₀ organoheteryl, C₂₅₋₈₀ organyl, C₂₅₋₈₀ organoheteryl, C₂₅₋₆₀ organyl, C₂₅₋₆₀ organoheteryl, C₂₅₋₅₀ organyl, C₂₅₋₅₀ organoheteryl, C₂₅₋₄₀ organyl, C₂₅₋₄₀ organoheteryl, C₂₅₋₃₀ organyl, C₂₅₋₃₀ organoheteryl, C₃₀₋₁₀₀ organyl, C₃₀₋₁₀₀ organoheteryl, C₃₀₋₈₀ organyl, C₃₀₋₈₀ organoheteryl, C₃₀₋₆₀ organyl, C₃₀₋₆₀ organoheteryl, C₃₀₋₅₀ organyl, C₃₀₋₅₀ organoheteryl, C₃₀₋₄₀ organyl, C₃0-40 organoheteryl, C₃₀₋₃₅ organyl, or C₃₀₋₃₅ organoheteryl. In some embodiments, the TBM is selected from C₆₋₄₀ organyl and C₆₋₄₀ organoheteryl. In some embodiments, the TBM comprises 1 to 15 nitrogen atoms, such as 3 to 10 nitrogen atoms. In some embodiments, the TBM comprises 1 to 10 oxygen atoms, such as 1 to 5 oxygen atoms. In some embodiments, the TBM comprises 1 to 10 halogen atoms, such as 1 to 8 halogen atoms independently selected from fluorine and chlorine. In some embodiments, the TBM comprises at least 15 atoms, such as at least 20 atoms, at least 25 atoms, at least 30 atoms, at least 35 atoms, at least 40 atoms, at least 45 atoms, at least 50 atoms, at least 55 atoms, at least 60 atoms, at least 65 atoms, at least 70 atoms, at least 75 atoms, at least 80 atoms, at least 85 atoms, at least 90 atoms, at least 95 atoms, at least 100 atoms, at least 105 atoms, at least 110 atoms, at least 120 atoms, at least 125 atoms, or at least 130 atoms. In some embodiments, the TBM comprises 10 to 60 carbon atoms, 10 to 80 hydrogen atoms, 0 to 5 chlorine atoms, 0 to 10 fluorine atoms, 1 to 15 nitrogen atoms, 1 to 15 oxygen atoms, and 0 to 3 sulfur atoms. In some embodiments, the TBM consists of 10 to 60 carbon atoms, 10 to 80 hydrogen atoms, 0 to 5 chlorine atoms, 0 to 10 fluorine atoms, 1 to 15 nitrogen atoms, 1 to 15 oxygen atoms, and 0 to 3 sulfur atoms. In some embodiments, the TBM comprises 10 to 40 carbon atoms, 10 to 50 hydrogen atoms, 0 to 3 chlorine atoms, 0 to 6 fluorine atoms, 1 to 12 nitrogen atoms, 1 to 10 oxygen atoms, and 0 to 3 sulfur atoms. In some embodiments, the TBM consists 10 to 40 carbon atoms, 10 to 50 hydrogen atoms, 0 to 3 chlorine atoms, 0 to 6 fluorine atoms, 1 to 12 nitrogen atoms, 1 to 10 oxygen atoms, and 0 to 3 sulfur atoms. In some embodiments, the molecular weight of the TBM is at least 50 Da, such as at least 60 Da, at least 70 Da, at least 80 Da, at least 90 Da, at least 100 Da, at least 125 Da, at least 150 Da, at least 175 Da, at least 200 Da, at least 225 Da, at least 250 Da, at least 300 Da, at least 350 Da, at least 400 Da, at least 450 Da, at least 500 Da, at least 550 Da, at least 600 Da, at least 650 Da, at least 700 Da, at least 750 Da, at least 800 Da, at least 850 Da, at least 900 Da, at least 950 Da, or at least 1000 Da. In some embodiments, the molecular weight of the TBM is less than 1000 Da, less than 150 Da, less than 175 Da, less than 200 Da, less than 225 Da, less than 250 Da, less than 300 Da, less than 350 Da, less than 400 Da, less than 450 Da, less than 500 Da, less than 550 Da, less than 600 Da, less than 650 Da, less than 700 Da, less than 750 Da, less than 800 Da, less than 850 Da, less than 900 Da, or less than 950 Da. In some embodiments, the molecular weight of the TBM is 50 to 1000 Da, such as 50 to 950 Da, 50 to 900 Da, 50 to 850 Da, 50 to 800 Da, 50 to 750 Da, 50 to 700 Da, 50 to 650 Da, 50 to 600 Da, 50 to 550 Da, 50 to 500 Da, 50 to 450 Da, 50 to 400 Da, 50 to 350 Da, 50 to 300 Da, 50 to 250 Da, or 50 to 200 Da. In some embodiments, the molecular weight of the TBM is 100 to 1000 Da, such as 100 to 950 Da, 100 to 900 Da, 100 to 850 Da, 100 to 800 Da, 100 to 750 Da, 100 to 700 Da, 100 to 650 Da, 100 to 600 D a, 100 to 550 Da, 100 to 500 Da, 100 to 450 Da, 100 to 400 Da, 100 to 350 Da, 100 to 300 Da, 100 to 250 Da, or 100 to 200 Da. In some embodiments, the molecular weight of the TBM is 150 to 1000 Da, such as 150 to 950 Da, 150 to 900 Da, 150 to 850 Da, 150 to 800 Da, 150 to 750 Da, 150 to 700 Da, 150 to 650 Da, 150 to 600 Da, 150 to 550 Da, 150 to 500 Da, 150 to 450 Da, 150 to 400 Da, 150 to 350 Da, 150 to 300 Da, 150 to 250 Da, or 150 to 200 Da. In some embodiments, the molecular weight of the TBM is 200 to 1000 D a, such as 200 to 950 Da, 200 to 900 Da, 200 to 850 Da, 200 to 800 Da, 200 to 750 Da, 200 to 700 Da, 200 to 650 Da, 200 to 600 Da, 200 to 550 Da, 200 to 500 Da, 200 to 450 Da, 200 to 400 Da, 200 to 350 Da, 200 to 300 Da, or 200 to 250 Da. In some embodiments, the molecular weight of the TBM is 250 to 1000 Da, such as 250 to 950 Da, 250 to 900 Da, 250 to 850 Da, 250 to 800 Da, 250 to 750 Da, 250 to 700 Da, 250 to 650 Da, 250 to 600 Da, 250 to 550 Da, 250 to 500 Da, 250 to 450 Da, 250 to 400 Da, 250 to 350 Da, or 250 to 300 Da. In some embodiments, the molecular weight of the TBM is 300 to 1000 Da, such as 300 to 950 Da, 300 to 900 Da, 300 to 850 Da, 300 to 800 Da, 300 to 750 Da, 300 to 700 Da, 300 to 650 Da, 300 to 600 Da, 300 to 550 Da, 300 to 500 Da, 300 to 450 Da, 300 to 400 Da, or 300 to 350 Da. In some embodiments, the molecular weight of the TBM is 350 to 1000 Da, such as 350 to 950 Da, 350 to 900 Da, 350 to 850 Da, 350 to 800 Da, 350 to 750 Da, 350 to 700 Da, 350 to 650 Da, 350 to 600 Da, 350 to 550 Da, 350 to 500 Da, 350 to 450 Da, or 350 to 400 Da. In some embodiments, the molecular weight of the TBM is 400 to 1000 Da, such as 400 to 950 Da, 400 to 900 Da, 400 to 850 Da, 400 to 800 Da, 400 to 750 Da, 400 to 700 Da, 400 to 650 Da, 400 to 600 Da, 400 to 550 Da, 400 to 500 Da, or 400 to 450 Da. In some embodiments, the molecular weight of the TBM is 450 to 1000 Da, such as 450 to 950 Da, 450 to 900 Da, 450 to 850 Da, 450 to 800 Da, 450 to 750 Da, 450 to 700 Da, 450 to 650 Da, 450 to 600 Da, 450 to 550 Da, or 450 to 500 Da. In some embodiments, the molecular weight of the TBM is 500 to 1000 Da, such as 500 to 950 Da, 500 to 900 Da, 500 to 850 Da, 500 to 800 Da, 500 to 750 Da, 500 to 700 Da, 500 to 650 Da, 500 to 600 Da, or 500 to 550 Da. In some embodiments, the molecular weight of the TBM is 550 to 1000 Da, such as 550 to 950 Da, 550 to 900 Da, 550 to 850 Da, 550 to 800 Da, 550 to 750 Da, 550 to 700 Da, 550 to 650 Da, or 550 to 600 Da. In some embodiments, the molecular weight of the TBM is 600 to 1000 Da, such as 600 to 950 Da, 600 to 900 Da, 600 to 850 Da, 600 to 800 Da, 600 to 750 Da, 600 to 700 Da, or 600 to 650 Da.

In some embodiments, for a compound or modified polypeptide described herein, such as a compound of Formula (I′), (I), (II), (III), or (IV) or a modified polypeptide of Formula (V′) or (V), the TBM is selected from C₁₋₆ alkyl, C₂₋₆ alkenyl, C₂₋₆ alkynyl, 2—to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), —S(═O)(═NR²²)N(R²²)(R²³), and —OCH₂C(O)OR²²; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), ═NR²², ═C(R²¹)₂, —C(O)OR²², —OC(O)N(R²²)(R²³), —N(R²²)C(O)N(R²²)(R²³), —N(R²²)C(O)OR²², —N(R²²)S(O)₂R²², —C(O)R²², —S(O)R²², —OC(O)R²², —C(O)N(R²²)(R²³), —C(O)C(O)N(R²²)(R²³), —N(R²²)C(O)R²², —S(O)₂R²², —S(O)(NR²²)R²², —S(O)₂N(R²²)(R²³), —S(═O)(═NR²²)N(R²²)(R²³), and R³⁰;
R²¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more R³⁰; or two R²¹ are taken together with the carbon atom to which they are attached to form C₃₋₁₂ carbocycle or 3- to 12-membered heterocycle, each of which is optionally substituted with one or more R³⁰;
R²² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more R³⁰;
R²³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more R³⁰; or R²² and R²³ attached to the same nitrogen atom form 3- to 10 membered heterocycle optionally substituted with one or more R³⁰;
R³⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), —S(═O)(═NR³²)N(R³²)(R³³), and —OCH₂C(O)OR³²; wherein two R³⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR³², —SR³², —N(R³²)(R³³), ═NR³², ═C(R³¹)₂, —C(O)OR³², —OC(O)N(R³²)(R³³), —N(R³²)C(O)N(R³²)(R³³), —N(R³²)C(O)OR³², —N(R³²)S(O)₂R³², —C(O)R³², —S(O)R³², —OC(O)R³², —C(O)N(R³²)(R³³), —C(O)C(O)N(R³²)(R³³), —N(R³²)C(O)R³², —S(O)₂R³², —S(O)(NR³²)R³², —S(O)₂N(R³²)(R³³), and —S(═O)(═NR³²)N(R³²)(R³³);
R³¹ is independently selected at each occurrence from hydrogen, halogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or two R³¹ are taken together with the carbon atom to which they are attached to form C₃₋₁₂ carbocycle or 3- to 12-membered heterocycle, each of which is optionally substituted with one or more substituents selected from halogen, C₁₋₆ alkyl, C₁₋₆ haloalkyl, —OH, —OCH₃, —NH₂, —NHCH₃, and —N(CH₃)₂;
R³² is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; and
R³³ is independently selected at each occurrence from hydrogen, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle), wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one or more substituents selected from halogen, C₁₋₆ alkyl, C₁₋₆ haloalkyl, —OH, —OCH₃, —NH₂, —NHCH₃, and —N(CH₃)₂; or R³² and R³³ attached to the same nitrogen atom form 3- to 10 membered heterocycle optionally substituted with one or more substituents selected from halogen, C₁₋₆ alkyl, C₁₋₆ haloalkyl, —OH, —OCH₃, —NH₂, —NHCH₃, and —N(CH₃)₂.

In some embodiments, for a compound or modified polypeptide described herein, such as a compound of Formula (I′), (I), (II), (III), or (IV) or a modified polypeptide of Formula (V′) or (V), the TBM is not:

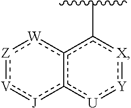

wherein:
W is N, C(R¹⁷), N(R^{17b}), C(R¹⁷)₂, C(O), S(O), or S(O)₂;
Z is N, C(R¹⁷), N(R^{17b}), C(R¹⁷)₂, C(O), S(O), or S(O)₂; wherein W and Z are not both selected from C(O), S(O), and S(O)₂;
V and J are each independently selected from N, C(R¹⁹), C(R¹⁷), N(R¹⁹), N(R^{17b}), C(R¹⁹)(R¹⁷), and C(R¹⁷)₂; wherein exactly one of V and J is C(R¹⁹), N(R¹⁹), or C(R¹⁹)(R¹⁷)
U is N, C(R¹⁷), N(R^{17b}), C(R¹⁷)₂, S(O), S(O)₂, or C(O);
Y is N, C(R¹⁸), N(R^{17b}), C(R¹⁸)(R¹⁷), S(O), S(O)₂, or C(O);
X is N, C(R¹⁷), N(R^{17b}), or C(R¹⁷)₂;
R¹⁷ is independently selected at each occurrence from hydrogen, halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R⁴⁰;
R^{17b} is independently selected at each occurrence from hydrogen, —CN, C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), —OR¹², —SR¹², —C(O)OR¹², —OC(O)N(R¹²)(R¹³), —C(O)R¹², —S(O)R¹², —OC(O)R¹², —C(O)N(R¹²)(R¹³), —C(O)C(O)N(R¹²)(R¹³), —S(O)₂R¹², —S(O)(NR¹²)R¹², —S(O)₂N(R¹²)(R¹³), and —S(═O)(═NR¹²)N(R¹²)(R¹³), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R⁴⁰;
R¹⁸ is selected from halogen, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR¹², —SR¹², —N(R¹²)(R¹³), ═NR¹², ═C(R¹⁴)₂, —C(O)OR¹², —OC(O)N(R¹²)(R¹³), —N(R¹²)C(O)N(R¹²)(R¹³), —N(R¹²)C(O)OR¹², —N(R¹²)S(O)₂R¹², —C(O)R¹², —S(O)R¹², —OC(O)R¹², —C(O)N(R¹²)(R¹³), —C(O)C(O)N(R¹²)(R¹³), —N(R¹²)C(O)R¹², —S(O)₂R¹², —S(O)(NR¹²)R¹², —S(O)₂N(R¹²)(R¹³), —S(═O)(═NR¹²)N(R¹²)(R¹³), and —OCH₂C(O)OR¹², wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), and -(2- to 6-membered heteroalkyl)-(3- to 12-membered heterocycle) are optionally substituted with one, two, or three R⁴⁰;
R¹⁹ is selected from C₆₋₁₀ aryl and 5- to 10-membered heteroaryl, each of which is optionally substituted with one, two, three, four, or five R⁴⁰;
R¹² is independently selected at each occurrence from hydrogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), wherein C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle) are optionally substituted with one, two, or three R⁴⁰;
R¹³ is independently selected at each occurrence from hydrogen, C₁₋₆ alkyl, and C₁₋₆ haloalkyl; or R¹² and R¹³ attached to the same nitrogen atom form 3- to 10-membered heterocycle optionally substituted with one, two, or three R⁴⁰;
R¹⁴ is independently selected at each occurrence from hydrogen, halogen, C₁₋₆ alkyl, C₂₋₆ alkenyl, C₂₋₆ alkynyl, —C₀₋₆ alkyl-(C₃₋₁₂ carbocycle), and —C₀₋₆ alkyl-(3- to 12-membered heterocycle), or two R¹⁴ are taken together with the carbon atom to which they are attached to form C₃₋₁₂ carbocycle or 3- to 12-membered heterocycle, wherein C₁₋₆ alkyl, C₂₋₆ alkenyl, C₂₋₆ alkynyl, —C₀₋₆ alkyl-(C₃₋₁₂ carbocycle), —C₀₋₆ alkyl-(3- to 12-membered heterocycle), C₃₋₁₂ carbocycle, and 3- to 12-membered heterocycle are optionally substituted with one, two, or three R⁴⁰;
R⁴⁰ is independently selected at each occurrence from halogen, oxo, —CN, C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), —OR⁴², —SR⁴², —N(R⁴²)(R⁴³), ═NR⁴², ═C(R⁴¹)₂, —C(O)OR⁴², —OC(O)N(R⁴²)(R⁴³), —N(R⁴²)C(O)N(R⁴²)(R⁴³), —N(R⁴²)C(O)OR⁴², —N(R⁴²)S(O)₂R⁴², —C(O)R⁴², —S(O)R⁴², —OC(O)R⁴², —C(O)N(R⁴²)(R⁴³), —C(O)C(O)N(R⁴²)(R⁴³), —N(R⁴²)C(O)R⁴², —S(O)₂R⁴², —S(O)(NR⁴²)R⁴², —S(O)₂N(R⁴²)(R⁴³), —S(═O)(═NR⁴²)N(R⁴²)(R⁴³), and —OCH₂C(O)OR⁴²; wherein two R⁴⁰ attached to the same or adjacent atoms optionally join to form C₃₋₁₂ carbocycle or 3- to 12-membered heterocycle; wherein C₁₋₆ alkyl, C₂₋₆ alkenyl, C₂₋₆ alkynyl, 2- to 6-membered heteroalkyl, 2- to 6-membered heteroalkenyl, 2- to 6-membered heteroalkynyl, —C₀₋₆ alkyl-(C₃₋₁₂ carbocycle), -(2- to 6-membered heteroalkyl)-(C₃₋₁₂ carbocycle), —C₀₋₆ alkyl-(3- to 12-membered heterocycle), -(2- to 6-membered heteroalkyl)-(3- to 12-membered heterocycle), C₃₋₁₂ carbocycle, and 3- to 12-membered heterocycle are optionally substituted with one or more substituents independently selected from halogen, oxo, —CN, C₁₋₆ alkyl, C₁₋₆ haloalkyl, C₁₋₆ alkoxy, C₁₋₆ haloalkoxy, —OR⁴², —SR⁴², —N(R⁴²)(R⁴³), ═NR⁴², ═C(R⁴¹)₂, —C(O)OR⁴², —OC(O)N(R⁴²)(R⁴³), —N(R⁴²)C(O)N(R⁴²)(R⁴³), —N(R⁴²)C(O)OR⁴², —N(R⁴²)S(O)₂R⁴², —C(O)R⁴², —S(O)R⁴², —OC(O)R⁴², —C(O)N(R⁴²)(R⁴³), —C(O)C(O)N(R⁴²)(R⁴³), —N(R⁴²)C(O)R⁴², —S(O)₂R⁴², —S(O)(NR⁴²)R⁴², —S(O)₂N(R⁴²)(R⁴³), and —S(═O)(═NR⁴²)N(R⁴²)(R⁴³);
R⁴¹ is independently selected at each occurrence from hydrogen, halogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle), or two R⁴¹ are taken together with the carbon atom to which they are attached to form C₃₋₁₂ carbocycle or 3- to 12-membered heterocycle, each of which is optionally substituted with one, two, or three substituents independently selected from halogen, C₁₋₃ alkyl, C₁₋₃ haloalkyl, and —OH;
R⁴² is independently selected at each occurrence from hydrogen, C₁₋₆ alkyl, C₁₋₆ haloalkyl, —C₀₋₆ alkyl-(C₃₋₁₂ carbocycle), and —C₀₋₆ alkyl-(3- to 12-membered heterocycle);
R⁴³ is independently selected at each occurrence from hydrogen and C₁₋₆ alkyl; or R⁴² and R⁴³ attached to the same nitrogen atom form 3- to 10 membered heterocycle; and
[symbol] indicates a single or double bond such that all valences are satisfied.

In certain aspects, the present disclosure provides a method of modifying a non-Ras Target Protein, comprising contacting the Target Protein with an effective amount of a compound described herein, such as a compound of Formula (I′), (I), (II), (III), or (IV), or a pharmaceutically acceptable salt or solvate thereof. In some embodiments, the present disclosure provides a covalently modified amino acid residue in a non-Ras polypeptide, wherein the non-Ras polypeptide is selected from a non-Ras Target Protein described herein.

A Target Protein described herein may be selected from an oxidoreductase, a transferase, a hydrolase, a lyase, an isomerase, a ligase, and a translocase. In some embodiments, the Target Protein is an alpha protein, a beta protein, an alpha and beta protein (a/b), an alpha and beta protein (a+b), a multi-domain protein, a membrane protein, a cell surface protein, a cytosolic protein, a nuclear protein, a secreted protein, a coiled-coil protein, a peptide, or a small protein. In some embodiments, the Target Protein is an enzyme. In some embodiments, the Target Protein is an oxidoreductase (EC 1), such as an oxidoreductase having a classification code selected from EC 1.1, EC 1.2, EC 1.3, EC 1.4, EC 1.5, EC 1.6, EC 1.7, EC 1.8, EC 1.9, EC 1.10, EC 1.11, EC 1.12, EC 1.13, EC 1.14, EC 1.15, EC 1.16, EC 1.17, EC 1.18, EC 1.19, EC 1.20, EC 1.21, EC 1.22, EC 1.23, EC 1.97, EC 1.98, and EC 1.99. In some embodiments, the Target Protein is a transferase (EC 2), such as a transferase having a classification code selected from EC 2.1, EC 2.2, EC 2.3, EC 2.4, EC 2.5, EC 2.6, EC 2.7, EC 2.8, EC 2.9, and EC 2.10. In some embodiments, the Target Protein is a hydrolase (EC 3), such as a hydrolase having a classification code selected from EC 3.1, EC 3.2, EC 3.3, EC 3.4, EC 3.5, EC 3.6, EC 3.7, EC 3.8, EC 3.9, EC 3.10, EC 3.11, EC 3.12, and EC 3.13. In some embodiments, the Target Protein is a lyase (EC 4), such as a lyase having a classification code selected from EC 4.1, EC 4.2, EC 4.3, EC 4.4, EC 4.5, EC 4.6, EC 4.7, EC 4.8, EC 4.98, and EC 4.99. In some embodiments, the Target Protein is an isomerase (EC 5), such as an isomerase having a classification code selected from EC 5.1, EC 5.2, EC 5.3, EC 5.4, EC 5.5, EC 5.6, and EC 5.99. In some embodiments, the Target Protein is a ligase (EC 6), such as a ligase having a classification code selected from EC 6.1, EC 6.2, EC 6.3, EC 6.4, EC 6.5, EC 6.6, and EC 6.7. In some embodiments, the Target Protein is a translocase (EC 7), such as a translocase having a classification code selected from EC 7.1, EC 7.2, EC 7.3, EC 7.4, EC 7.5, and EC 7.6. In some embodiments, the Target Protein is a phosphotransferase, such as a kinase. In some embodiments, the kinase is selected from a tyrosine kinase, a receptor tyrosine kinase, a serine/threonine-specific kinase, a histidine kinase, and a dual-specificity kinase (e.g., a kinase that can act as both a tyrosine kinase and a serine/threonine kinase). In some embodiments, the Target Protein is a GPCR, a kinase, a protease, an ion channel, a nuclear receptor, or a transporter. In some embodiments, the Target Protein is a GPCR selected from an acetylcholine receptor, an adenosine receptor, an adrenergic receptor, a cannabinoid receptor, a dopamine receptor, a neurokinin receptor, a serotonin receptor, and a vasopressin and oxytocin receptor. In some embodiments, the Target Protein is a kinase in a family selected from AKT, PDKI, PKA, PKCa, PCKS, PIKK, CANM2, MAPKAPK, MSKb, RSKb, ERK, p38, MST, STE7, IRAK, RAF, STKR Type 1, Abl, Eph, InsR, JakB, PDGFR, Src, Syk, Tec, and VEGFR In some embodiments, the Target Protein is a kinase in a family selected from AKT, PDKI, AMPK, CHKI, PIM, RAD53, RSKb, CKi, AUR, STE7, RAF, Abl, EGFR, Eph, FGFR, InsR, PDGFR, Src, Tec, Tie, and VEGFR In some embodiments, the Target Protein is a kinase in a family selected from AKT, PKA, PIKK, RAD53, ERK, GSK, AUR, Meta, PAKB, STE11, IRAK, RAF, DDR, EGFR, Eph, JakB, and Met.

A Target Protein described herein may be selected from 1-acylglycerol-3-phosphate O-acyltransferase beta, 11-beta-hydroxysteroid dehydrogenase 1, 11-beta-hydroxysteroid dehydrogenase 2, 14-3-3 protein sigma, 15-hydroxyprostaglandin dehydrogenase[NAD+], 17-beta-hydroxysteroid dehydrogenase 14, 2′-deoxynucleoside 5′-phosphate N-hydrolase 1, 2-acylglycerol O-acyltransferase 2, 2-oxoglutarate receptor 1, 25-hydroxyvitamin D-1 alpha hydroxylase, mitochondrial, 3-beta-hydroxysteroid dehydrogenase/delta 5-->4-isomerase type 1, 3-beta-hydroxysteroid-delta(8), delta(7)-isomerase, 3-hydroxyanthranilate 3,4-dioxygenase, 3-keto-steroid reductase, 3-phosphoinositide dependent protein kinase-1, 4-hydroxyphenylpyruvate dioxygenase, 40S ribosomal protein S27, 5′(3′)-deoxyribonucleotidase, cytosolic type, 5′(3′)-deoxyribonucleotidase, mitochondrial, 5′-nucleotidase, 5-lipoxygenase activating protein, 6-O-methylguanine-DNA methyltransferase, 6-phosphofructo-2-kinase/fructose-2,6-bisphosphatase 1, 6-phosphofructo-2-kinase/fructose-2,6-bisphosphatase 2, 6-phosphofructo-2-kinase/fructose-2,6-bisphosphatase 3, 7,8-dihydro-8-oxoguanine triphosphatase, 7-dehydrocholesterol reductase, A disintegrin and metalloproteinase with thrombospondin motifs 13, ADAM10, ADAM17, ADAMTS1, ADAMTS4, ADAMTS5, AICAR transformylase, ALK tyrosine kinase receptor, AMP deaminase 1, AMP deaminase 3, AMP-activated protein kinase, alpha-1 subunit, AMP-activated protein kinase, alpha-2 subunit, AMP-activated protein kinase, beta-1 subunit, ATP-binding cassette sub-family G member 2, ATP-citrate synthase, ATP-dependent RNA helicase DDX3X, ATP-sensitive inward rectifier potassium channel 1, ATPase family AAA domain-containing protein 2, ATPase family AAA domain-containing protein 2B, Acetyl-CoA carboxylase 1, Acetyl-CoA carboxylase 2, Acetyl-coenzyme A transporter 1, Acetylcholine receptor protein alpha chain, Acetylcholinesterase, Acid ceramidase, Acidic mammalian chitinase, Acrosin, Activin receptor type-1, Activin receptor type-1B, Activin receptor type-2A, Activin receptor type-2B, Acyl coenzyme A:cholesterol acyltransferase, Acyl coenzyme A:cholesterol acyltransferase 1, Acyl coenzyme A:cholesterol acyltransferase 2, Acyl-CoA desaturase, Acyl-protein thioesterase 1, Acyl-protein thioesterase 2, Adaptor-associated kinase, Adenosine A1 receptor, Adenosine A2a receptor, Adenosine A2b receptor, Adenosine A3 receptor, Adenosine deaminase, Adenosine kinase, Adenosylhomocysteinase, Adenosylhomocysteinase 2, Adenylate cyclase type 10, Adiponectin receptor protein 1, Adiponectin receptor protein 2, Advanced glycosylation end product-specific receptor, Alcohol dehydrogenase class III, Aldehyde dehydrogenase, Aldehyde dehydrogenase lAl, Aldehyde dehydrogenase dimeric NADP-preferring, Aldehyde oxidase, Aldehyde reductase, Aldo-keto reductase family 1 member B10, Aldo-keto reductase family 1 member C1, Aldo-keto reductase family 1 member C2, Aldo-keto reductase family 1 member C4, Aldo-keto-reductase family 1 member C3, Aldose reductase, Alkaline ceramidase 2, Alkaline phosphatase placental-like, Alkaline phosphatase, tissue-nonspecific isozyme, Alpha enolase, Alpha-(1,3)-fucosyltransferase 7, Alpha-1,3-mannosyl-glycoprotein 2-beta-N-acetylglucosaminyltransferase, Alpha-1,6-mannosyl-glycoprotein 2-beta-N-acetylglucosaminyltransferase, Alpha-Ia adrenergic receptor, Alpha-lb adrenergic receptor, Alpha-ld adrenergic receptor, Alpha-2a adrenergic receptor, Alpha-2b adrenergic receptor, Alpha-2c adrenergic receptor, Alpha-L-fucosidase I, Alpha-crystallin B chain, Alpha-galactosidase A, Alpha-ketoglutarate-dependent dioxygenase FTO, Alpha-ketoglutarate-dependent dioxygenase alkB homolog 3, Alpha-mannosidase 2A1, Alpha-synuclein, Amiloride-sensitive cation channel 2, neuronal, Amiloride-sensitive cation channel 3, Amine oxidase, copper containing, Aminopeptidase B, Aminopeptidase N, Anandamide amidohydrolase, Androgen Receptor, Angiotensin II type 2 (AT-2) receptor, Angiotensin-converting enzyme, Angiotensin-converting enzyme 2, Ankyrin repeat and protein kinase domain-containing protein 1, Anoctamin-1, Apelin receptor, Apoptosis regulator BAX, Apoptosis regulator Bcl-2, Apoptosis regulator Bcl-W, Apoptosis regulator Bcl-X, Appetite-regulating hormone, Arachidonate 12-lipoxygenase, Arachidonate 15-lipoxygenase, Arachidonate 15-lipoxygenase, type II, Arachidonate 5-lipoxygenase, Arginase-1, Aryl hydrocarbon receptor, Arylacetamide deacetylase, Arylamine N-acetyltransferase 1, Asparagine synthetase, Autotaxin, Axin-2, BMP-2-inducible protein kinase, BR serine/threonine-protein kinase 1, BR serine/threonine-protein kinase 2, Baculoviral IAP repeat-containing protein 2, Baculoviral IAP repeat-containing protein 3, Baculoviral IAP repeat-containing protein 5, Baculoviral IAP repeat-containing protein 8, Basic fibroblast growth factor, Bcl-2-like protein 10, Bcl-2-related protein A1, Bc12-antagonist of cell death (BAD), Beta amyloid A4 protein, Beta secretase 2, Beta-1 adrenergic receptor, Beta-1,4-galactosyltransferase 1, Beta-1,4-mannosyl-glycoprotein 4-beta-N-acetylglucosaminyltransferase, Beta-2 adrenergic receptor, Beta-3 adrenergic receptor, Beta-chymotrypsin, Beta-galactosidase, Beta-galactoside alpha-2,6-sialyltransferase 1, Beta-glucocerebrosidase, Beta-glucosidase, Beta-glucosidase cytosolic, Beta-glucuronidase, Beta-hexosaminidase subunit alpha, Beta-hexosaminidase subunit beta, Beta-secretase 1, Betaine transporter, Betaine--homocysteine S-methyltransferase 1, Bifunctional methylenetetrahydrofolate dehydrogenase/cyclohydrolase, mitochondrial, Bifunctional protein NCOAT, Bile acid receptor FXR, Bile acid transporter, Bile salt export pump, Biotin--protein ligase, Bloom syndrome protein, Bombesin receptor subtype-3, Bone morphogenetic protein 1, Bone morphogenetic protein receptor type-iA, Bone morphogenetic protein receptor type-1B, Bone morphogenetic protein receptor type-2, Bradykinin B1 receptor, Bradykinin B2 receptor, Branched-chain-amino-acid aminotransferase, mitochondrial, Branched-chain-amino-acid transferase, Breakpoint cluster region protein, Breast cancer type 1 susceptibility protein, Bromodomain adjacent to zinc finger domain protein 2A, Bromodomain adjacent to zinc finger domain protein 2B, Bromodomain and PHD finger-containing protein 3, Bromodomain and WD repeat-containing protein 1, Bromodomain testis-specific protein, Bromodomain-containing protein 1, Bromodomain-containing protein 2, Bromodomain-containing protein 3, Bromodomain-containing protein 4, Bromodomain-containing protein 7, Bromodomain-containing protein 8, Bromodomain-containing protein 9, Butyrylcholinesterase, C—C chemokine receptor type 1, C—C chemokine receptor type 2, C—C chemokine receptor type 3, C—C chemokine receptor type 4, C—C chemokine receptor type 5, C—C chemokine receptor type 6, C—C chemokine receptor type 8, C—C chemokine receptor type 9, C—C motif chemokine 2, C—X—C chemokine receptor type 3, C—X—C chemokine receptor type 4, C—X—C chemokine receptor type 7, C—X3—C chemokine receptor 1, C-terminal-binding protein 2, C3a anaphylatoxin chemotactic receptor, C5a anaphylatoxin chemotactic receptor, CCR4-NOT transcription complex subunit 7, CD209 antigen, CD22, CD44 antigen, CD81 antigen, CDC45-related protein, CDGSH iron-sulfur domain-containing protein 1, CDK-interacting protein 1, CMP-N-acetylneuraminate-beta-1,4-galactoside alpha-2,3-sialyltransferase, CMP-N-acetylneuraminate-beta-galactosamide-alpha-2,3-sialyltransferase 1, CREB-binding protein, CaM kinase I alpha, CaM kinase I delta, CaM kinase I gamma, CaM kinase II alpha, CaM kinase II beta, CaM kinase II delta, CaM kinase II gamma, CaM kinase IV, CaM-kinase kinase alpha, CaM-kinase kinase beta, Calcitonin gene-related peptide 1, Calcitonin gene-related peptide type 1 receptor, Calcitonin receptor, Calcium release-activated calcium channel protein 1, Calcium sensing receptor, Calcium-activated potassium channel subunit alpha-1, Calmodulin, Calpain 1, Calpain 2, Canalicular multispecific organic anion transporter 1, Cannabinoid CBi receptor, Cannabinoid CB2 receptor, Carbonic anhydrase I, Carbonic anhydrase II, Carbonic anhydrase III, Carbonic anhydrase IV, Carbonic anhydrase IX, Carbonic anhydrase VA, Carbonic anhydrase VB, Carbonic anhydrase VI, Carbonic anhydrase VII, Carbonic anhydrase XII, Carbonic anhydrase XIII, Carbonic anhydrase XIV, Carbonyl reductase [NADPH]1, Carboxylesterase 2, Carboxypeptidase Ai, Carboxypeptidase B, Carboxypeptidase B2 isoform A, Carboxypeptidase M, Carboxypeptidase N, catalytic subunit, Carnitine O-palmitoyltransferase 1, liver isoform, Carnitine O-palmitoyltransferase 1, muscle isoform, Carnitine palmitoyltransferase 2, Casein kinase I alpha, Casein kinase I delta, Casein kinase I epsilon, Casein kinase I gamma 1, Casein kinase I gamma 2, Casein kinase I isoform alpha-like, Casein kinase I isoform gamma-3, Casein kinase II alpha, Casein kinase II alpha (prime), Caspase-1, Caspase-10, Caspase-14, Caspase-2, Caspase-3, Caspase-4, Caspase-5, Caspase-6, Caspase-7, Caspase-8, Caspase-9, Cat eye syndrome critical region protein 2, Catechol O-methyltransferase, Catenin beta-1, Cathepsin B, Cathepsin D, Cathepsin E, Cathepsin F, Cathepsin G, Cathepsin H, Cathepsin K, Cathepsin L, Cathepsin L2, Cathepsin S, Cathepsin Z, Cell cycle checkpoint protein RAD1, Cell division cycle 2-like protein kinase 6, Cell division cycle 7-related protein kinase, Cell division protein kinase 8, Cellular tumor antigen p53, Centromere-associated protein E, Ceramide glucosyltransferase, Chaperone activity of bcl complex-like, mitochondrial, Cholecystokinin A receptor, Cholecystokinin B receptor, Cholesteryl ester transfer protein, Choline kinase alpha, Chondroitin sulfate N-acetylgalactosaminyltransferase 1, Chromobox protein homolog 1, Chromobox protein homolog 7, Chromobox protein homolog 8, Chymase, Citron Rho-interacting kinase, Clathrin heavy chain 1, Coagulation factor III, Coagulation factor IX, Coagulation factor V, Coagulation factor VII, Coagulation factor VIII, Coagulation factor X, Coagulation factor XI, Coagulation factor XII, Coagulation factor XIII, Collagen type IV alpha-3-binding protein, Complement Cls, Complement factor B, Complement factor D, Core-binding factor subunit beta, Corticotropin releasing factor receptor 1, Cyclin T1, Cyclin-dependent kinase 1, Cyclin-dependent kinase 13, Cyclin-dependent kinase 2, Cyclin-dependent kinase 3, Cyclin-dependent kinase 4, Cyclin-dependent kinase 5, Cyclin-dependent kinase 6, Cyclin-dependent kinase 7, Cyclin-dependent kinase 9, Cyclin-dependent kinase-like 1, Cyclin-dependent kinase-like 2, Cyclin-dependent kinase-like 3, Cyclin-dependent kinase-like 5, Cyclooxygenase-1, Cyclooxygenase-2, Cyclophilin A, Cyclophilin B, Cystathionine beta-synthase, Cysteinyl leukotriene receptor 1, Cysteinyl leukotriene receptor 2, Cystic fibrosis transmembrane conductance regulator, Cystine/glutamate transporter, Cystinyl aminopeptidase, Cytidine deaminase, Cytochrome P450 1IB1, Cytochrome P450 11B2, Cytochrome P450 17A1, Cytochrome P450 19A1, Cytochrome P450 lAl, Cytochrome P450 1A2, Cytochrome P450 1B1, Cytochrome P450 21, Cytochrome P450 24A1, Cytochrome P450 26A1, Cytochrome P450 26B1, Cytochrome P450 2A13, Cytochrome P450 2A6, Cytochrome P450 2B6, Cytochrome P450 2C19, Cytochrome P450 2C8, Cytochrome P450 2C9, Cytochrome P450 2D6, Cytochrome P450 2E1, Cytochrome P450 2J2, Cytochrome P450 3A4, Cytochrome P450 3A5, Cytochrome P450 3A7, Cytochrome P450 4F2, Cytochrome P450 51, Cytochrome b-245 heavy chain, Cytochrome b-c1 complex subunit 7, Cytochrome c oxidase subunit 2, Cytohesin-2, Cytohesin-3, Cytosolic phospholipase A2, D-amino-acid oxidase, D-aspartate oxidase, DNA (cytosine-5)-methyltransferase 1, DNA (cytosine-5)-methyltransferase 3A, DNA (cytosine-5)-methyltransferase 3B, DNA dC->dU-editing enzyme APOBEC-3G, DNA damage-inducible transcript 3 protein, DNA ligase 1, DNA polymerase alpha subunit, DNA polymerase beta, DNA polymerase delta subunit 1, DNA polymerase eta, DNA polymerase gamma subunit 1, DNA polymerase kappa, DNA polymerase lambda, DNA polymerase nu, DNA repair and recombination protein RAD54-like, DNA repair protein RAD51 homolog 1, DNA topoisomerase I, DNA topoisomerase II alpha, DNA-(apurinic or apyrimidinic site) lyase, DNA-3-methyladenine glycosylase, DNA-dependent protein kinase, DNA-directed DNA/RNA polymerase mu, DNA-directed RNA polymerase I subunit RPAl, Death-associated protein kinase 1, Death-associated protein kinase 2, Death-associated protein kinase 3, Dehydrogenase/reductase SDR family member 9, Delta opioid receptor, Deoxycytidine kinase, Deoxyribonuclease gamma, Deoxyribonuclease-1, Diacylglycerol O-acyltransferase 1, Diacylglycerol O-acyltransferase 2, Diamine oxidase, Dihydrofolate reductase, Dihydroorotate dehydrogenase, Dipeptidyl peptidase I, Dipeptidyl peptidase II, Dipeptidyl peptidase IV, Dipeptidyl peptidase IX, Dipeptidyl peptidase VIII, Discoidin domain-containing receptor 2, Disintegrin and metalloproteinase domain-containing protein 33, Disintegrin and metalloproteinase domain-containing protein 8, Disks large homolog 4, Dopamine D1 receptor, Dopamine D2 receptor, Dopamine D3 receptor, Dopamine D4 receptor, Dopamine D5 receptor, Dopamine beta-hydroxylase, Dopamine transporter, Double-strand break repair protein MRE11A, Dual serine/threonine and tyrosine protein kinase, Dual specificity mitogen-activated protein kinase kinase 1, Dual specificity mitogen-activated protein kinase kinase 2, Dual specificity mitogen-activated protein kinase kinase 3, Dual specificity mitogen-activated protein kinase kinase 4, Dual specificity mitogen-activated protein kinase kinase 5, Dual specificity mitogen-activated protein kinase kinase 6, Dual specificity mitogen-activated protein kinase kinase 7, Dual specificity phosphatase 22, Dual specificity phosphatase Cdc25A, Dual specificity phosphatase Cdc25B, Dual specificity phosphatase Cdc25C, Dual specificity protein kinase CLK2, Dual specificity protein kinase CLK3, Dual specificity protein kinase CLK4, Dual specificity protein kinase TTK, Dual specificity protein phosphatase 1, Dual specificity protein phosphatase 10, Dual specificity protein phosphatase 15, Dual specificity protein phosphatase 2, Dual specificity protein phosphatase 23, Dual specificity protein phosphatase 26, Dual specificity protein phosphatase 3, Dual specificity protein phosphatase 6, Dual specificity protein phosphatase CDC14A, Dual specificity testis-specific protein kinase 1, Dual specificity testis-specific protein kinase 2, Dual specificity tyrosine-phosphorylation-regulated kinase 1B, Dual specificity tyrosine-phosphorylation-regulated kinase 4, Dual specificty protein kinase CLK1, Dual-specificity tyrosine-phosphorylation regulated kinase 1A, Dual-specificity tyrosine-phosphorylation regulated kinase 2, Dual-specificity tyrosine-phosphorylation regulated kinase 3, Dynamin-1, Dynamin-2, E3 SUMO-protein ligase CBX4, E3 ubiquitin-protein ligase NEDD4, E3 ubiquitin-protein ligase TRIM33, EROl-like protein alpha, Ectonucleoside triphosphate diphosphohydrolase 1, Ectonucleoside triphosphate diphosphohydrolase 2, Ectonucleotide pyrophosphatase/phosphodiesterase family member 1, Ectonucleotide pyrophosphatase/phosphodiesterase family member 3, Egl nine homolog 1, Egl nine homolog 3, Elastase 1, Elongation factor 1-alpha 1, Elongation factor 1-alpha 2, Elongation of very long chain fatty acids protein 1, Elongation of very long chain fatty acids protein 3, Elongation of very long chain fatty acids protein 6, Emopamil-binding protein-like, Endo-beta-N-acetylglucosaminidase, Endoplasmic reticulum aminopeptidase 1, Endoplasmic reticulum aminopeptidase 2, Endoplasmic reticulum mannosyl-oligosaccharide 1,2-alpha-mannosidase, Endoplasmin, Endothelial PAS domain-containing protein 1, Endothelial lipase, Endothelin receptor ET-A, Endothelin receptor ET-B, Endothelin-converting enzyme 1, Enteropeptidase, Ephrin type-A receptor 1, Ephrin type-A receptor 2, Ephrin type-A receptor 3, Ephrin type-A receptor 4, Ephrin type-A receptor 5, Ephrin type-A receptor 6, Ephrin type-A receptor 7, Ephrin type-A receptor 8, Ephrin type-B receptor 1, Ephrin type-B receptor 2, Ephrin type-B receptor 3, Ephrin type-B receptor 4, Ephrin type-B receptor 6, Epidermal growth factor receptor erbB1, Epithelial discoidin domain-containing receptor 1, Epoxide hydratase, Epoxide hydrolase 1, Epsin-1, Equilibrative nucleoside transporter 1, Estradiol 17-beta-dehydrogenase 1, Estradiol 17-beta-dehydrogenase 2, Estradiol 17-beta-dehydrogenase 3, Estrogen receptor alpha, Estrogen receptor beta, Estrogen-related receptor alpha, Estrogen-related receptor beta, Estrogen-related receptor gamma, Eukaryotic initiation factor 4A-I, Eukaryotic translation initation factor, Eukaryotic translation initiation factor 2-alpha kinase 1, Eukaryotic translation initiation factor 2-alpha kinase 3, Eukaryotic translation initiation factor 2-alpha kinase 4, Excitatory amino acid transporter 1, Excitatory amino acid transporter 2, Excitatory amino acid transporter 3, Exportin-1, Ezrin, FK506 binding protein 4, FK506-binding protein 1A, Farnesyl diphosphate synthase, Fatty acid binding protein adipocyte, Fatty acid binding protein epidermal, Fatty acid binding protein intestinal, Fatty acid binding protein muscle, Fatty acid desaturase 1, Fatty acid synthase, Fatty acid transport protein 2, Fatty acid transport protein 4, Fatty acid-binding protein, liver, Fibroblast activation protein alpha, Fibroblast growth factor receptor 1, Fibroblast growth factor receptor 2, Fibroblast growth factor receptor 3, Fibroblast growth factor receptor 4, Flap endonuclease 1, Focal adhesion kinase 1, Folate receptor alpha, Folate receptor beta, Folate transporter 1, Follicle stimulating hormone receptor, Formyl peptide receptor 1, Free fatty acid receptor 1, Free fatty acid receptor 2, Fructose-1,6-bisphosphatase, Fucosyltransferase 4, Fucosyltransferase 5, Fucosyltransferase 6, Fucosyltransferase 9, Furin, G protein-coupled receptor 44, G protein-coupled receptor kinase 4, G protein-coupled receptor kinase 5, G protein-coupled receptor kinase 7, G-protein coupled bile acid receptor 1, G-protein coupled estrogen receptor 1, G-protein coupled receptor 120, G-protein coupled receptor 183, G-protein coupled receptor 35, G-protein coupled receptor 39, G-protein coupled receptor 4, G-protein coupled receptor 55, G-protein coupled receptor 81, G-protein coupled receptor 84, G-protein coupled receptor kinase 2, GABA receptor alpha-1 subunit, GABA receptor alpha-2 subunit, GABA receptor alpha-3 subunit, GABA receptor alpha-4 subunit, GABA receptor alpha-5 subunit, GABA receptor alpha-6 subunit, GABA receptor beta-3 subunit, GABA receptor rho-1 subunit, GABA receptor rho-2 subunit, GABA transporter 1, GABA transporter 2, GABA transporter 3, GABA-B receptor 1, GAR transformylase, Galactokinase, Galanin receptor 1, Galanin receptor 2, Galanin receptor 3, Galectin-1, Galectin-3, Galectin-4, Galectin-7, Galectin-8, Galectin-9, Gamma-amino-N-butyrate transaminase, Gamma-butyrobetaine dioxygenase, Gastric inhibitory polypeptide, Gastric inhibitory polypeptide receptor, Gastrin releasing peptide receptor, Geranylgeranyl pyrophosphate synthetase, Ghrelin O-acyltransferase, Ghrelin receptor, Glucagon, Glucagon receptor, Glucagon-like peptide 1 receptor, Glucocorticoid receptor, Glucokinase regulatory protein, Glucose transporter, Glucose-6-phosphate 1-dehydrogenase, Glucose-6-phosphate translocase, Glucose-dependent insulinotropic receptor, Glutamate (NMDA) receptor subunit zeta 1, Glutamate [NMDA]receptor subunit epsilon 2, Glutamate [NMDA]receptor subunit epsilon 4, Glutamate carboxypeptidase II, Glutamate receptor ionotropic kainate 1, Glutamate receptor ionotropic kainate 2, Glutamate receptor ionotropic kainate 3, Glutamate receptor ionotropic kainate 5, Glutamate receptor ionotropic, AMPA 1, Glutamate receptor ionotropic, AMPA 2, Glutamate receptor ionotropic, AMPA 3, Glutamate receptor ionotropic, AMPA 4, Glutaminase kidney isoform, mitochondrial, Glutaminyl-peptide cyclotransferase, Glutaminyl-peptide cyclotransferase-like protein, Glutathione S-transferase A1, Glutathione S-transferase A2, Glutathione S-transferase Mu 1, Glutathione S-transferase Mu 2, Glutathione S-transferase Pi, Glutathione reductase, Glutathione transferase omega 1, Glyceraldehyde-3-phosphate dehydrogenase liver, Glycine receptor subunit alpha-1, Glycine receptor subunit alpha-2, Glycine receptor subunit alpha-3, Glycine transporter 1, Glycine transporter 2, Glycogen synthase kinase-3 alpha, Glycogen synthase kinase-3 beta, Glyoxalase I, Gonadotropin-releasing hormone receptor, Group IID secretory phospholipase A2, Group IIE secretory phospholipase A2, Group IIF secretory phospholipase A2, Group X secretory phospholipase A2, Growth factor receptor-bound protein 2, Growth factor receptor-bound protein 7, Guanine nucleotide-binding protein G(i), alpha-1 subunit, Guanine nucleotide-binding protein G(k), alpha-3 subunit, Guanine nucleotide-binding protein G(o), alpha subunit 1, HERG, HLA-DR antigens-associated invariant chain, H1M74 nicotinic acid GPCR, HMG-CoA reductase, Heat shock cognate 71 kDa protein, Heat shock factor protein 1, Heat shock protein 75 kDa, mitochondrial, Heat shock protein HSP 90-alpha, Heat shock protein HSP 90-beta, Heat shock protein beta-1, Hematopoietic cell protein-tyrosine phosphatase 70Z-PEP, Hematopoietic prostaglandin D synthase, Heme oxygenase 1, Heparanase, Hepatic lipase, Hepatocyte growth factor, Hepatocyte growth factor activator, Hepatocyte growth factor receptor, Hepatocyte nuclear factor 4-alpha, Heterogeneous nuclear ribonucleoprotein Ai, Hexokinase type I, Hexokinase type II, Hexokinase type IV, High affinity immunoglobulin gamma Fc receptor I, High-affinity choline transporter, Histamine H1 receptor, Histamine H2 receptor, Histamine H3 receptor, Histamine H4 receptor, Histidine triad nucleotide-binding protein 1, Histone H1.0, Histone acetyltransferase GCN5, Histone acetyltransferase KAT5, Histone acetyltransferase KAT8, Histone acetyltransferase PCAF, Histone acetyltransferase p300, Histone chaperone ASF1A, Histone deacetylase 1, Histone deacetylase 10, Histone deacetylase 11, Histone deacetylase 2, Histone deacetylase 3, Histone deacetylase 4, Histone deacetylase 5, Histone deacetylase 6, Histone deacetylase 7, Histone deacetylase 8, Histone deacetylase 9, Histone lysine demethylase PHF8, Histone-arginine methyltransferase CARMI, Histone-lysine N-methyltransferase EZHI, Histone-lysine N-methyltransferase EZH2, Histone-lysine N-methyltransferase MLL, Histone-lysine N-methyltransferase SETD2, Histone-lysine N-methyltransferase SETD7, Histone-lysine N-methyltransferase SETDBI, Histone-lysine N-methyltransferase SMYD3, Histone-lysine N-methyltransferase SUV39H1, Histone-lysine N-methyltransferase SUV39H2, Histone-lysine N-methyltransferase SUV420H2, Histone-lysine N-methyltransferase, H3 lysine-79 specific, Histone-lysine N-methyltransferase, H3 lysine-9 specific 3, Histone-lysine N-methyltransferase, H3 lysine-9 specific 5, Homeodomain-interacting protein kinase 1, Homeodomain-interacting protein kinase 2, Homeodomain-interacting protein kinase 3, Homeodomain-interacting protein kinase 4, Hormonally up-regulated neu tumor-associated kinase, Hormone sensitive lipase, Huntingtin, Hydroxyacid oxidase 2, Hydroxycarboxylic acid receptor 2, Hypoxanthine-guanine phosphoribosyltransferase, Hypoxia-inducible factor 1 alpha, Hypoxia-inducible factor 1-alpha inhibitor, Hypoxia-inducible factor prolyl 4-hydroxylase, Hypoxia-inducible factor prolyl hydroxylase 1, Ileal bile acid transporter, Indoleamine 2,3-dioxygenase, Induced myeloid leukemia cell differentiation protein Mcl-1, Inhibitor of apoptosis protein 3, Inhibitor of growth protein 2, Inhibitor of nuclear factor kappa B kinase alpha subunit, Inhibitor of nuclear factor kappa B kinase beta subunit, Inhibitor of nuclear factor kappa B kinase epsilon subunit, Inosine-5′-monophosphate dehydrogenase 1, Inosine-5′-monophosphate dehydrogenase 2, Insulin receptor, Insulin receptor-related protein, Insulin-degrading enzyme, Insulin-like growth factor I receptor, Insulin-like growth factor II receptor, Integrin alpha-1, Integrin alpha-2, Integrin alpha-4, Integrin alpha-IIb, Intercellular adhesion molecule-1, Interferon-alpha/beta receptor alpha chain, Interferon-induced, double-stranded RNA-activated protein kinase, Interleukin-1 beta, Interleukin-1 receptor-associated kinase 1, Interleukin-1 receptor-associated kinase 3, Interleukin-1 receptor-associated kinase 4, Interleukin-2, Interleukin-2 receptor alpha chain, Interleukin-6 receptor subunit beta, Interleukin-8, Interleukin-8 receptor A, Interleukin-8 receptor B, Intermediate conductance calcium-activated potassium channel protein 4, Intestinal alkaline phosphatase, Inward rectifier potassium channel 4, Isocitrate dehydrogenase[NADP]cytoplasmic, Isoprenylcysteine carboxyl methyltransferase, Kallikrein 1, Kallikrein 14, Kallikrein 2, Kallikrein 4, Kallikrein 5, Kallikrein 6, Kallikrein 7, Kallikrein 8, Kappa opioid receptor, Kelch-like ECH-associated protein 1, Ketohexokinase, Kinesin heavy chain isoform 5A, Kinesin heavy chain isoform 5C, Kinesin-like protein 1, Kinesin-like protein KIF20A, Kinesin-like protein KIF23, Kinesin-like protein KIFC1, Kininogen-1, Krueppel-like factor 10, Kynureninase, Kynurenine 3-monooxygenase, Kynurenine--oxoglutarate transaminase 3, Kynurenine--oxoglutarate transaminase I, Kynurenine/alpha-aminoadipate aminotransferase, mitochondrial, L-lactate dehydrogenase A chain, L-lactate dehydrogenase B chain, L-type amino acid transporter 1, LDL-associated phospholipase A2, LIM domain kinase 1, LIM domain kinase 2, LXR-alpha, LXR-beta, Lactase-phlorizin hydrolase, Laforin, LanC-like protein 2, Lanosterol synthase, Legumain, Lethal(3)malignant brain tumor-like protein 1, Lethal(3)malignant brain tumor-like protein 3, Lethal(3)malignant brain tumor-like protein 4, Leucine aminopeptidase, Leucine-rich repeat serine/threonine-protein kinase 2, Leucyl-tRNA synthetase, Leukocyte adhesion glycoprotein LFA-1 alpha, Leukocyte adhesion molecule-1, Leukocyte common antigen, Leukocyte elastase, Leukocyte proteinase 3, Leukocyte tyrosine kinase receptor, Leukotriene A4 hydrolase, Leukotriene B4 receptor 1, Lipoprotein lipase, Lipoxin A4 receptor, Liver glycogen phosphorylase, Long-chain fatty acid transport protein 1, Low affinity immunoglobulin gamma Fc region receptor II-a, Low affinity immunoglobulin gamma Fc region receptor III-B, Low affinity neurotrophin receptor p75NTR, Low affinity sodium-glucose cotransporter, Low molecular weight phosphotyrosine protein phosphatase, Low-density lipoprotein receptor-related protein 6, Luteinizing hormone/Choriogonadotropin receptor, Lymphocyte antigen 96, Lymphocyte differentiation antigen CD38, Lymphoid enhancer-binding factor 1, Lysine-specific demethylase 2A, Lysine-specific demethylase 2B, Lysine-specific demethylase 3A, Lysine-specific demethylase 4A, Lysine-specific demethylase 4B, Lysine-specific demethylase 4C, Lysine-specific demethylase 4D, Lysine-specific demethylase 4D-like, Lysine-specific demethylase 5A, Lysine-specific demethylase 5B, Lysine-specific demethylase 5C, Lysine-specific demethylase 6B, Lysine-specific demethylase 7, Lysine-specific histone demethylase 1, Lysine-specific histone demethylase 1B, Lysophosphatidic acid receptor 4, Lysophosphatidic acid receptor 5, Lysophosphatidic acid receptor 6, Lysophosphatidic acid receptor Edg-2, Lysophosphatidic acid receptor Edg-4, Lysophosphatidic acid receptor Edg-7, Lysosomal Pro-X carboxypeptidase, Lysosomal acid lipase/cholesteryl ester hydrolase, Lysosomal alpha-glucosidase, Lysosomal alpha-mannosidase, Lysosomal protective protein, Lysyl-tRNA synthetase, MAP kinase ERKI, MAP kinase ERK2, MAP kinase p38 alpha, MAP kinase p38 beta, MAP kinase p38 delta, MAP kinase p38 gamma, MAP kinase signal-integrating kinase 2, MAP kinase-activated protein kinase 2, MAP kinase-activated protein kinase 3, MAP kinase-activated protein kinase 5, MAP kinase-interacting serine/threonine-protein kinase MNKI, MAP/microtubule affinity-regulating kinase 2, MAP/microtubule affinity-regulating kinase 4, MBT domain-containing protein 1, Macrophage colony stimulating factor receptor, Macrophage migration inhibitory factor, Macrophage-expressed gene 1 protein, Macrophage-stimulating protein receptor, Malate dehydrogenase cytoplasmic, Malate dehydrogenase, mitochondrial, Malonyl-CoA decarboxylase, Maltase-glucoamylase, Mannose-6-phosphate isomerase, Mas-related G-protein coupled receptor member Xi, Mas-related G-protein coupled receptor member X2, Maternal embryonic leucine zipper kinase, Matriptase, Matrix metalloproteinase 10, Matrix metalloproteinase 11, Matrix metalloproteinase 12, Matrix metalloproteinase 13, Matrix metalloproteinase 14, Matrix metalloproteinase 15, Matrix metalloproteinase 16, Matrix metalloproteinase 17, Matrix metalloproteinase 26, Matrix metalloproteinase 3, Matrix metalloproteinase 7, Matrix metalloproteinase 8, Matrix metalloproteinase 9, Matrix metalloproteinase-1, Matrix metalloproteinase-2, Matrix metalloproteinase-20, Matrix metalloproteinase-24, Matrix metalloproteinase-25, Max-like protein X, Melanin-concentrating hormone receptor 1, Melanin-concentrating hormone receptor 2, Melanocortin receptor 1, Melanocortin receptor 3, Melanocortin receptor 4, Melanocortin receptor 5, Melatonin receptor 1A, Melatonin receptor 1B, Membrane-bound transcription factor site-1 protease, Menin, Metabotropic glutamate receptor 1, Metabotropic glutamate receptor 2, Metabotropic glutamate receptor 3, Metabotropic glutamate receptor 4, Metabotropic glutamate receptor 5, Metabotropic glutamate receptor 6, Metabotropic glutamate receptor 7, Metabotropic glutamate receptor 8, Metastin receptor, Methionine aminopeptidase 1, Methionine aminopeptidase 2, Methionine synthase, Methionyl-tRNA synthetase, Methyl-CpG-binding domain protein 2, Microsomal triglyceride transfer protein large subunit, Microtubule-associated protein 2, Microtubule-associated protein tau, Microtubule-associated serine/threonine-protein kinase 1, Microtubule-associated serine/threonine-protein kinase 3, Mineralocorticoid receptor, Misshapen-like kinase 1, Mitogen-activated protein kinase 15, Mitogen-activated protein kinase 4, Mitogen-activated protein kinase 6, Mitogen-activated protein kinase 7, Mitogen-activated protein kinase kinase kinase 1, Mitogen-activated protein kinase kinase kinase 10, Mitogen-activated protein kinase kinase kinase 11, Mitogen-activated protein kinase kinase kinase 12, Mitogen-activated protein kinase kinase kinase 13, Mitogen-activated protein kinase kinase kinase 14, Mitogen-activated protein kinase kinase kinase 15, Mitogen-activated protein kinase kinase kinase 2, Mitogen-activated protein kinase kinase kinase 3, Mitogen-activated protein kinase kinase kinase 4, Mitogen-activated protein kinase kinase kinase 5, Mitogen-activated protein kinase kinase kinase 6, Mitogen-activated protein kinase kinase kinase 7, Mitogen-activated protein kinase kinase kinase 8, Mitogen-activated protein kinase kinase kinase 9, Mitogen-activated protein kinase kinase kinase kinase 1, Mitogen-activated protein kinase kinase kinase kinase 2, Mitogen-activated protein kinase kinase kinase kinase 3, Mitogen-activated protein kinase kinase kinase kinase 4, Mitogen-activated protein kinase kinase kinase kinase 5, Mitotic checkpoint serine/threonine-protein kinase BUB1, Mixed lineage kinase 7, Monoacylglycerol lipase ABHD12, Monoacylglycerol lipase ABHD6, Monoamine oxidase A, Monoamine oxidase B, Monocarboxylate transporter 1, Monocarboxylate transporter 4, Monoglyceride lipase, Motilin receptor, Mu opioid receptor, Mucosa-associated lymphoid tissue lymphoma translocation protein 1, Multidrug and toxin extrusion protein 1, Multidrug and toxin extrusion protein 2, Multidrug resistance-associated protein 1, Multidrug resistance-associated protein 4, Multidrug resistance-associated protein 5, Muscarinic acetylcholine receptor M1, Muscarinic acetylcholine receptor M2, Muscarinic acetylcholine receptor M3, Muscarinic acetylcholine receptor M4, Muscarinic acetylcholine receptor M5, Muscle glycogen phosphorylase, Muscle glycogen synthase, Muscle, skeletal receptor tyrosine protein kinase, Myc proto-oncogene protein, Myelin transcription factor 1, Myelin-associated glycoprotein, Myeloperoxidase, Myoglobin, Myosin IIIA, Myosin light chain kinase, Myosin light chain kinase family member 4, Myosin light chain kinase, smooth muscle, Myosin-IIIB, Myotonin-protein kinase, N(G),N(G)-dimethylarginine dimethylaminohydrolase 1, N-acylneuraminate-9-phosphatase, N-acylsphingosine-amidohydrolase, N-arachidonyl glycine receptor, N-lysine methyltransferase SETD8, N-lysine methyltransferase SMYD2, NACHT, LRR and PYD domains-containing protein 3, NAD kinase, NAD-dependent deacetylase sirtuin 1, NAD-dependent deacetylase sirtuin 2, NAD-dependent deacetylase sirtuin 3, NAD-dependent protein deacetylase sirtuin-6, NAD-dependent protein deacylase sirtuin-5, mitochondrial, NADP-dependent malic enzyme, NADPH oxidase 1, NADPH oxidase 4, NADPH oxidase 5, NEDD8-activating enzyme E1 regulatory subunit, NF-kappa-B essential modulator, NF-kappaB inhibitor alpha, NT-3 growth factor receptor, NUAK family SNF1-like kinase 1, NUAK family SNF1-like kinase 2, Natural resistance-associated macrophage protein 2, Nck adaptor protein 1, Neprilysin, Nerve growth factor receptor Trk-A, Neurogenic locus notch homolog protein 1, Neurogenic locus notch homolog protein 2, Neurogenic locus notch homolog protein 3, Neurogenic locus notch homolog protein 4, Neurokinin 1 receptor, Neurokinin 2 receptor, Neurokinin 3 receptor, Neuromedin B receptor, Neuromedin-U receptor 2, Neuronal acetylcholine receptor protein alpha-4 subunit, Neuronal acetylcholine receptor protein alpha-7 subunit, Neuronal acetylcholine receptor subunit alpha-3, Neuropeptide FF receptor 1, Neuropeptide FF receptor 2, Neuropeptide S receptor, Neuropeptide Y receptor type 1, Neuropeptide Y receptor type 2, Neuropeptide Y receptor type 4, Neuropeptide Y receptor type 5, Neuropeptides B/W receptor type 1, Neuropilin-1, Neurotensin receptor 1, Neurotensin receptor 2, Neurotensin receptor 3, Neurotrophic tyrosine kinase receptor type 2, Neutral amino acid transporter B(0), Neutral ceramidase, Neutral cholesterol ester hydrolase 1, Nicotinamide N-methyltransferase, Nicotinamide phosphoribosyltransferase, Niemann-Pick C1 protein, Niemann-Pick C1-like protein 1, Nischarin, Nitric oxide synthase, inducible, Nitric-oxide synthase, brain, Nitric-oxide synthase, endothelial, Nociceptin receptor, Non-receptor tyrosine-protein kinase TNKI1, Non-secretory ribonuclease, Non-specific lipid-transfer protein, Norepinephrine transporter, Nuclear factor NF-kappa-B p105 subunit, Nuclear factor NF-kappa-B p65 subunit, Nuclear factor erythroid 2-related factor 2, Nuclear receptor ROR-alpha, Nuclear receptor ROR-beta, Nuclear receptor ROR-gamma, Nuclear receptor subfamily 0 group B member 2, Nuclear receptor subfamily 1 group D member 1, Nuclear receptor subfamily 1 group D member 2, Nuclear receptor subfamily 1 group I member 3, Nuclear receptor subfamily 2 group C member 2, Nuclear receptor subfamily 4 group A member 2, Nucleophosmin, Nucleosome-remodeling factor subunit BPTF, Nucleotide-binding oligomerization domain-containing protein 1, Nucleotide-binding oligomerization domain-containing protein 2, Oligopeptide transporter small intestine isoform, Opioid growth factor receptor-like protein 1, Orexin receptor 1, Orexin receptor 2, Ornithine aminotransferase, mitochondrial, Ornithine decarboxylase, Orphan nuclear receptor LRH-1, Oxoeicosanoid receptor 1, Oxytocin receptor, P-glycoprotein 1, P-selectin, P-selectin glycoprotein ligand 1, P2X purinoceptor 1, P2X purinoceptor 2, P2X purinoceptor 3, P2X purinoceptor 4, P2X purinoceptor 7, PAS domain-containing serine/threonine-protein kinase, PDZ-binding kinase, PH domain leucine-rich repeat-containing protein phosphatase 1, PI3-kinase p110-alpha subunit, P13-kinase p110-beta subunit, P13-kinase p¹10-delta subunit, P13-kinase p110-gamma subunit, P14-kinase alpha subunit, P14-kinase beta subunit, PI4-kinase type II, PITSLRE serine/threonine-protein kinase CDC2L1, PITSLRE serine/threonine-protein kinase CDC2L2, POU domain, class 2, transcription factor 1, POU domain, class 2, transcription factor 2, Palmitoleoyl-protein carboxylesterase NOTUM, Pancreatic alpha-amylase, Pancreatic lipase, Pantothenate kinase 1, Pantothenate kinase 2, mitochondrial, Pantothenate kinase 3, Parathyroid hormone receptor, Patatin-like phospholipase domain-containing protein 2, Pepsin A, Pepsinogen C, Peptide N-myristoyltransferase 1, Peptide N-myristoyltransferase 2, Peptide deformylase mitochondrial, Peptidyl-glycine alpha-amidating monooxygenase, Peptidyl-prolyl cis-trans isomerase D, Peptidyl-prolyl cis-trans isomerase FKBP5, Peptidyl-prolyl cis-trans isomerase NIMA-interacting 1, Peptidyl-prolyl cis-trans isomerase NIMA-interacting 2, Peregrin, Perforin-1, Period circadian protein homolog 2, Peripheral plasma membrane protein CASK, Peroxisome proliferator-activated receptor alpha, Peroxisome proliferator-activated receptor delta, Peroxisome proliferator-activated receptor gamma, Phenylalanine-4-hydroxylase, Phenylethanolamine N-methyltransferase, Phosphatidylcholine:ceramide cholinephosphotransferase 1, Phosphatidylcholine:ceramide cholinephosphotransferase 2, Phosphatidylinositol 3,4,5-trisphosphate 5-phosphatase 2, Phosphatidylinositol 3-kinase catalytic subunit type 3, Phosphatidylinositol-4-phosphate 3-kinase C2 domain-containing beta polypeptide, Phosphatidylinositol-4-phosphate 3-kinase C2 domain-containing subunit alpha, Phosphatidylinositol-4-phosphate 3-kinase C2 domain-containing subunit gamma, Phosphatidylinositol-4-phosphate 5-kinase type-1 alpha, Phosphatidylinositol-4-phosphate 5-kinase type-1 gamma, Phosphatidylinositol-5-phosphate 4-kinase type-2 beta, Phosphatidylinositol-5-phosphate 4-kinase type-2 gamma, Phosphodiesterase 10A, Phosphodiesterase 11A, Phosphodiesterase 1A, Phosphodiesterase 1B, Phosphodiesterase 1C, Phosphodiesterase 2A, Phosphodiesterase 3A, Phosphodiesterase 3B, Phosphodiesterase 4A, Phosphodiesterase 4B, Phosphodiesterase 4C, Phosphodiesterase 4D, Phosphodiesterase 5A, Phosphodiesterase 6A, Phosphodiesterase 6C, Phosphodiesterase 6D, Phosphodiesterase 7A, Phosphodiesterase 7B, Phosphodiesterase 8A, Phosphodiesterase 8B, Phosphodiesterase 9A, Phosphoenolpyruvate carboxykinase cytosolic, Phosphoethanolamine/phosphocholine phosphatase, Phosphoglycerate mutase 1, Phospholipase A2 group 1B, Phospholipase A2 group IIA, Phospholipase A2 group IIC, Phospholipase A2 group V, Phospholipase C-gamma-1, Phospholipase D1, Phospholipase D2, Phosphomannomutase 2, Phosphorylase kinase gamma subunit 1, Phosphorylase kinase gamma subunit 2, Photoreceptor-specific nuclear receptor, Placenta growth factor, Plasma kallikrein, Plasma retinol-binding protein, Plasminogen, Plasminogen activator inhibitor-1, Platelet activating factor receptor, Platelet-activating factor acetylhydrolase IB beta subunit, Platelet-derived growth factor receptor alpha, Platelet-derived growth factor receptor beta, Platelet-derived growth factor subunit A, Platelet-derived growth factor subunit B, Pleckstrin homology domain-containing family A member 1, Poly [ADP-ribose]polymerase 10, Poly [ADP-ribose]polymerase 14, Poly [ADP-ribose]polymerase 15, Poly [ADP-ribose]polymerase 2, Poly [ADP-ribose]polymerase 3, Poly [ADP-ribose]polymerase 4, Poly [ADP-ribose]polymerase 6, Poly [ADP-ribose]polymerase-1, Poly(A)-specific ribonuclease PARN, Polycomb protein EED, Porphobilinogen synthase, Potassium channel subfamily K member 10, Potassium channel subfamily K member 2, Potassium channel subfamily K member 3, Potassium channel subfamily K member 4, Potassium channel subfamily K member 9, Potassium channel, inwardly rectifying, subfamily J, member 11, Potassium voltage-gated channel subfamily B member 1, Potassium voltage-gated channel subfamily B member 2, Potassium/sodium hyperpolarization-activated cyclic nucleotide-gated channel 1, Potassium/sodium hyperpolarization-activated cyclic nucleotide-gated channel 2, Potassium/sodium hyperpolarization-activated cyclic nucleotide-gated channel 3, Potassium/sodium hyperpolarization-activated cyclic nucleotide-gated channel 4, Pregnane X receptor, Prenyl protein specific protease, Presenilin 1, Presenilin 2, Presequence protease, mitochondrial, Probable DNA dC->dU-editing enzyme APOBEC-3A, Probable G-protein coupled receptor 139, Probable G-protein coupled receptor 142, Probable G-protein coupled receptor 174, Probable G-protein coupled receptor 34, Probable G-protein coupled receptor 52, Probable G-protein coupled receptor 88, Probable JmjC domain-containing histone demethylation protein 2C, Probable global transcription activator SNF2L2, Probable protein-cysteine N-palmitoyltransferase porcupine, Probable ubiquitin carboxyl-terminal hydrolase FAF-X, Progesterone receptor, Programmed cell death 1 ligand 1, Programmed cell death protein 4, Prohormone convertase 1, Prohormone convertase 2, Prokineticin receptor 1, Proliferating cell nuclear antigen, Proline-rich AKTI substrate 1, Prolyl 4-hydroxylase subunit alpha-1, Prolyl endopeptidase, Prostaglandin E synthase, Prostaglandin E synthase 2, Prostanoid DP receptor, Prostanoid EP1 receptor, Prostanoid EP2 receptor, Prostanoid EP3 receptor, Prostanoid EP4 receptor, Prostanoid FP receptor, Prostanoid IP receptor, Prostasin, Prostate specific antigen, Prostatic acid phosphatase, Proteasome Macropain subunit, Proteasome Macropain subunit MB1, Proteasome component C5, Proteasome subunit beta type-10, Proteasome subunit beta type-8, Proteasome subunit beta type-9, Protection of telomeres protein 1, Protein Mdm4, Protein Wnt-3a, Protein arginine N-methyltransferase 3, Protein arginine N-methyltransferase 5, Protein arginine N-methyltransferase 6, Protein arginine N-methyltransferase 7, Protein arginine N-methyltransferase 8, Protein cereblon, Protein delta homolog 1, Protein kinase C alpha, Protein kinase C beta, Protein kinase C delta, Protein kinase C epsilon, Protein kinase C eta, Protein kinase C gamma, Protein kinase C iota, Protein kinase C mu, Protein kinase C nu, Protein kinase C theta, Protein kinase C zeta, Protein kinase NI, Protein kinase N2, Protein max, Protein phosphatase 1D, Protein phosphatase 1G, Protein phosphatase 2C alpha, Protein phosphatase methylesterase 1, Protein polybromo-1, Protein prune homolog, Protein tyrosine kinase 2 beta, Protein tyrosine phosphatase type IVA 1, Protein tyrosine phosphatase type IVA 2, Protein-arginine N-methyltransferase 1, Protein-arginine deiminase type-1, Protein-arginine deiminase type-2, Protein-arginine deiminase type-3, Protein-arginine deiminase type-4, Protein-glutamine gamma-glutamyltransferase, Protein-glutamine gamma-glutamyltransferase 6, Protein-glutamine gamma-glutamyltransferase K, Protein-glutamine glutamyltransferase E, Protein-tyrosine phosphatase 1B, Protein-tyrosine phosphatase 1C, Protein-tyrosine phosphatase 1E, Protein-tyrosine phosphatase 2C, Protein-tyrosine phosphatase 4A3, Protein-tyrosine phosphatase GI, Protein-tyrosine phosphatase LC-PTP, Proteinase activated receptor 4, Proteinase-activated receptor 1, Proteinase-activated receptor 2, Proto-oncogene c-JUN, Proto-oncogene protein Wnt-3, Proto-oncogene tyrosine-protein kinase MER, Proto-oncogene tyrosine-protein kinase ROS, Proto-oncogene vav, Proton-coupled folate transporter, Protoporphyrinogen oxidase, Purine nucleoside phosphorylase, Purinergic receptor P2Y1, Purinergic receptor P2Y11, Purinergic receptor P2Y12, Purinergic receptor P2Y14, Purinergic receptor P2Y2, Puromycin-sensitive aminopeptidase, Putative N-acetylglucosamine-6-phosphate deacetylase, Putative P2Y purinoceptor 10, Putative uncharacterized serine/threonine-protein kinase SgK110, Pyrimidinergic receptor P2Y4, Pyrimidinergic receptor P2Y6, Pyroglutamylated RFamide peptide receptor, Pyruvate dehydrogenase kinase isoform 1, Pyruvate dehydrogenase kinase isoform 2, Pyruvate dehydrogenase kinase isoform 3, Pyruvate dehydrogenase kinase isoform 4, Pyruvate kinase isozymes MI/M2, Pyruvate kinase isozymes R/L, Quinone reductase 1), Quinone reductase 2, RAS guanyl releasing protein 3, RAS guanyl-releasing protein 1, RNA polymerase II subunit A C-terminal domain phosphatase SSU72, RNase L, Rap guanine nucleotide exchange factor 3, Rap guanine nucleotide exchange factor 4, Ras-related C3 botulinum toxin substrate 1, Receptor protein-tyrosine kinase erbB-2, Receptor protein-tyrosine kinase erbB-4, Receptor tyrosine-protein kinase erbB-3, Receptor-interacting serine/threonine-protein kinase 1, Receptor-interacting serine/threonine-protein kinase 3, Receptor-interacting serine/threonine-protein kinase 4, Receptor-type tyrosine-protein phosphatase F (LAR), Receptor-type tyrosine-protein phosphatase R, Receptor-type tyrosine-protein phosphatase S, Receptor-type tyrosine-protein phosphatase alpha, Receptor-type tyrosine-protein phosphatase beta, Receptor-type tyrosine-protein phosphatase eta, Receptor-type tyrosine-protein phosphatase gamma, Receptor-type tyrosine-protein phosphatase mu, Regulator of G-protein signaling 16, Regulator of G-protein signaling 19, Regulator of G-protein signaling 8, Renin, Replication protein A 70 kDa DNA-binding subunit, Retinoblastoma-associated protein, Retinoic acid receptor alpha, Retinoic acid receptor beta, Retinoic acid receptor gamma, Retinoid X receptor alpha, Retinoid X receptor beta, Retinoid X receptor gamma, Rho GDP-dissociation inhibitor 1, Rho-associated protein kinase 1, Rho-associated protein kinase 2, Rhodopsin kinase, Ribonuclease H1, Ribonucleoside-diphosphate reductase M1 chain, Ribosomal protein S6 kinase 1, Ribosomal protein S6 kinase alpha 1, Ribosomal protein S6 kinase alpha 2, Ribosomal protein S6 kinase alpha 3, Ribosomal protein S6 kinase alpha 4, Ribosomal protein S6 kinase alpha 5, Ribosomal protein S6 kinase alpha 6, RuvB-like 1, S-100 protein beta chain, S-adenosylmethionine decarboxylase 1, S-methyl-5-thioadenosine phosphorylase, S-methylnethionine--homocysteine S-methyltransferase BHMT2, SH2 domain-containing protein 1A, SNF-related serine/threonine-protein kinase, SPRY domain-containing SOCS box protein 2, SPS1/STE20-related protein kinase YSK4, STE20/SPS1-related proline-alanine-rich protein kinase, SUMO-activating enzyme subunit 1, SUMO-conjugating enzyme UBC9, Salivary alpha-amylase, Scavenger receptor class B member 1, Secreted frizzled-related protein 1, Segment polarity protein dishevelled homolog DVL-1, Segment polarity protein dishevelled homolog DVL-3, Selectin E, Sentrin-specific protease 1, Sentrin-specific protease 2, Serine hydroxymethyltransferase, cytosolic, Serine palmitoyltransferase 1, Serine palmitoyltransferase 2, Serine protease hepsin, Serine racemase, Serine-protein kinase ATM, Serine-protein kinase ATR, Serine/threonine protein kinase NLK, Serine/threonine-protein kinase 10, Serine/threonine-protein kinase 11, Serine/threonine-protein kinase 16, Serine/threonine-protein kinase 17A, Serine/threonine-protein kinase 17B, Serine/threonine-protein kinase 2, Serine/threonine-protein kinase 24, Serine/threonine-protein kinase 25, Serine/threonine-protein kinase 32A, Serine/threonine-protein kinase 32B, Serine/threonine-protein kinase 32C, Serine/threonine-protein kinase 33, Serine/threonine-protein kinase 35, Serine/threonine-protein kinase 36, Serine/threonine-protein kinase 38, Serine/threonine-protein kinase 38-like, Serine/threonine-protein kinase A-Raf, Serine/threonine-protein kinase AKT, Serine/threonine-protein kinase AKT2, Serine/threonine-protein kinase AKT3, Serine/threonine-protein kinase Aurora-A, Serine/threonine-protein kinase Aurora-B, Serine/threonine-protein kinase Aurora-C, Serine/threonine-protein kinase B-raf, Serine/threonine-protein kinase Chkl, Serine/threonine-protein kinase Chk2, Serine/threonine-protein kinase D2, Serine/threonine-protein kinase DCLK1, Serine/threonine-protein kinase DCLK2, Serine/threonine-protein kinase DCLK3, Serine/threonine-protein kinase EEF2K, Serine/threonine-protein kinase GAK, Serine/threonine-protein kinase ICK, Serine/threonine-protein kinase ILK-1, Serine/threonine-protein kinase LATS1, Serine/threonine-protein kinase LATS2, Serine/threonine-protein kinase MAK, Serine/threonine-protein kinase MARKI, Serine/threonine-protein kinase MRCK beta, Serine/threonine-protein kinase MRCK gamma, Serine/threonine-protein kinase MRCK-A, Serine/threonine-protein kinase MST1, Serine/threonine-protein kinase MST2, Serine/threonine-protein kinase MST4, Serine/threonine-protein kinase NEK2, Serine/threonine-protein kinase NEK6, Serine/threonine-protein kinase NEK7, Serine/threonine-protein kinase NEK9, Serine/threonine-protein kinase NIMI, Serine/threonine-protein kinase Nekl, Serine/threonine-protein kinase Neki1, Serine/threonine-protein kinase Nek3, Serine/threonine-protein kinase Nek4, Serine/threonine-protein kinase Nek5, Serine/threonine-protein kinase OSRI, Serine/threonine-protein kinase PAK 1, Serine/threonine-protein kinase PAK 2, Serine/threonine-protein kinase PAK 3, Serine/threonine-protein kinase PAK 4, Serine/threonine-protein kinase PAK6, Serine/threonine-protein kinase PAK7, Serine/threonine-protein kinase PCTAIRE-1, Serine/threonine-protein kinase PCTAIRE-2, Serine/threonine-protein kinase PCTAIRE-3, Serine/threonine-protein kinase PFTAIRE-1, Serine/threonine-protein kinase PFTAIRE-2, Serine/threonine-protein kinase PIMI, Serine/threonine-protein kinase PIM2, Serine/threonine-protein kinase PIM3, Serine/threonine-protein kinase PLK1, Serine/threonine-protein kinase PLK2, Serine/threonine-protein kinase PLK3, Serine/threonine-protein kinase PLK4, Serine/threonine-protein kinase PRKX, Serine/threonine-protein kinase PRP4 homolog, Serine/threonine-protein kinase RAF, Serine/threonine-protein kinase RIO1, Serine/threonine-protein kinase R102, Serine/threonine-protein kinase R103, Serine/threonine-protein kinase RIPK2, Serine/threonine-protein kinase SBK1, Serine/threonine-protein kinase SIKI, Serine/threonine-protein kinase SIK2, Serine/threonine-protein kinase SIK3, Serine/threonine-protein kinase SMG1, Serine/threonine-protein kinase SRPK1, Serine/threonine-protein kinase SRPK2, Serine/threonine-protein kinase SRPK3, Serine/threonine-protein kinase Sgkl, Serine/threonine-protein kinase Sgk2, Serine/threonine-protein kinase Sgk3, Serine/threonine-protein kinase TAO1, Serine/threonine-protein kinase TAO2, Serine/threonine-protein kinase TAO3, Serine/threonine-protein kinase TBK1, Serine/threonine-protein kinase TNNI3K, Serine/threonine-protein kinase ULKI, Serine/threonine-protein kinase ULK2, Serine/threonine-protein kinase ULK3, Serine/threonine-protein kinase VRK2, Serine/threonine-protein kinase WEE1, Serine/threonine-protein kinase WNK3, Serine/threonine-protein kinase c-TAK1, Serine/threonine-protein kinase haspin, Serine/threonine-protein kinase mTOR, Serine/threonine-protein kinase receptor R3, Serine/threonine-protein kinase tousled-like 1, Serine/threonine-protein kinase tousled-like 2, Serine/threonine-protein kinase/endoribonuclease IRE1, Serotonin 1a (5-HTla) receptor, Serotonin 1b (5-HT1b) receptor, Serotonin id (5-HT1d) receptor, Serotonin 1e (5-HTle) receptor, Serotonin 1f (5-HTlf) receptor, Serotonin 2a (5-HT2a) receptor, Serotonin 2b (5-HT2b) receptor, Serotonin 2c (5-HT2c) receptor, Serotonin 3a (5-HT3a) receptor, Serotonin 4 (5-HT4) receptor, Serotonin 5a (5-HT5a) receptor, Serotonin 6 (5-HT6) receptor, Serotonin 7 (5-HT7) receptor, Serotonin transporter, Serum albumin, Serum paraoxonase/arylesterase 1, Short transient receptor potential channel 3, Short transient receptor potential channel 6, Sialidase 1, Sialidase 2, Sialidase 3, Sialidase 4, Sigma opioid receptor, Signal transducer and activator of transcription 1-alpha/beta, Signal transducer and activator of transcription 3, Signal transducer and activator of transcription 5A, Signal transducer and activator of transcription 5B, Signal transducer and activator of transcription 6, Small conductance calcium-activated potassium channel protein 1, Small conductance calcium-activated potassium channel protein 2, Small conductance calcium-activated potassium channel protein 3, Small ubiquitin-related modifier 1, Smoothened homolog, Sni-specific diacylglycerol lipase alpha, Sni-specific diacylglycerol lipase beta, Sodium channel protein type I alpha subunit, Sodium channel protein type II alpha subunit, Sodium channel protein type III alpha subunit, Sodium channel protein type IV alpha subunit, Sodium channel protein type IX alpha subunit, Sodium channel protein type V alpha subunit, Sodium channel protein type VIII alpha subunit, Sodium channel protein type X alpha subunit, Sodium-dependent neutral amino acid transporter B(0)AT2, Sodium-dependent proline transporter, Sodium/glucose cotransporter 1, Sodium/glucose cotransporter 2, Sodium/hydrogen exchanger 1, Sodium/hydrogen exchanger 2, Sodium/nucleoside cotransporter 1, Sodium/nucleoside cotransporter 2, Sodium/potassium-transporting ATPase alpha-1 chain, Sodium/potassium/calcium exchanger 6, mitochondrial, Solute carrier family 12 member 5, Solute carrier family 13 member 2, Solute carrier family 13 member 3, Solute carrier family 13 member 5, Solute carrier family 15 member 2, Solute carrier family 2, facilitated glucose transporter member 2, Solute carrier family 2, facilitated glucose transporter member 3, Solute carrier family 22 member 1, Solute carrier family 22 member 11, Solute carrier family 22 member 12, Solute carrier family 22 member 16, Solute carrier family 22 member 2, Solute carrier family 22 member 3, Solute carrier family 22 member 6, Solute carrier family 22 member 8, Solute carrier family 28 member 3, Solute carrier organic anion transporter family member 1A2, Solute carrier organic anion transporter family member 1B1, Solute carrier organic anion transporter family member 1B3, Solute carrier organic anion transporter family member 2A1, Solute carrier organic anion transporter family member 2B1, Solute carrier organic anion transporter family member 4C1, Somatostatin receptor 1, Somatostatin receptor 2, Somatostatin receptor 3, Somatostatin receptor 4, Somatostatin receptor 5, Sonic hedgehog protein, Spermidine/spermine N(1)-acetyltransferase 1, Sphingolipid delta(4)-desaturase DES1, Sphingosine 1-phosphate receptor Edg-1, Sphingosine 1-phosphate receptor Edg-3, Sphingosine 1-phosphate receptor Edg-5, Sphingosine 1-phosphate receptor Edg-6, Sphingosine 1-phosphate receptor Edg-8, Sphingosine kinase 1, Sphingosine kinase 2, Sphingosine-1-phosphate lyase 1, Splicing factor 3B subunit 3, Squalene synthetase, Stearoyl-CoA desaturase 5, Stem cell growth factor receptor, Steroid 5-alpha-reductase 1, Steroid 5-alpha-reductase 2, Steroidogenic factor 1, Sterol regulatory element-binding protein 2, Steryl-sulfatase, Subtilisin/kexin type 5, Subtilisin/kexin type 6, Subtilisin/kexin type 7, Succinate receptor 1, Succinate semialdehyde dehydrogenase, Sucrase-isomaltase, Sulfonylurea receptor 1, Sulfonylurea receptor 2, Sulfotransferase lAl, Superoxide dismutase, T-cell protein-tyrosine phosphatase, T-cell surface antigen CD4, TGF-beta receptor type I, TGF-beta receptor type II, TNF receptor-associated factor 6, TNF-alpha, TRAF2- and NCK-interacting kinase, Tachykinin-3, Tankyrase-1, Tankyrase-2, Taste receptor type 2 member 31, Telomerase reverse transcriptase, Terminal deoxynucleotidyltransferase, Testis-specific androgen-binding protein, Testis-specific serine/threonine-protein kinase 1, Testis-specific serine/threonine-protein kinase 2, Testis-specific serine/threonine-protein kinase 3, Thiopurine S-methyltransferase, Thioredoxin, Thioredoxin, mitochondrial, Threonyl-tRNA synthetase, Thrombin, Thrombopoietin receptor, Thromboxane A2 receptor, Thromboxane-A synthase, Thymidine kinase, cytosolic, Thymidine kinase, mitochondrial, Thymidine phosphorylase, Thymidylate kinase, Thymidylate synthase, Thyroid hormone receptor alpha, Thyroid hormone receptor beta-1, Thyroid peroxidase, Thyroid stimulating hormone receptor, Tissue-type plasminogen activator, Toll-like receptor 2, Toll-like receptor 4, Toll-like receptor 7, Toll-like receptor 8, Toll-like receptor 9, Trace amine-associated receptor 1, Trace amine-associated receptor 5, Transcription activator BRGI, Transcription factor ETV6, Transcription factor HES-1, Transcription initiation factor IIA alpha and bTranscription initiation factor IIA alpha and beta chains, Transcription initiation factor TFIID subunit 1, Transcription initiation factor TFIID subunit 1-like, Transcription intermediary factor 1-alpha, Transcriptional regulator ERG, Transforming protein RhoA, Transforming protein p21/H-Ras-1, Transient receptor potential cation channel subfamily A member 1, Transient receptor potential cation channel subfamily M member 2, Transient receptor potential cation channel subfamily M member 6, Transient receptor potential cation channel subfamily M member 8, Transient receptor potential cation channel subfamily V member 3, Transient receptor potential cation channel subfamily V member 4, Transitional endoplasmic reticulum ATPase, Transketolase, Translocator protein, Transmembrane domain-containing protein TMIGD3, Transmembrane protease serine 1ID, Transmembrane protease serine 4, Transmembrane protease serine 6, Transthyretin, Tripeptidyl-peptidase 2, Trypsin I, Trypsin III, Tryptase beta-1, Tryptase gamma, Tryptophan 2,3-dioxygenase, Tryptophan 5-hydroxylase 1, Tryptophan 5-hydroxylase 2, Tubulin beta-3 chain, Tumor necrosis factor receptor R1, Tumor suppressor p53-binding protein 1, Type-1 angiotensin II receptor, Tyrosinase, Tyrosine kinase non-receptor protein 2, Tyrosine- and threonine-specific cdc2-inhibitory kinase, Tyrosine-protein kinase ABL, Tyrosine-protein kinase ABL2, Tyrosine-protein kinase BLK, Tyrosine-protein kinase BMX, Tyrosine-protein kinase BRK, Tyrosine-protein kinase BTK, Tyrosine-protein kinase CSK, Tyrosine-protein kinase CTK, Tyrosine-protein kinase FER, Tyrosine-protein kinase FES, Tyrosine-protein kinase FGR, Tyrosine-protein kinase FRK, Tyrosine-protein kinase FYN, Tyrosine-protein kinase HCK, Tyrosine-protein kinase ITK/TSK, Tyrosine-protein kinase JAKI, Tyrosine-protein kinase JAK2, Tyrosine-protein kinase JAK3, Tyrosine-protein kinase LCK, Tyrosine-protein kinase Lyn, Tyrosine-protein kinase SRC, Tyrosine-protein kinase SYK, Tyrosine-protein kinase Srms, Tyrosine-protein kinase TEC, Tyrosine-protein kinase TIE-2, Tyrosine-protein kinase TXK, Tyrosine-protein kinase TYK2, Tyrosine-protein kinase YES, Tyrosine-protein kinase ZAP-70, Tyrosine-protein kinase receptor FLT3, Tyrosine-protein kinase receptor RET, Tyrosine-protein kinase receptor TYRO3, Tyrosine-protein kinase receptor Tie-1, Tyrosine-protein kinase receptor UFO, Tyrosine-protein phosphatase non-receptor type 14, Tyrosine-protein phosphatase non-receptor type 18, Tyrosine-protein phosphatase non-receptor type 3, Tyrosine-protein phosphatase non-receptor type 5, Tyrosine-protein phosphatase non-receptor type 9, Tyrosyl-DNA phosphodiesterase 1, Tyrosyl-DNA phosphodiesterase 2, UDP-N-acetylglucosamine--peptide N-acetylglucosaminyltransferase 110 kDa subunit, UDP-glucose 4-epimerase, UDP-glucuronosyltransferase 1-1, UDP-glucuronosyltransferase 1-10, UDP-glucuronosyltransferase 1-3, UDP-glucuronosyltransferase 1-6, UDP-glucuronosyltransferase 1-7, UDP-glucuronosyltransferase 1-8, UDP-glucuronosyltransferase 1-9, UDP-glucuronosyltransferase 1A4, UDP-glucuronosyltransferase 2B17, UDP-glucuronosyltransferase 2B7, Ubiquitin carboxyl-terminal hydrolase 1, Ubiquitin carboxyl-terminal hydrolase 10, Ubiquitin carboxyl-terminal hydrolase 13, Ubiquitin carboxyl-terminal hydrolase 14, Ubiquitin carboxyl-terminal hydrolase 2, Ubiquitin carboxyl-terminal hydrolase 21, Ubiquitin carboxyl-terminal hydrolase 4, Ubiquitin carboxyl-terminal hydrolase 5, Ubiquitin carboxyl-terminal hydrolase 7, Ubiquitin carboxyl-terminal hydrolase isozyme Li, Ubiquitin carboxyl-terminal hydrolase isozyme L3, Ubiquitin carboxyl-terminal hydrolase isozyme L5, Ubiquitin-like domain-containing CTD phosphatase 1, Ubiquitin-like modifier-activating enzyme 6, Ubiquitin-like modifier-activating enzyme 7, Ubiquitin-like modifier-activating enzyme ATG7, Ubl carboxyl-terminal hydrolase 18, Uncharacterized aarF domain-containing protein kinase 4, Uracil nucleotide/cysteinyl leukotriene receptor, Uracil-DNA glycosylase, Urea transporter 1, Uridine 5′-monophosphate synthase, Uridine phosphorylase 1, Urokinase plasminogen activator surface receptor, Urokinase-type plasminogen activator, Urotensin II receptor, V-type proton ATPase subunit B, brain isoform, Vanilloid receptor, Vascular endothelial growth factor A, Vascular endothelial growth factor receptor 1, Vascular endothelial growth factor receptor 2, Vascular endothelial growth factor receptor 3, Vasoactive intestinal polypeptide receptor 1, Vasoactive intestinal polypeptide receptor 2, Vasopressin Vla receptor, Vasopressin V1b receptor, Vasopressin V2 receptor, Vesicular acetylcholine transporter, Vitamin D receptor, Vitamin D-binding protein, Vitamin K-dependent protein C, Vitronectin receptor alpha, Voltage-dependent anion-selective channel protein 2, Voltage-gated L-type calcium channel alpha-1C subunit, Voltage-gated L-type calcium channel alpha-1D subunit, Voltage-gated N-type calcium channel alpha-1B subunit, Voltage-gated T-type calcium channel alpha-1G subunit, Voltage-gated T-type calcium channel alpha-1H subunit, Voltage-gated T-type calcium channel alpha-II subunit, Voltage-gated calcium channel alpha2/delta subunit 1, Voltage-gated potassium channel beta subunit Mink, Voltage-gated potassium channel subunit Kvi.3, Voltage-gated potassium channel subunit Kvi.5, Voltage-gated potassium channel subunit Kv4.3, Voltage-gated potassium channel subunit Kv7.1, Voltage-gated potassium channel subunit Kv7.2, Voltage-gated potassium channel subunit Kv7.3, Voltage-gated potassium channel subunit Kv7.4, Voltage-gated potassium channel subunit Kv7.5, Von Hippel-Lindau disease tumor suppressor, WD repeat-containing protein 5, Weel-like protein kinase 2, Werner syndrome ATP-dependent helicase, X-box-binding protein 1, Xanthine dehydrogenase, Zinc finger protein GLI1, Zinc finger protein GLI2, c-Jun N-terminal kinase 1, c-Jun N-terminal kinase 2, c-Jun N-terminal kinase 3, cAMP-dependent protein kinase alpha-catalytic subunit, cAMP-dependent protein kinase beta-1 catalytic subunit, cGMP-dependent protein kinase 1 beta, cGMP-dependent protein kinase 2, dCTP pyrophosphatase 1, dUTP pyrophosphatase, myosin light chain kinase 2, and p53-binding protein Mdm-2.

In some embodiments, a modified Target Protein described herein is formed by contacting a precursor compound with an amino acid residue (e.g., serine) of an unmodified Target Protein, wherein the precursor compound comprises a moiety susceptible to reacting with a nucleophilic amino acid residue (e.g., a nucleophilic serine residue). In some embodiments, the modified Target Protein is formed by contacting a compound disclosed herein, such as a compound of Formula (I), (I), (II), (III), or (IV), with an amino acid (e.g., serine) residue of an unmodified Target Protein. In some embodiments, the modified Target Protein is formed by contacting a precursor compound with an amino acid (e.g., serine) residue of an unmodified Target Protein, wherein the precursor compound comprises a staying group and a leaving group, and wherein said contacting results in release of the leaving group and formation of said modified Target Protein. In some embodiments, the precursor compound is a compound disclosed herein, such as a compound of Formula (I), (I), (II), (III), or (IV). In some embodiments, the leaving group is selected from

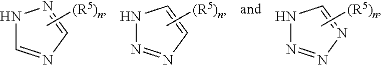

or a salt or tautomer thereof, wherein R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and n is 0, 1, or 2.

In some embodiments, a modified Target Protein is formed by contacting a precursor compound with an amino acid (e.g., serine) residue of an unmodified Target Protein, wherein the precursor compound comprises a staying group and a leaving group, and wherein said leaving group separates from remainder of the precursor compound with an electron pair that previously formed a covalent bond between the leaving group and the remainder of the precursor compound after contacting the precursor compound with the unmodified Target Protein. In some embodiments, a modified Target Protein is formed by contacting a precursor compound with an amino acid (e.g., serine) residue of an unmodified Target Protein, wherein the precursor compound comprises a staying group and a leaving group, and wherein said contacting results in release of the leaving group and formation of said modified Target Protein. Release of the leaving group can be ascertained by a variety of methods known in the art, including without limitation mass spectroscopy. In particular, the molecular weight of the leaving group can be determined by the following formula:

L=[U+P]−[M]

wherein:
L is the molecular weight of the leaving group; U is the molecular weight of an unmodified Target Protein; P is the molecular weight of a subject precursor compound used to modify the unmodified Target Protein; M is the molecular weight of the modified Target Protein covalently bound to the precursor excluding the leaving group.

The molecular weight of the modified Target Protein can be ascertained by mass spectroscopy. In some embodiments, a subject compound, upon contacting an unmodified Target Protein, yields a leaving group of a molecular weight less than about 200 Da, such as less than about 190, 180, 170, 160, 150, 140, 130, 120, 110, 100, 90, 80, 70, 60, 50, 40, 30, 20 Da or less. One or more compound described herein, such as a compound of Table 1, yields, upon contacting a Target Protein, a leaving group of molecular weight less than 120 Da.

In some embodiments, a modified Target Protein of the present disclosure exhibits a reduced signaling output. A reduction of signaling output can be ascertained by a wide variety of methods known in the art. For example, phosphorylation of a substrate or a specific amino acid residue thereof can be detected and/or quantified using one or more techniques, such as kinase activity assays, phospho-specific antibodies, Western blot, enzyme-linked immunosorbent assays (ELISA), cell-based ELISA, intracellular flow cytometry, mass spectrometry, or multi-analyte profiling. In some embodiments, the reduction in Target Protein signaling output can be evidenced by comparing a measure of signaling output to that of a control unmodified Target Protein that is not covalently bonded to a compound disclosed herein. For example, a control Target Protein, as described herein, can be a Target Protein (e.g., wildtype or mutated) that is not complexed with a compound of the present disclosure. The increase or reduction in signaling output can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the control Target Protein. In some embodiments, a reduction in Target Protein interaction with a Target Protein-pathway signaling protein is established by observing a reduced interaction with one or more Target Protein-pathway signaling proteins.

Signaling output measured in terms of IC50 values can be obtained and a ratio of IC50 against one mutant relative to another mutant can be calculated. For instance, a selective reduction of Target Protein mutant A signaling output can be evidenced by a ratio greater than one. In particular, a selective reduction of Target Protein mutant A signaling relative to Target Protein mutant B signaling is evidenced when the ratio of IC50 (against Target Protein mutant B) to IC50 (against Target Protein mutant A) is greater than 1. One or more compound disclosed herein, such as a compound of Table 1, may exhibit selective inhibition of one Target Protein mutant relative to another Target Protein mutant by at least 1-fold, and in some instances greater than 2-, 3—, 4- or 5-fold. In some embodiments, a compound of the present disclosure exhibits an IC50 against the Target Protein of less than 500 nM, such as less than 100 nM, 50 nM, 10 nM or even less. Exemplary compounds, such as a compound of Table 1, may covalently label the Target Protein by at least 1%, 10%, 20%, 50%.

In some embodiments, a compound of the present disclosure selectively labels a serine residue as compared to (i) an aspartate residue of the Target Protein, and/or (ii) a valine residue of the Target Protein, by at least 0.1, 0.2, 0.3, 0.4, 0.5, 1, 2, 3, 4, 5, 10 fold or more, when assayed under comparable conditions. In some embodiments, a compound of the present disclosure contacts the serine residue of an unmodified Target Protein in vitro. In some embodiments, a compound of the present disclosure contacts the serine residue of an unmodified Target Protein in vivo.

The compounds of Formula (I), (I), (II), (III), and (IV) disclosed herein, or a pharmaceutically acceptable salt or solvate thereof, are Target Protein inhibitors and have a wide range of applications in therapeutics, diagnostics, and other biomedical research. In some embodiments, a compound disclosed herein covalently modifies a Target Protein, such as a BTK protein. In some embodiments, a Target Protein, such as BTK, is contacted with a compound disclosed herein to form a modified Target Protein.

In some embodiments, STW is selected from

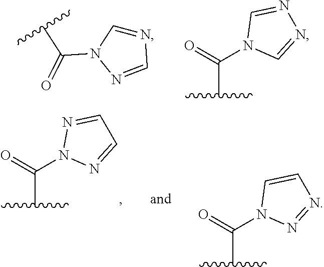

In some embodiments, STW is selected from

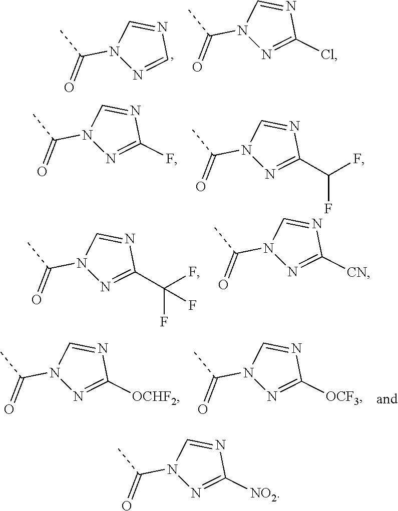

In some embodiments, STW is selected from

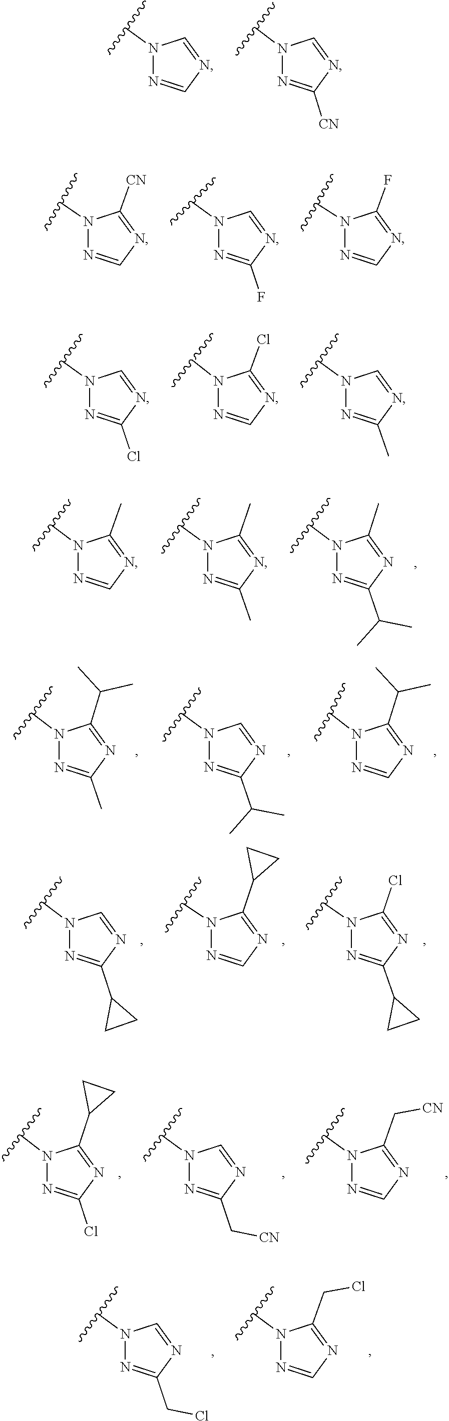

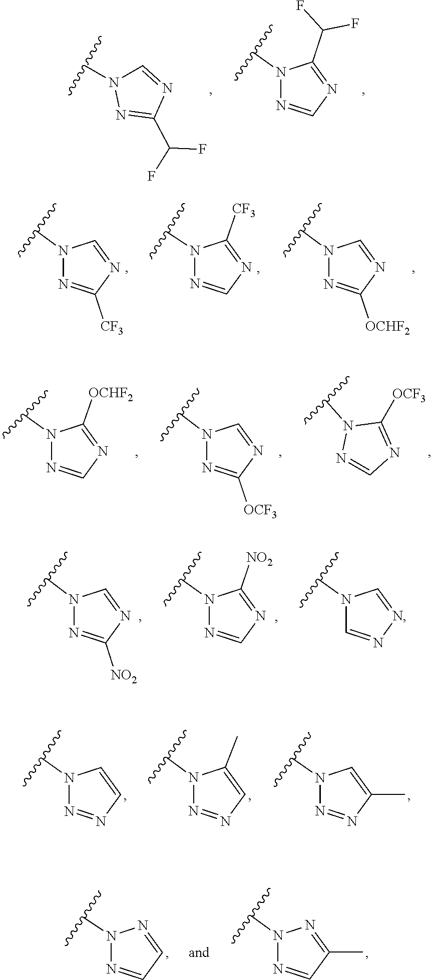

In some embodiments, STW is

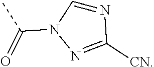

In some embodiments, for a compound described herein, R⁵ is independently selected at each occurrence from R³⁰.

In some embodiments, a compound of Formula (I), (I), (II), (III), or (IV) reversibly binds to the Target Protein when STW is replaced with hydrogen. In some embodiments, the compound reversibly binds to the Target Protein with an IC₅₀ of less than 1000 nM as assessed by an HTRF assay when STW is replaced with hydrogen.

In some embodiments, a compound described herein, such as a compound of Formula (I), (I), (II), (III), or (IV) is provided as a substantially pure stereoisomer. In some embodiments, the stereoisomer is provided in at least 80% enantiomeric excess, such as at least 85%, at least 90%, at least 95%, at least 96%, at least 97%, at least 98%, at least 99%, or at least 99.9% enantiomeric excess.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases or inorganic or organic acids to form a pharmaceutically acceptable salt. In some embodiments, such salts are prepared in situ during the final isolation and purification of the compounds described herein, or by separately reacting a purified compound in its free form with a suitable acid or base, and isolating the salt thus formed.

In some embodiments, the compounds described herein exist as solvates. In some embodiments are methods of treating diseases by administering such solvates. Further described herein are methods of treating diseases by administering such solvates as pharmaceutical compositions.

J Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydr of(ran, or 4MOa. In addition, the compounds provided herein exist in unsolvated as well as solvated forms. In general, the solvated forms are considered equivalent to the unsolvated forms for the purposes of the compounds and methods provided herein.

The chemical entities described herein can be synthesized according to one or more illustrative schemes herein and/or techniques known in the art. Materials used herein are either commercially available or prepared by synthetic methods generally known in the art.

In some embodiments, a compound of the present disclosure, for example, a compound of a formula given in Table 1, was synthesized according to one of the general routes outlined in Example 1 or by methods generally known in the art. In some embodiments, exemplary compounds may include, but are not limited to, a compound selected from Table 1, or a salt or solvate thereof.

TABLE 1

| | | | [M + |
|---|---|---|---|
| No. | Structure | Chemical Name | H]⁺ |
| 101 | 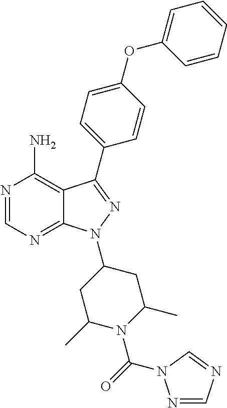 [SMILES: CC1CC(n2nc(-c3ccc(Oc4ccccc4)cc3)c3c(N)ncnc32)CC(C)N1C(=O)n1cncn1] | (4-(4-amino-3-(4-phenoxyphenyl)-1H- pyrazolo[3,4-d]pyrimidin-1-yl)-2,6- dimethylpiperidin-1-yl)(1H- 1,2,4-triazol-1-yl)methanone | 510.2 |
| 102 | 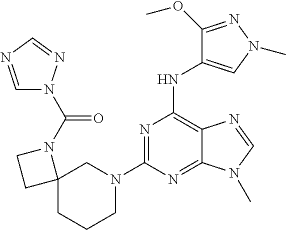 [SMILES: COc1nn(C)cc1Nc1nc(N2CCCC3(CCN3C(=O)n3cncn3)C2)nc2c1ncn2C] | (6-(6-((3-methoxy-1-methyl- 1H-pyrazol-4-yl)amino)-9-methyl- 9H-purin-2-yl)-1,6- diazaspiro[3.5]nonan-1-yl) (1H-1,2,4-triazol-1-yl)methanone | 479.2 |
| 103 | 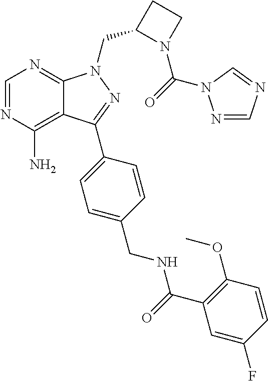 [SMILES: COc1ccc(F)cc1C(=O)NCc1ccc(-c2nn(C[C@@H]3CCN3C(=O)n3cncn3)c3ncnc(N)c23)cc1] | (S)-N-(4-(1-((1-(1H-1,2,4-triazole-1- carbonyl)azetidin-2-yl)methyl)- 4-amino-1H-pyrazolo[3,4-d] pyrimidin-3-yl)benzyl)-5- fluoro-2-methoxybenzamide | 557.2 |
| 104 | 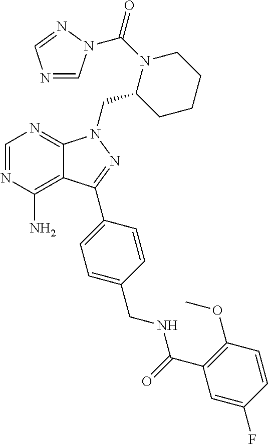 | (R)-N-(4-(1-((1-(1H-1,2,4-triazole-1- carbonyl)piperidin-2-yl)methyl)- 4-amino-1H-pyrazolo[3,4-d] pyrimidin-3-yl)benzyl)-5- fluoro-2-methoxybenzamide | 585.2 |
| 105 | 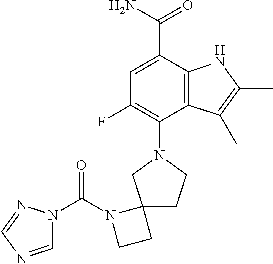 [SMILES: Cc1[nH]c2c(C(N)=O)cc(F)c(N3CCC4(CCN4C(=O)n4cncn4)C3)c2c1C] | 4-(1-(1H-1,2,4-triazole-1- carbonyl)-1,6-diazaspiro[3.4] octan-6-yl)-5-fluoro-2,3- dimethyl-1H-indole-7-carboxamide | 412.2 |
| 106 | 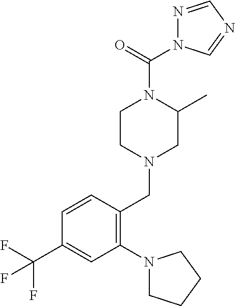 [SMILES: CC1CN(Cc2ccc(C(F)(F)F)cc2N2CCCC2)CCN1C(=O)n1cncn1] | (2-methyl-4-(2-(pyrrolidin-1-yl)-4- (trifluoromethyl)benzyl)piperazin- 1-yl)(1H-1,2,4-triazol-1-yl)methanone | 423.2 |
| 107 | 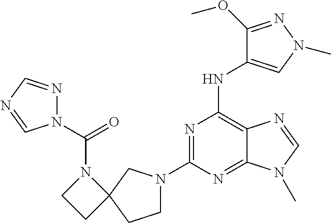 | (6-(6-((3-methoxy-1-methyl-1H- pyrazol-4-yl)amino)-9-methyl- 9H-purin-2-yl)-1,6-diazaspiro[3.4] octan-1-yl)(1H-1,2,4-triazol- 1-yl)methanone | 465.2 |
| 108 | 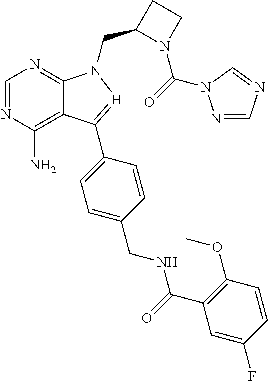 | (R)-N-(4-(1-((1-(1H-1,2,4-triazole-1- carbonyl)azetidin-2-yl)methyl)-4- amino-1H-pyrazolo[3,4-d] pyrimidin-3-yl)benzyl)-5-fluoro- 2-methoxybenzamide | 557.2 |
| 109 | 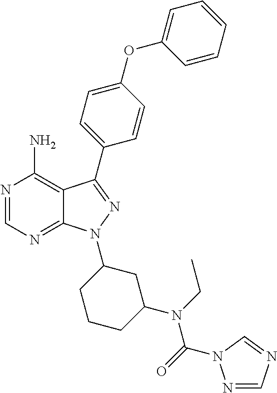 [SMILES: CCN(C(=O)n1cncn1)C1CCCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin- 1-yl)cyclohexyl)-N-ethyl-1H-1,2,4- triazole-1-carboxamide | 524.2 |
| 110 | 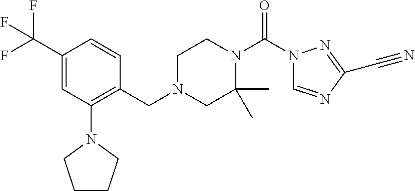 [SMILES: CC1(C)CN(Cc2ccc(C(F)(F)F)cc2N2CCCC2)CCN1C(=O)n1cnc(C#N)n1] | 1-(2,2-dimethyl-4-(2-(pyrrolidin-1-yl)- 4-(trifluoromethyl)benzyl)piperazine- 1-carbonyl)-1H-1,2,4-triazole- 3-carbonitrile | 462.5 |
| 111 | 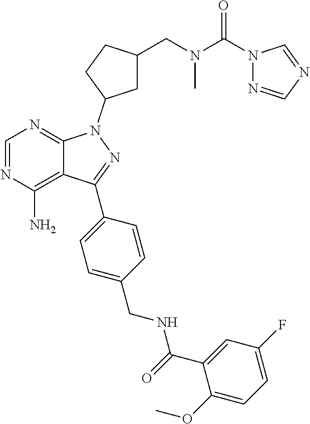 | N-((3-(4-amino-3-(4-((5-fluoro-2- methoxybenzamido)methyl)phenyl)- 1H-pyrazolo[3,4-d]pyrimidin-1- yl)cyclopentyl)methyl)-N-methyl-1H- 1,2,4-triazole-1-carboxamide | 599.3 |
| 112 | 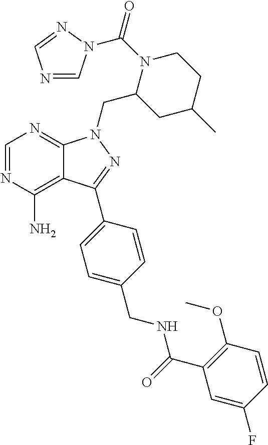 [SMILES: COc1ccc(F)cc1C(=O)NCc1ccc(-c2nn(CC3CC(C)CCN3C(=O)n3cncn3)c3ncnc(N)c23)cc1] | N-(4-(4-amino-1-((4-methyl-1-(1H- 1,2,4-triazole-1-carbonyl)piperidin- 2-yl)methyl)-1H-pyrazolo[3,4-d] pyrimidin-3-yl)benzyl)-5- fluoro-2-methoxybenzamide | 599.4 |
| 113 | 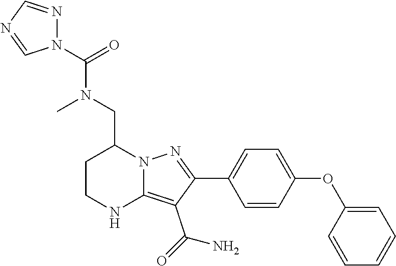 [SMILES: CN(CC1CCNc2c(C(N)=O)c(-c3ccc(Oc4ccccc4)cc3)nn21)C(=O)n1cncn1] | 7-((N-methyl-1H-1,2,4-triazole-1- carboxamido)methyl)-2-(4- phenoxyphenyl)-4,5,6,7-tetrahydro- pyrazolo[1,5-a]pyrimidine- 3-carboxamide | 473.2 |
| 114 | 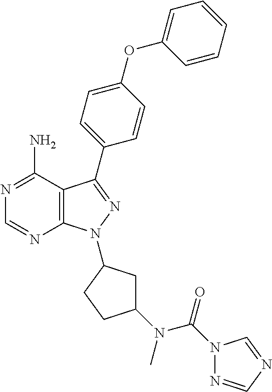 [SMILES: CN(C(=O)n1cncn1)C1CCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin- 1-yl)cyclopentyl)-N-methyl-1H- 1,2,4-triazole-1-carboxamide | 496.2 |
| 115 | 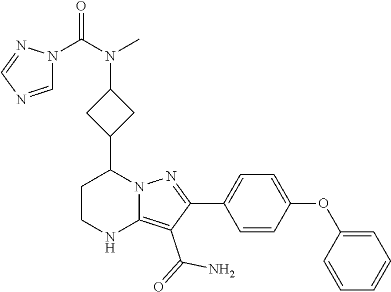 [SMILES: CN(C(=O)n1cncn1)C1CC(C2CCNc3c(C(N)=O)c(-c4ccc(Oc5ccccc5)cc4)nn32)C1] | 7-(3-(N-methyl-1H-1,2,4-triazole-1- carboxamido)cyclobutyl)-2-(4- phenoxyphenyl)-4,5,6,7- tetrahydropyrazolo[1,5-a]pyrimidine-3- carboxamide | 513.3 |
| 116 | 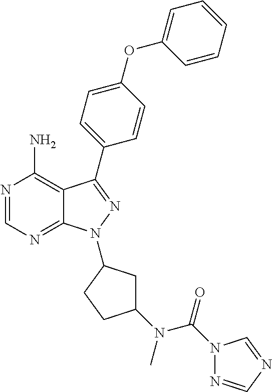 [SMILES: CN(C(=O)n1cncn1)C1CCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin- 1-yl)cyclopentyl)-N-methyl-1H- 1,2,4-triazole-1-carboxamide | 496.2 |
| 117 | 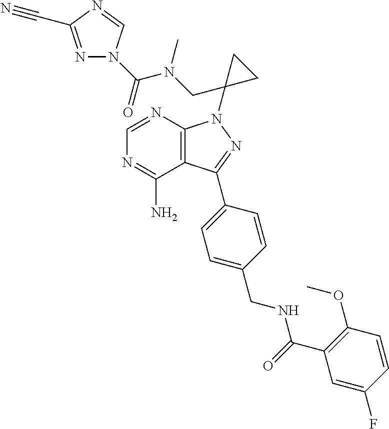 [SMILES: COc1ccc(F)cc1C(=O)NCc1ccc(-c2nn(C3(CN(C)C(=O)n4cnc(C#N)n4)CC3)c3ncnc(N)c23)cc1] | N-((1-(4-amino-3-(4-((5-fluoro-2- methoxybenzamido)methyl)phenyl)- 1H-pyrazolo[3,4-d]pyrimidin-1- yl)cyclopropyl)methyl)-3-cyano-N- methyl-1H-1,2,4-triazole-1- carboxamide | 596.3 |
| 118 | 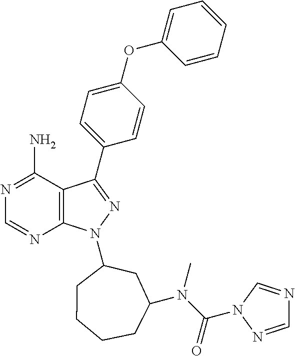 [SMILES: CN(C(=O)n1cncn1)C1CCCCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin- 1-yl)cycloheptyl)-N-methyl-1H-1,2,4- triazole-1-carboxamide | 524.3 |
| 119 | 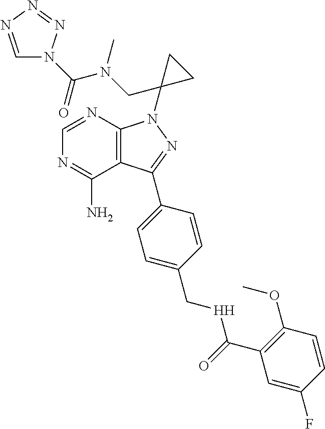 | N-((1-(4-amino-3-(4-((5-fluoro-2- methoxybenzamido)methyl)phenyl)- 1H-pyrazolo[3,4-d]pyrimidin-1- yl)cyclopropyl)methyl)-N-methyl-1H- tetrazole-1-carboxamide | 572.3 |
| 120 | 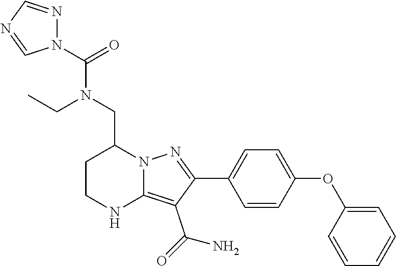 [SMILES: CCN(CC1CCNc2c(C(N)=O)c(-c3ccc(Oc4ccccc4)cc3)nn21)C(=O)n1cncn1] | 7-((N-ethyl-1H-1,2,4-triazole-1- carboxamido)methyl)-2-(4- phenoxyphenyl)-4,5,6,7-tetrahydro- pyrazolo[1,5-a]pyrimidine- 3-carboxamide | 487.0 |
| 121 | 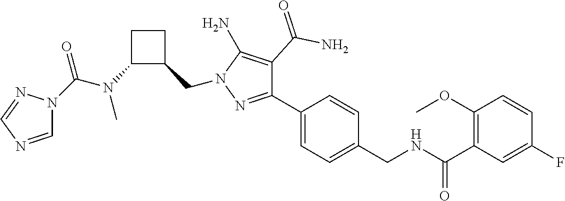 | N-((1R,2S)-2-((5-amino-4- carbamoyl-3-(4-((5-fluoro-2- methoxybenzamido)methyl) phenyl)-1H-pyrazol-1-yl)methyl) cyclobutyl)-N-methyl- 1H-1,2,4-triazole-1-carboxamide | 576.3 |
| 122 | 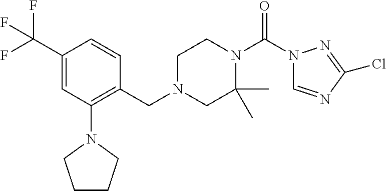 [SMILES: CC1(C)CN(Cc2ccc(C(F)(F)F)cc2N2CCCC2)CCN1C(=O)n1cnc(Cl)n1] | (3-chloro-1H-1,2,4-triazol-1-yl)(2,2- dimethyl-4-(2-(pyrrolidin-1-yl)-4- (trifluoromethyl)benzyl)piperazin-1- yl)methanone | 471.3 |
| 123 | 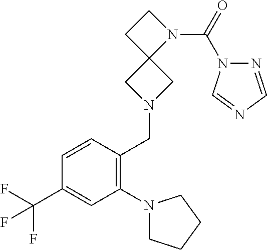 [SMILES: O=C(N1CCC12CN(Cc1ccc(C(F)(F)F)cc1N1CCCC1)C2)n1cncn1] | (6-(2-(pyrrolidin-1-yl)-4- (trifluoromethyl)benzyl)-1,6- diazaspiro[3.3]heptan-1-yl)(1H-1,2,4- triazol-1-yl)methanone | 421.2 |
| 124 | 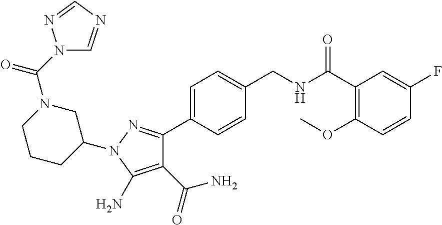 [SMILES: COc1ccc(F)cc1C(=O)NCc1ccc(-c2nn(C3CCCN(C(=O)n4cncn4)C3)c(N)c2C(N)=O)cc1] | 1-(1-(1H-1,2,4-triazole-1-carbonyl) piperidin-3-yl)-5-amino-3- (4-((5-fluoro-2-methoxybenzamido) methyl)phenyl)-1H- pyrazole-4-carboxamide | 562.4 |
| 125 | 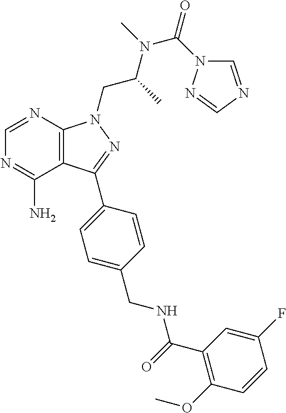 | (R)-N-(1-(4-amino-3-(4-((5-fluoro-2- methoxybenzamido)methyl) phenyl)-1H-pyrazolo[3,4-d] pyrimidin-1-yl)propan-2-yl)-N- methyl-1H-1,2,4-triazole-1- carboxamide | 559.3 |
| 126 | 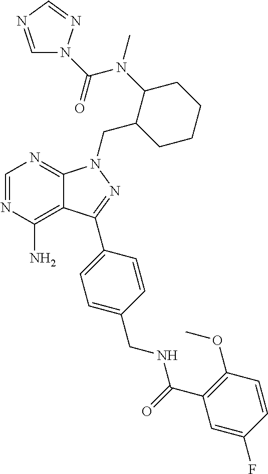 | N-(2-((4-amino-3-(4-((5-fluoro-2- methoxybenzamido)methyl)phenyl)- 1H-pyrazolo[3,4-d]pyrimidin-1-yl) methyl)cyclohexyl)-N-methyl-1H- 1,2,4-triazole-1-carboxamide | 613.3 |
| 127 | 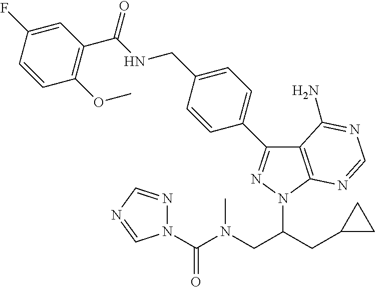 [SMILES: COc1ccc(F)cc1C(=O)NCc1ccc(-c2nn(C(CC3CC3)CN(C)C(=O)n3cncn3)c3ncnc(N)c23)cc1] | N-(4-(4-amino-3-(4-((5-fluoro-2- methoxybenzamido)methyl)phenyl)- 1H-pyrazolo[3,4-d]pyrimidin-1-yl)-3- cyclopropylpropyl)-N-methyl- 1H-1,2,4-triazole-1-carboxamide | 599.3 |
| 128 | 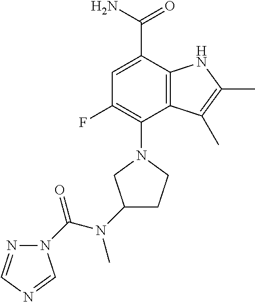 [SMILES: Cc1[nH]c2c(C(N)=O)cc(F)c(N3CCC(N(C)C(=O)n4cncn4)C3)c2c1C] | 5-fluoro-2,3-dimethyl-4-(3-(N- methyl-1H-1,2,4-triazole-1- carboxamido)pyrrolidin-1-yl)-1H- indole-7-carboxamide | 400.2 |
| 129 | 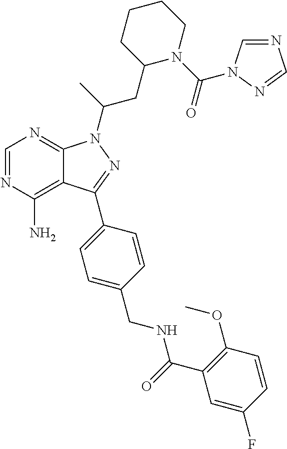 [SMILES: COc1ccc(F)cc1C(=O)NCc1ccc(-c2nn(C(C)CC3CCCCN3C(=O)n3cncn3)c3ncnc(N)c23)cc1] | N-(4-(1-(1-(1-(1H-1,2,4-triazole-1- carbonyl)piperidin-2-yl)propan-2- yl)-4-amino-1H-pyrazolo[3,4-d] pyrimidin-3-yl)benzyl)-5-fluoro-2- methoxybenzamide | 613.4 |
| 130 | 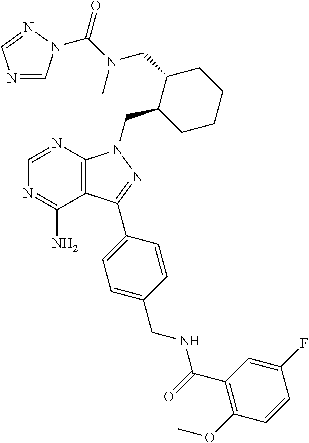 [SMILES: COc1ccc(F)cc1C(=O)NCc1ccc(-c2nn(C[C@@H]3CCCC[C@H]3CN(C)C(=O)n3cncn3)c3ncnc(N)c23)cc1] | N-(((1R,2R)-2-((4-amino-3-(4-((5- fluoro-2-methoxybenzamido) methyl)phenyl)-1H-pyrazolo [3,4-d]pyrimidin-1-yl)methyl) cyclohexyl)methyl)-N-methyl- 1H-1,2,4-triazole-1-carboxamide | 627.4 |
| 131 | 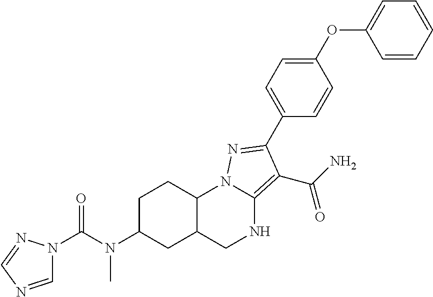 [SMILES: CN(C(=O)n1cncn1)C1CCC2C(CNc3c(C(N)=O)c(-c4ccc(Oc5ccccc5)cc4)nn32)C1] | 7-(N-methyl-1H-1,2,4-triazole-1- carboxamido)-2-(4-phenoxyphenyl)- 4,5,5a,6,7,8,9,9a-octahydropyrazolo [1,5-a]quinazoline-3-carboxamide | 513.3 |
| 132 | 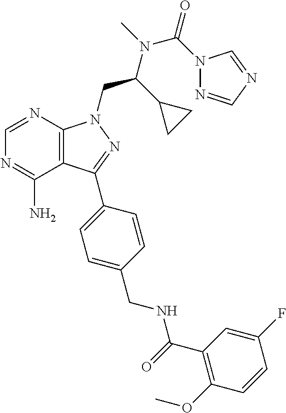 | (S)-N-(2-(4-amino-3-(4-((5-fluoro-2- methoxybenzamido)methyl)phenyl)- 1H-pyrazolo[3,4-d]pyrimidin-1-yl)-1- cyclopropylethyl)-N-methyl-1H-1,2,4- triazole-1-carboxamide | 585.3 |
| 133 | 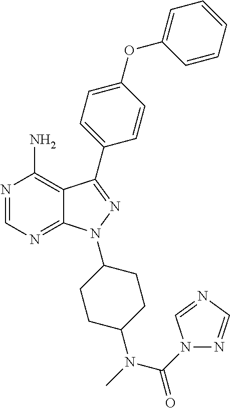 [SMILES: CN(C(=O)n1cncn1)C1CCC(n2nc(-c3ccc(Oc4ccccc4)cc3)c3c(N)ncnc32)CC1] | N-(4-(4-amino-3-(4-phenoxyphenyl)- 1H-pyrazolo[3,4-d]pyrimidin-1-yl) cyclohexyl)-N-methyl-1H-1,2,4- triazole-1-carboxamide | 510.2 |
| 134 | 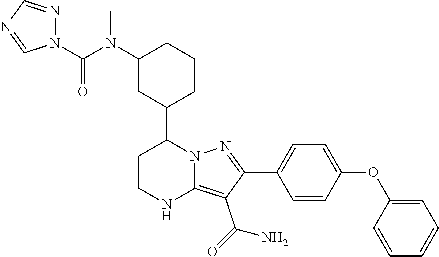 [SMILES: CN(C(=O)n1cncn1)C1CCCC(C2CCNc3c(C(N)=O)c(-c4ccc(Oc5ccccc5)cc4)nn32)C1] | 7-(3-(N-methyl-1H-1,2,4-triazole-1- carboxamido)cyclohexyl)-2-(4- phenoxyphenyl)-4,5,6,7- tetrahydropyrazolo[1,5-a]pyrimidine- 3-carboxamide | 541.3 |
| 135 | 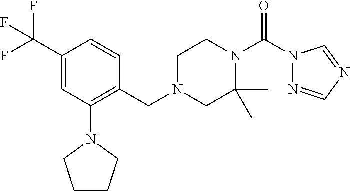 [SMILES: CC1(C)CN(Cc2ccc(C(F)(F)F)cc2N2CCCC2)CCN1C(=O)n1cncn1] | (2,2-dimethyl-4-(2-(pyrrolidin-1-yl)-4- (trifluoromethyl)benzyl)piperazin-1-yl) (1H-1,2,4-triazol-1-yl)methanone | 437.2 |
| 136 | 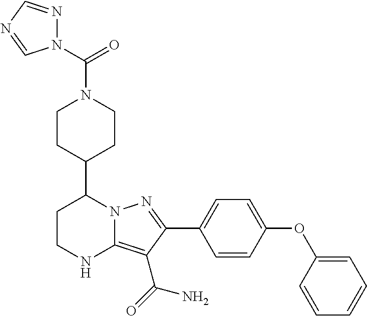 [SMILES: NC(=O)c1c(-c2ccc(Oc3ccccc3)cc2)nn2c1NCCC2C1CCN(C(=O)n2cncn2)CC1] | 7-(1-(1H-1,2,4-triazole-1-carbonyl) piperidin-4-yl)-2-(4-phenoxyphenyl)- 4,5,6,7-tetrahydropyrazolo[1,5-a] pyrimidine-3-carboxamide | 513.3 |
| 137 | 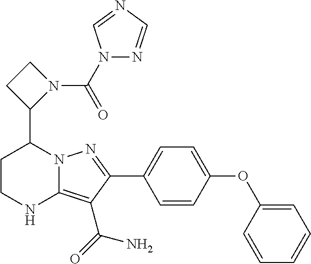 [SMILES: NC(=O)c1c(-c2ccc(Oc3ccccc3)cc2)nn2c1NCCC2C1CCN1C(=O)n1cncn1] | 7-(1-(1H-1,2,4-triazole-1-carbonyl) azetidin-2-yl)-2-(4-phenoxyphenyl)- 4,5,6,7-tetrahydropyrazolo[1,5-a] pyrimidine-3-carboxamide | 485.2 |
| 138 | 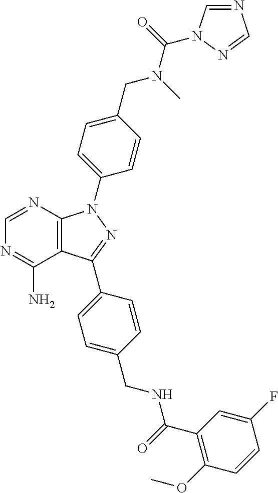 | N-(4-(4-amino-3-(4-((5-fluoro-2- methoxybenzamido)methyl)phenyl)- 1H-pyrazolo[3,4-d]pyrimidin-1-yl) benzyl)-N-methyl-1H-1,2,4- triazole-1-carboxamide | 607.3 |
| 139 | 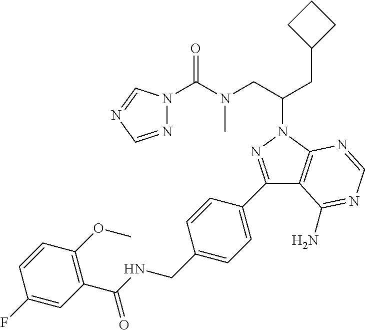 [SMILES: COc1ccc(F)cc1C(=O)NCc1ccc(-c2nn(C(CC3CCC3)CN(C)C(=O)n3cncn3)c3ncnc(N)c23)cc1] | N-(2-(4-amino-3-(4-((5-fluoro-2- methoxybenzamido)methyl)phenyl)- 1H-pyrazolo[3,4-d]pyrimidin-1-yl)-3- cyclobutylpropyl)-N-methyl-1H- 1,2,4-triazole-1-carboxamide | 613.4 |
| 140 | 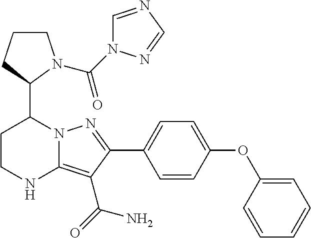 [SMILES: NC(=O)c1c(-c2ccc(Oc3ccccc3)cc2)nn2c1NCCC2[C@H]1CCCN1C(=O)n1cncn1] | 7-((R)-1-(1H-1,2,4-triazole-1- carbonyl)pyrrolidin-2-yl)-2-(4- phenoxyphenyl)-4,5,6,7- tetrahydropyrazolo[1,5-a] pyrimidine-3-carboxamide | 499.2 |
| 141 | 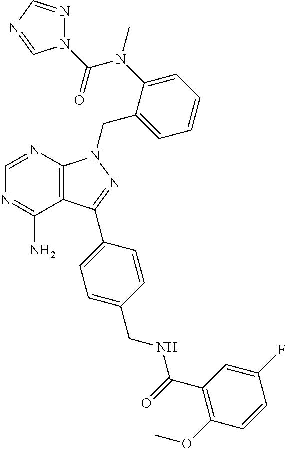 | N-(2-((4-amino-3-(4-((5-fluoro-2- methoxybenzamido)methyl)phenyl)- 1H-pyrazolo[3,4-d]pyrimidin-1- yl)methyl)phenyl)-N-methyl- 1H-1,2,4-triazole-1-carboxamide | 607.3 |
| 142 | 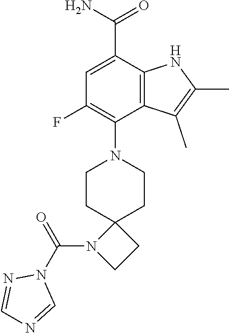 [SMILES: Cc1[nH]c2c(C(N)=O)cc(F)c(N3CCC4(CC3)CCN4C(=O)n3cncn3)c2c1C] | 4-(1-(1H-1,2,4-triazole-1-carbonyl)- 1,7-diazaspiro[3.5]nonan-7-yl)-5- fluoro-2,3-dimethyl-1H-indole-7- carboxamide | 426.2 |
| 143 | 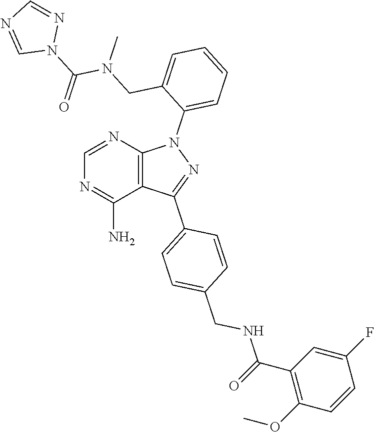 | N-(2-(4-amino-3-(4-((5-fluoro-2- methoxybenzamido)methyl)phenyl)- 1H-pyrazolo[3,4-d]pyrimidin-1-yl) benzyl)-N-methyl-1H-1,2,4- triazole-1-carboxamide | 607.3 |
| 144 | 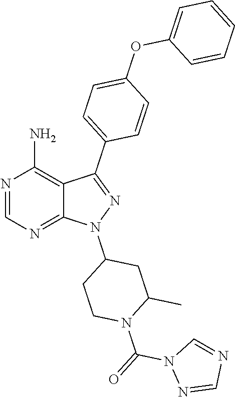 [SMILES: CC1CC(n2nc(-c3ccc(Oc4ccccc4)cc3)c3c(N)ncnc32)CCN1C(=O)n1cncn1] | (4-(4-amino-3-(4-phenoxyphenyl)- 1H-pyrazolo[3,4-d]pyrimidin-1-yl)-2- methylpiperidin-1-yl)(1H-1,2,4- triazol-1-yl)methanone | 496.2 |
| 145 | 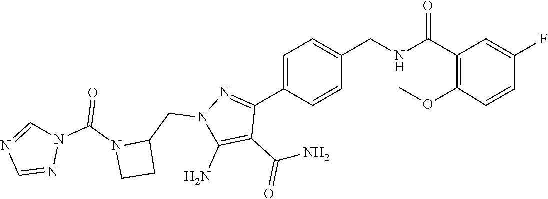 [SMILES: COc1ccc(F)cc1C(=O)NCc1ccc(-c2nn(CC3CCN3C(=O)n3cncn3)c(N)c2C(N)=O)cc1] | 1-((1-(1H-1,2,4-triazole-1-carbonyl) azetidin-2-yl)methyl)-5-amino-3- (4-((5-fluoro-2-methoxybenzamido) methyl)phenyl)-1H-pyrazole-4- carboxamide | 548.2 |
| 146 | 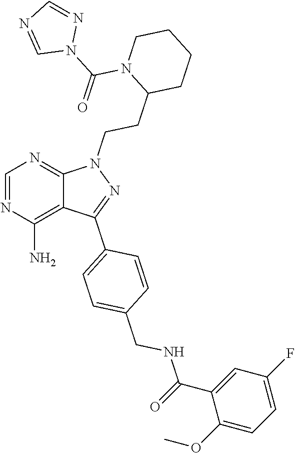 | N-(4-(1-(2-(1H-1,2,4-triazole-1- carbonyl)piperidin-2-yl)ethyl)-4- amino-1H-pyrazolo[3,4-d]pyrimidin- 3-yl)benzyl)-5-fluoro-2- methoxybenzamide | 599.3 |
| 147 | 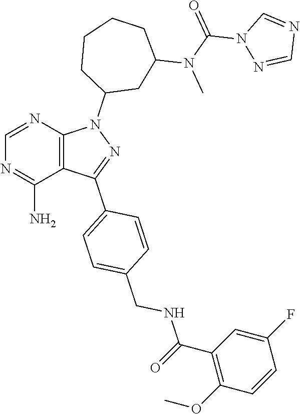 | N-(3-(4-amino-3-(4-((5-fluoro-2- methoxybenzamido)methyl)phenyl)- 1H-pyrazolo[3,4-d]pyrimidin-1-yl) cycloheptyl)-N-methyl-1H-1,2,4- triazole-1-carboxamide | 613.4 |
| 148 | 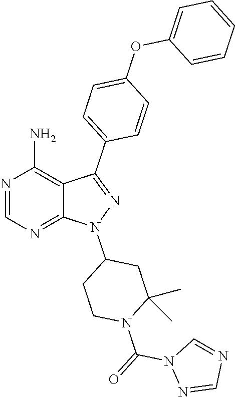 [SMILES: CC1(C)CC(n2nc(-c3ccc(Oc4ccccc4)cc3)c3c(N)ncnc32)CCN1C(=O)n1cncn1] | (4-(4-amino-3-(4-phenoxyphenyl)- 1H-pyrazolo[3,4-d]pyrimidin-1-yl)- 2,2-dimethylpiperidin-1-yl)(1H- 1,2,4-triazol-1-yl)methanone | 510.2 |
| 149 | 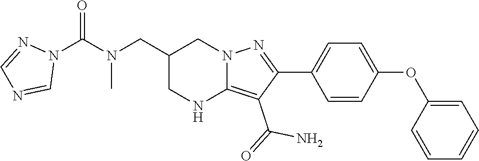 [SMILES: CN(CC1CNc2c(C(N)=O)c(-c3ccc(Oc4ccccc4)cc3)nn2C1)C(=O)n1cncn1] | 6-((N-methyl-1H-1,2,4-triazole-1- carboxamido)methyl)-2-(4- phenoxyphenyl)-4,5,6,7- tetrahydropyrazolo[1,5-a] pyrimidine-3-carboxamide | 473.2 |
| 150 | 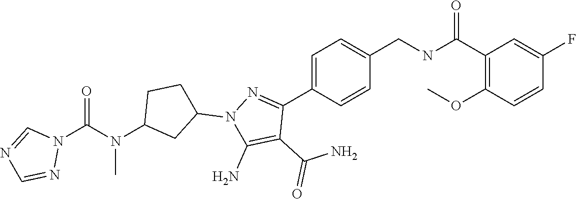 [SMILES: COc1ccc(F)cc1C(=O)NCc1ccc(-c2nn(C3CCC(N(C)C(=O)n4cncn4)C3)c(N)c2C(N)=O)cc1] | N-(3-(5-amino-4-carbamoyl-3-(4- ((5-fluoro-2-methoxybenzamido) methyl)phenyl)-1H-pyrazol-1- yl)cyclopentyl)-N-methyl-1H- 1,2,4-triazole-1-carboxamide | 576.3 |
| 151 | 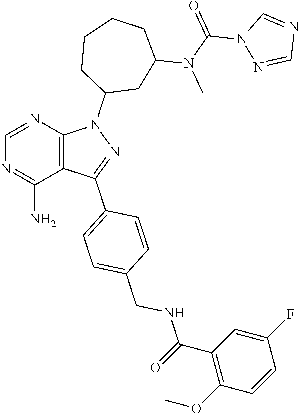 | N-(3-(4-amino-3-(4-((5-fluoro-2- methoxybenzamido)methyl)phenyl)- 1H-pyrazolo[3,4-d]pyrimidin-1-yl) cycloheptyl)-N-methyl-1H-1,2,4- triazole-1-carboxamide | 613.3 |
| 152 | 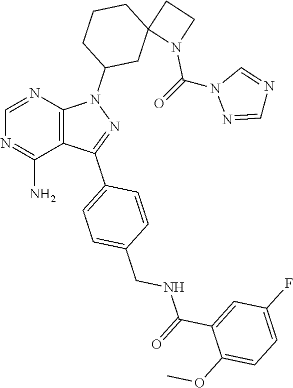 | N-(4-(1-(1-(1H-1,2,4-triazole-1- carbonyl)-1-azaspiro[3.5] nonan-6-yl)-4-amino-1H- pyrazolo[3,4-d]pyrimidin-3-yl) benzyl)-5-fluoro-2- methoxybenzamide | 611.4 |
| 153 | 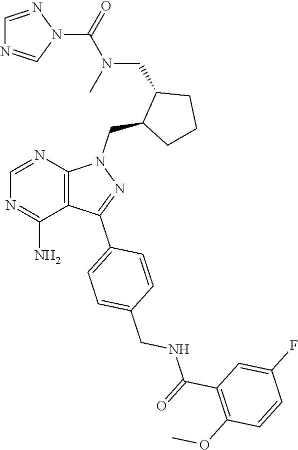 | N-(((1R,2R)-2-((4-amino-3-(4-((5- fluoro-2-methoxybenzamido) methyl)phenyl)-1H-pyrazolo[3,4-d] pyrimidin-1-yl)methyl)cyclopentyl) methyl)-N-methyl-1H-1,2,4- triazole-1-carboxamide | 613.4 |
| 154 | 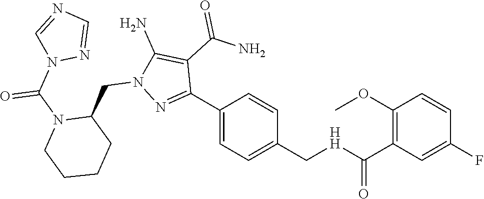 | (R)-1-((1-(1H-1,2,4-triazole-1- carbonyl)piperidin-2-yl)methyl)-5- amino-3-(4-((5-fluoro-2- methoxybenzamido)methyl)phenyl)- 1H-pyrazole-4-carboxamide | 576.4 |
| 155 | 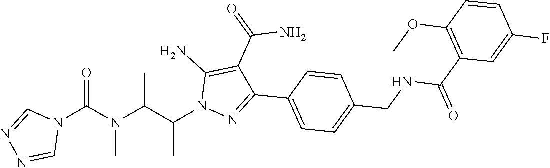 [SMILES: COc1ccc(F)cc1C(=O)NCc1ccc(-c2nn(C(C)C(C)N(C)C(=O)n3cnnc3)c(N)c2C(N)=O)cc1] | N-(3-(5-amino-4-carbamoyl-3-(4- ((5-fluoro-2-methoxybenzamido) methyl)phenyl)-1H-pyrazol-1-yl) butan-2-yl)-N-methyl-4H-1,2,4- triazole-4-carboxamide | 564.3 |
| 156 | 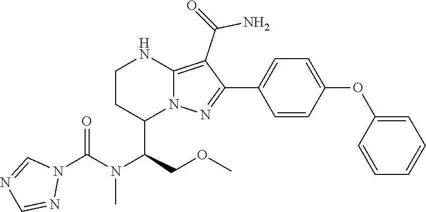 [SMILES: COC[C@H](C1CCNc2c(C(N)=O)c(-c3ccc(Oc4ccccc4)cc3)nn21)N(C)C(=O)n1cncn1] | 7-((S)-2-methoxy-1-(N-methyl-1H- 1,2,4-triazole-1-carboxamido)ethyl)- 2-(4-phenoxyphenyl)-4,5,6,7- tetrahydropyrazolo[1,5-a] pyrimidine-3-carboxamide | 517.3 |
| 157 | 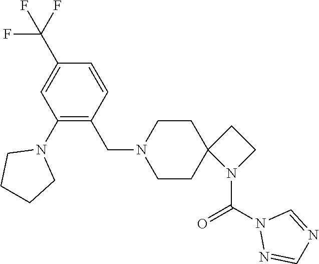 [SMILES: O=C(N1CCC12CCN(Cc1ccc(C(F)(F)F)cc1N1CCCC1)CC2)n1cncn1] | (7-(2-(pyrrolidin-1-yl)-4- (trifluoromethyl)benzyl)-1,7- diazaspiro[3.5]nonan-1-yl)(1H- 1,2,4-triazol-1-yl)methanone | 449.2 |
| 158 | 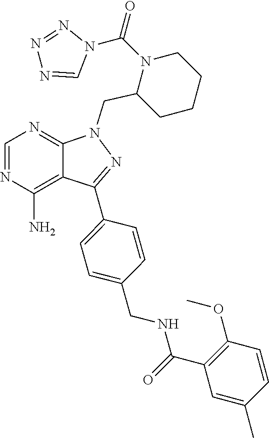 | N-(4-(1-((1-(1H-tetrazole-1- carbonyl)piperidin-2-yl)methyl)-4- amino-1H-pyrazolo[3,4-d] pyrimidin-3-yl)benzyl)-2- methoxy-5-methylbenzamide | 582.3 |
| 159 | 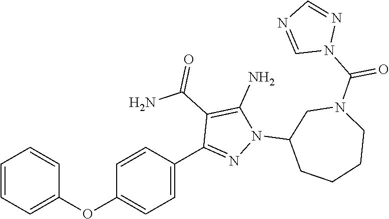 [SMILES: NC(=O)c1c(-c2ccc(Oc3ccccc3)cc2)nn(C2CCCCN(C(=O)n3cncn3)C2)c1N] | 1-(1-(1H-1,2,4-triazole-1-carbonyl) azepan-3-yl)-5-amino-3-(4- phenoxyphenyl)-1H-pyrazole-4- carboxamide | 487.2 |
| 160 | 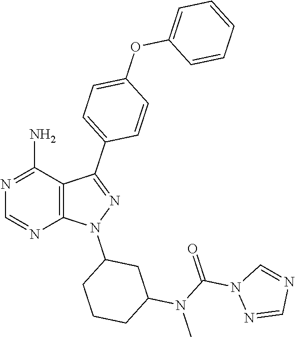 [SMILES: CN(C(=O)n1cncn1)C1CCCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin-1-yl) cyclohexyl)-N-methyl-1H-1,2,4- triazole-1-carboxamide | 510.3 |
| 161 | 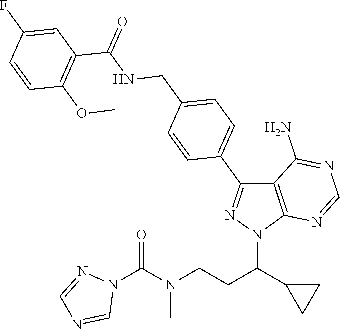 [SMILES: COc1ccc(F)cc1C(=O)NCc1ccc(-c2nn(C(CCN(C)C(=O)n3cncn3)C3CC3)c3ncnc(N)c23)cc1] | N-(3-(4-amino-3-(4-((5-fluoro-2- methoxybenzamido)methyl) phenyl)-1H-pyrazolo[3,4-d] pyrimidin-1-yl)-3-cyclopropyl)- N-methyl-1H-1,2,4-triazole-1- carboxamide | 599.3 |
| 162 | 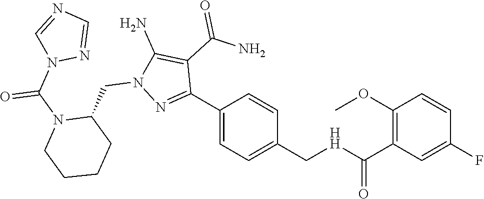 | (S)-1-((1H-1,2,4-triazole-1- carbonyl)piperidin-2-yl)methyl)-5- amino-3-(4-((5-fluoro-2- methoxybenzamido)methyl) phenyl)-1H-pyrazole-4- carboxamide | 576.4 |
| 163 | 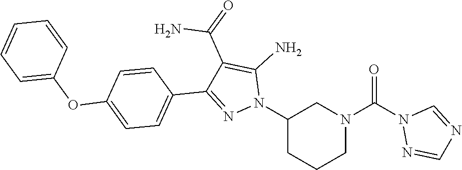 [SMILES: NC(=O)c1c(-c2ccc(Oc3ccccc3)cc2)nn(C2CCCN(C(=O)n3cncn3)C2)c1N] | 1-(1-(1H-1,2,4-triazole-1-carbonyl) piperidin-3-yl)-5-amino-3-(4- phenoxyphenyl)-1H-pyrazole-4- carboxamide | 473.2 |
| 164 | 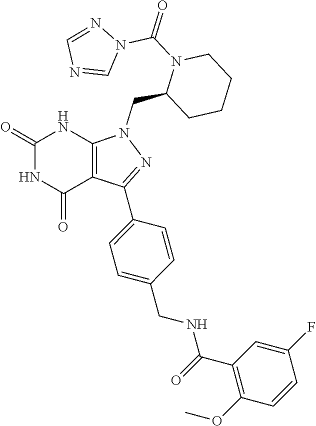 | (S)-N-(4-(1-((1-(1H-1,2,4-triazole-1- carbonyl)piperidin-2-yl)methyl-4,6- dioxo-4,5,6,7-tetrahydro-1H- pyrazolo[3,4-d]pyrimidin-3-yl) benzyl)-5-fluoro-2- methoxybenzamide | 602.5 |
| 165 | 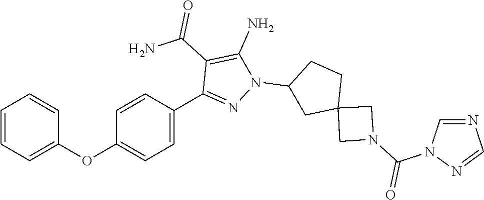 [SMILES: NC(=O)c1c(-c2ccc(Oc3ccccc3)cc2)nn(C2CCC3(C2)CN(C(=O)n2cncn2)C3)c1N] | 1-(2-(1H-1,2,4-triazole-1-carbonyl)-2- azaspiro[3.4]octan-6-yl)-5-amino-3- (4-phenoxyphenyl)-1H-pyrazole-4- carboxamide | 499.2 |
| 166 | 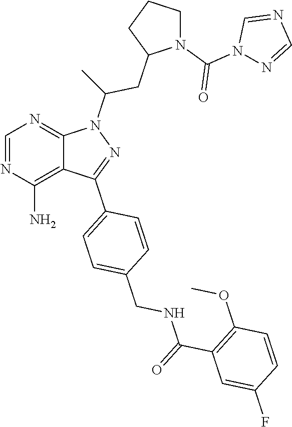 | N-(4-(1-(1-(1-(1H-1,2,4-triazole-1- carbonyl)pyrrolidin-2-yl)propan-2-yl)- 4-amino-1H-pyrazolo[3,4-d]pyrimidin- 3-yl)benzyl)-5-fluoro-2- methoxybenzamide | 599.3 |
| 167 | 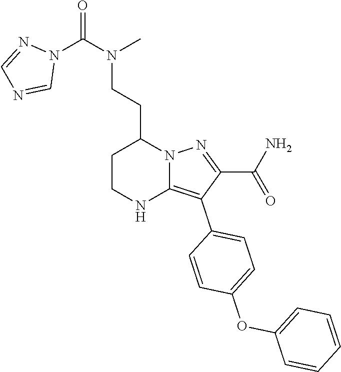 [SMILES: CN(CCC1CCNc2c(-c3ccc(Oc4ccccc4)cc3)c(C(N)=O)nn21)C(=O)n1cncn1] | 7-(2-(N-methyl-1H-1,2,4-triazole-1- carboxamido)ethyl)-3-(4-phenoxy- phenyl)-4,5,6,7-tetrahydro- pyrazolo[1,5-a]pyrimidine-2- carboxamide | 487.2 |
| 168 | 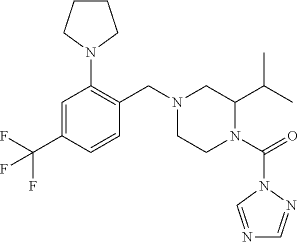 [SMILES: CC(C)C1CN(Cc2ccc(C(F)(F)F)cc2N2CCCC2)CCN1C(=O)n1cncn1] | (2-isopropyl-4-(2-(pyrrolidin-1-yl)-4- (trifluoromethyl)benzyl)piperazin-1-yl) (1H-1,2,4-triazol-1-yl)methanone | 451.3 |
| 169 | 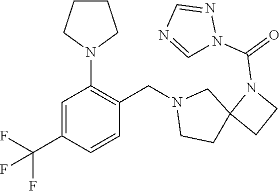 [SMILES: O=C(N1CCC12CCN(Cc1ccc(C(F)(F)F)cc1N1CCCC1)C2)n1cncn1] | (6-(2-(pyrrolidin-1-yl)-4- (trifluoromethyl)benzyl)-1,6- diazaspiro[3.4]octan-1-yl)(1H- 1,2,4-triazol-1-yl)methanone | 435.2 |
| 170 | 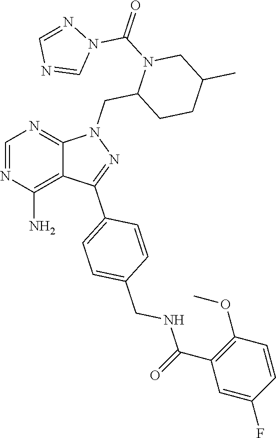 [SMILES: COc1ccc(F)cc1C(=O)NCc1ccc(-c2nn(CC3CCC(C)CN3C(=O)n3cncn3)c3ncnc(N)c23)cc1] | N-(4-(4-amino-1-((5-methyl-1- (1H-1,2,4-triazole-1-carbonyl) piperdin-2-yl)methyl)-1H- pyrazolo[3,4-d]pyrimidin-3-yl) benzyl)-5-fluoro-2- methoxybenzamide | 599.3 |
| 171 | 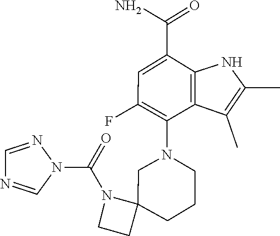 [SMILES: Cc1[nH]c2c(C(N)=O)cc(F)c(N3CCCC4(CCN4C(=O)n4cncn4)C3)c2c1C] | 4-(1-(1H-1,2,4-triazole-1- carbonyl)-1,6-diazaspiro[3.5] nonan-6-yl)-5-fluoro-2,3- dimethyl-1H-indole-7- carboxamide | 426.2 |
| 172 | 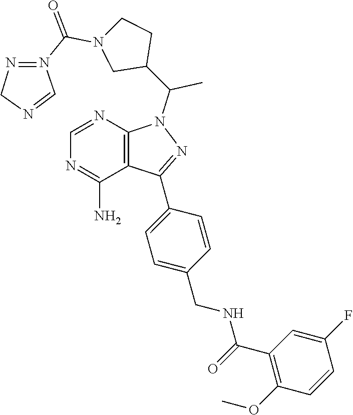 | N-(4-(1-(1-(1-(1H-1,2,4-triazole- 1-carbonyl)pyrrolidin-3-yl)ethyl)- 4-amino-1H-pyrazolo[3,4-d] pyrimidin-3-yl)benzyl)-5- fluoro-2-methoxybenzamide | 585.3 |
| 173 | 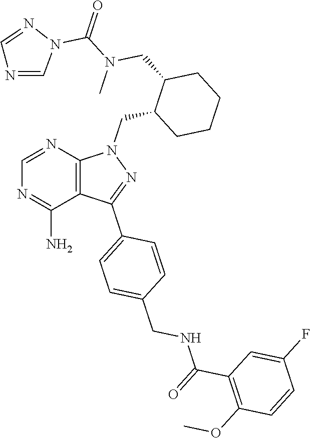 [SMILES: COc1ccc(F)cc1C(=O)NCc1ccc(-c2nn(C[C@H]3CCCC[C@H]3CN(C)C(=O)n3cncn3)c3ncnc(N)c23)cc1] | N-(((1R,2S)-2-((4-amino-3-(4-((5- fluoro-2-methoxybenzamido) methyl)phenyl)-1H-pyrazolo[3,4-d] pyrimidin-1-yl)methyl) cyclohexyl)methyl)-N-methyl- 1H-1,2,4-triazole-1-carboxamide | 627.4 |
| 174 | 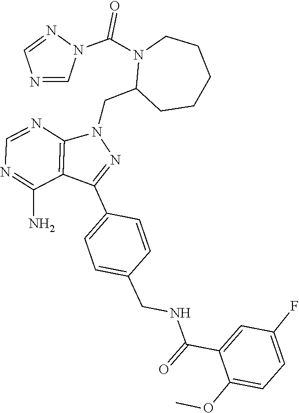 [SMILES: COc1ccc(F)cc1C(=O)NCc1ccc(-c2nn(CC3CCCCCN3C(=O)n3cncn3)c3ncnc(N)c23)cc1] | N-(4-(1-((1-(1H-1,2,4-triazole-1- carbonyl)azepan-2-yl)methyl)-4- amino-1H-pyrazolo[3,4-d] pyrimidin-3-yl)benzyl)-5- fluoro-2-methoxybenzamide | 599.3 |
| 175 | 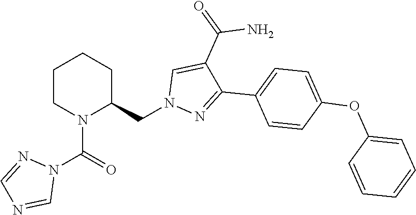 [SMILES: NC(=O)c1cn(C[C@@H]2CCCCN2C(=O)n2cncn2)nc1-c1ccc(Oc2ccccc2)cc1] | (S)-1-((1-(1H-1,2,4-triazole-1- carbonyl)piperidin-2-yl)methyl)-3- (4-phenoxyphenyl)-1H- pyrazole-4-carboxamide | 472.2 |
| 176 | 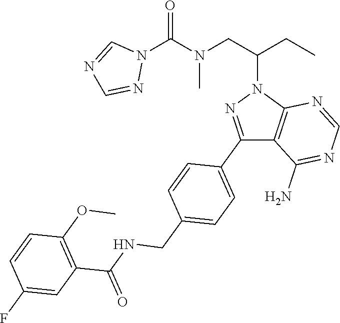 [SMILES: CCC(CN(C)C(=O)n1cncn1)n1nc(-c2ccc(CNC(=O)c3cc(F)ccc3OC)cc2)c2c(N)ncnc21] | N-(2-(4-amino-3-(4-((5-fluoro-2- methoxybenzamido)methyl)phenyl)- 1H-pyrazolo[3,4-d]pyrimidin-1-yl) butyl)-N-methyl-1H-1,2,4- triazole-1-carboxamide | 573.3 |
| 177 | 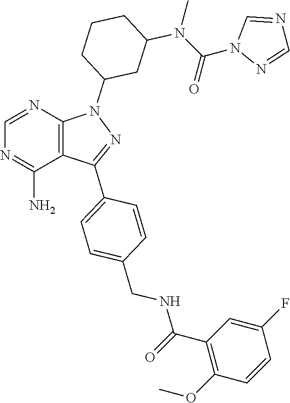 | N-(3-(4-amino-3-(4-((5-fluoro-2- methoxybenzamido)methyl) phenyl)-1H-pyrazolo[3,4-d] pyrimidin-1-yl)cyclohexyl)- N-methyl-1H-1,2,4-triazole-1- carboxamide | 599.3 |
| 178 | 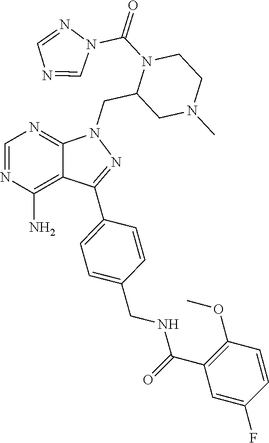 | N-(4-(4-amino-1-((4-methyl-1- (1H-1,2,4-triazole-1-carbonyl) piperazin-2-yl)methyl)-1H- pyrazolo[3,4-d]pyrimidin-3-yl) benzyl)-5-fluoro-2- methoxybenzamide | 600.3 |
| 179 | 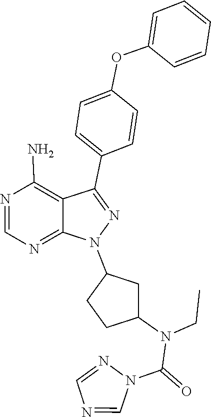 [SMILES: CCN(C(=O)n1cncn1)C1CCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin-1- yl)cyclopentyl)-N-ethyl-1H-1,2,4- triazole-1-carboxamide | 510.2 |
| 180 | 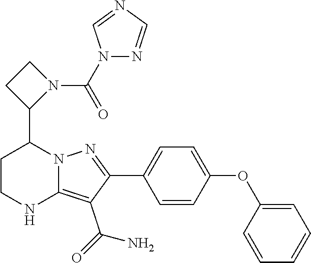 [SMILES: NC(=O)c1c(-c2ccc(Oc3ccccc3)cc2)nn2c1NCCC2C1CCN1C(=O)n1cncn1] | 7-(1-(1H-1,2,4-triazole-1-carbonyl) azetidin-2-yl)-2-(4-phenoxy- phenyl)-4,5,6,7-tetrahydropyrazolo [1,5-a]pyrimidine-3-carboxamide | 485.2 |
| 181 | 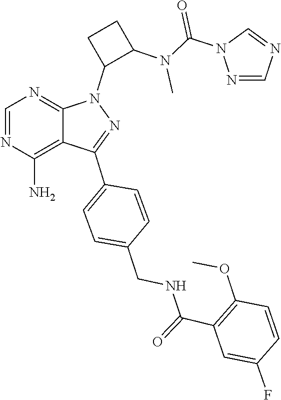 [SMILES: COc1ccc(F)cc1C(=O)NCc1ccc(-c2nn(C3CCC3N(C)C(=O)n3cncn3)c3ncnc(N)c23)cc1] | N-(2-(4-amino-3-(4-((5-fluoro-2- methoxybenzamido)methyl) phenyl)-1H-pyrazolo[3,4-d] pyrimidin-1-yl)cyclobutyl)- N-methyl-1H-1,2,4-triazole-1- carboxamide | 571.4 |
| 182 | 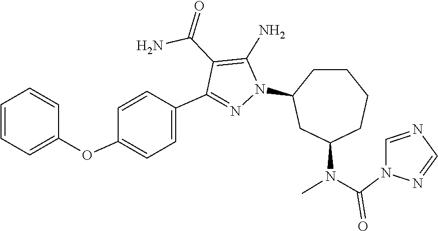 [SMILES: CN(C(=O)n1cncn1)[C@@H]1CCCC[C@H](n2nc(-c3ccc(Oc4ccccc4)cc3)c(C(N)=O)c2N)C1] | N-((1R,3S)-3-(5-amino-4-carbamoyl- 3-(4-phenoxyphenyl)-1H- pyrazol-1-yl)cycloheptyl)-N-methyl- 1H-1,2,4-triazole-1-carboxamide | 515.3 |
| 183 | 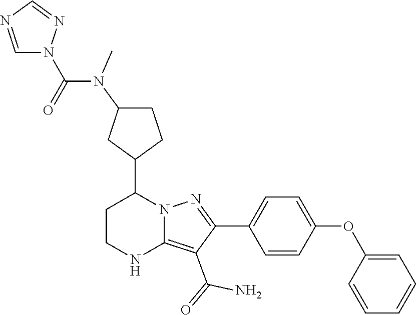 [SMILES: CN(C(=O)n1cncn1)C1CCC(C2CCNc3c(C(N)=O)c(-c4ccc(Oc5ccccc5)cc4)nn32)C1] | 7-(3-(N-methyl-1H-1,2,4-triazole-1- carboxamido)cyclopentyl)-2-(4- phenoxyphenyl)-4,5,6,7- tetrahydropyrazolo[1,5-a] pyrimidine-3-carboxamide | 527.3 |
| 184 | 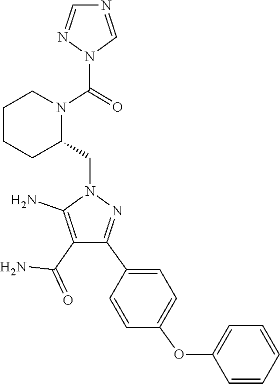 [SMILES: NC(=O)c1c(-c2ccc(Oc3ccccc3)cc2)nn(C[C@@H]2CCCCN2C(=O)n2cncn2)c1N] | (S)-1-((1-(1H-1,2,4-triazole-1- carbonyl)piperidin-2-yl)methyl)- 5-amino-3-(4-phenoxyphenyl)- 1H-pyrazole-4-carboxamide | 487.2 |
| 185 | 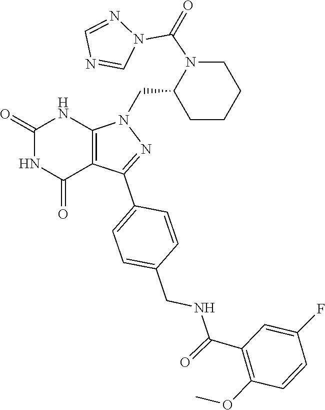 | (R)-N-(4-(1-((1-(1H-1,2,4-triazole- 1-carbonyl)piperidin-2-yl)methyl)- 4,6-dioxo-4,5,6,7-tetrahydro-1H- pyrazolo[3,4-d]pyrimidin-3-yl) benzyl)-5-fluoro-2- methoxybenzamide | 602.5 |
| 186 | 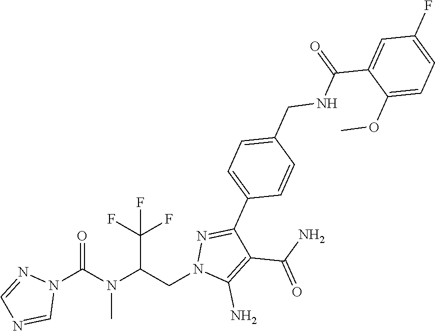 [SMILES: COc1ccc(F)cc1C(=O)NCc1ccc(-c2nn(CC(N(C)C(=O)n3cncn3)C(F)(F)F)c(N)c2C(N)=O)cc1] | N-(3-(5-amino-4-carbamoyl-3-(4- ((5-fluoro-2-methoxybenzamido) methyl)phenyl)-1H-pyrazol-1- yl)-1,1,1-trifluoropropan-2-yl)- N-methyl-1H-1,2,4-triazole-1- carboxamide | 604.3 |
| 187 | 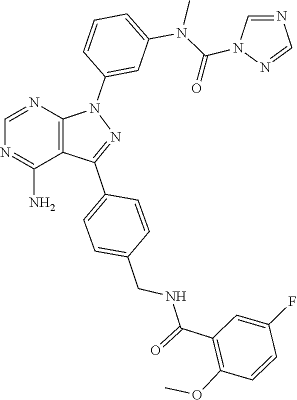 | N-(3-(4-amino-3-(4-((5-fluoro-2- methoxybenzamido)methyl) phenyl)-1H-pyrazolo[3,4-d] pyrimidin-1-yl)phenyl)-N- methyl-1H-1,2,4-triazole-1- carboxamide | 593.3 |
| 188 | 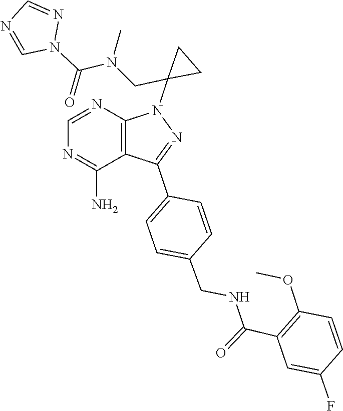 [SMILES: COc1ccc(F)cc1C(=O)NCc1ccc(-c2nn(C3(CN(C)C(=O)n4cncn4)CC3)c3ncnc(N)c23)cc1] | N-((1-(4-amino-3-(4-((5-fluoro-2- methoxybenzamido)methyl)phenyl)- 1H-pyrazolo[3,4-d]pyrimidin-1- yl)cyclopropyl)methyl)-N- methyl-1H-1,2,4-triazole-1- carboxamide | 571.3 |
| 189 | 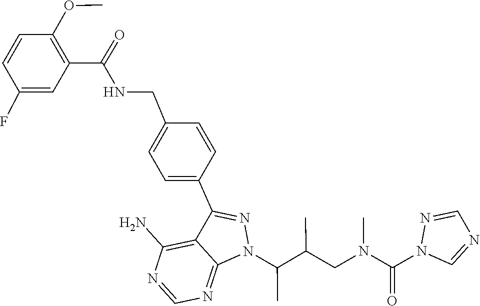 [SMILES: COc1ccc(F)cc1C(=O)NCc1ccc(-c2nn(C(C)C(C)CN(C)C(=O)n3cncn3)c3ncnc(N)c23)cc1] | N-(3-(4-amino-3-(4-((5-fluoro-2- methoxybenzamido)methyl)phenyl)- 1H-pyrazolo[3,4-d]pyrimidin-1-yl)- 2-methylbutyl)-N-methyl-1H-1,2,4- triazole-1-carboxamide | 587.3 |
| 190 | 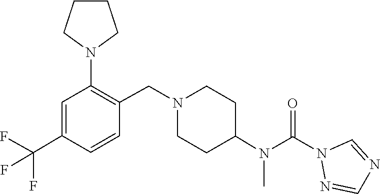 [SMILES: CN(C(=O)n1cncn1)C1CCN(Cc2ccc(C(F)(F)F)cc2N2CCCC2)CC1] | N-methyl-N-(1-(2-(pyrrolidin-1-yl)- 4-(trifluoromethyl)benzyl)piperidin- 4-yl)-1H-1,2,4-triazole-1-carboxamide | 437.2 |
| 191 | 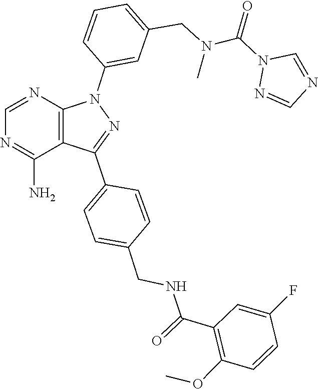 [SMILES: COc1ccc(F)cc1C(=O)NCc1ccc(-c2nn(-c3cccc(CN(C)C(=O)n4cncn4)c3)c3ncnc(N)c23)cc1] | N-(3-(4-amino-3-(4-((5-fluoro-2- methoxybenzamido)methyl)phenyl)- 1H-pyrazolo[3,4-d]pyrimidin-1-yl) benzyl)-N-methyl-1H-1,2,4- triazole-1-carboxamide | 607.3 |
| 192 | 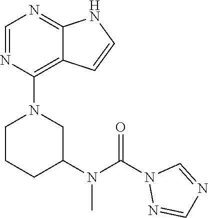 [SMILES: CN(C(=O)n1cncn1)C1CCCN(c2ncnc3[nH]ccc23)C1] | N-(1-(7H-pyrrolo[2,3-d]pyrimidin-4- yl)piperidin-3-yl)-N-methyl-1H- 1,2,4-triazole-1-carboxamide | 327.2 |
| 193 | 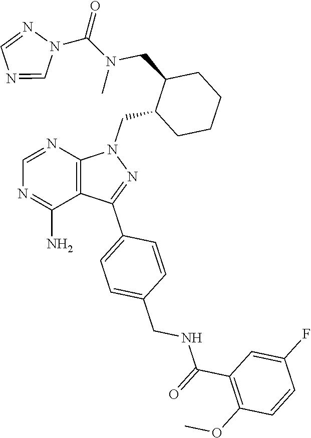 | N-(((1S,2S)-2-((4-amino-3-(4-((5- fluoro-2-methoxybenzamido) methyl)phenyl)-1H-pyrazolo [3,4-d]pyrimidin-1-yl)methyl) cyclohexyl)methyl)-N-methyl- 1H-1,2,4-triazole-1-carboxamide | 627.4 |
| 194 | 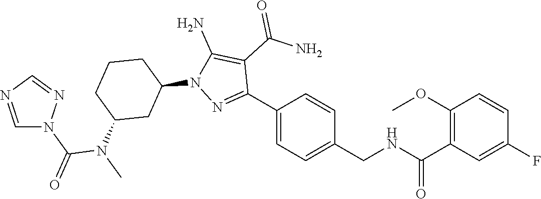 [SMILES: COc1ccc(F)cc1C(=O)NCc1ccc(-c2nn([C@@H]3CCC[C@@H](N(C)C(=O)n4cncn4)C3)c(N)c2C(N)=O)cc1] | N-((1R,3R)-3-(5-amino-4- carbamoyl-3-(4-((5-fluoro-2- methoxybenzamido)methyl) phenyl)-1H-pyrazol-1-yl) cyclohexyl)-N-methyl-1H- 1,2,4-triazole-1-carboxamide | 590.3 |
| 195 | 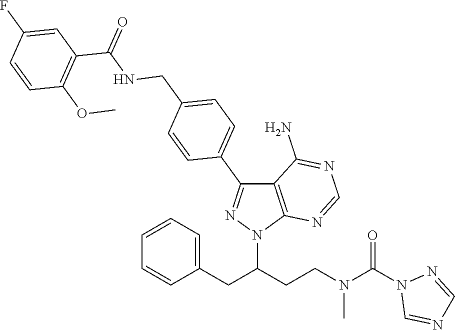 [SMILES: COc1ccc(F)cc1C(=O)NCc1ccc(-c2nn(C(CCN(C)C(=O)n3cncn3)Cc3ccccc3)c3ncnc(N)c23)cc1] | N-(3-(4-amino-3-(4-((5-fluoro-2- methoxybenzamido)methyl) phenyl)-1H-pyrazolo[3,4-d] pyrimidin-1-yl)-4-phenylbutyl)- N-methyl-1H-1,2,4-triazole-1- carboxamide | 649.4 |
| 196 | 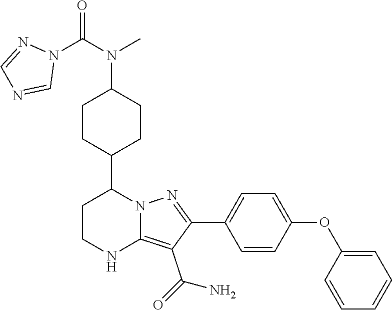 [SMILES: CN(C(=O)n1cncn1)C1CCC(C2CCNc3c(C(N)=O)c(-c4ccc(Oc5ccccc5)cc4)nn32)CC1] | 7-(4-(N-methyl-1H-1,2,4-triazole-1- carboxamido)cyclohexyl)-2-(4- phenoxyphenyl)-4,5,6,7- tetrahydropyrazolo[1,5-a] pyrimidine-3-carboxamide | 541.3 |
| 197 | 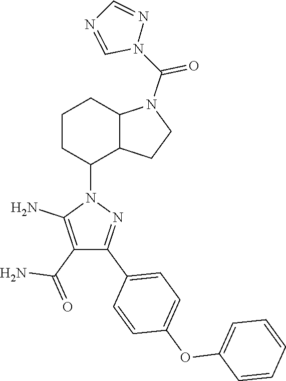 [SMILES: NC(=O)c1c(-c2ccc(Oc3ccccc3)cc2)nn(C2CCCC3C2CCN3C(=O)n2cncn2)c1N] | 1-(1-(1H-1,2,4-triazole-1- carbonyl)octahydro-1H-indol-4- yl)-5-amino-3-(4-phenoxy- phenyl)-1H-pyrazole-4-carboxamide | 513.3 |
| 198 | 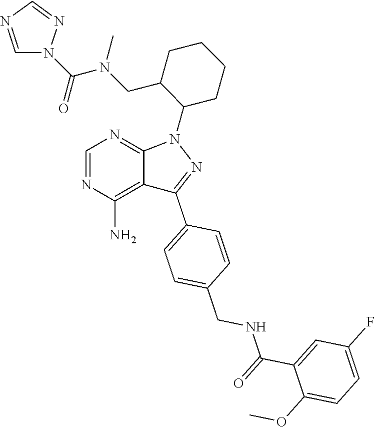 | N-((2-(4-amino-3-(4-((5-fluoro-2- methoxybenzamido)methyl) phenyl)-1H-pyrazolo[3,4-d] pyrimidin-1-yl)cyclohexyl) methyl)-N-methyl-1H-1,2,4- triazole-1-carboxamide | 613.4 |
| 199 | 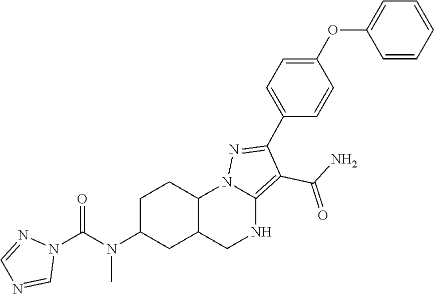 [SMILES: CN(C(=O)n1cncn1)C1CCC2C(CNc3c(C(N)=O)c(-c4ccc(Oc5ccccc5)cc4)nn32)C1] | 7-(N-methyl-1H-1,2,4-triazole-1- carboxamido)-2-(4-phenoxyphenyl)- 4,5,5a,6,7,8,9,9a-octahydro- pyrazolo[1,5-a]quinazoline-3- carboxamide | 513.3 |
| 200 | 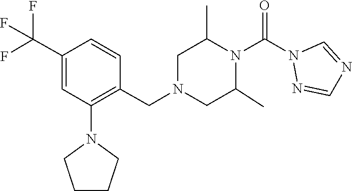 [SMILES: CC1CN(Cc2ccc(C(F)(F)F)cc2N2CCCC2)CC(C)N1C(=O)n1cncn1] | (2,6-dimethyl-4-(2-(pyrrolidin-1-yl)- 4-(trifluoromethyl)benzyl) piperazin-1-yl)(1H-1,2,4- triazol-1-yl)methanone | 437.2 |
| 201 | 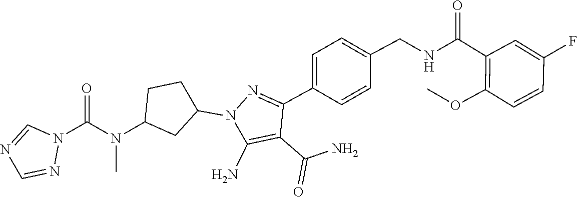 [SMILES: COc1ccc(F)cc1C(=O)NCc1ccc(-c2nn(C3CCC(N(C)C(=O)n4cncn4)C3)c(N)c2C(N)=O)cc1] | N-(3-(5-amino-4-carbamoyl-3-(4- ((5-fluoro-2-methoxybenzamido) methyl)phenyl)-1H-pyrazol-1- yl)cyclopentyl)-N-methyl-1H- 1,2,4-triazole-1-carboxamide | 576.3 |
| 202 | 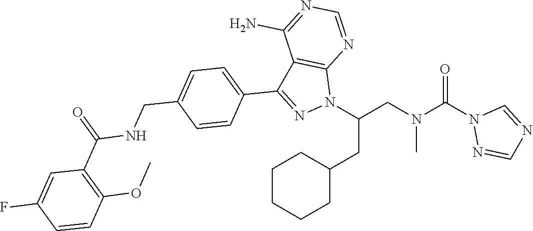 [SMILES: COc1ccc(F)cc1C(=O)NCc1ccc(-c2nn(C(CC3CCCCC3)CN(C)C(=O)n3cncn3)c3ncnc(N)c23)cc1] | N-(2-(4-amino-3-(4-((5-fluoro-2- methoxybenzamido)methyl)phenyl)- 1H-pyrazolo[3,4-d]pyrimidin-1-yl)- 3-cyclohexylpropyl)-N-methyl- 1H-1,2,4-triazole-1-carboxamide | 641.4 |
| 203 | 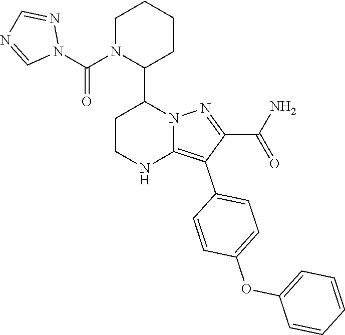 [SMILES: NC(=O)c1nn2c(c1-c1ccc(Oc3ccccc3)cc1)NCCC2C1CCCCN1C(=O)n1cncn1] | 7-(1-(1H-1,2,4-triazole-1-carbonyl) piperidin-2-yl)-3-(4-phenoxy- phenyl)-4,5,6,7-tetrahydro- pyrazolo[1,5-a]pyrimidine-2- carboxamide | 513.3 |
| 204 | 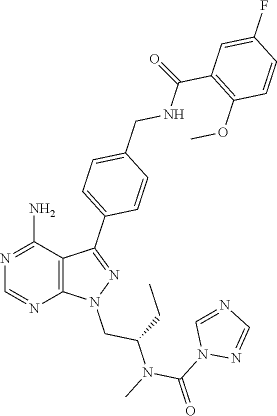 [SMILES: CC[C@@H](Cn1nc(-c2ccc(CNC(=O)c3cc(F)ccc3OC)cc2)c2c(N)ncnc21)N(C)C(=O)n1cncn1] | (S)-N-(1-(4-amino-3-(4-((5-fluoro-2- methoxybenzamido)methyl)phenyl)- 1H-pyrazolo[3,4-d]pyrimidin-1-yl) butan-2-yl)-N-methyl-1H-1,2,4- triazole-1-carboxamide | 573.3 |
| 205 | 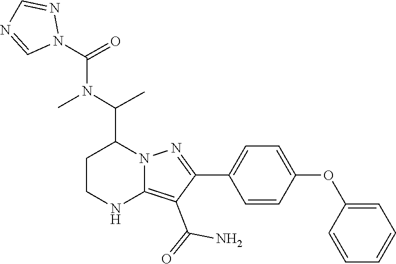 [SMILES: CC(C1CCNc2c(C(N)=O)c(-c3ccc(Oc4ccccc4)cc3)nn21)N(C)C(=O)n1cncn1] | 7-(1-(N-methyl-1H-1,2,4-triazole-1- carboxamido)ethyl)-2-(4-phenoxy- phenyl)-4,5,6,7-tetrahydro- pyrazolo[1,5-a]pyrimidine-3- carboxamide | 487.2 |
| 206 | 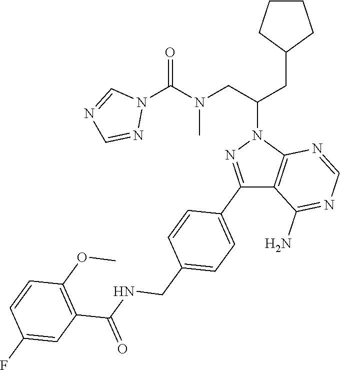 [SMILES: COc1ccc(F)cc1C(=O)NCc1ccc(-c2nn(C(CC3CCCC3)CN(C)C(=O)n3cncn3)c3ncnc(N)c23)cc1] | N-(2-(4-amino-3-(4-((5-fluoro-2- methoxybenzamido)methyl)phenyl)- 1H-pyrazolo[3,4-d]pyrimidin-1-yl)-3- cyclopentylpropyl)-N-methyl-1H- 1,2,4-triazole-1-carboxamide | 627.4 |
| 207 | 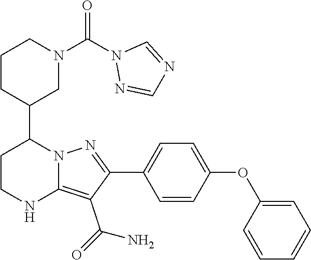 [SMILES: NC(=O)c1c(-c2ccc(Oc3ccccc3)cc2)nn2c1NCCC2C1CCCN(C(=O)n2cncn2)C1] | 7-(1-(1H-1,2,4-triazole-1-carbonyl) piperidin-3-yl)-2-(4-phenoxy- phenyl)-4,5,6,7-tetrahydro- pyrazolo[1,5-a]pyrimidine-3- carboxamide | 513.3 |
| 208 | 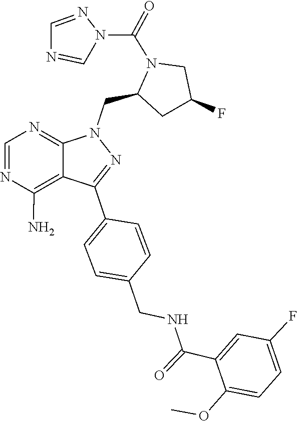 | N-(4-(4-amino-1-(((2S,4S)-4-fluoro- 1-(1H-1,2,4-triazole-1-carbonyl) pyrrolidin-2-yl)methyl)-1H- pyrazolo[3,4-d]pyrimidin-3-yl) benzyl)-5-fluoro-2- methoxybenzamide | 589.3 |
| 209 | 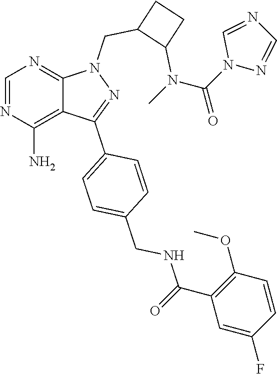 | N-(2-((4-amino-3-(4-((5-fluoro-2- methoxybenzamido)methyl)phenyl)- 1H-pyrazolo[3,4-d]pyrimidin-1- yl)methyl)cyclobutyl)-N-methyl- 1H-1,2,4-triazole-1-carboxamide | 585.4 |
| 210 | 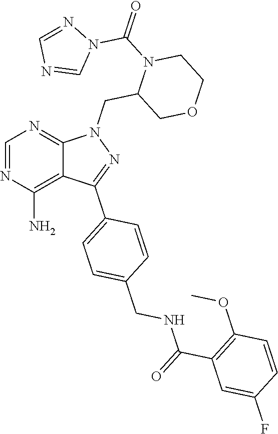 | N-(4-(1-((4-(1H-1,2,4-triazole-1- carbonyl)morpholin-3-yl)methyl)- 4-amino-1H-pyrazolo[3,4-d] pyrimidin-3-yl)benzyl)-5- fluoro-2-methoxybenzamide | 587.2 |
| 211 | 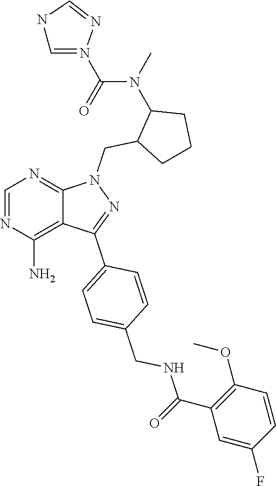 | N-(2-((4-amino-3-(4-((5-fluoro-2- methoxybenzamido)methyl)phenyl)- 1H-pyrazolo[3,4-d]pyrimidin-1- yl)methyl)cyclopentyl)-N-methyl- 1H-1,2,4-triazole-1-carboxamide | 599.4 |
| 212 | 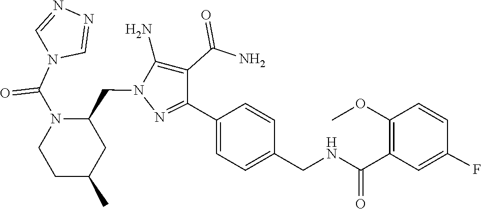 | 5-amino-3-(4-((5-fluoro-2- methoxybenzamido)methyl)phenyl)- 1-(((2R,4S)-4-methyl-1-(4H-1,2,4- triazole-4-carbonyl)piperidin-2-yl) methyl)-1H-pyrazole-4-carboxamide | 590.3 |
| 213 | 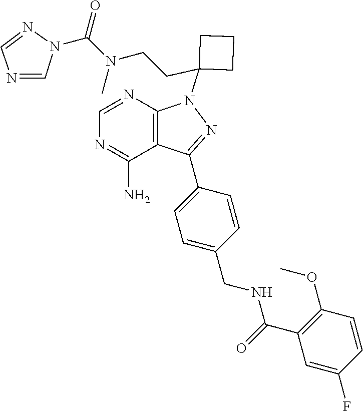 | N-(2-(1-(4-amino-3-(4-((5-fluoro-2- methoxybenzamido)methyl)phenyl)- 1H-pyrazolo[3,4-d]pyrimidin-1- yl)cyclobutyl)ethyl)-N-methyl- 1H-1,2,4-triazole-1-carboxamide | 599.3 |
| 214 | 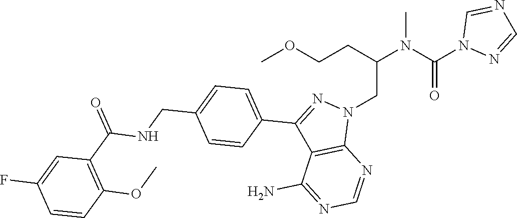 [SMILES: COCCC(Cn1nc(-c2ccc(CNC(=O)c3cc(F)ccc3OC)cc2)c2c(N)ncnc21)N(C)C(=O)n1cncn1] | N-(1-(4-amino-3-(4-((5-fluoro-2- methoxybenzamido)methyl) phenyl)-1H-pyrazolo[3,4-d] pyrimidin-1-yl)-4-methoxy- butan-2-yl)-N-methyl-1H-1,2,4- triazole-1-carboxamide | 603.3 |
| 215 | 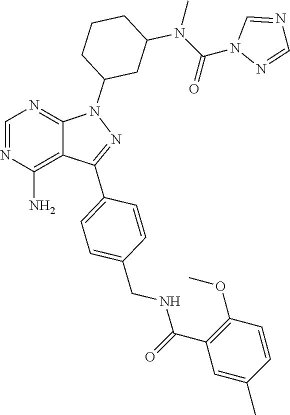 | N-(3-(4-amino-3-(4-((2-methoxy-5- methylbenzamido)methyl)phenyl)- 1H-pyrazolo[3,4-d]pyrimidin-1-yl) cyclohexyl)-N-methyl-1H-1,2,4- triazole-1-carboxamide | 595.3 |
| 216 | 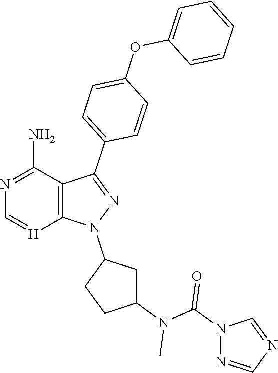 | N-(3-(4-amino-3-(4-phenoxyphenyl)- 1H-pyrazolo[3,4-d]pyrimidin-1-yl) cyclopentyl)-N-methyl-1H-1,2,4- triazole-1-carboxamide | 496.2 |
| 217 | 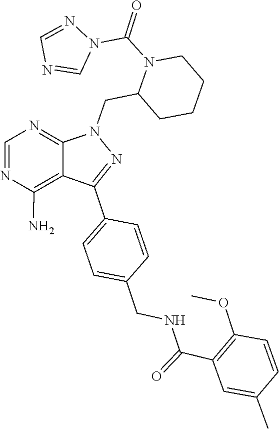 | N-(4-(1-((1-(1H-1,2,4-triazole-1- carbonyl)piperidin-2-yl)methyl)-4- amino-1H-pyrazolo[3,4-d] pyrimidin-3-yl)benzyl)-2- methoxy-5-methylbenzamide | 581.3 |
| 218 | 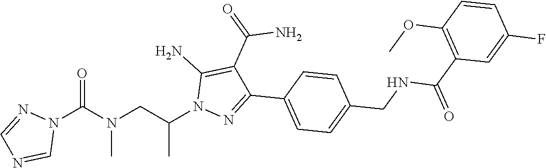 [SMILES: COc1ccc(F)cc1C(=O)NCc1ccc(-c2nn(C(C)CN(C)C(=O)n3cncn3)c(N)c2C(N)=O)cc1] | N-(2-(5-amino-4-carbamoyl-3-(4- ((5-fluoro-2-methoxybenzamido) methyl)phenyl)-1H-pyrazol-1-yl) propyl)-N-methyl-1H-1,2,4- triazole-1-carboxamide | 550.3 |
| 219 | 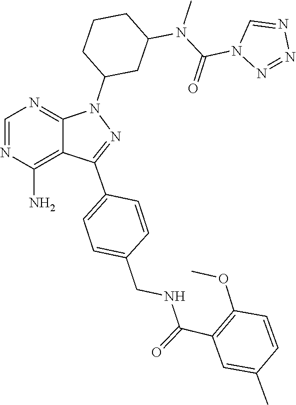 | N-(3-(4-amino-3-(4-((2-methoxy-5- methylbenzamido)methyl)phenyl)- 1H-pyrazolo[3,4-d]pyrimidin-1-yl) cyclohexyl)-N-methyl-1H- tetrazole-1-carboxamide | 596.3 |
| 220 | 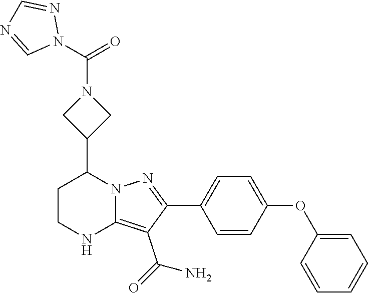 [SMILES: NC(=O)c1c(-c2ccc(Oc3ccccc3)cc2)nn2c1NCCC2C1CN(C(=O)n2cncn2)C1] | 7-(1-(1H-1,2,4-triazole-1-carbonyl) azetidin-3-yl)-2-(4-phenoxyphenyl)- 4,5,6,7-tetrahydropyrazolo[1,5-a] pyrimidine-3-carboxamide | 485.2 |
| 221 | 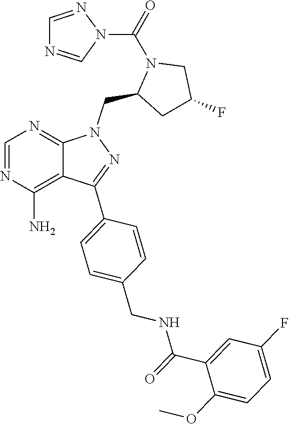 [SMILES: COc1ccc(F)cc1C(=O)NCc1ccc(-c2nn(C[C@@H]3C[C@@H](F)CN3C(=O)n3cncn3)c3ncnc(N)c23)cc1] | N-(4-(4-amino-1-(((2S,4R)-4- fluoro-1-(1H-1,2,4-triazole-1- carbonyl)pyrrolidin-2-yl)methyl)- 1H-pyrazolo[3,4-d]pyrimidin-3- yl)benzyl)-5-fluoro-2- methoxybenzamide | 589.3 |
| 222 | 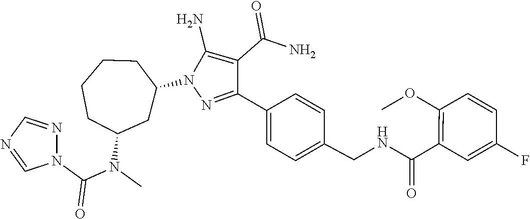 | N-((1R,3S)-3-(5-amino-4- carbamoyl-3-(4-((5-fluoro-2- methoxybenzamido)methyl) phenyl)-1H-pyrazol-1-yl) cycloheptyl)-N-methyl-1H- 1,2,4-triazole-1-carboxamide | 604.4 |
| 223 | 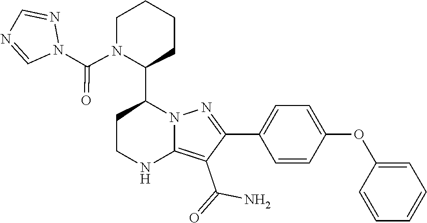 [SMILES: NC(=O)c1c(-c2ccc(Oc3ccccc3)cc2)nn2c1NCC[C@H]2[C@@H]1CCCCN1C(=O)n1cncn1] | (S)-7-((S)-1-(1H-1,2,4-triazole-1- carbonyl)piperidin-2-yl)-2-(4- phenoxyphenyl)-4,5,6,7- tetrahydropyrazolo[1,5-a] pyrimidine-3-carboxamide | 513.3 |
| 224 | 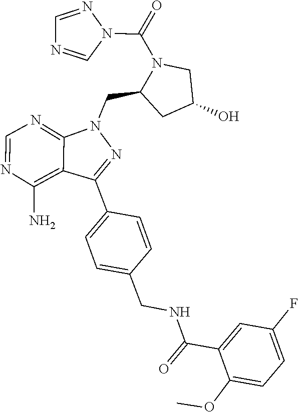 | N-(4-(4-amino-1-(((2S,4R)-4- hydroxy-1-(1H-1,2,4-triazole-1- carbonyl)pyrrolidin-2-yl)methyl)- 1H-pyrazolo[3,4-d]pyrimidin-3- yl)benzyl)-5-fluoro-2- methoxybenzamide | 587.3 |
| 225 | 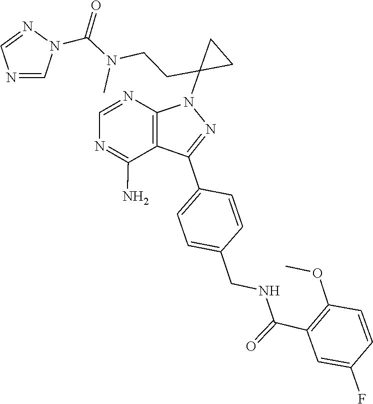 | N-(2-(1-(4-amino-3-(4-((5-fluoro-2- methoxybenzamido)methyl) phenyl)-1H-pyrazolo[3,4-d] pyrimidin-1-yl)cyclopropyl) ethyl)-N-methyl-1H-1,2,4- triazole-1-carboxamide | 585.3 |
| 226 | 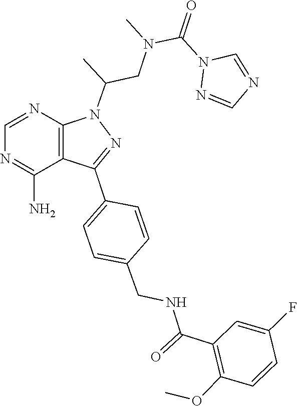 | N-(2-(4-amino-3-(4-((5-fluoro-2- methoxybenzamido)methyl) phenyl)-1H-pyrazolo[3,4-d] pyrimidin-1-yl)propyl)-N- methyl-1H-1,2,4-triazole-1- carboxamide | 559.4 |
| 227 | 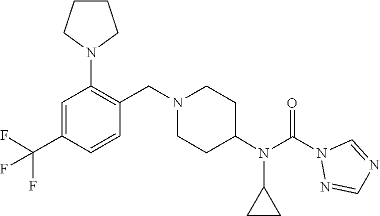 [SMILES: O=C(N(C1CC1)C1CCN(Cc2ccc(C(F)(F)F)cc2N2CCCC2)CC1)n1cncn1] | N-cyclopropyl-N-(1-(2-(pyrrolidin- 1-yl)-4-(trifluoromethyl)benzyl) piperidin-4-yl)-1H-1,2,4-triazole- 1-carboxamide | 463.3 |
| 228 | 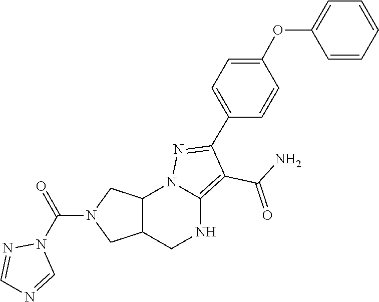 [SMILES: NC(=O)c1c(-c2ccc(Oc3ccccc3)cc2)nn2c1NCC1CN(C(=O)n3cncn3)CC12] | 2-(4-phenoxyphenyl)-7-(1H-1,2,4- triazole-1-carbonyl)-5,5a,6,7,8,8a- hexahydro-4H-pyrazolo[1,5-a] pyrrolo[3,4-e]pyrimidine-3- carboxamide | 471.2 |
| 229 | 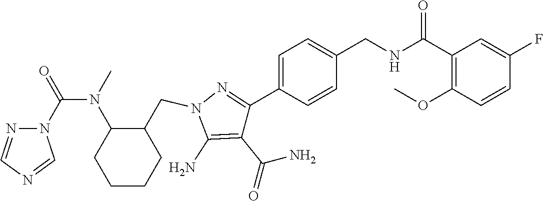 [SMILES: COc1ccc(F)cc1C(=O)NCc1ccc(-c2nn(CC3CCCCC3N(C)C(=O)n3cncn3)c(N)c2C(N)=O)cc1] | N-(2-((5-amino-4-carbamoyl-3-(4- ((5-fluoro-2-methoxybenzamido) methyl)phenyl)-1H-pyrazol-1- yl)methyl)cyclohexyl)-N-methyl- 1H-1,2,4-triazole-1-carboxamide | 604.4 |
| 230 | 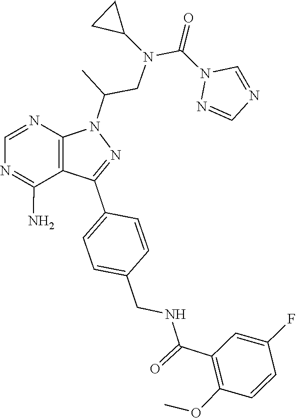 | N-(2-(4-amino-3-(4-((5-fluoro-2- methoxybenzamido)methyl)phenyl)- 1H-pyrazolo[3,4-d]pyrimidin-1-yl) propyl)-N-cyclopropyl-1H- 1,2,4-triazole-1-carboxamide | 585.3 |
| 231 | 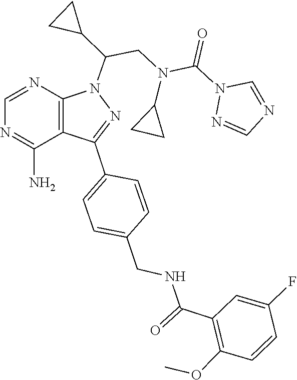 | N-(2-(4-amino-3-(4-((5-fluoro-2- methoxybenzamido)methyl) phenyl)-1H-pyrazolo[3,4-d] pyrimidin-1-yl)-2-cyclopropyl- ethyl)-N-cyclopropyl-1H-1,2,4- triazole-1-carboxamide | 611.4 |
| 232 | 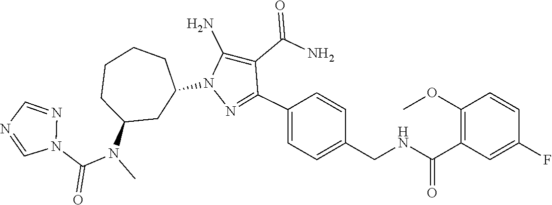 | N-((1S,3S)-3-(5-amino-4- carbamoyl-3-(4-((5-fluoro-2- methoxybenzamido)methyl) phenyl)-1H-pyrazol-1-yl) cycloheptyl)-N-methyl-1H- 1,2,4-triazole-1-carboxamide | 604.4 |
| 233 | 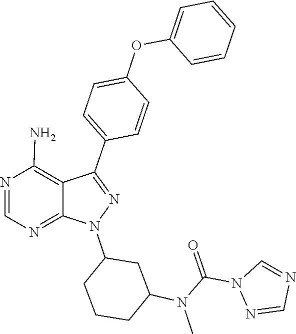 [SMILES: CN(C(=O)n1cncn1)C1CCCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin-1- yl)cyclohexyl)-N-methyl-1H-1,2,4- triazole-1-carboxamide | 510.2 |
| 234 | 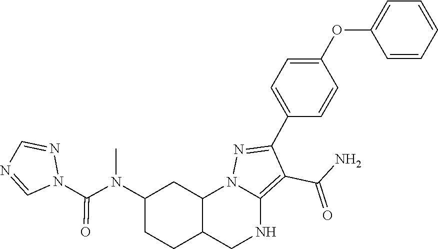 [SMILES: CN(C(=O)n1cncn1)C1CCC2CNc3c(C(N)=O)c(-c4ccc(Oc5ccccc5)cc4)nn3C2C1] | 8-(N-methyl-1H-1,2,4-triazole-1- carboxamido)-2-(4-phenoxyphenyl)- 4,5,5a,6,7,8,9,9a-octahydro- pyrazolo[1,5-a]quinazoline-3- carboxamide | 513.3 |
| 235 | 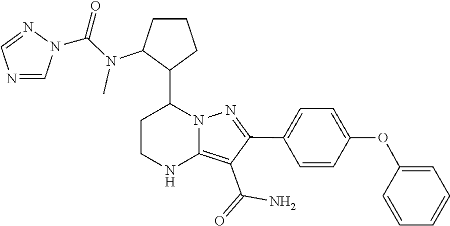 [SMILES: CN(C(=O)n1cncn1)C1CCCC1C1CCNc2c(C(N)=O)c(-c3ccc(Oc4ccccc4)cc3)nn21] | 7-(2-(N-methyl-1H-1,2,4-triazole-1- carboxamido)cyclopentyl)-2-(4- phenoxyphenyl)-4,5,6,7- tetrahydropyrazolo[1,5-a] pyrimidine-3-carboxamide | 527.3 |
| 236 | 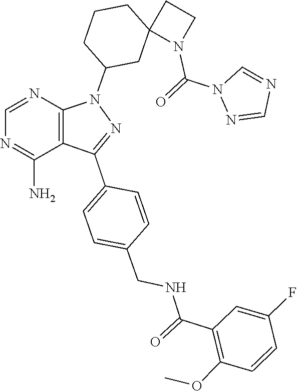 | N-(4-(1-(1-(1H-1,2,4-triazole-1- carbonyl)-1-azaspiro[3.5]nonan-6- yl)-4-amino-1H-pyrazolo[3,4-d] pyrimidin-3-yl)benzyl)-5-fluoro-2- methoxybenzamide | 611.4 |
| 237 | 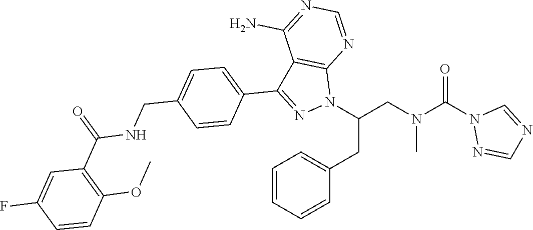 [SMILES: COc1ccc(F)cc1C(=O)NCc1ccc(-c2nn(C(Cc3ccccc3)CN(C)C(=O)n3cncn3)c3ncnc(N)c23)cc1] | N-(2-(4-amino-3-(4-((5-fluoro-2- methoxybenzamido)methyl) phenyl)-1H-pyrazolo[3,4-d] pyrimidin-1-yl)-3-phenylpropyl)- N-methyl-1H-1,2,4-triazole-1- carboxamide | 635.4 |
| 238 | 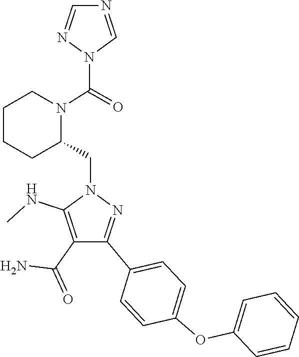 [SMILES: CNc1c(C(N)=O)c(-c2ccc(Oc3ccccc3)cc2)nn1C[C@@H]1CCCCN1C(=O)n1cncn1] | (S)-1-((1-(1H-1,2,4-triazole-1- carbonyl)piperidin-2-yl)methyl)-5- (methylamino)-3-(4-phenoxyphenyl)- 1H-pyrazole-4-carboxamide | 501.3 |
| 239 | 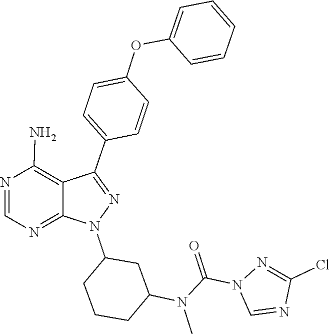 [SMILES: CN(C(=O)n1cnc(Cl)n1)C1CCCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin-1-yl) cyclohexyl)-3-chloro-N-methyl- 1H-1,2,4-triazole-1-carboxamide | 544.2 |
| 240 | 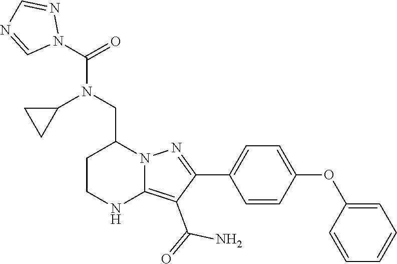 [SMILES: NC(=O)c1c(-c2ccc(Oc3ccccc3)cc2)nn2c1NCCC2CN(C(=O)n1cncn1)C1CC1] | 7-((N-cyclopropyl-1H-1,2,4-triazole- 1-carboxamido)methyl)-2-(4- phenoxyphenyl)-4,5,6,7-tetrahydro- pyrazolo[1,5-a]pyrimidine-3- carboxamide | 499.2 |
| 241 | 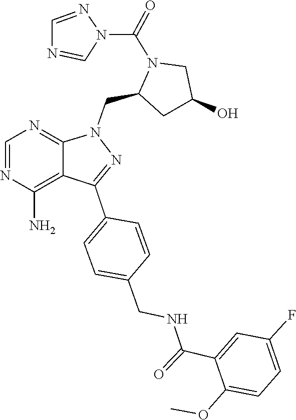 | N-(4-(4-amino-1-(((2S,4S)-4- hydroxy-1-(1H-1,2,4-triazole-1- carbonyl)pyrrolidin-2-yl)methyl)- 1H-pyrazolo[3,4-d]pyrimidin-3- yl)benzyl)-5-fluoro-2- methoxybenzamide | 587.3 |
| 242 | 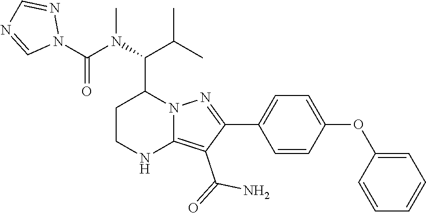 [SMILES: CC(C)[C@H](C1CCNc2c(C(N)=O)c(-c3ccc(Oc4ccccc4)cc3)nn21)N(C)C(=O)n1cncn1] | 7-((R)-2-methyl-1-(N-methyl-1H- 1,2,4-triazole-1-carboxamido) propyl)-2-(4-phenoxyphenyl)- 4,5,6,7-tetrahydropyrazolo[1,5-a] pyrimidine-3-carboxamide | 515.3 |
| 243 | 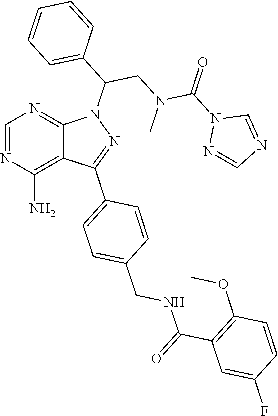 [SMILES: COc1ccc(F)cc1C(=O)NCc1ccc(-c2nn(C(CN(C)C(=O)n3cncn3)c3ccccc3)c3ncnc(N)c23)cc1] | N-(2-(4-amino-3-(4-((5-fluoro-2- methoxybenzamido)methyl) phenyl)-1H-pyrazolo[3,4-d] pyrimidin-1-yl)-2-phenylethyl)- N-methl-1H-1,2,4-triazole-1- carboxamide | 621.4 |
| 244 | 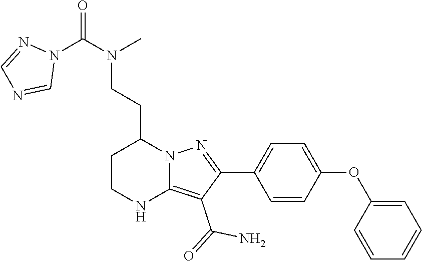 [SMILES: CN(CCC1CCNc2c(C(N)=O)c(-c3ccc(Oc4ccccc4)cc3)nn21)C(=O)n1cncn1] | 7-(2-(N-methyl-1H-1,2,4-triazole- 1-carboxamido)ethyl)-2-(4- phenoxyphenyl)-4,5,6,7- tetrahydropyrazolo[1,5-a] pyrimidine-3-carboxamide | 487.2 |
| 245 | 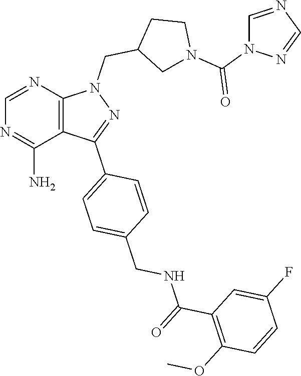 [SMILES: COc1ccc(F)cc1C(=O)NCc1ccc(-c2nn(CC3CCN(C(=O)n4cncn4)C3)c3ncnc(N)c23)cc1] | N-(4-(1-((1-(1H-1,2,4-triazole-1- carbonyl)pyrrolidin-3-yl)methyl)- 4-amino-1H-pyrazolo[3,4-d] pyrimidin-3-yl)benzyl)-5- fluoro-2-methoxybenzamide | 571.3 |
| 246 | 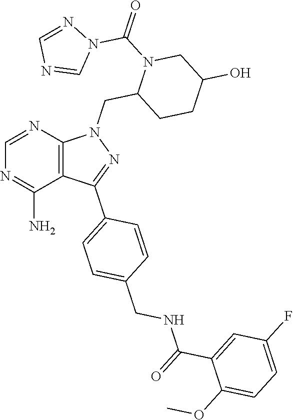 [SMILES: COc1ccc(F)cc1C(=O)NCc1ccc(-c2nn(CC3CCC(O)CN3C(=O)n3cncn3)c3ncnc(N)c23)cc1] | N-(4-(4-amino-1-((5-hydroxy-1- (1H-1,2,4-triazole-1-carbonyl) piperidin-2-yl)methyl)-1H- pyrazolo[3,4-d]pyrimidin-3-yl) benzyl)-5-fluoro-2- methoxybenzamide | 601.4 |
| 247 | 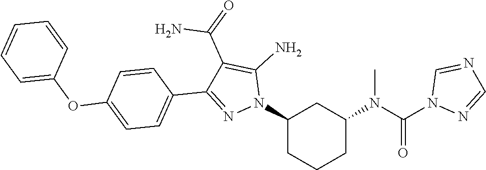 [SMILES: CN(C(=O)n1cncn1)[C@@H]1CCC[C@@H](n2nc(-c3ccc(Oc4ccccc4)cc3)c(C(N)=O)c2N)C1] | N-((1R,3R)-3-(5-amino-4-carbamoyl- 3-(4-phenoxyphenyl)-1H-pyrazol-1- yl)cyclohexyl)-N-methyl-1H-1,2,4- triazole-1-carboxamide | 501.3 |
| 248 | 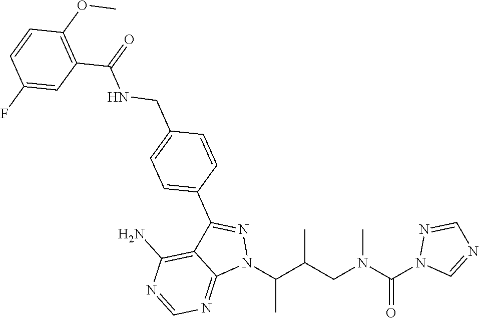 [SMILES: COc1ccc(F)cc1C(=O)NCc1ccc(-c2nn(C(C)C(C)CN(C)C(=O)n3cncn3)c3ncnc(N)c23)cc1] | N-(3-(4-amino-3-(4-((5-fluoro-2- methoxybenzamido)methyl)phenyl)- 1H-pyrazolo[3,4-d]pyrimidin-1-yl)- 2-methylbutyl)-N-methyl-1H-1,2,4- triazole-1-carboxamide | 587.3 |
| 249 | 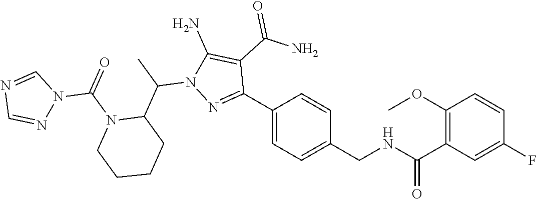 [SMILES: COc1ccc(F)cc1C(=O)NCc1ccc(-c2nn(C(C)C3CCCCN3C(=O)n3cncn3)c(N)c2C(N)=O)cc1] | 1-(1-(1-(1H-1,2,4-triazole-1- carbonyl)piperidin-2-yl)ethyl)-5- amino-3-(4-((5-fluoro-2- methoxybenzamido)methyl) phenyl)-1H-pyrazole-4- carboxamide | 590.3 |
| 250 | 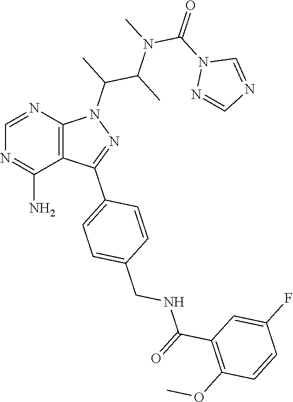 [SMILES: COc1ccc(F)cc1C(=O)NCc1ccc(-c2nn(C(C)C(C)N(C)C(=O)n3cncn3)c3ncnc(N)c23)cc1] | N-(3-(4-amino-3-(4-((5-fluoro-2- methoxybenzamido)methyl)phenyl)- 1H-pyrazolo[3,4-d]pyrimidin-1- yl)butan-2-yl)-N-methyl-1H- 1,2,4-triazole-1-carboxamide | 573.8 |
| 251 | 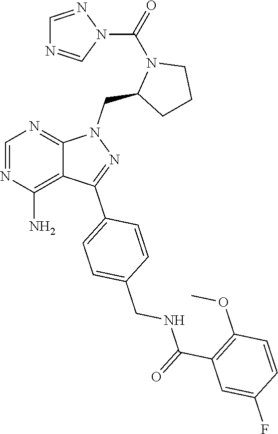 [SMILES: COc1ccc(F)cc1C(=O)NCc1ccc(-c2nn(C[C@@H]3CCCN3C(=O)n3cncn3)c3ncnc(N)c23)cc1] | (S)-N-(4-(1-((1-(1H-1,2,4-triazole-1- carbonyl)pyrrolidin-2-yl)methyl)- 4-amino-1H-pyrazolo[3,4-d] pyrimidin-3-yl)benzyl)-5- fluoro-2-methoxybenzamide | 571.2 |
| 252 | 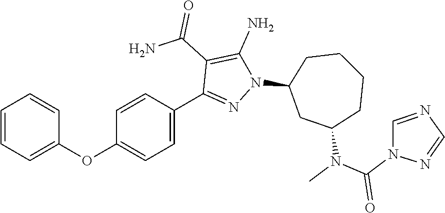 [SMILES: CN(C(=O)n1cncn1)[C@H]1CCCC[C@H](n2nc(-c3ccc(Oc4ccccc4)cc3)c(C(N)=O)c2N)C1] | N-((1S,3S)-3-(5-amino-4-carbamoyl- 3-(4-phenoxyphenyl)-1H-pyrazol-1- yl)cycloheptyl)-N-methyl-1H-1,2,4- triazole-1-carboxamide | 515.3 |
| 253 | 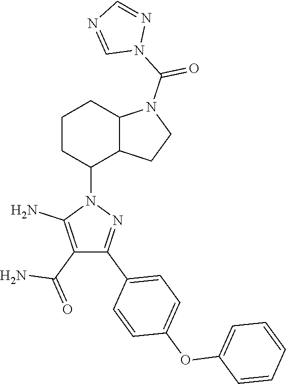 [SMILES: NC(=O)c1c(-c2ccc(Oc3ccccc3)cc2)nn(C2CCCC3C2CCN3C(=O)n2cncn2)c1N] | 1-(1-(1H-1,2,4-triazole-1- carbonyl)octahydro-1H-indol-4- yl)-5-amino-3-(4-phenoxyphenyl)- 1H-pyrazole-4-carboxamide | 513.3 |
| 254 | 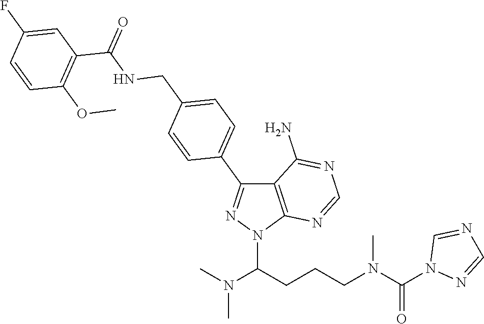 [SMILES: COc1ccc(F)cc1C(=O)NCc1ccc(-c2nn(C(CCCN(C)C(=O)n3cncn3)N(C)C)c3ncnc(N)c23)cc1] | N-(4-(4-amino-3-(4-((5-fluoro-2- methoxybenzamido)methyl) phenyl)-1H-pyrazolo[3,4-d] pyrimidin-1-yl)-4-(dimethyl- amino)butyl)-N-methyl-1H-1,2,4- triazole-1-carboxamide | 616.4 |
| 255 | 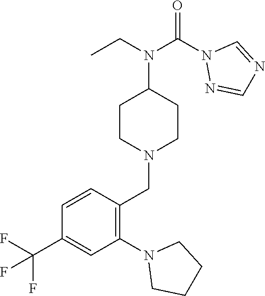 [SMILES: CCN(C(=O)n1cncn1)C1CCN(Cc2ccc(C(F)(F)F)cc2N2CCCC2)CC1] | N-ethyl-N-(1-(2-(pyrrolidin-1-yl)-4- (trifluoromethyl)benzyl)piperidin-4- yl)-1H-1,2,4-triazole-1-carboxamide | 451.3 |
| 256 | 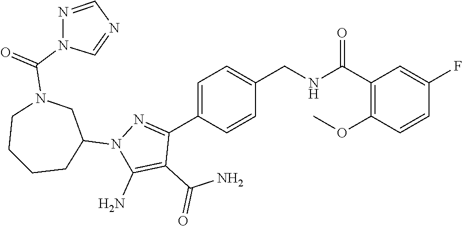 [SMILES: COc1ccc(F)cc1C(=O)NCc1ccc(-c2nn(C3CCCCN(C(=O)n4cncn4)C3)c(N)c2C(N)=O)cc1] | 1-(1-(1H-1,2,4-triazole-1-carbonyl) azepan-3-yl)-5-amino-3-(4-((5- fluoro-2-methoxybenzamido)methyl) phenyl)-1H-pyrazole-4-carboxamide | 576.3 |
| 257 | 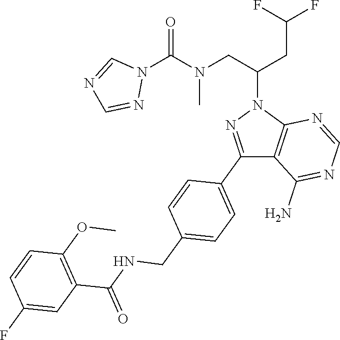 [SMILES: COc1ccc(F)cc1C(=O)NCc1ccc(-c2nn(C(CC(F)F)CN(C)C(=O)n3cncn3)c3ncnc(N)c23)cc1] | N-(2-(4-amino-3-(4-((5-fluoro-2- methoxybenzamido)methyl)phenyl)- 1H-pyrazolo[3,4-d]pyrimidin-1-yl)- 4,4-difluorobutyl)-N-methyl-1H- 1,2,4-triazole-1-carboxamide | 609.3 |
| 258 | 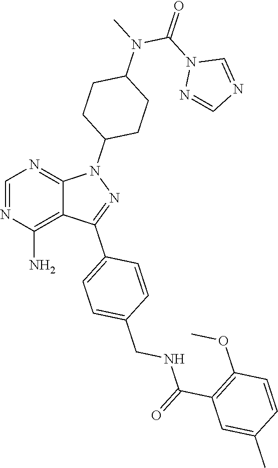 [SMILES: COc1ccc(C)cc1C(=O)NCc1ccc(-c2nn(C3CCC(N(C)C(=O)n4cncn4)CC3)c3ncnc(N)c23)cc1] | N-(4-(4-amino-3-(4-((2-methoxy-5- methylbenzamido)methyl)phenyl)- 1H-pyrazolo[3,4-d]pyrimidin-1-yl) cyclohexyl)-N-methyl-1H-1,2,4- triazole-1-carboxamide | 595.3 |
| 259 | 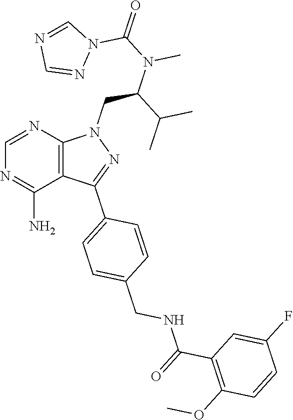 [SMILES: COc1ccc(F)cc1C(=O)NCc1ccc(-c2nn(C[C@H](C(C)C)N(C)C(=O)n3cncn3)c3ncnc(N)c23)cc1] | (S)-N-(1-(4-amino-3-(4-((5-fluoro- 2-methoxybenzamido)methyl) phenyl)-1H-pyrazolo[3,4-d] pyrimidin-1-yl)-3-methylbutan- 2-yl)-N-methyl-1H-1,2,4- triazole-1-carboxamide | 587.3 |
| 260 | 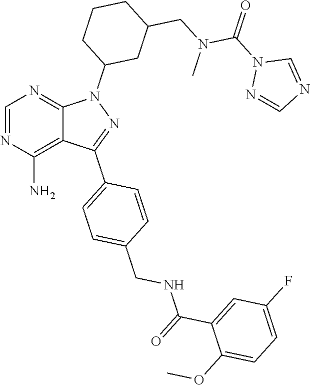 [SMILES: COc1ccc(F)cc1C(=O)NCc1ccc(-c2nn(C3CCCC(CN(C)C(=O)n4cncn4)C3)c3ncnc(N)c23)cc1] | N-((3-(4-amino-3-(4-((5-fluoro-2- methoxybenzamido)methyl)phenyl)- 1H-pyrazolo[3,4-d]pyrimidin-1- yl)cyclohexyl)methyl)-N-methyl- 1H-1,2,4-triazole-1-carboxamide | 613.4 |
| 261 | 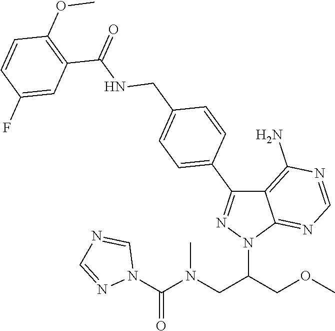 [SMILES: COCC(CN(C)C(=O)n1cncn1)n1nc(-c2ccc(CNC(=O)c3cc(F)ccc3OC)cc2)c2c(N)ncnc21] | N-(2-(4-amino-3-(4-((5-fluoro-2- methoxybenzamido)methyl)phenyl)- 1H-pyrazolo[3,4-d]pyrimidin-1-yl)-3- methoxypropyl)-N-methyl-1H- 1,2,4-triazole-1-carboxamide | 589.3 |
| 262 | 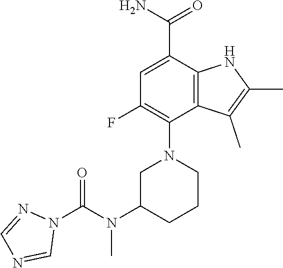 [SMILES: Cc1[nH]c2c(C(N)=O)cc(F)c(N3CCCC(N(C)C(=O)n4cncn4)C3)c2c1C] | 5-fluoro-2,3-dimethyl-4-(3-(N- methyl-1H-1,2,4-triazole-1- carboxamido)piperidin-1-yl)- 1H-indole-7-carboxamide | 414.2 |
| 263 | 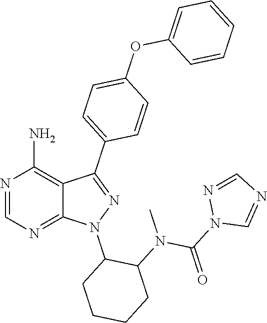 [SMILES: CN(C(=O)n1cncn1)C1CCCCC1n1nc(-c2ccc(Oc3ccccc3)cc2)c2c(N)ncnc21] | N-(2-(4-amino-3-(4-phenoxyphenyl)- 1H-pyrazolo[3,4-d]pyrimidin-1-yl) cyclohexyl)-N-methyl-1H-1,2,4- triazole-1-carboxamide | 510.2 |
| 264 | 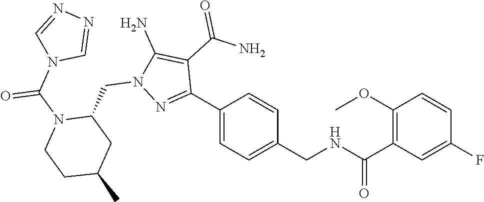 [SMILES: COc1ccc(F)cc1C(=O)NCc1ccc(-c2nn(C[C@@H]3C[C@@H](C)CCN3C(=O)n3cnnc3)c(N)c2C(N)=O)cc1] | 5-amino-3-(4-((5-fluoro-2- methoxybenzamido)methyl)phenyl)- 1-(((2S,4S)-4-methyl-1-(4H-1,2,4- triazole-4-carbonyl)piperidin-2-yl) methyl)-1H-pyrazole-4- carboxamide | 590.3 |
| 265 | 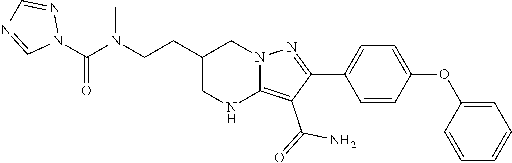 [SMILES: CN(CCC1CNc2c(C(N)=O)c(-c3ccc(Oc4ccccc4)cc3)nn2C1)C(=O)n1cncn1] | 6-(2-(N-methyl-1H-1,2,4-triazole-1- carboxamido)ethyl)-2-(4-phenoxy- phenyl)-4,5,6,7-tetrahydro- pyrazolo[1,5-a]pyrimidine-3- carboxamide | 487.2 |
| 266 | 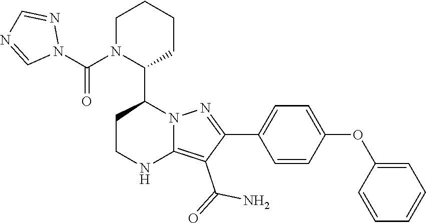 [SMILES: NC(=O)c1c(-c2ccc(Oc3ccccc3)cc2)nn2c1NCC[C@H]2[C@H]1CCCCN1C(=O)n1cncn1] | (S)-7-((R)-1-(1H-1,2,4-triazole-1- carbonyl)piperidin-2-yl)-2-(4- phenoxyphenyl)-4,5,6,7- tetrahydropyrazolo[1,5-a] pyrimidine-3-carboxamide | 513.3 |
| 267 | 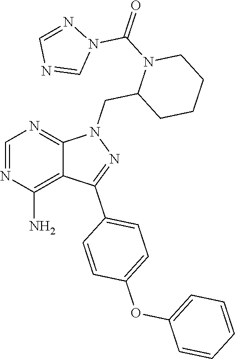 [SMILES: Nc1ncnc2c1c(-c1ccc(Oc3ccccc3)cc1)nn2CC1CCCCN1C(=O)n1cncn1] | (2-((4-amino-3-(4-phenoxyphenyl)- 1H-pyrazolo[3,4-d]pyrimidin-1- yl)methyl)piperidin-1-yl)(1H-1,2,4- triazol-1-yl)methanone | 496.2 |
| 268 | 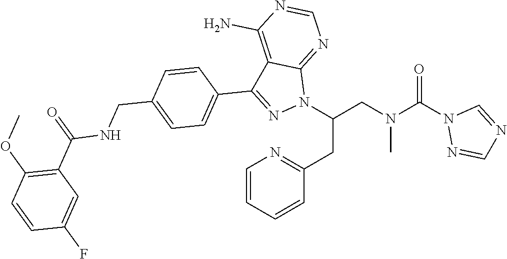 [SMILES: COc1ccc(F)cc1C(=O)NCc1ccc(-c2nn(C(Cc3ccccn3)CN(C)C(=O)n3cncn3)c3ncnc(N)c23)cc1] | N-(2-(4-amino-3-(4-((5-fluoro-2- methoxybenzamido)methyl) phenyl)-1H-pyrazolo[3,4-d] pyrimidin-1-yl)-3-(pyridin-2- yl)propyl)-N-methyl-1H-1,2,4- triazole-1-carboxamide | 636.4 |
| 269 | 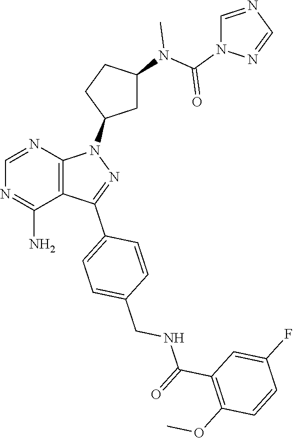 [SMILES: COc1ccc(F)cc1C(=O)NCc1ccc(-c2nn([C@H]3CC[C@@H](N(C)C(=O)n4cncn4)C3)c3ncnc(N)c23)cc1] | N-((1R,3S)-3-(4-amino-3-(4-((5- fluoro-2-methoxybenzamido) methyl)phenyl)-1H-pyrazolo [3,4-d]pyrimidin-1-yl)cyclopentyl)- N-methyl-1H-1,2,4-triazole-1- carboxamide | 585.3 |
| 270 | 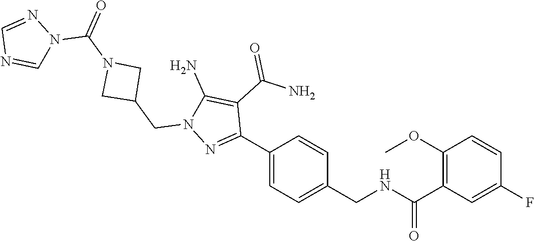 [SMILES: COc1ccc(F)cc1C(=O)NCc1ccc(-c2nn(CC3CN(C(=O)n4cncn4)C3)c(N)c2C(N)=O)cc1] | 1-((1-(1H-1,2,4-triazole-1-carbonyl) azetidin-3-yl)methyl)-5-amino-3-(4- ((5-fluoro-2-methoxybenzamido) methyl)phenyl)-1H-pyrazole-4- carboxamide | 548.3 |
| 271 | 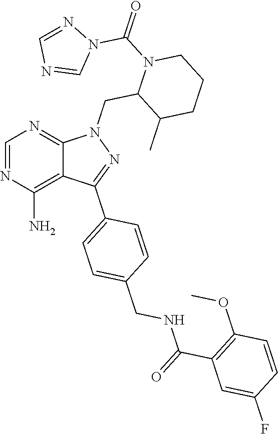 [SMILES: COc1ccc(F)cc1C(=O)NCc1ccc(-c2nn(CC3C(C)CCCN3C(=O)n3cncn3)c3ncnc(N)c23)cc1] | N-(4-(4-amino-1-((3-methyl-1- (1H-1,2,4-triazole-1-carbonyl) piperidin-2-yl)methyl)-1H- pyrazolo[3,4-d]pyrimidin-3- yl)benzyl)-5-fluoro-2- methoxybenzamide | 599.3 |
| 272 | 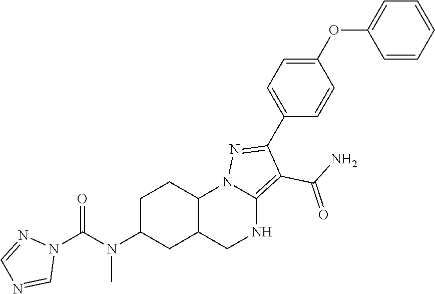 [SMILES: CN(C(=O)n1cncn1)C1CCC2C(CNc3c(C(N)=O)c(-c4ccc(Oc5ccccc5)cc4)nn32)C1] | 7-(N-methyl-1H-1,2,4-triazole-1- carboxamido)-2-(4-phenoxyphenyl)- 4,5,5a,6,7,8,9,9a-octahydropyrazolo [1,5-a]quinazoline-3-carboxamide | 513.3 |
| 273 | 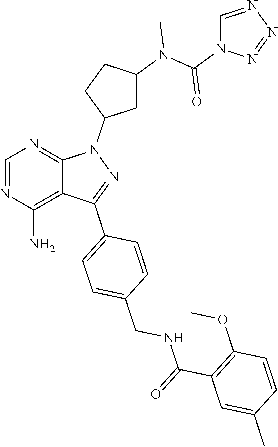 [SMILES: COc1ccc(C)cc1C(=O)NCc1ccc(-c2nn(C3CCC(N(C)C(=O)n4cnnn4)C3)c3ncnc(N)c23)cc1] | N-(3-(4-amino-3-(4-((2-methoxy-5- methylbenzamido)methyl)phenyl)-1H- pyrazolo[3,4-d]pyrimidin-1-yl) cyclopentyl)-N-methyl-1H- tetrazole-1-carboxamide | 582.3 |
| 274 | 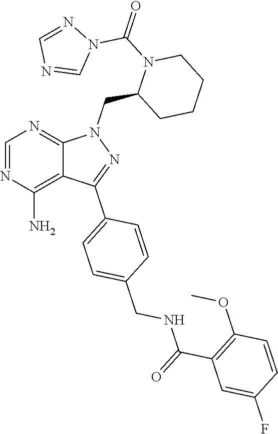 [SMILES: COc1ccc(F)cc1C(=O)NCc1ccc(-c2nn(C[C@@H]3CCCCN3C(=O)n3cncn3)c3ncnc(N)c23)cc1] | (S)-N-(4-(1-((1-(1H-1,2,4-triazole-1- carbonyl)piperidin-2-yl)methyl)-4- amino-1H-pyrazolo[3,4-d]pyrimidin- 3-yl)benzyl)-5-fluoro-2- methoxybenzamide | 585.2 |
| 275 | 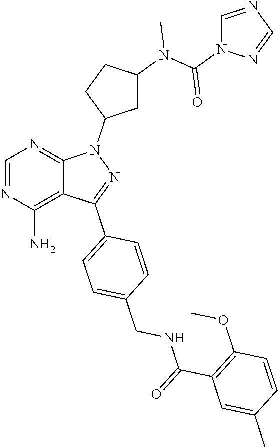 [SMILES: COc1ccc(C)cc1C(=O)NCc1ccc(-c2nn(C3CCC(N(C)C(=O)n4cncn4)C3)c3ncnc(N)c23)cc1] | N-(3-(4-amino-3-(4-((2-methoxy-5- methylbenzamido)methyl)phenyl)- 1H-pyrazolo[3,4-d]pyrimidin-1- yl)cyclopentyl)-N-methyl-1H- 1,2,4-triazole-1-carboxamide | 581.3 |
| 276 | 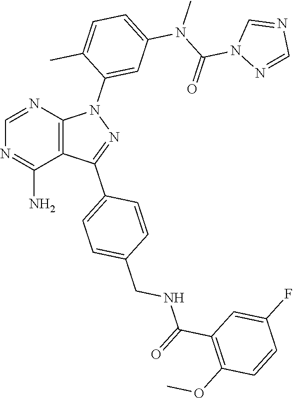 [SMILES: COc1ccc(F)cc1C(=O)NCc1ccc(-c2nn(-c3cc(N(C)C(=O)n4cncn4)ccc3C)c3ncnc(N)c23)cc1] | N-(3-(4-amino-3-(4-((5-fluoro-2- methoxybenzamido)methyl)phenyl)- 1H-pyrazolo[3,4-d]pyrimidin-1-yl)- 4-methylphenyl)-N-methyl-1H- 1,2,4-triazole-1-carboxamide | 607.3 |
| 277 | 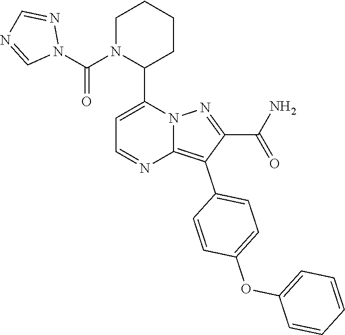 [SMILES: NC(=O)c1nn2c(C3CCCCN3C(=O)n3cncn3)ccnc2c1-c1ccc(Oc2ccccc2)cc1] | 7-(1-(1H-1,2,4-triazole-1-carbonyl) piperidin-2-yl)-3-(4-phenoxy- phenyl)pyrazolo[1,5-a]pyrimidine- 2-carboxamide | 509.2 |
| 278 | 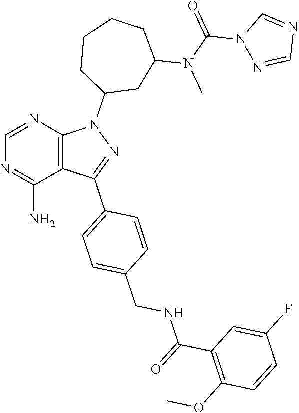 [SMILES: COc1ccc(F)cc1C(=O)NCc1ccc(-c2nn(C3CCCCC(N(C)C(=O)n4cncn4)C3)c3ncnc(N)c23)cc1] | N-(3-(4-amino-3-(4-((5-fluoro-2- methoxybenzamido)methyl) phenyl)-1H-pyrazolo[3,4-d] pyrimidin-1-yl)cycloheptyl)- N-methyl-1H-1,2,4-triazole-1- carboxamide | 613.4 |
| 279 | 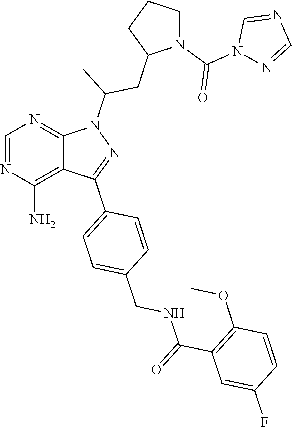 [SMILES: COc1ccc(F)cc1C(=O)NCc1ccc(-c2nn(C(C)CC3CCCN3C(=O)n3cncn3)c3ncnc(N)c23)cc1] | N-(4-(1-(1-(1-(1H-1,2,4-triazole-1- carbonyl)pyrrolidin-2-yl)propan-2- yl)-4-amino-1H-pyrazolo[3,4-d] pyrimidin-3-yl)benzyl)-5-fluoro- 2-methoxybenzamide | 599.3 |
| 280 | 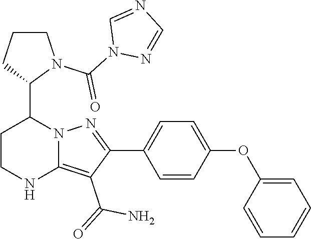 [SMILES: NC(=O)c1c(-c2ccc(Oc3ccccc3)cc2)nn2c1NCCC2[C@@H]1CCCN1C(=O)n1cncn1] | 7-((S)-1-(1H-1,2,4-triazole-1- carbonyl)pyrrolidin-2-yl)-2-(4- phenoxyphenyl)-4,5,6,7- tetrahydropyrazolo[1,5-a] pyrimidine-3-carboxamide | 499.2 |
| 281 | 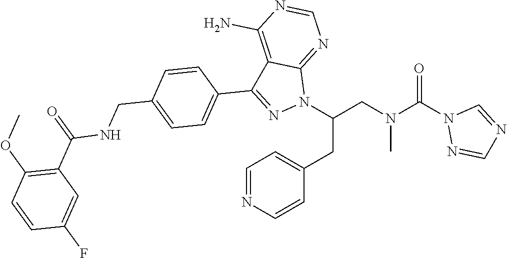 [SMILES: COc1ccc(F)cc1C(=O)NCc1ccc(-c2nn(C(Cc3ccncc3)CN(C)C(=O)n3cncn3)c3ncnc(N)c23)cc1] | N-(4-(4-amino-3-(4-((5-fluoro-2- methoxybenzamido)methyl)phenyl)- 1H-pyrazolo[3,4-d]pyrimidin-1-yl)-3- (pyridin-4-yl)propyl)-N-methyl-1H- 1,2,4-triazole-1-carboxamide | 636.4 |
| 282 | 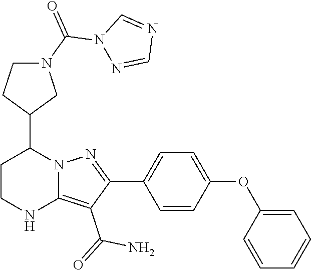 [SMILES: NC(=O)c1c(-c2ccc(Oc3ccccc3)cc2)nn2c1NCCC2C1CCN(C(=O)n2cncn2)C1] | 7-(1-(1H-1,2,4-triazole-1- carbonyl)pyrrolidin-3-yl)-2-(4- phenoxyphenyl)-4,5,6,7- tetrahydropyrazolo[1,5-a] pyrimidine-3-carboxamide | 499.2 |
| 283 | 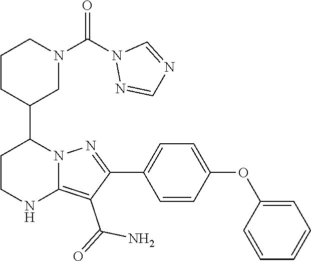 [SMILES: NC(=O)c1c(-c2ccc(Oc3ccccc3)cc2)nn2c1NCCC2C1CCCN(C(=O)n2cncn2)C1] | 7-(1-(1H-1,2,4-triazole-1-carbonyl) piperidin-3-yl)-2-(4-phenoxyphenyl)- 4,5,6,7-tetrahydropyrazolo[1,5-a] pyrimidine-3-carboxamide | 513.3 |
| 284 | 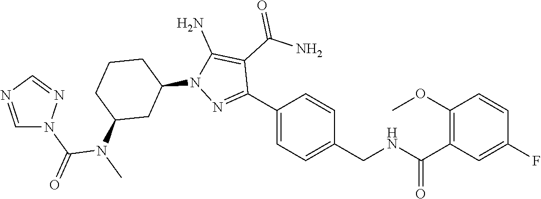 [SMILES: COc1ccc(F)cc1C(=O)NCc1ccc(-c2nn([C@@H]3CCC[C@H](N(C)C(=O)n4cncn4)C3)c(N)c2C(N)=O)cc1] | N-((1S,3R)-3-(5-amino-4-carbamoyl- 3-(4-((5-fluoro-2-methoxy- benzamido)methyl)phenyl)-1H- pyrazol-1-yl)cyclohexyl)-N-methyl- 1H-1,2,4-triazole-1-carboxamide | 590.3 |
| 285 | 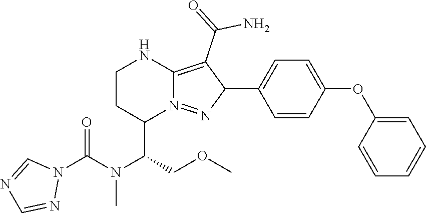 | 7-((R)-2-methoxy-1-(N-methyl-1H- 1,2,4-triazole-1-carboxamido) ethyl)-2-(4-phenoxyphenyl)- 4,5,6,7-tetrahydropyrazolo [1,5-a]pyrimidine-3-carboxamide | 517.3 |
| 286 | 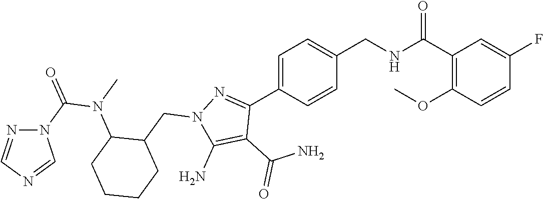 [SMILES: COc1ccc(F)cc1C(=O)NCc1ccc(-c2nn(CC3CCCCC3N(C)C(=O)n3cncn3)c(N)c2C(N)=O)cc1] | N-(2-((5-amino-4-carbamoyl-3-(4- ((5-fluoro-2-methoxybenzamido) methyl)phenyl)-1H-pyrazol-1- yl)methyl)cyclohexyl)-N-methyl- 1H-1,2,4-triazole-1-carboxamide | 604.4 |
| 287 | 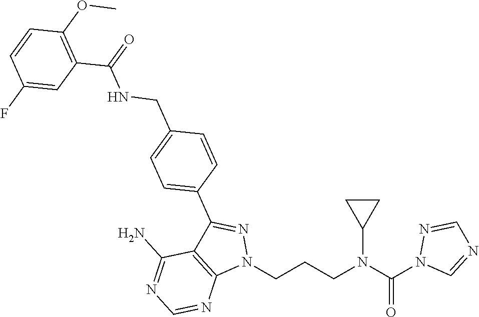 [SMILES: COc1ccc(F)cc1C(=O)NCc1ccc(-c2nn(CCCN(C(=O)n3cncn3)C3CC3)c3ncnc(N)c23)cc1] | N-(3-(4-amino-3-(4-((5-fluoro-2- methoxybenzamido)methyl)phenyl)- 1H-pyrazolo[3,4-d]pyrimidin-1-yl) propyl)-N-cyclopropyl-1H-1,2,4- triazole-1-carboxamide | 585.3 |
| 288 | 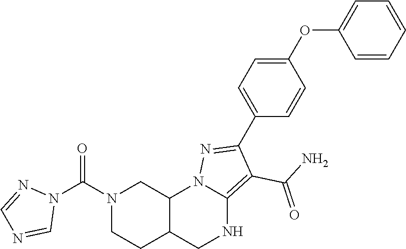 [SMILES: NC(=O)c1c(-c2ccc(Oc3ccccc3)cc2)nn2c1NCC1CCN(C(=O)n3cncn3)CC12] | 2-(4-phenoxyphenyl)-8-(1H-1,2,4- triazole-1-carbonyl)-4,5,5a,6,7,8,9,9a- octahydropyrazolo[1,5-a]pyrido [4,3-e]pyrimidine-3-carboxamide | 485.2 |
| 289 | 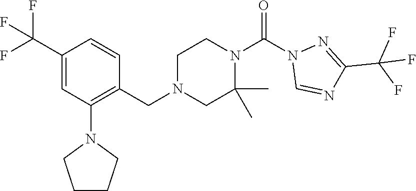 [SMILES: CC1(C)CN(Cc2ccc(C(F)(F)F)cc2N2CCCC2)CCN1C(=O)n1cnc(C(F)(F)F)n1] | (2,2-dimethyl-4-(2-(pyrrolidin-1-yl)- 4-(trifluoromethyl)benzyl)piperazin- 1-yl)(3-(trifluoromethyl)-1H- 1,2,4-triazol-1-yl)methanone | 505.5 |
| 290 | 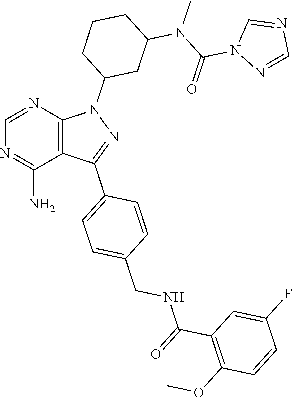 [SMILES: COc1ccc(F)cc1C(=O)NCc1ccc(-c2nn(C3CCCC(N(C)C(=O)n4cncn4)C3)c3ncnc(N)c23)cc1] | N-(3-(4-amino-3-(4-((5-fluoro-2- methoxybenzamido)methyl)phenyl)- 1H-pyrazolo[3,4-d]pyrimidin-1- yl)cyclohexyl)-N-methyl-1H- 1,2,4-triazole-1-carboxamide | 599.3 |
| 291 | 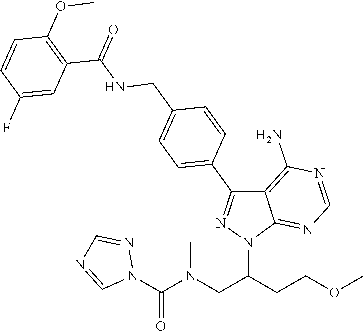 [SMILES: COCCC(CN(C)C(=O)n1cncn1)n1nc(-c2ccc(CNC(=O)c3cc(F)ccc3OC)cc2)c2c(N)ncnc21] | N-(2-(4-amino-3-(4-((5-fluoro-2- methoxybenzamido)methyl)phenyl)- 1H-pyrazolo[3,4-d]pyrimidin-1-yl)- 4-methoxybutyl)-N-methyl-1H- 1,2,4-triazole-1-carboxamide | 603.3 |
| 292 | 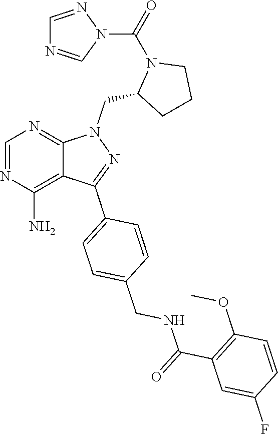 [SMILES: COc1ccc(F)cc1C(=O)NCc1ccc(-c2nn(C[C@H]3CCCN3C(=O)n3cncn3)c3ncnc(N)c23)cc1] | (R)-N-(4-(1-((1-(1H-1,2,4-triazole- 1-carbonyl)pyrrolidin-2-yl)methyl)-4- amino-1H-pyrazolo[3,4-d]pyrimidin- 3-yl)benzyl)-5-fluoro-2- methoxybenzamide | 571.2 |
| 293 | 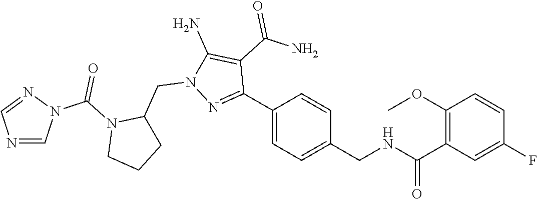 [SMILES: COc1ccc(F)cc1C(=O)NCc1ccc(-c2nn(CC3CCCN3C(=O)n3cncn3)c(N)c2C(N)=O)cc1] | 1-((1-(1H-1,2,4-triazole-1- carbonyl)pyrrolidin-2-yl)methyl)-5- amino-3-(4-((5-fluoro-2- methoxybenzamido)methyl) phenyl)-1H-pyrazole-4- carboxamide | 562.2 |
| 294 | 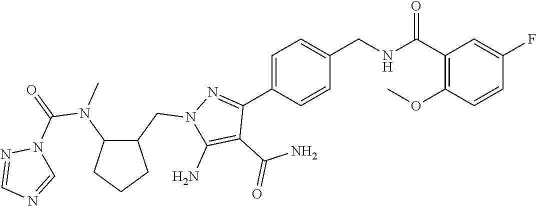 [SMILES: COc1ccc(F)cc1C(=O)NCc1ccc(-c2nn(CC3CCCC3N(C)C(=O)n3cncn3)c(N)c2C(N)=O)cc1] | N-(2-((5-amino-4-carbamoyl-3-(4- ((5-fluoro-2-methoxybenzamido) methyl)phenyl)-1H-pyrazol-1- yl)methyl)cyclopentyl)-N-methyl- 1H-1,2,4-triazole-1-carboxamide | 590.3 |
| 295 | 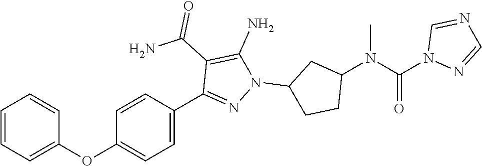 [SMILES: CN(C(=O)n1cncn1)C1CCC(n2nc(-c3ccc(Oc4ccccc4)cc3)c(C(N)=O)c2N)C1] | N-(3-(5-amino-4-carbamoyl-3-(4- phenoxyphenyl)-1H-pyrazol-1- yl)cyclopentyl)-N-methyl-1H- 1,2,4-triazole-1-carboxamide | 487.2 |
| 296 | 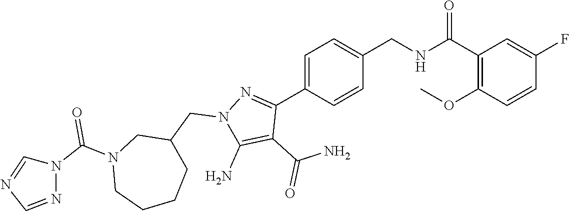 [SMILES: COc1ccc(F)cc1C(=O)NCc1ccc(-c2nn(CC3CCCCN(C(=O)n4cncn4)C3)c(N)c2C(N)=O)cc1] | 1-((1-(1H-1,2,4-triazole-1-carbonyl) azepan-3-yl)methyl)-5-amino-3- (4-((5-fluoro-2-methoxybenzamido) methyl)phenyl)-1H-pyrazole-4- carboxamide | 590.3 |
| 297 | 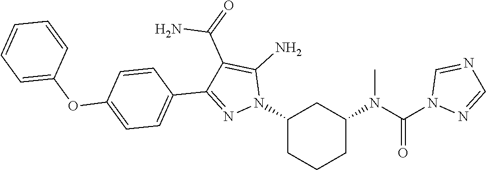 [SMILES: CN(C(=O)n1cncn1)[C@@H]1CCC[C@H](n2nc(-c3ccc(Oc4ccccc4)cc3)c(C(N)=O)c2N)C1] | N-((1R,3S)-3-(5-amino-4-carbamoyl- 3-(4-phenoxyphenyl)-1H-pyrazol-1- yl)cyclohexyl)-N-methyl-1H-1,2,4- triazole-1-carboxamide | 501.3 |
| 298 | 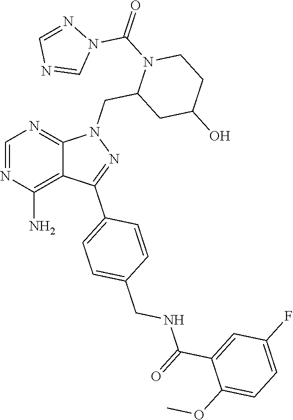 [SMILES: COc1ccc(F)cc1C(=O)NCc1ccc(-c2nn(CC3CC(O)CCN3C(=O)n3cncn3)c3ncnc(N)c23)cc1] | N-(4-(4-amino-1-((4-hydroxy-1- (1H-1,2,4-triazole-1-carbonyl) piperidin-2-yl)methyl)-1H- pyrazolo[3,4-d]pyrimidin-3-yl) benzyl)-5-fluoro-2- methoxybenzamide | 601.3 |
| 299 | 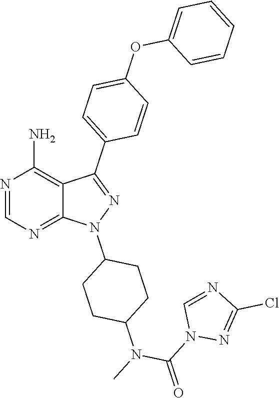 [SMILES: CN(C(=O)n1cnc(Cl)n1)C1CCC(n2nc(-c3ccc(Oc4ccccc4)cc3)c3c(N)ncnc32)CC1] | N-(4-(4-amino-3-(4-phenoxyphenyl)- 1H-pyrazolo[3,4-d]pyrimidin-1-yl) cyclohexyl)-3-chloro-N-methyl- 1H-1,2,4-triazole-1-carboxamide | 544.2 |
| 300 | 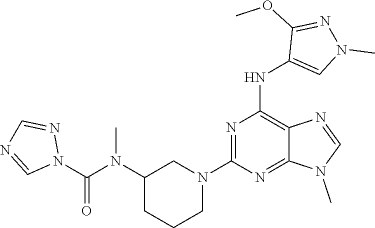 [SMILES: COc1nn(C)cc1Nc1nc(N2CCCC(N(C)C(=O)n3cncn3)C2)nc2c1ncn2C] | N-(1-(6-((3-methoxy-1-methyl-1H- pyrazol-4-yl)amino)-9-methyl-9H- purin-2-yl)piperidin-3-yl)-N-methyl- 1H-1,2,4-triazole-1-carboxamide | 467.2 |
| 301 | 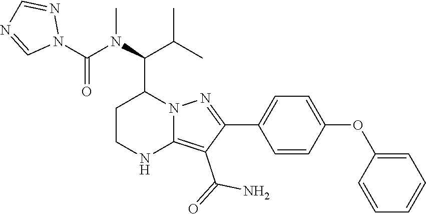 [SMILES: CC(C)[C@@H](C1CCNc2c(C(N)=O)c(-c3ccc(Oc4ccccc4)cc3)nn21)N(C)C(=O)n1cncn1] | 7-((S)-2-methyl-1-(N-methyl-1H- 1,2,4-triazole-1-carboxamido) propyl)-2-(4-phenoxyphenyl)- 4,5,6,7-tetrahydropyrazolo [1,5-a]pyrimidine-3-carboxamide | 515.3 |
| 302 | 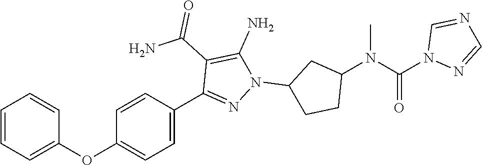 [SMILES: CN(C(=O)n1cncn1)C1CCC(n2nc(-c3ccc(Oc4ccccc4)cc3)c(C(N)=O)c2N)C1] | N-(3-(5-amino-4-carbamoyl-3-(4- phenoxyphenyl)-1H-pyrazol-1- yl)cyclopentyl)-N-methyl-1H- 1,2,4-triazole-1-carboxamide | 487.2 |
| 303 | 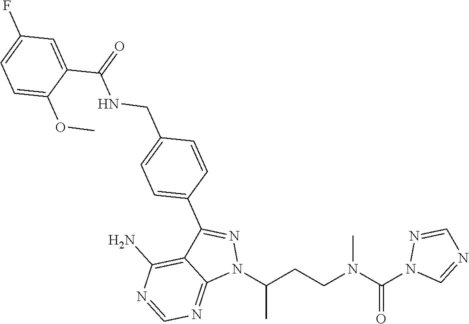 [SMILES: COc1ccc(F)cc1C(=O)NCc1ccc(-c2nn(C(C)CCN(C)C(=O)n3cncn3)c3ncnc(N)c23)cc1] | N-(3-(4-amino-3-(4-((5-fluoro-2- methoxybenzamido)methyl)phenyl)- 1H-pyrazolo[3,4-d]pyrimidin-1-yl) butyl)-N-methyl-1H-1,2,4- triazole-1-carboxamide | 573.3 |
| 304 | 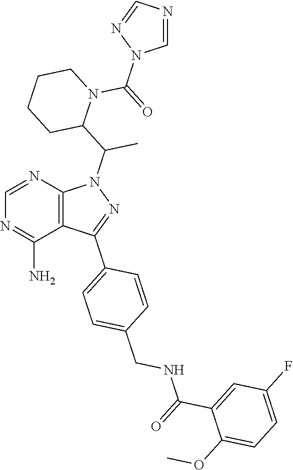 [SMILES: COc1ccc(F)cc1C(=O)NCc1ccc(-c2nn(C(C)C3CCCCN3C(=O)n3cncn3)c3ncnc(N)c23)cc1] | N-(4-(1-(1-(1-(1H-1,2,4-triazole-1- carbonyl)piperidin-2-yl)ethyl)-4- amino-1H-pyrazolo[3,4-d] pyrimidin-3-yl)benzyl)-5- fluoro-2-methoxybenzamide | 599.3 |
| 305 | 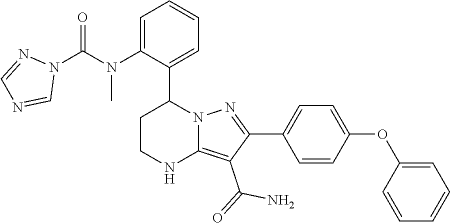 [SMILES: CN(C(=O)n1cncn1)c1ccccc1C1CCNc2c(C(N)=O)c(-c3ccc(Oc4ccccc4)cc3)nn21] | 7-(2-(N-methyl-1H-1,2,4-triazole-1- carboxamido)phenyl)-2-(4- phenoxyphenyl)-4,5,6,7- tetrahydropyrazolo[1,5-a] pyrimidine-3-carboxamide | 535.3 |
| 306 | 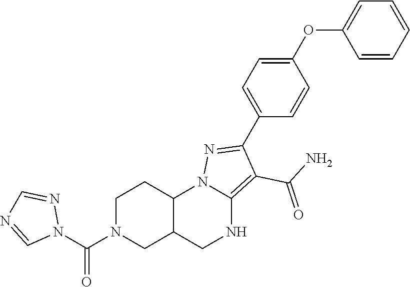 [SMILES: NC(=O)c1c(-c2ccc(Oc3ccccc3)cc2)nn2c1NCC1CN(C(=O)n3cncn3)CCC12] | 2-(4-phenoxyphenyl)-7-(1H-1,2,4- triazole-1-carbonyl)-4,5,5a,6,7,8,9,9a- octahydropyrazolo[1,5-a]pyrido[3,4- e]pyrimidine-3-carboxamide | 485.2 |
| 307 | 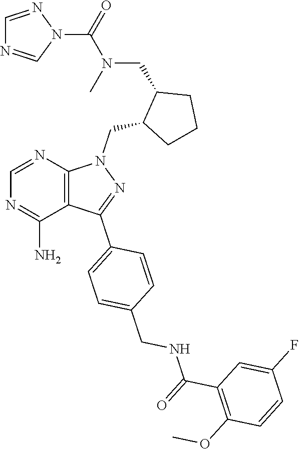 [SMILES: COc1ccc(F)cc1C(=O)NCc1ccc(-c2nn(C[C@H]3CCC[C@H]3CN(C)C(=O)n3cncn3)c3ncnc(N)c23)cc1] | N-(((1R,2S)-2-((4-amino-3-(4-((5- fluoro-2-methoxybenzamido) methyl)phenyl)-1H-pyrazolo [3,4-d]pyrimidin-1-yl)methyl) cyclopentyl)methyl)-N-methyl- 1H-1,2,4-triazole-1-carboxamide | 613.4 |
| 308 | 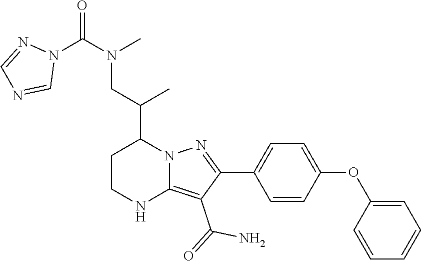 [SMILES: CC(CN(C)C(=O)n1cncn1)C1CCNc2c(C(N)=O)c(-c3ccc(Oc4ccccc4)cc3)nn21] | 7-(1-(N-methyl-1H-1,2,4-triazole-1- carboxamido)propan-2-yl)-2-(4- phenoxyphenyl)-4,5,6,7- tetrahydropyrazolo[1,5-a] pyrimidine-3-carboxamide | 501.3 |
| 309 | 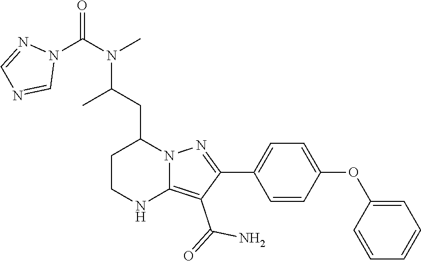 [SMILES: CC(CC1CCNc2c(C(N)=O)c(-c3ccc(Oc4ccccc4)cc3)nn21)N(C)C(=O)n1cncn1] | 7-(2-(N-methyl-1H-1,2,4-triazole-1- carboxamido)propyl)-2-(4- phenoxyphenyl)-4,5,6,7-tetrahydro- pyrazolo[1,5-a]pyrimidine-3- carboxamide | 501.3 |
| 310 | 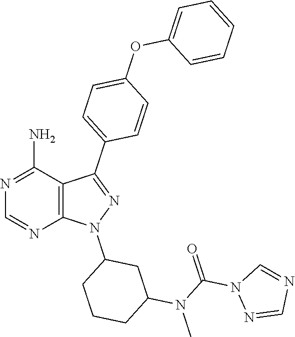 [SMILES: CN(C(=O)n1cncn1)C1CCCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin-1-yl) cyclohexyl)-N-methyl-1H-1,2,4- triazole-1-carboxamide | 510.3 |
| 311 | 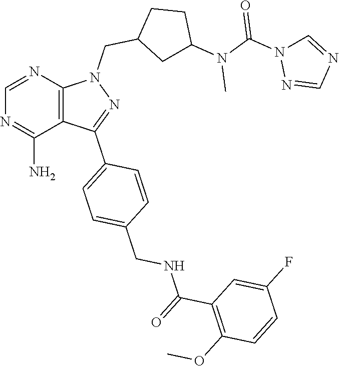 [SMILES: COc1ccc(F)cc1C(=O)NCc1ccc(-c2nn(CC3CCC(N(C)C(=O)n4cncn4)C3)c3ncnc(N)c23)cc1] | N-(3-((4-amino-3-(4-((5-fluoro-2- methoxybenzamido)methyl) phenyl)-1H-pyrazolo[3,4-d] pyrimidin-1-yl)methyl) cyclopentyl)-N-methyl-1H- 1,2,4-triazole-1-carboxamide | 599.3 |
| 312 | 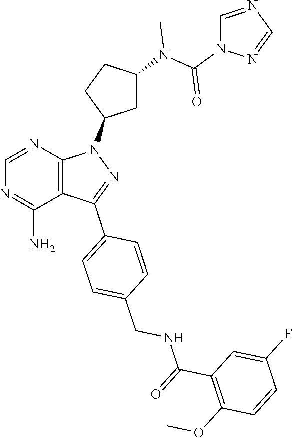 [SMILES: COc1ccc(F)cc1C(=O)NCc1ccc(-c2nn([C@H]3CC[C@H](N(C)C(=O)n4cncn4)C3)c3ncnc(N)c23)cc1] | N-((1S,3S)-3-(4-amino-3-(4-((5- fluoro-2-methoxybenzamido) methyl)phenyl)-1H-pyrazolo [3,4-d]pyrimidin-1-yl) cyclopentyl)-N-methyl-1H- 1,2,4-triazole-1-carboxamide | 585.3 |
| 313 | 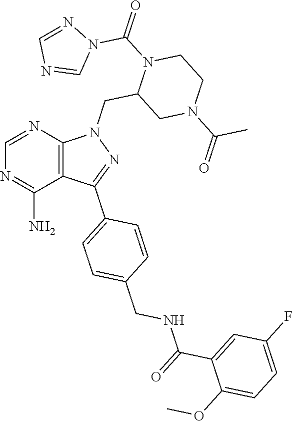 [SMILES: COc1ccc(F)cc1C(=O)NCc1ccc(-c2nn(CC3CN(C(C)=O)CCN3C(=O)n3cncn3)c3ncnc(N)c23)cc1] | N-(4-(1-((4-acetyl-1-(1H-1,2,4- triazole-1-carbonyl)piperazin-2- yl)methyl)-4-amino-1H- pyrazolo[3,4-d]pyrimidin-3-yl) benzyl)-5-fluoro-2- methoxybenzamide | 628.5 |
| 314 | 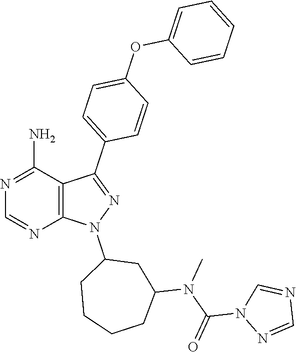 [SMILES: CN(C(=O)n1cncn1)C1CCCCC(n2nc(-c3ccc(Oc4ccccc4)cc3)c3c(N)ncnc32)C1] | N-(3-(4-amino-3-(4-phenoxyphenyl)- 1H-pyrazolo[3,4-d]pyrimidin-1-yl) cycloheptyl)-N-methyl-1H-1,2,4- triazole-1-carboxamide | 524.3 |
| 315 | 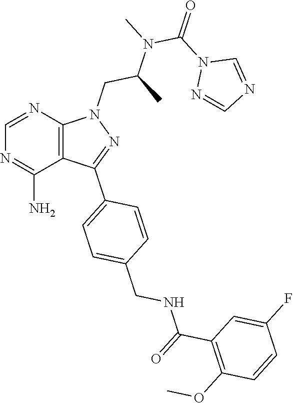 [SMILES: COc1ccc(F)cc1C(=O)NCc1ccc(-c2nn(C[C@H](C)N(C)C(=O)n3cncn3)c3ncnc(N)c23)cc1] | (S)-N-(1-(4-amino-3-(4-((5-fluoro-2- methoxybenzamido)methyl) phenyl)-1H-pyrazolo[3,4-d] pyrimidin-1-yl)propan-2-yl)- N-methyl-1H-1,2,4-triazole-1- carboxamide | 559.3 |
| 316 | 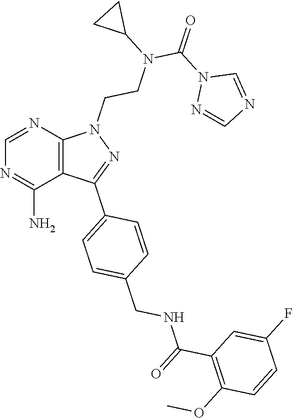 [SMILES: COc1ccc(F)cc1C(=O)NCc1ccc(-c2nn(CCN(C(=O)n3cncn3)C3CC3)c3ncnc(N)c23)cc1] | N-(2-(4-amino-3-(4-((5-fluoro-2- methoxybenzamido)methyl) phenyl)-1H-pyrazolo[3,4-d] pyrimidin-1-yl)ethyl)-N- cyclopropyl-1H-1,2,4- triazole-1-carboxamide | 571.2 |

Compounds of Table 1 are depicted with flat, wedged, and/or hashed wedged bonds. It is understood that compounds depicted in Table 1 encompass all
possible stereoisomers, including atropisomers, of the compounds of Table 1. In some instances, the relative stereochemistry at one or more stereocenters
of a compound has been determined; in some instances, the absolute stereochemistry has been determined. In some instances, a single compound number
represents a mixture of stereoisomers, including atropisomers. In some instances, a single compound number represents a single stereoisomer.

Compounds of Table 1 are depicted with flat, wedged, and/or hashed wedged bonds. It is understood that compounds depicted in Table 1 encompass all possible stereoisomers, including atropisomers, of the compounds of Table 1. In some instances, the relative stereochemistry at one or more stereocenters of a compound has been determined; in some instances, the absolute stereochemistry has been determined. In some instances, a single compound number represents a mixture of stereoisomers, including atropisomers. In some instances, a single compound number represents a single stereoisomer.

In some embodiments, the compounds of the present disclosure exhibit one or more functional characteristics disclosed herein. For example, a subject compound binds to a Target Protein or a mutant form thereof. In some embodiments, a subject compound binds specifically and also inhibits a Target Protein or a mutant form thereof. In some embodiments, a subject compound selectively inhibits a mutant Target Protein relative to a wildtype Target Protein. In some embodiments, a subject compound selectively inhibits a wildtype Target Protein relative to a mutant Target Protein. In some embodiments, the IC50 of a subject compound for a Target Protein is less than about 5 μM, less than about 1 μM, less than about 50 nM, less than about 10 nM, less than about 1 nM, less than about 0.5 nM, less than about 100 μM, or less than about 50 μM, as measured in an in vitro assay known in the art or exemplified herein. In some embodiments, a subject compound covalently binds to the Target Protein.

It shall be understood that different aspects of the disclosure can be appreciated individually, collectively, or in combination with each other. Various aspects described herein may be applied to any of the particular applications disclosed herein. The compositions of matter, including compounds of any formulae disclosed in the compound section, of the present disclosure may be utilized in the method section, including methods of use and production disclosed herein, or vice versa.

The compounds described herein, or a pharmaceutically acceptable salt or solvate thereof, are Target Protein inhibitors capable of inhibiting a Target Protein. Target Protein being inhibited can be mutants or wild-type. Compounds, including pharmaceutically acceptable salts or solvates thereof, disclosed herein have a wide range of applications in therapeutics, diagnostics, and other biomedical research.

In certain aspects, the present disclosure provides a method of treating cancer in a subject in need thereof, comprising administering to the subject a therapeutically effective amount of a compound described herein, or a pharmaceutically acceptable salt or solvate thereof.

In certain aspects, the present disclosure provides a method of treating cancer mediated by the Target Protein, comprising inhibiting the Target Protein of said subject by administering to said subject a compound, wherein the compound is characterized in that upon contacting the Target Protein, the Target Protein activity or function is inhibited (e.g., partially inhibited or completely inhibited), such that said inhibited Target Protein exhibits reduced signaling output (e.g., compared to a corresponding Target Protein not contacted by the compound).

In certain aspects, the present disclosure provides a method of modulating activity of a Target Protein, comprising contacting a Target Protein with an effective amount of a compound described herein, or a pharmaceutically acceptable salt or solvate thereof, thereby modulating the activity of the Target Protein.

In certain aspects, the present disclosure provides a method of inhibiting cell growth, comprising administering an effective amount of a compound described herein, or a pharmaceutically acceptable salt or solvate thereof, to a cell expressing the Target Protein, thereby inhibiting growth of said cells. In some embodiments, the subject method comprises administering an additional agent to said cell.

In certain aspects, the present disclosure provides a method of treating a disease mediated at least in part by a Target Protein in a subject in need thereof, comprising administering to the subject an effective amount of a compound disclosed herein, or a pharmaceutically acceptable salt or solvate thereof. In some embodiments, the disease is cancer, such as a solid tumor or a hematological cancer. In some embodiments, the method further comprises administering an additional agent to the subject.

In certain aspects, the present disclosure provides a method of inhibiting activity of a Target Protein, comprising contacting the Target Protein with a compound disclosed herein, or a pharmaceutically acceptable salt or solvate thereof. In some embodiments, the compound exhibits an IC50 against the Target Protein of less than 10 μM, such as less than 5 μM, 1 μM, 500 nM, 100 nM, 50 nM, 10 nM, 5 nM, 1 nM, 500 μM, 50 μM, 10 μM or less.

In certain aspects, the present disclosure provides a method of treating a Target Protein-mediated cancer in a subject in need thereof, comprising administering to the subject an effective amount of a compound disclosed herein, such as a compound of Formula (I′) or (I), or a pharmaceutically acceptable salt or solvate thereof.

In some embodiments, the cancer is a solid tumor. In some embodiments, the cancer is a hematological cancer.

In some embodiments, the methods of treating cancer can be applied to treat a solid tumor or a hematological cancer. In some embodiments, the cancer being treated can be, without limitation, prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer selected from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound described herein, or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

Any of the treatment methods disclosed herein can be administered alone or in combination or in conjunction with another therapy or another agent. By “combination” it is meant to include (a) formulating a subject composition containing a subject compound together with another agent, or (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiments, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof. In one embodiment, a subject treatment method is combined with a chemotherapeutic agent.

In an aspect is provided a modified Target Protein comprising a compound described herein (or a remnant of a compound described herein wherein the remnant of said compound is modified from a standalone compound described herein upon covalently bonding to the amino acid) covalently bonded to the amino acid. In some embodiments, such covalently bonded modified Target Protein exhibits a reduced signaling output (e.g., compared to a corresponding unmodified Target Protein absent of the covalently bonded compound). A subject compound of the present disclosure encompasses a compound described herein immediately prior to covalently bonding the Target Protein as well as the resulting compound covalently bonded to the modified Target Protein. For example, a subject compound of the present disclosure can be covalently bonded to a Target Protein to form a modified Target Protein when a leaving group of the compound is displaced upon covalently bonding to an amino acid of the Target Protein. The compound prior to and subsequent to such covalent binding are all considered a subject compound of the present disclosure.

In some embodiments, the modified Target Protein described herein is formed by contacting a compound described herein with an aspartate residue of an unmodified Target Protein. In some embodiments, the modified Target Protein described herein is formed by contacting a compound described herein with a tyrosine residue of an unmodified Target Protein. In some embodiments, the modified Target Protein described herein is formed by contacting a compound described herein with a cysteine residue of an unmodified Target Protein. In some embodiments, the modified Target Protein described herein is formed by contacting a compound described herein with a lysine residue of an unmodified Target Protein. In some embodiments, the modified Target Protein described herein is formed by contacting a compound described herein with a histidine residue of an unmodified Target Protein. In some embodiments, the modified Target Protein described herein is formed by contacting a compound described herein with a serine residue of an unmodified Target Protein. In some embodiments, the compound comprises a staying group and a leaving group, and wherein said contacting results in release of the leaving group and formation of said modified Target Protein.

### Pharmaceutical Compositions and Methods of Administration

In an aspect is provided a pharmaceutical composition comprising a compound described herein, or a pharmaceutically acceptable salt or solvate thereof, and a pharmaceutically acceptable excipient.

In some embodiments, a compound described herein, or a pharmaceutically acceptable salt or solvate thereof, is administered to a subject in a biologically compatible form suitable for administration to treat or prevent diseases, disorders, or conditions. Administration of a compound described herein can be in any pharmacological form including a therapeutically effective amount of a compound described herein, or a pharmaceutically acceptable salt or solvate thereof, alone or in combination with a pharmaceutically acceptable carrier.

In some embodiments, a compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments of the methods described herein, a compound described herein is administered either alone or in combination with pharmaceutically acceptable carriers, excipients or diluents, in a pharmaceutical composition. Administration of a compound or composition described herein can be affected by any method that enables delivery of the compound to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, a compound described herein can be administered locally to the area in need of treatment, by, for example, local infusion during surgery, topical application such as creams or ointments, injection, catheter, or implant. The administration can also be by direct injection at the site of a diseased tissue or organ. In some embodiments, a compound described herein, or a pharmaceutically acceptable salt or solvate thereof, is administered orally.

In some embodiments of the methods described herein, a pharmaceutical composition suitable for oral administration is presented as a discrete unit such as a capsule, cachet or tablet, each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary, or paste.

J Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compound which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### EXAMPLES

The following examples are provided for illustrative purposes only and not to limit the scope of the claims provided herein. Unless noted otherwise, all materials, such as reagents, starting materials and solvents, were purchased from commercial suppliers, such as Sigma-Aldrich, VWR, and the like, and were used without further purification. Reactions were run under nitrogen atmosphere, unless noted otherwise. The progress of reactions was monitored by thin layer chromatography (TLC), analytical high performance liquid chromatography (anal. HPLC), and mass spectrometry, the details of which may be provided in specific examples.

Reactions were worked up as described specifically in each preparation; commonly, reaction mixtures were purified by extraction and other purification methods such as temperature- and solvent-dependent crystallization, and precipitation. In addition, reaction mixtures were routinely purified by preparative HPLC, for example, using Microsorb C18 or Microsorb BDS column packings and conventional eluents. Progress of reactions was typically monitored by liquid chromatography mass spectrometry (LCMS). Characterization of isomers was typically done by Nuclear Overhauser effect spectroscopy (NOE). Characterization of reaction products was routinely carried out by mass spectrometry and/or ¹H-NMR spectroscopy. For NMR measurement, samples were dissolved in deuterated solvent (CD₃OD, CDCl₃, or DMSO-d₆).

### Example 1a: Synthesis of N-(4-(1-((1-(1H-1,2,4-triazole-1-carbonyl)piperidin-2-yl)methyl)-4-amino-1H-pyrazolo[3,4-d]pyrimidin-3-yl)benzyl)-2-methoxy-5-methylbenzamide (217)

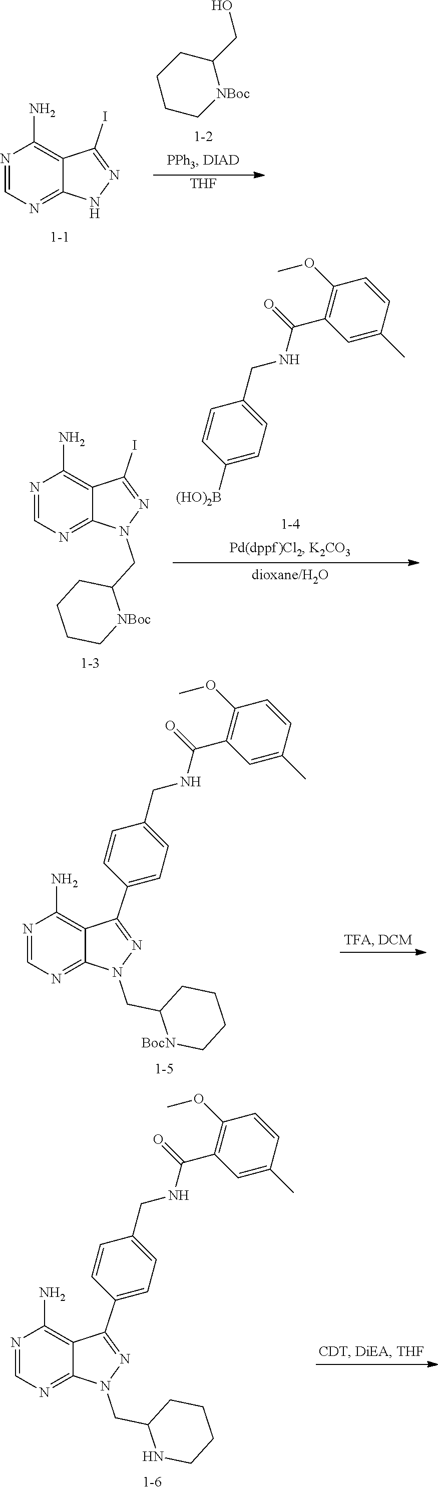

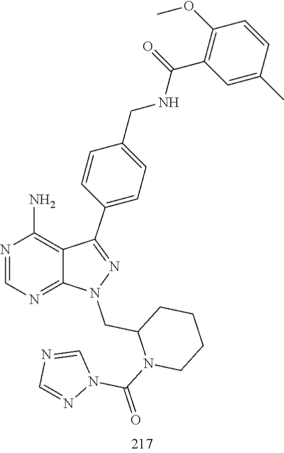 [SMILES: COc1ccc(C)cc1C(=O)NCc1ccc(-c2nn(CC3CCCCN3C(=O)n3cncn3)c3ncnc(N)c23)cc1]

Step 1: To a solution of 3-iodo-1H-pyrazolo[3,4-d]pyrimidin-4-amine (1-1, 300 mg, 1.0 eq) in THF (30 mL) in a three-neck bottle was added tert-butyl 2-(hydroxymethyl)piperidine-1-carboxylate (1-2, 420 mg, 1.7 eq) and PPh₃ (510 mg, 1.7 eq). Then, DIAD (390 mg, 1.7 eq) was added at −10° C. dropwise, and the resulting mixture was stirred for 2 hours from −10° C. to room temperature. The reaction mixture was diluted with ethyl acetate, then washed with water and brine. The organic layer was collected and dried over Na₂SO₄. After filtration, the filtrate was concentrated and the residue was purified by flash column chromatography on silica gel to afford tert-butyl 2-((4-amino-3-iodo-1H-pyrazolo[3,4-d]pyrimidin-1-yl)methyl) piperidine-1-carboxylate (1-3, 390 mg). ESI-MS m/z: 459.1 [M+H]⁺.

Step 2: To a stirred mixture of 1-3 (100 mg, 1.0 eq) and (4-((2-methoxy-5-methylbenzamido)methyl)phenyl)boronic acid (1-4, 100 mg, 1.5 eq) in 1,4-dioxane (10 mL) and water (1 mL) were added Pd(dppf)Cl₂.DCM (10 mg, 0.05 eq) and K₂CO₃ (62 mg, 2 eq) sequentially. The resulting mixture was degassed and back-filled with argon three times. The mixture was heated to 105° C. for 6 hours, then concentrated and purified by flash column chromatography on silica gel to afford tert-butyl 2-((4-amino-3-(4-((2-methoxy-5-methylbenzamido)methyl)phenyl)-1H-pyrazolo[3,4-d]pyrimidin-1-yl)methyl)piperidine-1-carboxylate (1-5, 125 mg). ESI-MS m/z: 586.3 [M+H]⁺.

Step 3: Compound 1-5 (125 mg) in a reaction tube was treated with 10% TFA in DCM (2 mL). The mixture was stirred at room temperature for 16 hours, then concentrated to provide crude N-(4-(4-amino-1-(piperidin-2-ylmethyl)-1H-pyrazolo[3,4-d]pyrimidin-3-yl)benzyl)-2-methoxy-5-methylbenzamide as a TFA salt (1-6, 250 mg).

Step 4: To a mixture of crude 1-6 (50 mg, 0.07 mmoL) in THF (1 mL) was added DIEA (50 μL, 0.29 mmol) followed by N,N′-carbonyl-di-(1,2,4-triazole) (CDT, 12 mg, 0.07 mmol). The resulting mixture was stirred at room temperature for 16 hours, then treated with sat. NH₄Cl solution (2 mL) and extracted with ethyl acetate (3×3 mL). The organic layers were combined and concentrated. The residue was purified by HPLC (10-100% ACN/water with 0.1% formic acid) to provide N-(4-(1-((1-(1H-1,2,4-triazole-1-carbonyl)piperidin-2-yl)methyl)-4-amino-1H-pyrazolo[3,4-d]pyrimidin-3-yl)benzyl)-2-methoxy-5-methylbenzamide as a white solid (217, 5 mg). LCMS m/z:581.2 [M+H]⁺.

### Example 1b: Synthesis of(7-(2-(pyrrolidin-1-yl)-4-(trifluoromethyl)benzyl)-1,7-diazaspiro[3.5]nonan-1-yl)(1H-1,2,4-triazol-1-yl)methanone (157)

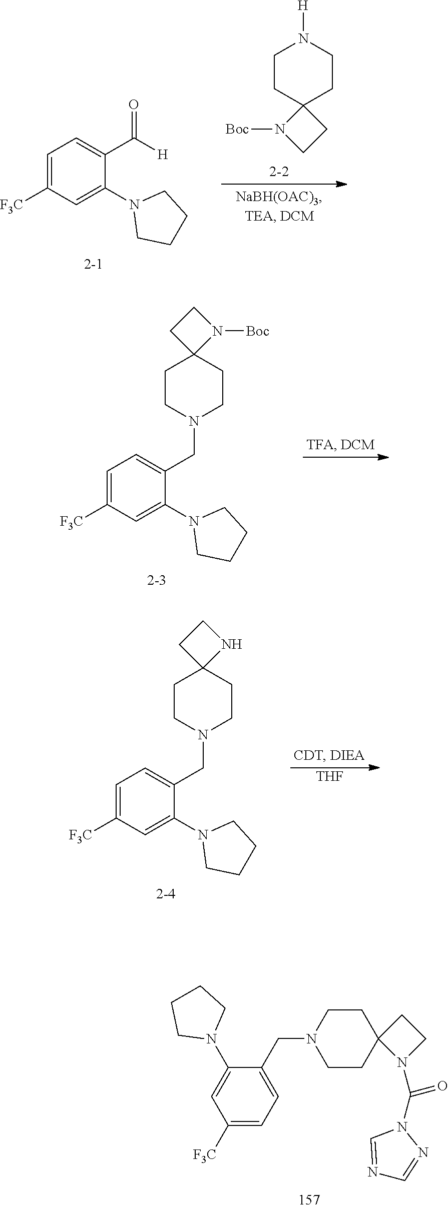 [SMILES: O=Cc1ccc(C(F)(F)F)cc1N1CCCC1 ; CC(C)(C)OC(=O)N1CCC12CCNCC2 ; CC(C)(C)OC(=O)N1CCC12CCN(Cc1ccc(C(F)(F)F)cc1N1CCCC1)CC2 ; FC(F)(F)c1ccc(CN2CCC3(CCN3)CC2)c(N2CCCC2)c1 ; "THF" ; O=C(N1CCC12CCN(Cc1ccc(C(F)(F)F)cc1N1CCCC1)CC2)n1cncn1]

Step 1: To a solution of 2-(pyrrolidin-1-yl)-4-(trifluoromethyl)benzaldehyde (2-1, 50 mg, 0.206 mmol, 1 eq) in DCM (10 mL) were added TEA (62 mg, 0.618 mmol, 3 eq) and NaBH(OAC)₃ (218.36 mg, 1.03 mmol, 5 eq). The resulting solution was stirred at 25° C. for 0.5 hour, then tert-butyl 1,7-diazaspiro[3.5]nonane-1-carboxylate (2-2, 93 mg, 0.412 mmol, 2 eq) was added. The reaction was stirred at room temperature for 16 hours, then partitioned between DCM and water. The organic layer was concentrated, and the residue was purified on a silica gel column eluting with ethyl acetate/petroleum ether (1:4) to afford tert-butyl 7-(2-(pyrrolidin-1-yl)-4-(trifluoromethyl)benzyl)-1,7-diazaspiro[3.5]nonane-1-carboxylate (2-3, 82 mg). ESI-MS m/z: 454.2 [M+H]⁺.

Step 2: To a stirred solution of 2-3 (82 mg, 0.181 mmol) in DCM (6 mL) was added trifluoroacetic acid (3 mL) and the resulting mixture was stirred at room temperature for 2 hours. The mixture was concentrated in vacuo. The residue was dissolved in DCM and treated with NH₃ in MeOH (7 N, 1 mL). The mixture was concentrated and the residue was purified by prep-TLC to afford 7-(2-(pyrrolidin-1-yl)-4-(trifluoromethyl)benzyl)-1,7-diazaspiro[3.5]nonane (2-4, 62 mg). ESI-MS m/z:354.2 [M+H]⁺.

Step 3: To a solution of 2-4 (62 mg, 0.175 mmol) in THF (4 mL) were added DIEA (68 mg, 0.525 mmol, 3 eq) and N,N′-carbonyl-di-(1,2,4-triazole) (CDT, 43 mg, 0.263 mmol, 1.5 eq). The resulting mixture was stirred at 25° C. under argon for 2 hours. The mixture was partitioned between ethyl acetate and water. The organic layer was collected and concentrated, and the residue was purified on a silica gel column to afford (7-(2-(pyrrolidin-1-yl)-4-(trifluoromethyl)benzyl)-1,7-diazaspiro[3.5]nonan-1-yl)(1H-1,2,4-triazol-1-yl)methanone (157, 27.5 mg). ¹HNMR (DMSO-d6, 400 MHz) δ 9.09 (s, 1H), 8.22 (s, 1H), 7.56 (d, J=7.6 Hz, 1H), 7.13 (d, J=7.6 Hz, 1H), 7.03 (s, 1H), 4.51-4.48 (m, 2H), 3.52 (s, 2H), 3.27-3.24 (m, 6H), 2.79-2.76 (m, 2H), 2.45-2.41 (m, 2H), 2.17-2.13 (m, 2H), 1.91-1.86 (m, 6H). ESI-MS m/z: 449.2 [M+H]⁺.

### Example 1c: Synthesis of N-methyl-N-(1-(2-(pyrrolidin-1-yl)-4-(trifluoromethyl)benzyl)piperidin-4-yl)-1H-1,2,4-triazole-1-carboxamide (190)

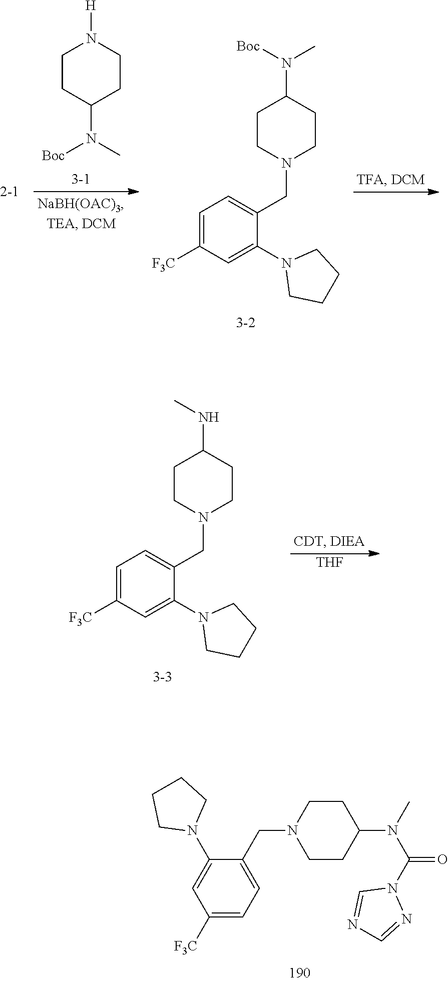 [SMILES: CN(C(=O)OC(C)(C)C)C1CCNCC1 ; CN(C(=O)OC(C)(C)C)C1CCN(Cc2ccc(C(F)(F)F)cc2N2CCCC2)CC1 ; CNC1CCN(Cc2ccc(C(F)(F)F)cc2N2CCCC2)CC1 ; "THF" ; CN(C(=O)n1cncn1)C1CCN(Cc2ccc(C(F)(F)F)cc2N2CCCC2)CC1]

Step 1: To a solution of 2-1 (50 mg, 0.206 mmol, 1 eq) in DCM (10 mL) were added TEA (62 mg, 0.618 mmol, 3 eq) and NaBH(OAC)₃ (218.36 mg, 1.03 mmol, 5 eq). The resulting solution was stirred at 25° C. for 0.5 hour before tert-butyl methyl(piperidin-4-yl)carbamate (3-1, 88 mg, 0.412 mmol, 2 eq) was added. The reaction was stirred at room temperature for 16 hours. The mixture was partitioned between DCM and water. The organic layer was concentrated and the residue was purified on a silica gel column eluting with ethyl acetate/petroleum (1:4) to afford tert-butyl methyl(1-(2-(pyrrolidin-1-yl)-4-(trifluoromethyl)benzyl)piperidin-4-yl)carbamate (3-2, 70 mg). ESI-MS m/z:442.2 [M+H]⁺.

Step 2: To a stirred solution of 3-2 (70 mg, 0.159 mmol) in DCM (6 mL) was added trifluoroacetic acid (3 mL) and the resulting mixture was stirred at room temperature for 2 hours. The mixture was concentrated in vacuo. The residue was dissolved in DCM and treated with NH₃ in MeOH (7 N, 1 mL). The mixture was concentrated, and the residue was purified by prep-TLC to afford N-methyl-1-(2-(pyrrolidin-1-yl)-4-(trifluoromethyl)benzyl)piperidin-4-amine (3-3, 42 mg). ESI-MS m/z:342.2 [M+H]⁺.

Step 3: To a solution of 3-3 (42 mg, 0.123 mmol) in THF (4 mL) was added DIEA (38 mg, 0.369 mmol, 3 eq) and N,N′-carbonyl-di-(1,2,4-triazole) (CDT, 30 mg, 0.185 mmol, 1.5 eq). The resulting mixture was stirred at 25° C. under argon for 2 hours. The mixture was partitioned between ethyl acetate and water. The organic layer was concentrated. The residue was purified on a silica gel column to afford N-methyl-N-(1-(2-(pyrrolidin-1-yl)-4-(trifluoromethyl)benzyl)piperidin-4-yl)-1H-1,2,4-triazole-1-carboxamide (190, 12 mg). ¹HNMR (DMSO-d6, 400 MHz) δ 9.03 (s, 1H), 8.23 (s, 1H), 7.56 (d, J=7.6 Hz, 1H), 7.11 (d, J=7.6 Hz, 1H), 7.03 (s, 1H), 3.53 (s, 3H), 3.27-3.23 (m, 4H), 2.96 (s, 3H), 2.89-2.86 (m, 2H), 2.03-1.96 (m, 2H), 1.91-1.81 (m, 6H), 1.74-1.72 (m, 2H). ESI-MS m/z: 437.2 [M+H]⁺.

### Example 1d: Synthesis of N-(3-(4-amino-3-(4-((2-methoxy-5-methylbenzamido)methyl)phenyl)-1H-pyrazolo[3,4-d]pyrimidin-1-yl)cyclohexyl)-N-methyl-1H-tetrazole-1-carboxamide (219)

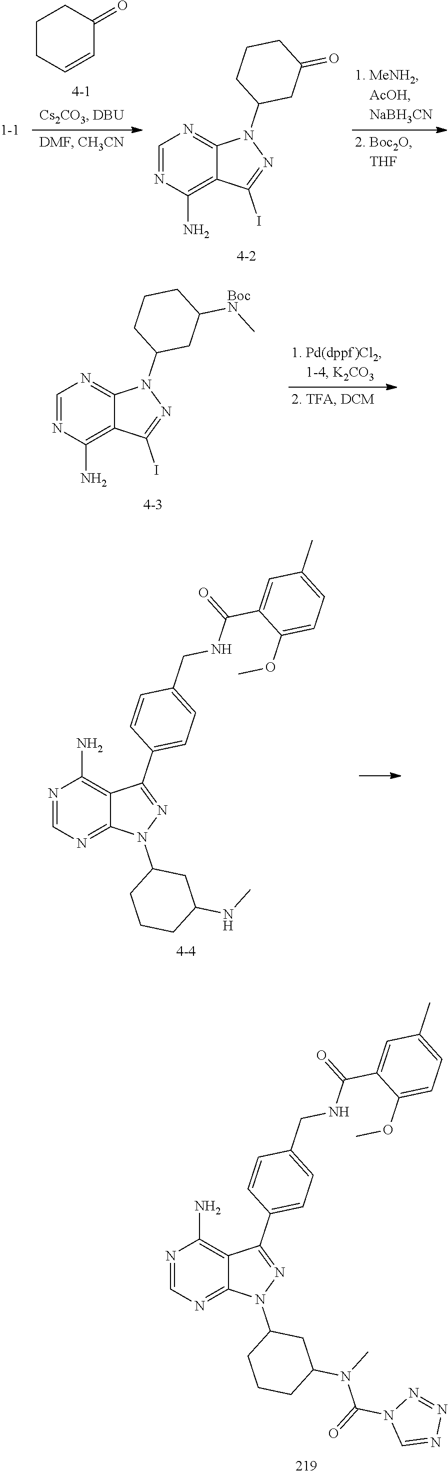 [SMILES: O=C1C=CCCC1 ; Nc1ncnc2c1c(I)nn2C1CCCC(=O)C1 ; CN(C(=O)OC(C)(C)C)C1CCCC(n2nc(I)c3c(N)ncnc32)C1 ; CNC1CCCC(n2nc(-c3ccc(CNC(=O)c4cc(C)ccc4OC)cc3)c3c(N)ncnc32)C1 ; COc1ccc(C)cc1C(=O)NCc1ccc(-c2nn(C3CCCC(N(C)C(=O)n4cnnn4)C3)c3ncnc(N)c23)cc1]

Step 1: To a solution of 1-1 (522 mg, 1.0 eq) in DCM/MeCN (2 mL:10 mL) were added cyclohex-2-en-1-one (4-1, 576 mg, 3.0 eq.) and DBU (912 mg, 3.0 eq). Cs₂CO₃ (~100 mg, 0.1 eq.) was added and the resulting mixture was stirred at 30° C. for 2 hours. The reaction mixture was poured into ice-water and extracted with ethyl acetate. The organic phase was washed with water and brine, then dried over Na₂SO₄. After filtration, the filtrate was concentrated and the residue was purified by flash column chromatography on silica gel to afford 3-(4-amino-3-iodo-1H-pyrazolo[3,4-d]pyrimidin-1-yl)cyclohexan-1-one (4-2, 400 mg). ESI-MS m/z: 358.01 [M+H]⁺.

Step 2: To a solution of 4-2 (251 mg, 1.0 eq) and methylamine hydrochloride (143 mg, 3.0 eq) in MeOH (18 ml) was added AcOH (84 mg, 2.0 eq). The mixture was stirred at room temperature for 1 hour before the addition of NaBH₃CN (180 mg, 4.0 eq). The resulting mixture was heated to 50° C. for 8 hours, then cooled to room temperature and poured into ice-water. The mixture was basified by NaHCO₃ (aq) and extracted with ethyl acetate. The organic phase was concentrated and the crude residue was dissolved in THF (28 mL). TEA (2.0 eq) and di-tert-butyl dicarbonate (1.0 eq) were added. The reaction was stirred for 2 hours at 30° C., then concentrated and the residue purified by flash column chromatography on silica gel to afford tert-butyl (3-(4-amino-3-iodo-1H-pyrazolo[3,4-d]pyrimidin-1-yl)cyclohexyl)(methyl)carbamate (4-3, 300 mg). ESI-MS m/z: 373.2 [M+H]⁺.

Step 3: To a stirred mixture of 4-3 (120 mg, 1.0 eq) and 1-4 (100 mg, 1.5 eq) in 1,4-dioxane (10 mL) and water (1 mL) were added Pd(dppf)Cl₂.DCM (10 mg, 0.05 eq) and potassium carbonate (62 mg, 2 eq) sequentially. The resulting mixture was degassed and back-filled with argon three times. The mixture was heated to 90° C. for 6 hours, then cooled to room temperature and concentrated. The residue was purified by flash column chromatography on silica gel to afford tert-butyl (3-(4-amino-3-(4-((2-methoxy-5-methylbenzamido)methyl)phenyl)-1H-pyrazolo[3,4-d]pyrimidin-1-yl)cyclohexyl)(methyl)carbamate (110 mg). ESI-MS m/z: 473.3 [M+H]⁺.

Step 4: tert-butyl (3-(4-amino-3-(4-((2-methoxy-5-methylbenzamido)methyl)phenyl)-1H-pyrazolo[3,4-d]pyrimidin-1-yl)cyclohexyl)(methyl)carbamate (107 mg) in a reaction tube was treated with 10% TFA in DCM (2 mL). The mixture was stirred at room temperature for 16 hours, then concentrated to provide crude N-(4-(4-amino-1-(3-(methylamino)cyclohexyl)-1H-pyrazolo[3,4-d]pyrimidin-3-yl)benzyl)-2-methoxy-5-methylbenzamide as a TFA salt (4-4, 150 mg). ESI-MS m/z: 500.3 [M+H]⁺.

Step 5: To a solution of triphosgene (5 mg, 0.017 mmol) in DCM (0.5 mL) in an ice bath was added tetrazole (0.45N in MeCN, 133 μL, 0.06 mmol) followed by DIEA (10.5 μL, 0.06 mmol). The resulting mixture was stirred at 0° C. for 1 hr before a solution of crude 4-4 (10 mg) and DIEA (20 μL, 0.11 mmol) in DCM (0.5 mL) was added via syringe. The mixture was allowed to warm to room temperature and stirred for 16 hours. The mixture was concentrated and the residue was purified by HPLC (10-70% MeCN in water with 0.1% formic acid) to provide N-(3-(4-amino-3-(4-((2-methoxy-5-methylbenzamido)methyl)phenyl)-1H-pyrazolo[3,4-d]pyrimidin-1-yl)cyclohexyl)-N-methyl-1H-tetrazole-1-carboxamide (219, 1.5 mg). ESI-MS m/z: 596.2 [M+H]⁺.

### Example 1e: Synthesis of (R)-N-(4-(1-((1-(1H-1,2,4-triazole-1-carbonyl)azetidin-2-yl)methyl)-4-amino-1H-pyrazolo[3,4-d]pyrimidin-3-yl)benzyl)-5-fluoro-2-methoxybenzamide (108)

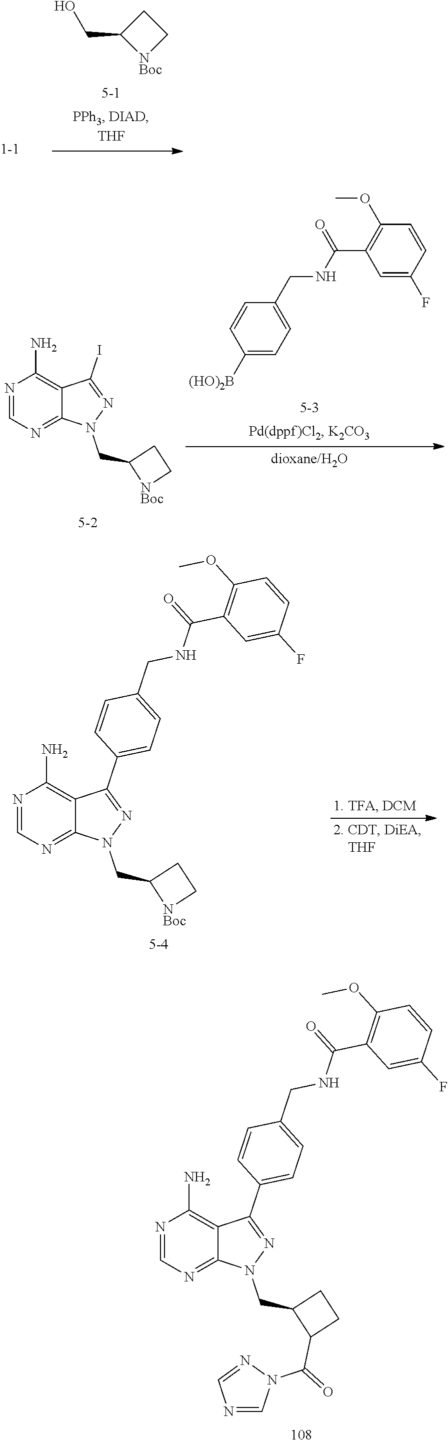 [SMILES: CC(C)(C)OC(=O)N1CC[C@@H]1CO ; CC(C)(C)OC(=O)N1CC[C@@H]1Cn1nc(I)c2c(N)ncnc21 ; COc1ccc(F)cc1C(=O)NCc1ccc(B(O)O)cc1 ; COc1ccc(F)cc1C(=O)NCc1ccc(-c2nn(C[C@H]3CCN3C(=O)OC(C)(C)C)c3ncnc(N)c23)cc1 ; COc1ccc(F)cc1C(=O)NCc1ccc(-c2nn(C[C@H]3CCC3C(=O)n3cncn3)c3ncnc(N)c23)cc1]

Step 1: To a solution of 1-1 (150 mg, 1.0 eq) in THF (20 mL) in a three-neck bottle were added tert-butyl (R)-2-(hydroxymethyl)azetidine-1-carboxylate (5-1, 183 mg, 1.7 eq.) and PPh₃ (255 mg, 1.7 eq.). Then, DIAD (195 mg, 1.7 eq) was added at −10° C. dropwise and the resulting mixture was stirred for 30 min. at −10° C. The reaction mixture was diluted with ethyl acetate, then washed with water and brine. The organic layer was collected and dried over Na₂SO₄. After filtration, the filtrate was concentrated and the residue was purified by flash column chromatography on silica gel to afford tert-butyl (R)-2-((4-amino-3-iodo-1H-pyrazolo[3,4-d]pyrimidin-1-yl)methyl)azetidine-1-carboxylate (5-2, 130 mg). ESI-MS m/z: 431.06 [M+H]⁺.

Step 2: To a stirred mixture of 5-2 (130 mg, 1.0 eq) and (4-((5-fluoro-2-methoxybenzamido)methyl)phenyl) boronic acid (5-3, 140 mg, 1.5 eq) in 1,4-dioxane (10 mL) and water (1 mL) were added Pd(dppf)Cl₂.DCM (12 mg, 0.05 eq) and potassium carbonate (83 mg, 2 eq) sequentially. The resulting mixture was degassed and back-filled with argon three times. The mixture was heated to 105° C. for 6 hours, then cooled to room temperature and concentrated. The residue was purified by flash column chromatography on silica gel to afford tert-butyl (R)-2-((4-amino-3-(4-((5-fluoro-2-methoxybenzamido)methyl)phenyl)-1H-pyrazolo[3,4-d]pyrimidin-1-yl)methyl)azetidine-1-carboxylate (5-4, 200 mg). ESI-MS m/z: 562.25 [M+H]⁺.

Step 3: To a solution of 5-4 (90 mg, 1.0 eq) in DCM (6 mL) was added TFA (2 mL). The reaction mixture was stirred at room temperature for 30 min, then solvent was removed under vacuum. The residue was dissolved in THF (9 mL), then DIEA (0.5 ml) and di(1H-1,2,4-triazol-1-yl) methanone (CDT, 40 mg, 1.2 eq) were added. The reaction mixture was stirred at room temperature for 10 min. The reaction mixture was diluted with ethyl acetate, then washed with water and brine. The organic phase was dried over Na₂SO₄. After filtration, the filtrate was concentrated and the residue was purified by flash column chromatography on silica gel to afford (R)-N-(4-(1-((1-(1H-1,2,4-triazole-1-carbonyl)azetidin-2-yl)methyl)-4-amino-1H-pyrazolo[3,4-d]pyrimidin-3-yl)benzyl)-5-fluoro-2-methoxybenzamide (108, 38 mg). 1HNMR (400 MHz, CDC13) δ 8.90 (s, 1H), 8.34-8.37 (m, 2H), 7.98-8.01 (m, 2H), 7.52-7.59 (m, 3H), 7.17-7.22 (m, 11), 6.96-7.00 (m, 1H), 5.59 (s, 2H), 4.89-5.02 (m, 2H), 4.76-4.78 (m, 2H), 3.98 (s, 3H), 2.26-2.56 (m, 2H), 1.68 (s, 4H). ESI-MS m/z: 557.2 [M+H]⁺.

### Example 1f: Synthesis of N-(2-(4-amino-3-(4-((5-fluoro-2-methoxybenzamido)methyl)phenyl)-1H-pyrazolo[3,4-d]pyrimidin-1-yl)ethyl)-N-cyclopropyl-1H-1,2,4-triazole-1-carboxamide (316)

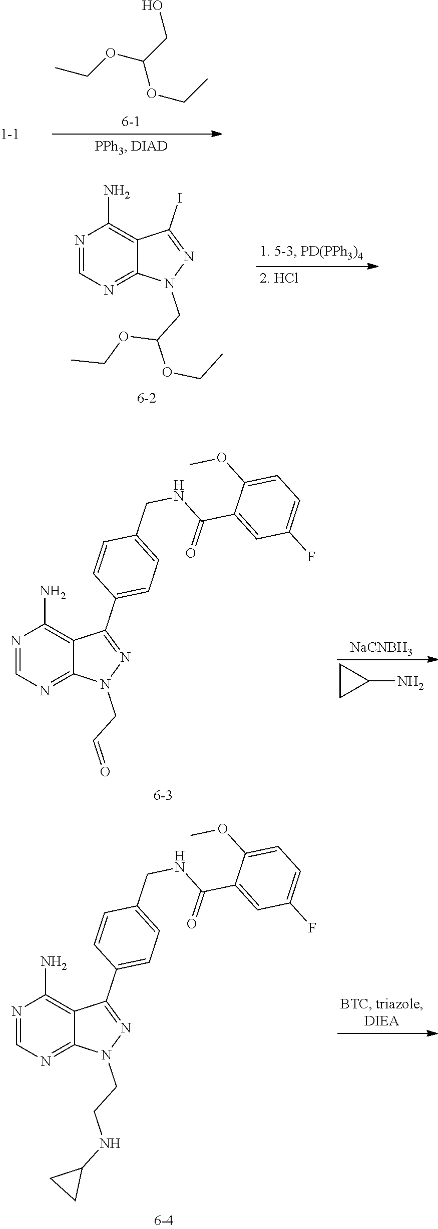

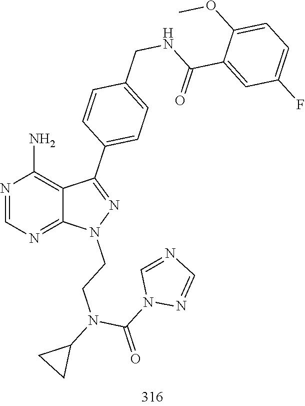 [SMILES: COc1ccc(F)cc1C(=O)NCc1ccc(-c2nn(CCN(C(=O)n3cncn3)C3CC3)c3ncnc(N)c23)cc1]

Step 1: To a stirred solution of 1-1 (522 mg, 2 mmol) and 2,2-diethoxyethan-1-ol (6-1, 375 mg, 2.8 mmol) in THF (15 mL) was added PPh₃ (734 mg, 2.8 mmol). The mixture was heated to 50° C. for 30 min, then DIAD (566 mg, 2.8 mmol) was added to the reaction at 50° C. and the resulting mixture was stirred for 5 hours at 50° C. The reaction was cooled to room temperature, then solvent was removed under vacuum and the residue was purified by flash column chromatography to afford 1-(2,2-diethoxyethyl)-3-iodo-1H-pyrazolo[3,4-d]pyrimidin-4-amine (6-2, 700 mg). ESI-MS m/z: 378.1 [M+H]⁺.

Step 2: To a stirred solution of 6-2 (120 mg, 0.318 mmol) and 5-3 (125 mg, 0.414 mmol) in toluene (6 mL)/ethanol (6 mL)/H₂O (3 mL) were added Na₂CO₃ (67 mg, 0.636 mmol) and Pd(PPh₃)₄ (20 mg) under argon. The mixture was heated to 100° C. for 16 hours, then solvent was removed and the residue was purified by flash column chromatography to afford N-(4-(4-amino-1-(2,2-diethoxyethyl)-1H-pyrazolo[3,4-d]pyrimidin-3-yl)benzyl)-5-fluoro-2-methoxybenzamide (130 mg). ESI-MS m/z: 509.2 [M+H]⁺.

Step 3: To a stirred solution of N-(4-(4-amino-1-(2,2-diethoxyethyl)-1H-pyrazolo[3,4-d]pyrimidin-3-yl)benzyl)-5-fluoro-2-methoxybenzamide (130 mg, 0.255 mmol) in acetone (15 mL) was added aqueous HCl solution (1N, 15 mL). The mixture was stirred at 80° C. for 4 hours, then cooled to room temperature. Aqueous NaHCO₃ solution was added to adjust the pH to 8, then the mixture was extracted with ethyl acetate. The organic layer was concentrated and the residue containing N-(4-(4-amino-1-(2-oxoethyl)-1H-pyrazolo[3,4-d]pyrimidin-3-yl)benzyl)-5-fluoro-2-methoxybenzamide (6-3) was used in the next step without purification (90 mg). ESI-MS m/z: 435.2 [M+H]⁺.

Step 4: To a stirred solution of crude 6-3 (60 mg, 0.138 mmol) and cyclopropanamine (24 mg, 0.415 mmol) in MeOH (10 mL) was added AcOH (2 drops). The mixture was stirred for 15 min at room temperature before the addition of NaCNBH₃ (52 mg, 0.828 mmol). The mixture was stirred for 16 hours at 45° C., then solvent was removed under vacuum and the residue was purified by flash column chromatography to afford N-(4-(4-amino-1-(2-(cyclopropylamino)ethyl)-1H-pyrazolo[3,4-d]pyrimidin-3-yl)benzyl)-5-fluoro-2-methoxybenzamide (6-4, 40 mg), ESI-MS m/z: 476.2 [M+H]⁺.

Step 5: To a stirred solution of 6-4 (40 mg, 0.084 mmol), 1H-1,2,4-triazole (58 mg, 0.84 mmol) and Et₃N (170 mg, 1.68 mmol) in DCM (10 mL) was added triphosgene (125 mg, 0.42 mmol) at 0° C. The mixture was stirred for 1 hour at 0° C., then warmed to room temperature. The mixture was washed with aqueous NaHCO₃ solution. The organic phase was collected and concentrated. The residue was purified by flash column chromatography to afford N-(2-(4-amino-3-(4-((5-fluoro-2-methoxybenzamido)methyl)phenyl)-1H-pyrazolo[3,4-d]pyrimidin-1-yl)ethyl)-N-cyclopropyl-1H-1,2,4-triazole-1-carboxamide (316, 20 mg). ¹H NMR (400 MHz, DMSO-d6) δ 8.78 (m, 1H), 8.12 (s, 1H), 7.99 (s, 1H), 7.6-7.4 (m 5H), 7.3-7.2 (m, 1H), 7.1 (m, 1H), 4.65-4.55 (m, 2H), 4.5 (d, J=6.0 Hz, 2H), 4.0 (m, 2H), 3.81 (s, 3H), 2.81 (m, 1H), 0.7-0.3 (m, 4H). ESI-MS m/z: 571.2 [M+H]⁺.

### Example 1g: Synthesis of 7-((N-ethyl-1H-1,2,4-triazole-1-carboxamido)methyl)-2-(4-phenoxyphenyl)-4,5,6,7-tetrahydropyrazolo[1,5-a]pyrimidine-3-carboxamide (120)

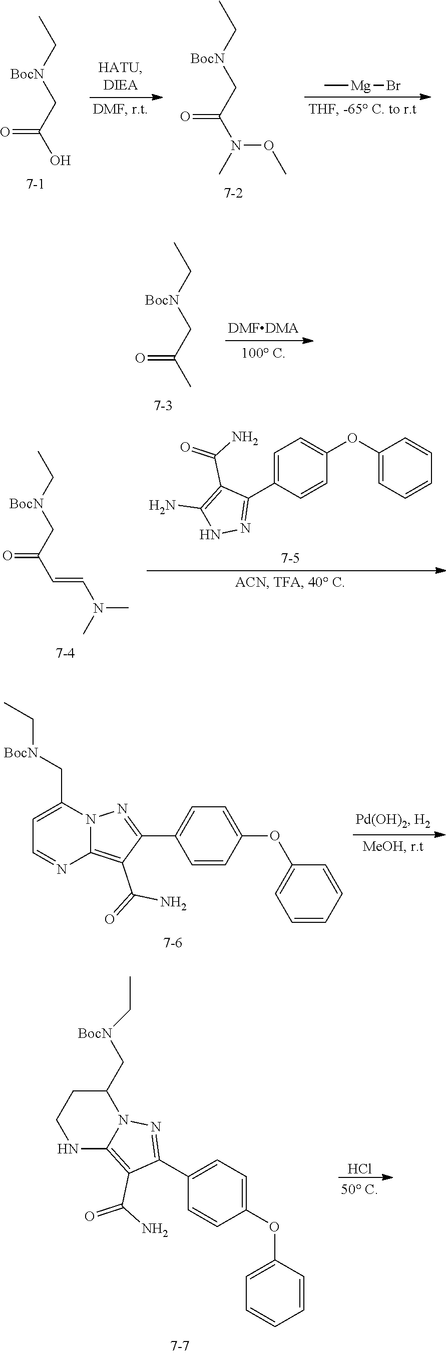

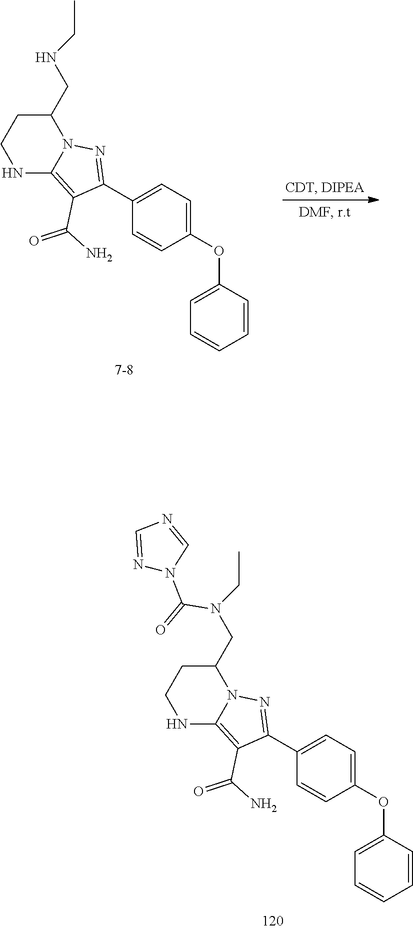 [SMILES: CCNCC1CCNc2c(C(N)=O)c(-c3ccc(Oc4ccccc4)cc3)nn21 ; CCN(CC1CCNc2c(C(N)=O)c(-c3ccc(Oc4ccccc4)cc3)nn21)C(=O)n1cncn1]

Step 1: To a stirred solution of N-(tert-butoxycarbonyl)-N-ethylglycine (7-1, 18 g, 88.56 mmol) in DMF (200 mL) were added N,O-dimethylhydroxylamine (13 g, 132.84 mmol), DIPEA (34 g, 265.7 mmol) and HATU (40 g, 106.3 mmol) under argon. The reaction mixture was stirred at room temperature for 1 hour, then diluted with ethyl acetate (200 mL) and washed with saturated LiCl aqueous solution (3×100 mL). The organic layer was dried over anhydrous Na₂SO₄, filtered and concentrated in vacuo. The crude product was purified by silica gel column (petroleum ether:EtOAc=1:1) to give tert-butyl ethyl(2-(methoxy(methyl)amino)-2-oxoethyl)carbamate (7-2) as a yellow oil (19.8 g). MS (ESI) m/z=247.2 [M+H]⁺.

Step 2: A solution of 7-2 (20 g, 81.2 mmol) in THF (200 mL) was cooled to −65° C. Methyl magnesium bromide (81.2 mL, 243.6 mmol) was added over 30 min. The mixture was stirred at −65° C. for 1.5 hours, and then warmed to 0° C. and stirred for another 1 hour. The mixture was quenched with sat. NH₄Cl aqueous solution (100 mL) and diluted with ethyl acetate (200 mL). The organic layer was washed with brine (3×100 mL), dried over anhydrous Na₂SO₄, filtered and concentrated in vacuo. The crude product was purified by silica gel column chromatography (petroleum ether:EtOAc=3:1) to give tert-butyl ethyl(2-oxopropyl)carbamate (7-3) as a yellow oil (12.4 g). ¹H NMR (400 MHz, CDCl₃) δ 3.97 (s, 111), 3.87 (s, 111), 3.30 (m, 2H), 2.13 (s, 311), 1.47 (s, 911), 1.11(m, 311).

Step 3: A solution of 7-3 (2 g, 10.0 mmol) in DMFDMA (7 mL, 30 mmol) was stirred at 100° C. for 24 hours, then concentrated to afford crude tert-butyl -(4-(dimethylamino)-2-oxobut-3-en-1-yl)(ethyl)carbamate (7-4), which was used directly in the next step without purification (2.2 g). MS (ESI) m/z=257.0 [M+H]⁺.

Step 4: To a solution of 5-amino-3-(4-phenoxyphenyl)-1H-pyrazole-4-carboxamide (7-5, 400 mg, 1.36 mmol) in ACN (7 mL) was added 7-4 (513 mg, 2.0 mmol). TFA (171 mg, 1.5 mmol) in ACN (2 mL) was added into the reaction dropwise. The mixture was stirred at room temperature for 15 min, then heated to 40° C. and stirred for 4 hours. The reaction mixture was then concentrated and the pH was adjusted to 7 with aqueous saturated NaHCO₃ solution. The mixture was extracted with DCM (3×20 mL), then the organic layer was dried over anhydrous Na₂SO₄, filtered and concentrated in vacuo. The residue was purified by silica gel column chromatography (petroleum ether:EtOAc=1:1) to give the desired product (7-6) as a yellow oil (300 mg). MS (ESI) m/z=488.1 [M+H]⁺.

Step 5: To a stirred solution of 7-6 (300 mg, 0.61 mmol) in MeOH (30 mL) was added Pd(011)₂ (150 mg). The reaction mixture was stirred at room temperature for 16 hours under H₂, then filtered and concentrated in vacuo to give tert-butyl ((3-carbamoyl-2-(4-phenoxyphenyl)-4,5,6,7-tetrahydropyrazolo[1,5-a]pyrimidin-7-yl)methyl)(ethyl)carbamate (7-7, 169 mg). MS (ESI) m/z=492.1 [M+H]⁺.

Step 6: To a stirred solution of 7-7 (260 mg, 0.53 mmol) was added 37% HCl (10 mL) and the resulting mixture was stirred at 50° C. for 2 h. The mixture was concentrated in vacuo, then 1OM NaOH aq. solution was added to the residue and the mixture stirred at 90° C. for 1 hour. The mixture was extracted with DCM (3×10 mL) and washed with water (10 mL). The organic layer was dried over anhydrous Na₂SO₄, filtered and concentrated in vacuo. The crude product was purified by silica gel column (DCM:NH₃-MeOH=10:1) to give 7-((ethylamino)methyl)-2-(4-phenoxyphenyl)-4,5,6,7-tetrahydropyrazolo[1,5-a]pyrimidine-3-carboxamide (7-8) as a white solid (190 mg). MS (ESI) m/z=392.0 [M+H]⁺. ¹H NMR (400 MHz, d₆-DMSO) δ 7.53-7.49 (m, 2H), 7.46-7.39 (m, 2H), 7.18 (t, J=7.6 Hz, 1H), 7.11-7.04 (m, 4H), 6.63 (s, 1H), 4.16-4.09 (m, 1H), 3.29 (d, J=3.2 Hz, 2H), 3.18 (d, J=5.2 Hz, 1H), 3.01 (dd, J=12.0, 4.8 Hz, 1H), 2.79 (dd, J=12.0, 7.7 Hz, 1H), 2.61-2.53 (m, 2H), 2.10-2.01 (m, 2H), 1.01 (t, J=7.1 Hz, 3H).

Step 7: To a stirred solution of 7-8 (50 mg, 0.127 mmol) and di(1H-1,2,4-triazol-1-yl)methanone (CDT, 21 mg, 0.127 mmol) in DMF (1 mL) was added DIPEA (50 mg, 0.383 mmol). The mixture was stirred at room temperature for 1 hour, then purified by prep-HPLC to give 7-((N-ethyl-1H-1,2,4-triazole-1-carboxamido)methyl)-2-(4-phenoxyphenyl)-4,5,6,7-tetrahydropyrazolo[1,5-a]pyrimidine-3-carboxamide (120) as a white solid (41.34 mg). ¹H NMR (400 MHz, d₆-DMSO) δ 8.75-8.83 (m, 1H), 8.22 (d, J=12 Hz, 1H), 7.55-7.37 (m, 4H), 7.17 (t, J=7.6 Hz, 1H), 7.06 (dd, J=15.2, 8.1 Hz, 4H), 6.68 (s, 1H), 4.54 (s, 1H), 3.80 (dd, J=14, 5.7 Hz, 11), 3.32 (s, 5H), 2.07 (s, 2H), 1.17 (s, 3H). MS (ESI) m/z=487.0 [M+H]⁺.

### Example 1h: Synthesis of trans-N-(3-(5-amino-4-carbamoyl-3-(4-((5-fluoro-2-methoxybenzamido)methyl)phenyl)-1H-pyrazol-1-yl)cyclohexyl)-N-methyl-1H-1,2,4-triazole-1-carboxamide (194)

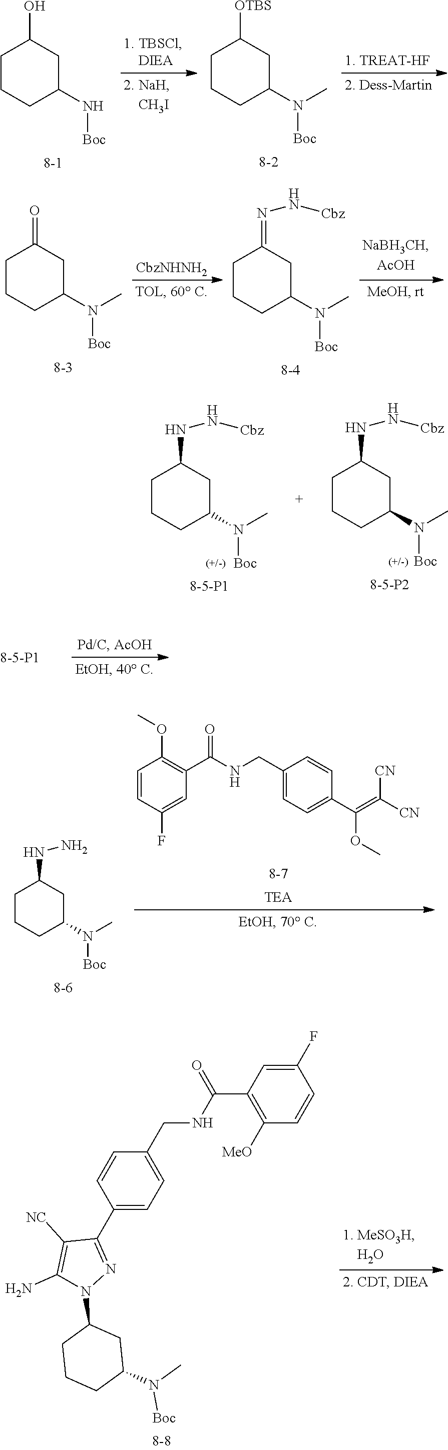

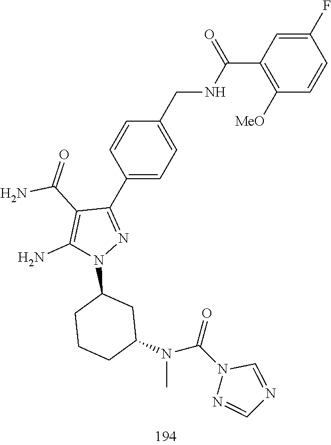 [SMILES: COc1ccc(F)cc1C(=O)NCc1ccc(-c2nn([C@@H]3CCC[C@@H](N(C)C(=O)n4cncn4)C3)c(N)c2C(N)=O)cc1]

Step 1: To a solution of tert-butyl (3-hydroxycyclohexyl)carbamate (8-1, 10 g, 41.45 mmol) in DMF (100 mL) was added TBSCl (8.4 g, 55.73 mmol). The mixture was stirred at room temperature for 20 min, then DIEA (12 g, 92.9 mmol) was added slowly at 0° C. The mixture was stirred at room temperature for 16 hours, then diluted with ethyl acetate (600 mL) and washed with brine (5×200 mL). The organic layer was dried (Na₂SO₄), filtered and concentrated under vacuum. The crude residue was purified by silica gel column chromatography to give tert-butyl (3-((tert-butyldimethylsilyl)oxy)cyclohexyl)carbamate as a yellow oil (12 g). ¹H NMR (400 MHz, d₆-DMSO) δ 6.47 (b, 1H), 3.57 (m, 1H), 3.23 (m, 1H), 1.55-1.90 (m, 4H), 1.33 (s, 9H), 0.90-1.25 (m, 3H), 0.83 (s, 9H), 0 (s, 6H).

Step 2: To a solution of tert-butyl (3-((tert-butyldimethylsilyl)oxy)cyclohexyl)carbamate (12 g, 39.51 mmol) in DMF (130 mL) was added NaH (1.42 g, 59.27 mmol) at 0° C. slowly and the resulting mixture was stirred for 1 hour. Then, CH₃I (8.41 g, 59.27 mmol) was added slowly at 0° C. The resulting mixture was stirred at room temperature for 5 hours, then quenched with water at 0° C. and diluted with ethyl acetate (700 mL). The mixture was washed with brine (4×200 mL), then the organic layer was dried (Na₂SO₄), filtered and concentrated. The crude residue was purified by silica gel column chromatography to give tert-butyl (3-((tert-butyldimethylsilyl)oxy)cyclohexyl)(methyl)carbamate (8-2) as a yellow oil (11 g). ¹H NMR (400 MHz, d₆-DMSO) δ 3.57 (m, 1H), 3.23 (m, 1H), 2.62 (s, 3H), 1.40-1.80 (m, 4H), 1.35 (s, 9H), 0.90-1.30 (m, 3H), 0.83 (s, 9H), 0 (s, 6H).

Step 3: To a solution of 8-2 (11 g, 31.11 mmol) in THF (110 mL) was added triethylamine trihydrofluoride (TREAT-HF, 15.0 g, 93.33 mmol) at room temperature. The mixture was stirred at room temperature for 2 hours, then diluted with ethyl acetate and washed with water and brine. The organic layer was dried (Na₂SO₄), filtered and concentrated under vacuum. The crude residue was purified by silica gel column chromatography (petroleum ether:EtOAc=5:1) to give tert-butyl (3-hydroxycyclohexyl)(methyl) carbamate as a yellow solid (5g). ¹H NMR (400 MHz, d₆-DMSO) δ 4.5 (br, 1H), 3.87 (br, 1H), 3.40 (m, 1H), 2.62 (s, 3H), 1.55-1.80 (m, 4H), 1.43 (s, 9H), 0.90-1.40 (m, 3H).

Step 4: To a solution of tert-butyl (3-hydroxycyclohexyl)(methyl)carbamate (5 g, 17.95 mmol) in DCM (80 mL) was added Dess-Martin reagent (15.22 g, 35.9 mmol) at room temperature. The reaction was stirred at room temperature for 16 hours, then quenched with water. The mixture was extracted with DCM, then the combined organic layer was dried over anhydrous Na₂SO₄, filtered and concentrated under vacuum. The crude residue was purified by silica gel column chromatography (petroleum ether:EtOAc=3:1) to give tert-butyl methyl(3-oxocyclohexyl)carbamate (8-3) as a yellow oil (3.5 g, yield: 70.0%). ¹H NMR (400 MHz, d₆-DMSO) δ 4.05 (br, 11), 3.75 (s, 3H), 2.32 (m, 11), 2.10 (m, 2H), 1.60-2.00 (m, 3H), 1.43 (s, 9H).

Step 5: A solution of 8-3 (5.4 g, 23 mmol) and CbzNHNH₂ (2.7 g, 23 mmol) in toluene (50 mL) was stirred at 60° C. for 1 hour. The mixture was concentrated in vacuo to provide crude benzyl-2-(3-((tert-butoxycarbonyl)(methyl)amino)cyclohexylidene) hydrazine-1-carboxylate (8-4, 9 g, yield: 100%), which was used directly in the next step without purification. MS (ESI) m/z=398.1 [M+Na]⁺.

Step 6: To a solution of 8-4 (9 g, 24 mmol) in MeOH (90 mL) were added AcOH (0.7 mL) and NaBH₃CN (7.56 g, 120 mmol). The reaction mixture was stirred at room temperature for 16 hours, then quenched with water (200 mL) and extracted with ethyl acetate (2×200 mL). The organic layer was dried (Na₂SO₄), filtered and concentrated in vacuo. The crude product was purified by silica gel column (EtOAc:petroleum ether=1:1) to give benzyl 2-(3-((tert-butoxycarbonyl)(methyl)amino)cyclohexyl)hydrazine-1-carboxylate (8-5-P1: racemic trans mixture, 2.2 g; 8-5-P2: racemic cis mixture, 5 g), each as a colorless oil. 8-5-P1: MS (ESI) m/z=400.0 [M+Na]⁺; 8-5-P2: MS (ESI) m/z=378.0 [M+H]⁺.

Step 7: To a solution of trans-benzyl 2-3-((tert-butoxycarbonyl)(methyl)amino)cyclohexyl)hydrazine-1-carboxylate (8-5-P1, 650 mg, 1.7 mmol) in EtOH (14 mL) were added AcOH (7 mL) and Pd/C (350 mg). The reaction mixture was stirred at 40° C. for 4 hours under hydrogen. The mixture was directly filtered through celite and the filtrate was concentrated in vacuo to provide crude trans-tert-butyl-3-hydrazineylcyclohexyl)(methyl)carbamate (8-6), which was used in the next step without purification. MS (ESI) m/z=244.0 [M+H]⁺.

Step 8: To a solution of 8-6 (420 mg, 1.7 mmol) in EtOH (30 mL) was added TEA (20 mL) and N-(4-(2,2-dicyano-1-methoxyvinyl)benzyl)-5-fluoro-2-methoxybenzamide (8-7, 630 mg, 1.7 mmol). The reaction mixture was stirred at 70° C. for 4 hours, then diluted with ethyl acetate (100 mL) and washed with brine (2×100 mL). The organic layer was dried (Na₂SO₄), filtered and concentrated in vacuo. The crude product was purified by silica gel column chromatography (petroleum ether:EtOAc=1:1) to give trans-tert-butyl (3-(5-amino-4-cyano-3-(4-((5-fluoro-2-methoxybenzamido)methyl)phenyl)-1H-pyrazol-1-yl)cyclohexyl)(methyl)carbamate (8-8) as a yellow oil (80 mg). MS (ESI) m/z=577.0 [M+H]⁺.

Step 9: A solution of 8-8 (80 mg, 0.33 mmol) and 5 drops of water in MeSO₃H (5 mL) was stirred at 70° C. for 16 hours, then diluted with ethyl acetate (40 mL) and washed with saturated aqueous NaOH solution (40 mL). The organic layer was dried (Na₂SO₄), filtered and concentrated in vacuo. The crude product was purified by prep-HPLC (NH₃-MeOH (7N):DCM=1:10) to give trans-5-amino-3-(4-((5-fluoro-2-methoxybenzamido)methyl)phenyl)-1-(3-(methylamino)cyclohexyl)-1H-pyrazole-4-carboxamide as a yellow oil (70 mg). MS (ESI) m/z=495.1 [M+H]⁺.

Step 10: To a solution of trans-5-amino-3-(4-((5-fluoro-2-methoxybenzamido)methyl)phenyl)-1-3-(methylamino)cyclohexyl)-1H-pyrazole-4-carboxamide (70 mg, 0.14 mmol) and di(1H-1,2,4-triazol-1-yl)methanone (CDT, 28 mg, 0.17 mmol) in DMF (1 mL) was added DIPEA (55 mg, 0.42 mmol). The reaction mixture was stirred at room temperature for 1 hour. The mixture was purified by prep-HPLC directly to give trans-N-(3-(5-amino-4-carbamoyl-3-(4-((5-fluoro-2-methoxybenzamido)methyl)phenyl)-1H-pyrazol-1-yl)cyclohexyl)-N-methyl-1H-1,2,4-triazole-1-carboxamide (194) as a white solid (18.24 mg). ¹HNMR(400 MHz, DMSO-d₆) δ 8.96 (s, 1H), 8.83 (t, J=6.0 Hz, 1H), 7.96 (s, 1H), 7.52 (dd, J=8.0, 4.0 Hz, 1H), 7.44-7.25 (m, 4H), 7.18 (dd, J=12.0, 4.0 Hz, 1H), 6.29 (d, J=8.0 Hz, 2H), 4.88 (s, 1H), 4.66 (s, 1H), 4.54 (d, J=4.0 Hz, 2H), 3.90 (s, 3H), 2.95 (s, 3H), 2.51-1.59 (m, 8H). MS (ESI) m/z=590.4 [M+H]⁺.

### Example 1i: Synthesis of cis-N-(3-(5-amino-4-carbamoyl-3-(4-((5-fluoro-2-methoxybenzamido)methyl)phenyl)-1H-pyrazol-1-yl)cyclohexyl)-N-methyl-1H-1,2,4-triazole-1-carboxamide (284)

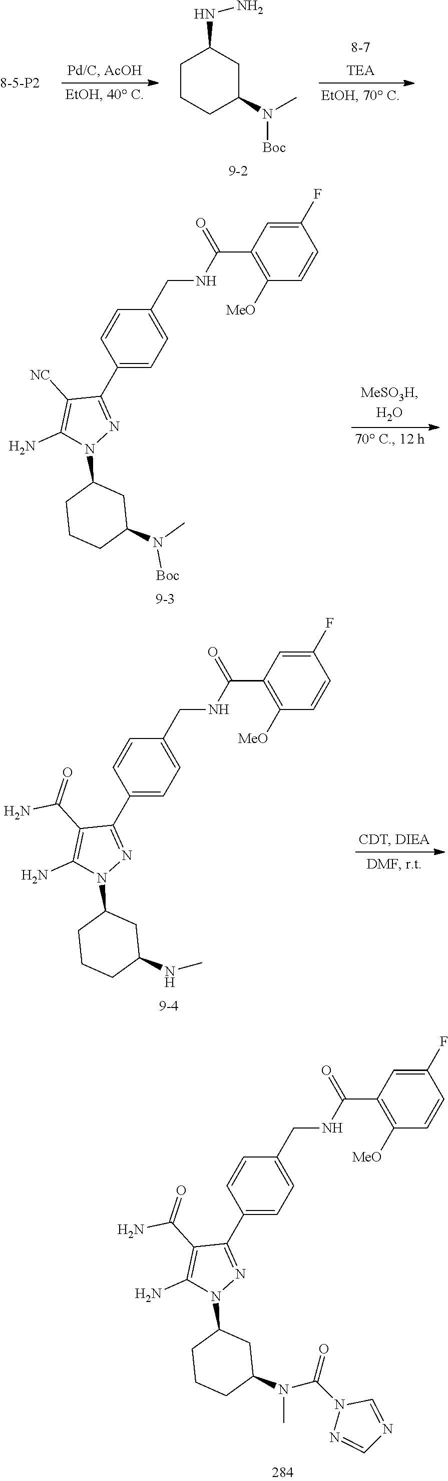 [SMILES: CN(C(=O)OC(C)(C)C)[C@H]1CCC[C@@H](NN)C1 ; COc1ccc(F)cc1C(=O)NCc1ccc(-c2nn([C@@H]3CCC[C@H](N(C)C(=O)OC(C)(C)C)C3)c(N)c2C#N)cc1 ; CN[C@H]1CCC[C@@H](n2nc(-c3ccc(CNC(=O)c4cc(F)ccc4OC)cc3)c(C(N)=O)c2N)C1 ; COc1ccc(F)cc1C(=O)NCc1ccc(-c2nn([C@@H]3CCC[C@H](N(C)C(=O)n4cncn4)C3)c(N)c2C(N)=O)cc1]

Step 1: To a solution of cis-benzyl 2-3-((tert-butoxycarbonyl)(methyl)amino)cyclohexyl)hydrazine-1-carboxylate (8-5-P2, 1 g, 2.6 mmol) in EtOH (20 mL) was added AcOH (10 mL) and Pd/C (500 mg). The reaction mixture was stirred at 40° C. for 4 hours under hydrogen. The mixture was directly filtered through celite and the filtrate was concentrated to provide crude cis-tert-butyl-3-hydrazineylcyclohexyl)(methyl)carbamate (9-2), which was used in the next step without purification. MS (ESI) m/z=244.0 [M+H]⁺.

Step 2: To a solution of 9-2 (640 mg, 2.6 mmol) in EtOH (40 mL) were added TEA (30 mL) and 8-7 (960 mg, 2.6 mmol). The reaction mixture was stirred at 70° C. for 4 hours, then diluted with ethyl acetate (100 mL) and washed with brine (2×100 mL). The organic layer was dried (Na₂SO₄), filtered and concentrated in vacuo. The residue was purified by silica gel column (EtOAc:petroleum ether=1:1) to give cis-tert-butyl (3-(5-amino-4-cyano-3-(4-((5-fluoro-2-methoxybenzamido)methyl)phenyl)-1H-pyrazol-1-yl)cyclohexyl)(methyl)carbamate as a yellow oil (9-3, 900 mg). MS (ESI) m/z=577.0 [M+H]⁺.

Step 3: A solution of 9-3 (150 mg, 0.62 mmol) and 5 drops of water in MeSO₃H (5 mL) was stirred at 70° C. for 16 hours, then diluted with ethyl acetate (40 mL) and washed with saturated aqueous NaOH solution (40 mL). The organic layer was dried (Na₂SO₄), filtered and concentrated in vacuo. The residue was purified by prep-HPLC (NH₃-MeOH (7N):DCM=1:10) to give cis-5-amino-3-(4-((5-fluoro-2-methoxybenzamido)methyl)phenyl)-1-(3-(methylamino)cyclohexyl)-1H-pyrazole-4-carboxamide as a yellow oil (9-4, 100 mg). MS (ESI) m/z=495.0 [M+H]⁺.

Step 4: To a solution of 9-4 (100 mg, 0.2 mmol) and (1H-1,2,4-triazol-1-yl)(4H-1,2,4-triazol-4-yl) methanone (CDT, 40 mg, 0.24 mmol) in DMF (1 mL) was added DIPEA (78 mg, 0.6 mmol). The reaction mixture was stirred at room temperature for 1 hour, then purified by prep-HPLC directly to give cis-N-(3-(5-amino-4-carbamoyl-3-(4-((5-fluoro-2-methoxybenzamido)methyl)phenyl)-1H-pyrazol-1-yl)cyclohexyl)-N-methyl-1H-1,2,4-triazole-1-carboxamide as a white solid (284, 40.56 mg). ¹H NMR (400 MHz, DMSO-d₆) δ 9.05 (s, 1H), 8.84 (t, J=6.0 Hz, 1H), 8.24 (s, 1H), 7.51 (dd, J=8.0, 4.0 Hz, 1H), 7.43 (dd, J=20.0, 12.0 Hz, 4H), 7.37-7.31 (m, 1H), 7.18 (dd, J=8.0, 4.0 Hz, 1H), 6.39 (s, 2H), 4.54 (d, J=8.0 Hz, 2H), 4.20 (s, 2H), 3.89 (s, 3H), 2.96 (s, 3H), 2.20-1.27 (m, 8H). MS (ESI) m/z=590.4 [M+H]⁺.

### Example 2: MGLL (NCBI Gene ID: 11343, Uniprot ID: Q99685) compound modification assay

Test compounds were prepared as 10 mM stock solutions in DMSO (Fisher cat #BP231-100). Human recombinant MGLL protein 1-303 containing K36A, L169S, L176S amino acid substitutions were produced at in house. On the day of the assay, MGLL protein was diluted to 2 μM in assay buffer (20 mM Hepes, pH 7.5, 150 mM NaCl, 1 mM MgCl₂, 1 mM DTT). For testing MGLL modification, compounds were diluted to 50X final test concentration in DMSO in 96-well storage plates. 2 μL of the diluted 50X compounds were added to appropriate wells in the PCR plate (Fisher cat #AB-0800). 49 μL of the stock protein solution was added to each well of the 96-well PCR plate to form a mixture. The plate was sealed well with aluminum plate seal and stored in a drawer at room temperature. After different incubation times, the reactions were quenched by adding 5 μL of 2% formic acid (Fisher cat #A117-50) in MilliQ H₂O to each well followed by mixing with a pipette. The plate was then resealed with aluminum seal and stored until mass spectrometry analysis.

The extent of covalent modification of MGLL proteins was determined by liquid chromatography electrospray mass spectrometry analysis of the intact proteins using a Thermo Q-Exactive Plus mass spectrometer. 20 μL of sample was injected onto a bioZen 3.6 μm Intact C4 column (Phenomenex cat #00B-4767-AN) placed in a column oven set to 40° C. and separated using an LC gradient from 20% to 60% solvent B. Solvent A was 0.1% formic acid and solvent B was 0.1% formic acid in acetonitrile. HESI source settings were set to 40, 5 and 1 for the sheath, auxiliary and sweep gas flow, respectively. The spray voltage was 4 kV, and the capillary temperature was 320° C. S-lens RF level was 50 and auxiliary gas heater temperature was set to 200° C. The mass spectrometry was acquired using a scan range from 650 to 1750 m/z using positive polarity at a mass resolution of 70,000, AGC target of 1e6 ions and maximum injection time of 250 ms. The recorded protein mass spectrum was deconvoluted from the raw data file using Protein Deconvolution v4.0 (Thermo). The protein mass and adduct masses were exported with their peak intensities. The peak intensities for the unmodified and modified protein were used to calculate the percent covalent modification of the MGLL protein based on the following equation: % MGLL protein modification=((MGLL-compound)/(MGLL)+(MGLL-Compound))*100.

Certain compounds disclosed herein are expected to covalently modify MGLL protein when assessed using the assay described above by at least 1%, such as by at least 5%, 10%, 20%, 30%, 40%, or at least 50%. For example, compound 133 was found to modify MGLL protein by over 50% at 4 hours when tested at a compound concentration of 3 μM.

### Example 3: BTK wildtype (NCBI Gene ID: 695, Uniprot ID: Q06187) and BTK C481S compound modification assay

Test compounds were prepared as 10 mM stock solutions in DMSO (Fisher cat #BP231-100). Human recombinant BTK wildtype protein 393-659 or BTK C481S protein 393-659 was produced in house or purchased from Saromics Biostructures AB (Lund, Sweden). The BTK protein was diluted to 1 μM in buffer (20 mM Hepes, pH 7.5, 150 mM NaCl, 1 mM MgCl₂, 1 mM DTT). For testing BTK or BTK C481S modification, compounds were diluted to 50X final test concentration in DMSO in 96-well storage plates. 2 μL of the diluted 50X compounds were added to appropriate wells in the PCR plate (Fisher cat #AB-0800). 49 μL of the stock protein solution was added to each well of the 96-well PCR plate to form a mixture. The plate was sealed well with aluminum plate seal and stored in a drawer at room temperature. After different incubation times, the reactions were quenched by adding 5 μL of 2% formic acid (Fisher cat #A117-50) in MilliQ H₂O to each well followed by mixing with a pipette. The plate was then resealed with aluminum seal and stored until mass spectrometry analysis.

The extent of covalent modification of BTK proteins was determined by liquid chromatography electrospray mass spectrometry analysis of the intact proteins using a Thermo Q-Exactive Plus mass spectrometer. 20 μL of sample was injected onto a bioZen 3.6 μm Intact C4 column (Phenomenex cat #OOB-4767-AN) placed in a column oven set to 40° C. and separated using an LC gradient from 20% to 60% solvent B. Solvent A was 0.1% formic acid and solvent B was 0.1% formic acid in acetonitrile. HESI source settings were set to 40, 5 and 1 for the sheath, auxiliary and sweep gas flow, respectively. The spray voltage was 4 kV, and the capillary temperature was 320° C. S-lens RF level was 50 and auxiliary gas heater temperature was set to 200° C. The mass spectrometry was acquired using a scan range from 650 to 1750 m/z using positive polarity at a mass resolution of 70,000, AGC target of 1e6 ions and maximum injection time of 250 ms. The recorded protein mass spectrum was deconvoluted from the raw data file using Protein Deconvolution v4.0 (Thermo). The protein mass, adduct masses and their corresponding peak intensities were exported to Excel (Microsoft). The peak intensities for the unmodified and modified protein were used to calculate the percent covalent modification of the BTK protein based on the following equation: % BTK protein modification=((BTK-compound)/(BTK)+(BTK-Compound))*100. The equation was used to calculate percent modification for both BTK wildtype protein and BTK C481S protein.

Table 2 shows the percent modification at 24 hours of selected compounds against BTK wildtype protein and BTK C481S protein using the assay described above. Compound numbers correspond to the numbers and structures provided in Table 1 and Example 1.

TABLE 2

| | <5% (++) | ≥5% (+++) |
|---|---|---|
| % BTK (wt) | 103, 131, 134, 154, 155, 156, 164, 180, | 104, 108, 111, 112, 113, 114, 115, 117, |
| Modification at 24 h | 185, 203, 205, 220, 242, 245, 249, 250, | 118, 119, 120, 121, 124, 125, 126, 127, |
| | 253, 266, 272, 280, 282, 285, 288, 292, | 128, 129, 130, 132, 136, 137, 138, 139, |
| | 304, 305, 313 | 140, 141, 143, 145, 146, 147, 149, 150, |
| | | 151, 152, 153, 158, 159, 160, 161, 162, |
| | | 163, 165, 166, 167, 170, 172, 173, 174, |
| | | 175, 176, 177, 178, 181, 182, 183, 184, |
| | | 186, 187, 188, 189, 191, 193, 194, 195, |
| | | 196, 197, 198, 199, 201, 202, 204, 206, |
| | | 207, 208, 209, 210, 211, 212, 213, 214, |
| | | 216, 217, 218, 219, 221, 222, 223, 224, |
| | | 225, 226, 228, 229, 230, 231, 232, 234, |
| | | 235, 236, 237, 238, 240, 241, 243, 244, |
| | | 246, 247, 248, 251, 252, 254, 256, 257, |
| | | 259, 260, 261, 262, 264, 265, 268, 269, |
| | | 270, 271, 274, 276, 277, 278, 279, 281, |
| | | 283, 284, 286, 287, 290, 291, 293, 294, |
| | | 295, 296, 297, 298, 302, 303, 306, 307, |
| | | 308, 309, 310, 311, 312, 314, 315, 316 |
| % BTK (C481S) | 103, 104, 108, 111, 112, 113, 114, 115, | 117, 119, 120, 121, 127, 132, 139, 140, |
| Modification at 24 h | 118, 124, 125, 126, 128, 129, 130, 131, | 150, 158, 159, 170, 176, 177, 186, 188, |
| | 134, 136, 137, 138, 141, 143, 145, 146, | 195, 202, 204, 206, 210, 217, 219, 222, |
| | 147, 149, 151, 152, 153, 154, 155, 156, | 226, 229, 231, 237, 238, 240, 243, 257, |
| | 160, 161, 162, 163, 164, 165, 166, 167, | 268, 269, 274, 276, 278, 281, 284, 286, |
| | 172, 173, 174, 175, 178, 180, 181, 182, | 287, 294, 297, 302, 310, 313, 316 |
| | 183, 184, 185, 187, 189, 191, 193, 194, | |
| | 196, 197, 198, 199, 201, 203, 205, 207, | |
| | 208, 209, 211, 212, 213, 214, 216, 218, | |
| | 220, 221, 223, 224, 225, 228, 230, 232, | |
| | 234, 235, 236, 241, 242, 244, 245, 246, | |
| | 247, 248, 249, 250, 251, 252, 253, 254, | |
| | 256, 259, 260, 261, 262, 264, 265, 266, | |
| | 270, 271, 272, 277, 279, 280, 282, 283, | |
| | 285, 288, 290, 291, 292, 293, 295, 296, | |
| | 298, 303, 304, 305, 306, 307, 308, 309, | |
| | 311, 312, 314, 315 | |

### Example 4: Kₙaet/K₁ determination for MGLL protein inhibitors

Test compounds were prepared as 10 mM stock solutions in DMSO (Fisher cat #BP231-100). Human MGLL protein 1-303 containing K36A, L169S, L176S amino acid substitutions was produced in house. MGLL protein was diluted to 1 μM in buffer (20 mM Hepes, pH 7.5, 150 mM NaCl, 1 mM MgCl₂, 1 mM DTT). For testing MGLL modification, compounds were diluted to 50X final test concentration in DMSO in 96-well storage plates. 1 μL of the diluted 50X compounds were added to appropriate wells in the PCR plate (Fisher cat #AB-0800). Increasing concentrations of compounds were added to separate wells, resulting in final assay compound concentrations of 3 μM, 10 μM, 30 μM and 100 μM. 49 μL of the stock protein solution was added to each well of the 96-well PCR plate. Reactions are mixed carefully. The plate was sealed well with aluminum plate seal and stored in a drawer at room temperature. After different incubation times, the reactions were quenched by adding 5 μL of 2% formic acid (Fisher cat #A117-50) in MilliQ H₂O to each well followed by mixing with a pipette. The plate was then resealed with aluminum seal and stored until mass spectrometry analysis.

The extent of covalent modification of MGLL proteins was determined by liquid chromatography electrospray mass spectrometry analysis of the intact proteins using a Thermo Q-Exactive Plus mass spectrometer. 20 μL of sample was injected onto a bioZen 3.6 μm Intact C4 column (Phenomenex cat #OOB-4767-AN) placed in a column oven set to 40° C. and separated using an LC gradient from 20% to 60% solvent B. Solvent A was 0.1% formic acid and solvent B was 0.1% formic acid in acetonitrile. HESI source settings were set to 40, 5 and 1 for the sheath, auxiliary and sweep gas flow, respectively. The spray voltage was 4 kV, and the capillary temperature was 320° C. S-lens RF level was 50 and auxiliary gas heater temperature was set to 200° C. The mass spectrometry was acquired using a scan range from 650 to 1750 m/z using positive polarity at a mass resolution of 70,000, AGC target of 1e6 ions and maximum injection time of 250 ms. The recorded protein mass spectrum was deconvoluted from the raw data file using Protein Deconvolution v4.0 (Thermo). The protein mass, adduct masses and their corresponding peak intensities were exported to Excel (Microsoft). The peak intensities for the unmodified and modified protein were used to calculate the percent covalent modification of the MGLL protein based on the following equation: % MGLL protein modification=((MGLL-compound)/(MGLL)+(MGLL-Compound))*100.

The K_{inact}/K_{I} is a rate constant describing the efficiency of covalent bond formation resulting from the potency (K₁) of the first reversible binding event and the maximum potential rate (K_{inact}) of inactivation. The percent MGLL protein modification was plotted as a function of time using Prism version 9.4.1 (GraphPad), and the observed rate of modification (K_{obs}), a first-order rate constant with units of inverse time, was estimated using nonlinear regression at each compound concentration. If the K_{obs} is unchanged by increasing the compound concentration higher than 3 μM, K₁ is lower than 3 μM. If K_{obs} is increasing with compound concentration, K₁ is higher than 3 μM. K_{inact}/K_{I} can be estimated by plotting K_{obs} values against each compound concentration used in separate reactions. The K_{obs} values are fitted to determine K_{inact} and K₁ using the following equation, K_{obs}=(K_{inact}×[compound conc])/(K₁+[compound conc]).

Table 3 shows the K_{obs} values of selected compounds against MGLL wildtype protein using the assay described above. Compound numbers correspond to the numbers and structures provided in Table 1 and Example 1.

TABLE 3

| | ≤50 M⁻¹ · s⁻¹ | >50 M⁻¹ · s⁻¹ |
|---|---|---|
| MGLL (wt) K_{obs} | 157, 169 | 106, 135, 190, 200 |

### Example 5: K_{inact}/K₁ determination for BTK protein inhibitors

Test compounds were prepared as 10 mM stock solutions in DMSO (Fisher cat #BP231-100). Human recombinant BTK wildtype protein 393-659 or BTK C481S protein 393-659 was produced in house or purchased from Saromics Biostructures AB (Lund, Sweden). BTK protein was diluted to 1 μM in buffer (20 mM Hepes, pH 7.5, 150 mM NaCl, 1 mM MgCl₂, 1 mM DTT). For testing BTK modification, compounds were diluted to 50× final test concentration in DMSO in 96-well storage plates. 1 μL of the diluted 50× compounds were added to appropriate wells in the PCR plate (Fisher cat #AB-0800). Increasing concentrations of compounds were added to separate wells, resulting in final assay compound concentrations of 3 μM, 10 μM, 30 μM and 100 μM. 49 μL of the stock protein solution was added to each well of the 96-well PCR plate. Reactions were mixed carefully. The plate was sealed well with aluminum plate seal and stored in a drawer at room temperature. After different incubation times, the reactions were quenched by adding 5 μL of 2% formic acid (Fisher cat #A117-50) in MilliQ H₂O to each well followed by mixing with a pipette. The plate was then resealed with aluminum seal and stored until mass spectrometry analysis.

The extent of covalent modification of BTK proteins was determined by liquid chromatography electrospray mass spectrometry analysis of the intact proteins using a Thermo Q-Exactive Plus mass spectrometer. 20 μL of sample was injected onto a bioZen 3.6 μm Intact C4 column (Phenomenex cat #OOB-4767-AN) placed in a column oven set to 40° C. and separated using a LC gradient from 20% to 60% solvent B. Solvent A was 0.1% formic acid and solvent B was 0.1% formic acid in acetonitrile. HESI source settings were set to 40, 5 and 1 for the sheath, auxiliary and sweep gas flow, respectively. The spray voltage was 4 kV, and the capillary temperature was 320° C. S-lens RF level was 50 and auxiliary gas heater temperature was set to 200° C. The mass spectrometry was acquired using a scan range from 650 to 1750 m/z using positive polarity at a mass resolution of 70,000, AGC target of 1e6 ions and maximum injection time of 250 ms. The recorded protein mass spectrum was deconvoluted from the raw data file using Protein Deconvolution v4.0 (Thermo). The protein mass, adduct masses and their corresponding peak intensities were exported to Excel (Microsoft). The peak intensities for the unmodified and modified protein were used to calculate the percent covalent modification of the BTK protein based on the following equation: % BTK protein modification=((BTK-compound)/(BTK)+(BTK-Compound))*100.

The K_{inact}/K_{I} is a rate constant describing the efficiency of covalent bond formation resulting from the potency (K₁) of the first reversible binding event and the maximum potential rate (K_{inact}) of inactivation. The percent MGLL protein modification was plotted as a function of time using Prism version 9.4.1 (GraphPad), and the observed rate of modification (K_{obs}), a first-order rate constant with units of inverse time, was estimated using nonlinear regression at each compound concentration. If the K_{obs} is unchanged by increasing the compound concentration higher than 3 μM, K₁ is lower than 3 μM. If K_{obs} is increasing with compound concentration, K₁ is higher than 3 μM. K_{iac}t/K_{I} can be estimated by plotting K_{obs} values against each compound concentration used in separate reactions. The K_{obs} values are fitted to determine K_{inact} and K₁ using the following equation, K_{obs}=(K_{inact}×[compound conc])/(K₁+[compound conc]).

Table 4 shows the K_{obs} values of selected compounds against BTK wildtype protein using the assay described above. Compound numbers correspond to the numbers and structures provided in Table 1 and Example 1.

TABLE 4

| | ≤50 M⁻¹ · s⁻¹ | >50 M⁻¹ · s⁻¹ |
|---|---|---|
| BTK (wt) K_{obs} | 111, 112, 113, 114, 115, 120, 124, 125, 129, 130, 131, | 109, 118, 121, 126, 127, |
| | 132, 134, 136, 137, 138, 140, 150, 152, 153, 154, 155, | 139, 141, 143, 146, 147, |
| | 156, 159, 160, 162, 163, 164, 165, 166, 167, 172, 173, | 149, 151, 158, 170, 177, |
| | 174, 175, 176, 178, 180, 181, 183, 184, 185, 188, 189, | 186, 187, 195, 198, 202, |
| | 191, 193, 194, 196, 197, 199, 201, 203, 204, 205, 207, | 206, 209, 211, 214, 216, |
| | 208, 210, 212, 217, 218, 220, 221, 223, 224, 226, 228, | 219, 222, 225, 229, 232, |
| | 230, 231, 234, 235, 237, 240, 241, 242, 243, 245, 247, | 236, 238, 239, 244, 246, |
| | 248, 249, 250, 253, 254, 256, 259, 260, 261, 264, 265, | 252, 257, 262, 274, 278, |
| | 266, 268, 269, 270, 271, 272, 277, 279, 280, 281, 282, | 284, 286, 302, 303, 306, |
| | 283, 285, 287, 288, 290, 291, 294, 295, 296, 298, 304, | 308, 310, 314 |
| | 305, 307, 309, 311, 312, 313, 315, 316 | |

### Example 6: BTK wildtype (accession: NP 000052) and BTK C481iS (accession: NP_000052) HTRF displacement assay

The ability of a compound to bind BTK wildtype or BTK C481iS proteins was measured using an HTRF displacement assay. The assay measures the ability of a compound to displace a tracer ligand bound to the active site of BTK wildtype or BTK C481 S proteins.

B Test compounds were prepared as 10 mM stock solutions in DMSO (Fisher cat #BP231-100). Compounds to be assayed were dispensed using an Echo 650 acoustic dispenser (Beckman Coulter) on a 384 well plate in 6 doses applying four-fold dilutions from the highest concentration of 5 M. N-terminal GST-tagged recombinant full-length BTK wildtype protein and BTK C481 S protein 2-659 were purchased from Camna Biosciences, Inc (Kobe, Japan). BTK wildtype or BTK C4815S protein was separately incubated with compound in assay buffer containing kinase tracer 239 (Thermo Fisher) and Mab anti GST-Th (PerkinEhiner). After a 1-hour incubation at room temperature, the HTRF signal was measured using SPARK plate reader (Tecan) using the TR fluorescence mode. The HTRF ratio is calculated for DMSO, no protein control and compound samples using the following equation: HTRF ratio=Emmision665/Emmision620×10\hat{ }4. The signal for the no protein control samples was used to subtract background noise from the DMSO and compound samples. Background subtracted HTRF ratios are used to calculate the percent inhibition, where DMSO controls are set to 0 percent inhibition. The percent inhibition is plotted as a function of compound concentration and IC50 values were calculated from a four-parameter logistic fit in Prism v9.4 (GraphPad), where the bottom and top are constrained to 0 and 100, respectively.

Table 5 shows the activity of selected compounds against BTK wildtype protein and BTK C4815S protein using the assay described above. Compound numbers correspond to the numbers and structures provided in Table 1 and Example 1.

TABLE 5

| | ≤1 μM (+++) | >1 μM (++) |
|---|---|---|
| BTK (wt) IC₅₀ | 101, 103, 105, 108, 109, 111, 114, 115, 116, 117, | 102, 104, 106, 107, 112, 113, |
| | 118, 119, 121, 124, 125, 126, 128, 130, 133, 134, | 120, 127, 129, 131, 132, 137, |
| | 136, 138, 141, 142, 144, 145, 146, 147, 148, 150, | 139, 140, 143, 149, 155, 156, |
| | 151, 152, 153, 154, 158, 159, 160, 161, 162, 163, | 166, 167, 174, 175, 176, 178, |
| | 164, 165, 170, 171, 172, 173, 177, 179, 181, 182, | 180, 185, 192, 195, 202, 203, |
| | 183, 184, 186, 187, 188, 189, 191, 193, 194, 196, | 206, 210, 214, 221, 223, 224, |
| | 197, 198, 199, 201, 204, 205, 207, 208, 209, 211, | 228, 230, 231, 234, 235, 237, |
| | 212, 213, 215, 216, 217, 218, 219, 220, 222, 225, | 240, 242, 243, 254, 257, 259, |
| | 226, 229, 232, 233, 236, 238, 239, 241, 244, 245, | 261, 265, 268, 271, 277, 279, |
| | 246, 247, 248, 249, 250, 251, 252, 253, 256, 258, | 280, 281, 283, 285, 291, 298, |
| | 260, 262, 263, 264, 266, 267, 269, 270, 272, 273, | 300, 301, 305, 313, 315, 316 |
| | 274, 275, 276, 278, 282, 284, 286, 287, 288, 290, | |
| | 292, 293, 294, 295, 296, 297, 299, 302, 303, 304, | |
| | 306, 307, 308, 309, 310, 311, 312, 314 | |
| BTK (C481S) IC₅₀ | 101, 103, 104, 105, 108, 109, 111, 114, 115, 116, | 102, 106, 107, 112, 113, 117, |
| | 118, 121, 124, 125, 126, 128, 130, 133, 134, 136, | 119, 120, 127, 129, 131, 132, |
| | 137, 138, 140, 142, 144, 145, 146, 147, 148, 150, | 139, 141, 143, 149, 156, 158, |
| | 151, 152, 153, 154, 155, 159, 160, 162, 163, 164, | 161, 167, 174, 175, 176, 178, |
| | 165, 166, 170, 171, 172, 173, 177, 179, 181, 182, | 180, 188, 192, 195, 202, 203, |
| | 183, 184, 185, 186, 187, 189, 191, 193, 194, 196, | 206, 210, 214, 217, 223, 224, |
| | 197, 198, 199, 201, 204, 205, 207, 208, 209, 211, | 225, 228, 231, 234, 235, 237, |
| | 212, 213, 215, 216, 218, 219, 220, 221, 222, 226, | 238, 240, 242, 243, 254, 257, |
| | 229, 230, 232, 233, 236, 239, 241, 244, 245, 246, | 259, 261, 268, 271, 276, 277, |
| | 247, 248, 249, 250, 251, 252, 253, 256, 258, 260, | 279, 281, 285, 291, 298, 301, |
| | 262, 263, 264, 265, 266, 267, 269, 270, 272, 273, | 305, 313, 315, 316 |
| | 274, 275, 278, 280, 282, 283, 284, 286, 287, 288, | |
| | 290, 292, 293, 294, 295, 296, 297, 299, 300, 302, | |
| | 303, 304, 306, 307, 308, 309, 310, 311, 312, 314 | |

The same assay was used to measure the activity of selected compounds of the formula TBM-L-hydrogen. Using the IC₅₀ value observed for a given reversible test compound, the K₁ of the test compound can be assessed using the formula K₁=IC₅₀/(1+([Tracer]/K_{d})), wherein the tracer concentration in the assay [Tracer]is 50 nM and the tracer K_{d} for BTK C481S is 54 nM. The compounds provided in Table 6 were found to reversibly bind to BTK C481S with a K₁ of less than 3 μM.

TABLE 6

| | BTK |
|---|---|
| Structure | C481S K_{I} |
| 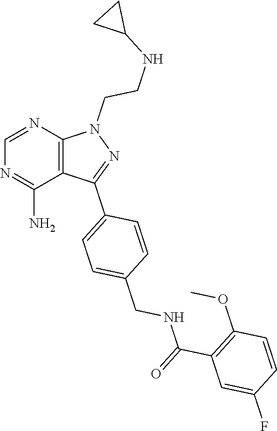 [SMILES: COc1ccc(F)cc1C(=O)NCc1ccc(-c2nn(CCNC3CC3)c3ncnc(N)c23)cc1] | <660 nM |
| 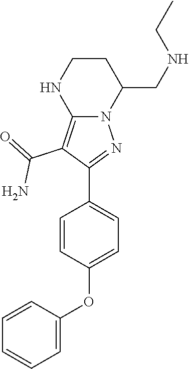 [SMILES: CCNCC1CCNc2c(C(N)=O)c(-c3ccc(Oc4ccccc4)cc3)nn21] | <1000 nM |
| 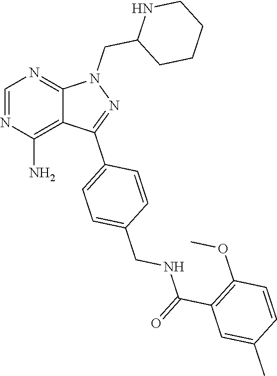 [SMILES: COc1ccc(C)cc1C(=O)NCc1ccc(-c2nn(CC3CCCCN3)c3ncnc(N)c23)cc1] | <600 nM |

### Example 7: BTK ADP-GLO enzymatic assay

Test compounds were prepared as 10 mM stock solutions in DMSO (Fisher cat #BP231-100). Compounds to be assayed were dispensed using an Echo 650 acoustic dispenser (Beckman Coulter) on a 384 well plate in 6 doses applying four-fold dilutions from the highest concentration of 5 μM. N-terminal GST-tagged recombinant full-length BTK wildtype protein and BTK C481S protein 2-659 were purchased from Carna Biosciences, Inc (Kobe, Japan). BTK wildtype or BTK C481S protein was separately incubated with compound in enzyme assay buffer that includes a substrate and ATP. The enzymatic reaction is incubated for two hours at room temperature. The reaction was stopped by adding ADP-GLO reagent (Promega) and incubating for an additional 30 minutes. ADP-GLO Detection reagent (Promega) was added and incubated for 30 minutes. The assay plate is read on a SPARK plate reader using the luminescence module (Tecan). The luminescence data was analyzed and IC50 enzyme inhibition was estimated using nonlinear regression in Prism version 9.4 (GraphPad).

### Example 8: Intrinsic reactivity assay

Understanding of the intrinsic reactivity of electrophilic warheads with nucleophilic molecules is an important parameter influencing the stability of a compound in biological matrices. Lower intrinsic reactivity also mitigates the risk of covalent off-target reactivity. A common target residue for covalent drugs is cysteine, an amino acid containing a nucleophilic thiol side chain. The assay described here measures the rate of a compound forming a covalent bond with Boc-protected cysteine methyl ester or cysteine methyl ester that can be used as a surrogate for cysteines found on proteins.

J Test compounds are prepared as 10 mM stock solutions in DMSO (Fisher cat #BP231-100). Compounds with molecular weight differing more than 3 Dalton are pooled and diluted to 5 μM in 100 mM potassium phosphate buffer, pH 7.4, containing 50 mM Boc-protected cysteine methyl ester (Combi-Blocks cat #QH-3424). Compounds incubated without Boc-protected cysteine methyl ester serve as controls. The reactions are incubated at 37° C., and reaction aliquots at different time intervals are sampled from the reaction and quenched with formic acid for subsequent LC-MS/MS analysis.

The LC-MS/MS analysis is done using a LC-40D×3 pump and SIL-40C×3 autosampler (Shimadzu) coupled to a QTRAP 6500+(Sciex). Compounds are separated on a XBridge Premier BEH column (Waters). The sample run time is about 4 minutes per injection. The normalized peak area for each compound is extracted from the mass spectrometry raw files, and the data for each compound are plotted against time in Prism version 9.4 (GraphPad). Nonlinear regression using one-phase decay is used to estimate the rate of covalent bond formation with Boc-protected cysteine methyl ester or cysteine methyl ester and parent compound half-life (t %).

As shown for each pair of warheads in Table 7, PG-L-STW forms a covalent bond with a suitably protected Cys-OMe at a rate characterized by an intrinsic rate constant (K) that is less than that of PG-L-STW′, wherein STW′ is unsubstituted acrylyl.

TABLE 7

| Warhead | K (min⁻¹) |
|---|---|
| 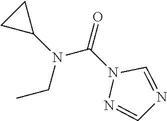 | <5 × 10⁻⁴ |
| 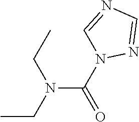 | <5 × 10⁻⁴ |
| 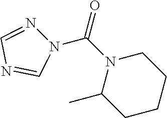 | <5 × 10⁻⁴ |
| 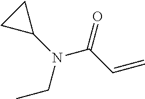 | 0.0159 |
| 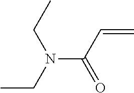 | 0.0012 |
| 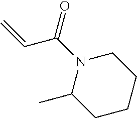 | 0.0029 |

### Example 9: BTK cellular assay

The ability of a compound of the present disclosure to inhibit BTK protein signaling can be demonstrated by inhibiting growth of a given BTK mutant cell line. For example, this assay can be also used to assess selective growth inhibition of a mutant BTK protein relative to a wildtype, or relative to a different mutant BTK protein.

J Stably transfected murine Ba/F3 (CVCL_0161) cell lines with BTK cDNA mutant constructs can be used to assess BTK cellular signaling in vitro, e.g., in response to an inhibitor compound of the present disclosure. This cellular assay can also be used to discern selective inhibition of a subject compound against certain types of BTK mutants, e.g., more potent inhibition against BTK C481S relative to other BTK wildtype or other BTK mutant variants. Ba/F3 culture medium is prepared with RPMI-1640 (e.g., with 10% FCS, 100 units/mL penicillin and 100 mg/mL streptomycin, and 1% glutamine. Ba/F3 cells are exposed to treatment with BTK inhibitors for 72 hours and proliferation inhibition is measured by accessing cell viability between wells treated with the test compounds and control wells. Proliferation assays are performed at a range of inhibitor concentrations (10 μM, 3 μM, 1.1 μM, 330 nM, 110 nM, 33 nM, 11 nM, 3 nM, 1 nM) in triplicate. Cell viability is measured with, for example, Cell Titer Glo and analyzed in comparison to the time zero measurements. The IC50 values are determined using the four-parameter fit. The resulting IC50 value is a measurement of the ability of the test compound to reduce cell growth of BTK-driven cells in vitro and/or in vivo. One or more compounds disclosed herein is expected to exhibit an IC50 value less than 5 μM, 1 μM, 100 nM, or even less, against one or more stably transfected Ba/F3 cell line with BTK wildtype or mutant construct.

### Example 10: MGLL cellular assay

J Proteomes (mouse brain membrane fraction or lysates from cells expressing human MGLL) are preincubated with varying concentrations of MGLL inhibitor at 37° C. After 30 min, 2 μM ActivX™ TAMRA-FP Serine Hydrolase Probe (Thermo Scientific #88318) is added and the mixture is incubated for another 30 min at 37° C. Reactions are quenched with LDS sample loading buffer containing 50 mM DTT and run on a 12% NuPAGE gel. Following gel imaging, serine hydrolase activity is determined by measuring fluorescent intensity of gel bands corresponding to MAGL using imaging software.

### Example 11: In vivo BTK inhibition

The in vivo reduction in BTK signaling output by a compound of the present disclosure is determined in a mouse tumor xenograft model, such as a BTK wt model or a BTK C481S model utilizing cells including a BTK C481S mutant.

J Tumor xenografts are established by administration of engineered cells with a BTK mutant construct (e.g., TMD-8 cells stably transfected with a BTK wildtype or mutant construct) or murine Ba/F3 cells stably transfected with a BTK wildtype or mutant construct into mice. Female 6- to 8-week-old athymic BALB/c nude (NCr) nu/nu mice are used for xenografts. The cells (e.g., approximately 5×10⁶) are harvested on the day of use and injected in growth-factor-reduced Matrigel/PBS (e.g., 50% final concentration in 100 μL). One flank is inoculated subcutaneously per mouse. Mice are monitored daily, weighed twice weekly, and caliper measurements begin when tumors become visible. For efficacy studies, animals are randomly assigned to treatment groups by an algorithm that assigns animals to groups to achieve best case distributions of mean tumor size with lowest possible standard deviation. Tumor volume can be calculated by measuring two perpendicular diameters using the following formula: (L×w²)/2, in which L and w refer to the length and width of the tumor, respectively. Percent tumor volume change can be calculated using the following formula: (V_{final}-Vᵢₙₜᵢₐₗ)Vᵢₙᵢₜᵢₐₗ×100. Percent of tumor growth inhibition (% TGI) can be calculated using the following formula: % TGI=100×(1-(average Vanai -Vii,tiₐ of treatment group)/(average V1i -Vii,tiₐ of control group). When tumors reach a threshold average size (e.g., approximately 200-400 mm³), mice are randomized into 3-10 mice per group and are treated with vehicle (e.g., 100% Labrasol®) or a compound disclosed herein, using, for example, a daily schedule by oral gavage. Results can be expressed as mean and standard deviation of the mean.

### Example 12: In vivo MGLL inhibition

Inhibitors are administered to wild-type C57Bl/6J by oral gavage in a vehicle formulation. Each animal is sacrificed 4 h following administration and brain proteomes are prepared and analyzed according to previously established methods, for example by homogenization in PBS buffer. 2 μM ActivX™ TAMRA-FP Serine Hydrolase Probe (Thermo Scientific #88318) is added to the brain homogenate and the mixture is incubated for 30 min at 37° C. The reaction is quenched with LDS sample loading buffer containing 50 mM DTT and run on a 12% NuPAGE gel. Following gel imaging, serine hydrolase activity is determined by measuring fluorescent intensity of gel bands corresponding to MAGL using imaging software.

### Example 13: Metabolic (Microsomal) Stability Assay

The metabolic stability of a test compound is assayed at 37° C. using pooled liver microsomes (mouse or human liver microsomes). An aliquot of 10 μL of 50 μM test compound is mixed with 490 μL of 0.611 mg/mL liver microsomes, then 50 μL of the mixtures are dispensed to the 96 well tubes and warmed at 37° C. for 10 minutes. The reactions are initiated by adding 50 μL of the pre-warmed NADPH regeneration system solution (add 1.2 μL solution, 240 μL solution B, mix with 10.56 ml KPBS) and then incubated at 37° C. The final incubation solution contains 100 mM potassium phosphate (pH 7.4), 1.3 mM NADP+, 3.3 mM glucose 6-phosphate, 0.4 Unit/mL of glucose 6-phosphate dehydrogenase, 3.3 mM magnesium chloride, 0.3 mg/mL liver microsomes and 0.5 μM test article. After 0, 15, 30 and 60 minutes in a shaking incubator, the reactions are terminated by adding 100 μL of acetonitrile containing 200 nM buspirone as an internal standard. All incubations are conducted in duplicate. Plates are vortexed vigorously by using Fisher Scientific microplate vortex mixer (Henry Troemner, US). Samples are then centrifuged at 3500 rpm for 10 minutes (4° C.) using Sorvall Legend XRT Centrifuge (Thermo Scientific, GE). Supernatants (40 μL) are transferred into clean 96-deep well plates. Each well is added with 160 μL of ultrapure water (Milli-Q, Millipore Corporation) with 0.1% (v/v) formic acid (Fisher Chemical), mixed thoroughly and subjected to LC/MS/MS analysis in MRM positive ionization mode.

All the samples are measured using a mass spectrometer (QTrap 5500 quadrupole/ion trap) coupled with a Shimadzu HPLC system. The HPLC system consisted of a Shimadzu series degasser, binary quaternary gradient pumps, column heater coupled to an autosampler, and a Phenomenex Gemini-NX, C18, 3.0 μm or Phenomenex Lunar, C8, 5.0 μM HPLC column (Phenomenex, Torrance, CA), and eluted with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+).

The half-life for the metabolic degradation of the test compound is calculated by plotting the time-course disappearance of the test compound during the incubation with liver microsomes. Each plot is fitted to a first-order equation for the elimination of the test compound (% remaining compound) versus time using non-linear regression (Equation 1).

$$\frac{{C}_{t}}{{C}_{0}} = {e}^{- k t} \qquad Equation 1$$

where Cₜ is the mean relative substrate concentration at time t and Co is the initial concentration (0.5 μM) at time 0. Note that the area ratio of the substrate peak to an internal standard peak is proportional to the analyte concentration and is used for regression analysis to derive a value of k.

The half-life t₁₁₂ for metabolic (microsome) stability is derived from the test compound elimination constant k using Equation 2 below.

$${t}_{1 / 2} = \frac{0.693}{k} \qquad Equation 2$$

### Example 14: CYP2C19 Inhibition Assay

J Some xenobiotics can inhibit cytochrome P450 (CYP) enzyme function, which alters their ability to metabolize drugs. Administration of a CYP inhibitor with a drug whose clearance is dependent on CYP metabolism can result in increased plasma concentrations of this concomitant drug, leading to potential toxicity. The inhibition of CYP2C19 by a test compound is assayed in human liver microsomes using S-Mephenytoin as a CYP2C19 substrate. The stock solution of the test compound or known CYP2C19 inhibitor as a positive control (10 mM) is diluted with KPBS to 40 μM. In a similar way, the stock solutions of the human liver microsomes and S-Mephenytoin are diluted with KPBS buffer. The pre-incubations are started by incubating a plate containing 25 μL human liver microsomes (final concentration of 0.2 mg/mL), 25 μL NADPH-generating system, and a 25 μL test compound (final concentration 10 μM) or the positive control for 30 min at 37±1° C. After the pre-incubation, 25 μL S-Mephenytoin (final concentration 200 μM) is added and incubated another 12 minutes at 37±1° C. for substrate metabolism. The reactions are terminated by addition of 100 μL of ice-cold acetonitrile containing an internal standard (buspirone). Precipitated proteins are removed by centrifugation at 3500 rpm for 10 minutes at 4° C. (Allegra 25R, Beckman Co. Fullerton, CA) and then aliquot of the supernatant is transferred to an assay plate.

J Samples are assessed using a mass spectrometer (QTrap 5500 quadrupole/ion trap) coupled with a Shimadzu HPLC system, following the manufacturer's instructions. The metabolism of S-Mephenytoin in human liver microsomes is monitored by LC/MS/MS as representative of CYP2C19 inhibitory activity. The amount of metabolite formed is assessed by the peak area ratio (metabolite/IS) and % inhibition at 10 μM is expressed as a percentage of the metabolite signal reduced compared to the control (i.e. an incubation that contained no inhibitor and represented 100% enzyme activity): % inhibition=(1-A/B)×100%, where A is the metabolite peak area ratio formed in the presence of test compound or inhibitor at 10 μM and B is the metabolite peak area ratio formed without test compound or inhibitor in the incubation.

### Example 15: Mouse and Human Protein Binding Assay to Assess Free Drug Concentration

J This assay can be used to determine the plasma protein binding of the test compound in the plasma of human and animal species using a Rapid Equilibrium Dialysis (RED) device for equilibrium dialysis and LC-MS/MS for sample analysis. Test compound is spiked in. The stock solution of the test compound is prepared at 5 mM concentration. One μL of 5 mM working solution is added into 1000 μL plasma to achieve a final concentration of 5 μM. The spiked plasma is placed on a rocker, and gently agitated for approximately 20 minutes. A volume of 300 μL of the plasma sample containing 5 μM test compound from each species is added to designate RED device donor chambers followed by addition of 500 μL of potassium phosphate buffer to the corresponding receiver chambers in duplicate. The RED device is then sealed with sealing tape and shaken at 150 RPM for 4 hours at 37° C. Post-dialysis donor and receiver compartment samples are prepared for LC-MS/MS analysis, including spiking samples with an internal standard for the bioanalytical analysis. Warfarin and propranolol are purchased from Sigma-Aldrich (St. Louis, MO), and used as positive controls for low and high plasma protein binding, respectively.

All the samples are analyzed using an Agilent Technologies 6430 Triple Quad LC/MS system. The HPLC system consists of an Agilent 1290 Infinity Liquid Chromatograph coupled to an autosampler (Agilent 1290 Infinity LC Injector HTC), and a Phenomenex Gemini-NX, C18, 3.0 μm or Phenomenex Lunar, C8, 5.0 μM HPLC column (Phenomenex, Torrance, CA), eluting with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+). The percentage of the test compound bound to plasma is calculated following Equation 3 and 4.

$$\% Free test compound = \frac{\begin{matrix}Peak ratio ( \frac{test compound}{Internal standard} ) , \\ receiver compartment\end{matrix}}{\frac{Peak ratio ( \frac{test compound}{Internal standard} ) ,}{donor compartment}} 100 \qquad Equation 3$$

$$\% Plasma protein bound test compound = 100 - \% Free test compound \qquad Equation 4$$

### Example 16: hERG (automated patch-clamp) Assay

The human ether-a-go-go related gene (hERG) encodes the voltage gated potassium channel in the heart (IKr) which is involved in cardiac repolarization. Inhibition of the hERG causes QT interval prolongation and can lead to potential fatal events in humans. It is thus important to assess hERG inhibition early in drug discovery. A hERG automated patch-clamp assay is done using a hERG CHO-K1 cell line using an incubation time of 5 min. The degree of hERG inhibition (%) is obtained by measuring the tail current amplitude, which is induced by a one second test pulse to −40 mV after a two second pulse to +20 mV, before and after drug incubation (the difference current is normalized to control and multiplied by 100 to obtain the percent of inhibition). The percent hERG inhibition is measured in the presence of 10 μM test compound.

### Example 17: Rat Oral Exposure (% F)

A pharmacokinetic profile for a test compound is measured by single dosing in jugular vein cannulated male Sprague-Dawley rats. Animal weights are typically over 200 grams, and animals are allowed to acclimate to their new environment for at least 3 days prior to the initiation of any studies. One set of animals is dosed intravenously (IV) with test compound (2 mg/kg in 20% HP-beta-CD or 20% Captisol, pH adjusted to -4 by citric acid). The IV dosing solution concentration is 0.4 mg/mL test compound. Blood is sampled at 5 minutes, 15 minutes, 30 minutes, 90 minutes, 360 minutes, and 24 hours following IV dosing. Another set of animals is dosed oral (po) with test compound (10 mg/kg in 20% HP-beta-CD or 20% Captisol, pH adjusted to -4 by citric acid). The oral dosing solution concentration is 1 mg/mL test compound. Blood is sampled at 15 minutes, 30 minutes, 90 minutes, 180 minutes, 360 minutes and 24 hours following oral (po) dosing. Blood samples (~0.2 mL/sample) are collected via the jugular vein, placed in tubes containing EDTA-K2 and stored on ice until centrifuged. The blood samples are centrifuged at approximately 6800g for 6 minutes at 2-8° C. and the resulting plasma is separated and stored frozen at approximately −80° C.

The plasma samples are analyzed using an Agilent Technologies 6430 Triple Quad LC/MS system, following the manufacturer's instructions. The analytes are detected with positive-mode electrospray ionization (ES+). A standard curve for each test compound is generated and used to measure test compound concentrations in the rat plasma samples. Based on the time course sampling, an area under the curve is calculated for the oral dose group and the intravenous dose group. Percentage rat bioavailability is calculated based on equation 5.

$$\% F ( rat ) = \frac{{AUC}_{po} * {Dose}_{IV}}{{AUC}_{V} * {Dose}_{po}} , \qquad Equation 5$$

where F is bioavailability, AUCₚₒ is area under curve of oral drug, AUC_{Iv} is area under curve of intravenous drug, Doseᵢᵥ is the intravenous dose and Doseₚₒ is the oral dose.

1-42. (canceled)

43. A method of modifying a non-Ras Target Protein, comprising contacting a serine residue of an unmodified non-Ras Target Protein with an effective amount of a compound of Formula (I), wherein the compound of Formula (I) comprises a Target Binding Moiety (TBM), a linker (L), and a Serine-Targeting Warhead (STW), TBM-L-STW (I), 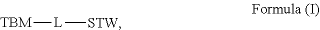
characterized in that:
(i) the compound is capable of forming a covalent bond with the serine residue of the non-Ras Target Protein;
(ii) TBM-L-hydrogen reversibly binds to the non-Ras Target Protein with a K₁ of less than 3 μM when assessed by a biochemical assay;
(iii) PG-L-STW forms a covalent bond with suitably-protected Cys-OMe at a rate characterized by an intrinsic rate constant (K) that is less than that of PG-L-STW′ when tested at the same conditions and a pH between 7 to 8, wherein STW′ is unsubstituted acrylyl, and wherein if PG is bound to a nitrogen atom of L, then PG is Boc, else PG is hydrogen; and
(iv) the compound modulates expression and/or activity of the non-Ras Target Protein upon binding to the non-Ras Target Protein.

44. The method of claim 43, wherein the biochemical assay is an HTRF displacement assay.

45. The method of claim 43, wherein K is less than 0.005 min⁻¹.

46. The method of claim 43, wherein L-STW comprises a ureylene functional group.

47. The method of claim 46, wherein the ureylene functional group comprises a 5- or 6-membered heteroaryl group comprising one, two, or three ring nitrogen atoms.

48. The method of claim 43, wherein the STW is a compound selected from 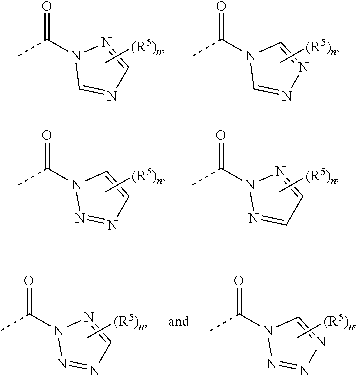
or a pharmaceutically acceptable salt thereof, wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and
n is 0, 1, or 2.

49. The method of claim 43, wherein the STW is a compound of Formula (II): 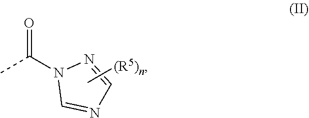
or a pharmaceutically acceptable salt thereof, wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and
n is 0, 1, or 2.

50. The method of claim 43, wherein L is a compound of Formula (III): 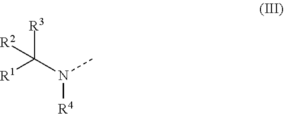
or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R3 are each independently selected from hydrogen and R⁶;
(d) R¹ is selected from hydrogen and R⁶; R² and R3, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R3 is selected from hydrogen and R⁶;
and wherein:
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

51. The method of claim 43, wherein L-STW is a compound of Formula (IV): 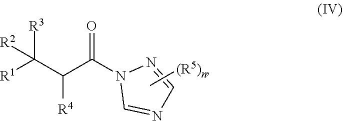
or a pharmaceutically acceptable salt thereof, wherein:
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶;
(b) R¹ and R⁴, together with the atoms to which they are attached, form a 7- to 12-membered spirocyclic heterocycloalkyl ring which is substituted with the TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with the TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with the TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with the TBM and optionally further substituted with one, two, or three R⁶; R² is selected from R⁶ and a bond to the TBM; and R³ is selected from hydrogen and R⁶;
and wherein:
R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl);
n is 0, 1, or 2;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

52. The method of claim 43, wherein the serine residue is an inactive serine.

53. The method of claim 43, wherein the non-Ras Target Protein is a kinase.

54. The method of claim 43, wherein the modified non-Ras Target Protein exhibits a reduced signaling output.

55. A covalently modified serine residue in a non-Ras polypeptide, wherein the modified serine residue is a compound of Formula (V): 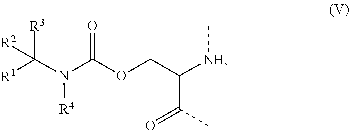
wherein:
the dashed bonds represent peptide bonds between the serine residue and adjacent amino acid residues of the polypeptide, respectively, or, if the serine residue is located at the N-terminus or C-terminus of the polypeptide, the dashed bond to the nitrogen atom represents a bond to a hydrogen atom or the dashed bond to the carbon atom represents a bond to a hydroxy group, respectively;
(a) R¹ and R⁴, together with the atoms to which they are attached, form a 4- to 8-membered monocyclic heterocycloalkyl ring which is optionally substituted with one, two, or three R⁶; and R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶;
(b) R¹ and R⁴, together with the atoms to which they are attached, form a 7- to 12-membered spirocyclic heterocycloalkyl ring which is substituted with TBM and optionally further substituted with one, two, or three R⁶; R² is selected from R⁶; and R³ is selected from hydrogen and R⁶;
(c) R¹ and R⁴, together with the atoms to which they are attached, form a 7- to 12-membered fused bicyclic heterocycloalkyl ring which is substituted with TBM and optionally further substituted with one, two, or three R⁶; and R² and R³ are each independently selected from hydrogen and R⁶;
(d) R¹ is selected from hydrogen and R⁶; R² and R³, together with the carbon atom to which they are attached, form a C₄₋₈ monocyclic cycloalkyl ring or a 4- to 8-membered monocyclic heterocycloalkyl ring, each of which is substituted with TBM and optionally further substituted with one, two, or three R⁶; and R⁴ is selected from R⁷; or
(e) R¹ and R⁴, together with the atoms to which they are attached, form a 4- to 8-membered monocyclic heterocycloalkyl ring which is substituted with TBM and optionally further substituted with one, two, or three R⁶; R² is selected from R⁶ and a bond to TBM; and R³ is selected from hydrogen and R⁶;
and wherein:
TBM is selected from C₆₋₄₀organyl and C₆₋₄₀organoheteryl;
R⁶ is independently selected at each occurrence from halogen, —CN, C₁₋₆ alkyl, and C₃₋₆ cycloalkyl, or two R⁶ attached to the same carbon atom form C₃₋₆ cycloalkyl, wherein C₁₋₆ alkyl and C₃₋₆ cycloalkyl are optionally substituted with one, two, or three substituents selected from halogen, —CN, C₁₋₆ alkyl, C₁₋₆ haloalkyl, C₃₋₆ cycloalkyl, —O(C₁₋₆ alkyl), and —O(C₁₋₆ haloalkyl); and
R⁷ is selected from C₁₋₆ alkyl and C₃₋₆ cycloalkyl, each of which is optionally substituted with one, two, or three substituents selected from halogen, —CN, C₁₋₆ alkyl, C₁₋₆ haloalkyl, —O(C₁₋₆ alkyl), and —O(C₁₋₆ haloalkyl).

56. The modified serine residue of claim 55, wherein the polypeptide exhibits reduced signaling output relative to the signaling output of the polypeptide prior to modification of the serine residue.

57. The modified serine residue of claim 56, wherein the reduced signaling output is evidenced by a reduction of cell growth or division of a tumor cell expressing the polypeptide.

58. The modified serine residue of claim 55, wherein the modified serine residue is formed by contacting an unmodified serine residue in the polypeptide with a precursor compound, wherein the precursor compound comprises a staying group and a leaving group, and wherein said contacting results in release of the leaving group and formation of said modified serine residue.

59. The modified serine residue of claim 58, wherein the leaving group is selected from 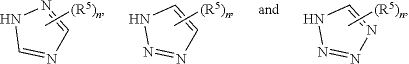
or a salt or tautomer thereof, wherein R⁵ is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and n is 0, 1, or 2.

60. The modified serine residue of claim 58, wherein the leaving group is 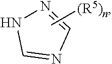
or a salt or tautomer thereof, wherein R is independently selected at each occurrence from halogen, —CN, C₁₋₆ alkyl, and C₃₋₆ cycloalkyl, wherein C₁₋₆ alkyl and C₃₋₆ cycloalkyl are optionally substituted with one, two, or three substituents selected from halogen, —CN, C₁₋₆ alkyl, —O(C₁₋₆ alkyl), and —O(C₁₋₆ haloalkyl); and n is 0, 1, or 2.

61. The modified serine residue of claim 55, wherein the serine residue is an inactive serine.

62. The modified serine residue of claim 55, wherein the non-Ras polypeptide is a kinase.
